(19) United States
(12) Patent Application Publication
(10) Pub. No.: US 2005/0147593 A1
(43) Pub. Date: Jul. 7, 2005

(54) EphA2, EphA4 and LMW-PTP and methods of treatment of hyperproliferative cell disorders

(75) Inventor: Michael S. Kinch, Laytonsville, MD (US)

Correspondence Address: JONES DAY, 222 EAST 41ST ST, NEW YORK, NY, 10017, US

(73) Assignee: MedImmune, Inc.

(21) Appl. No.: 11/004,795

(22) Filed: Dec. 3, 2004

Related U.S. Application Data
(60) Provisional application No. 60/527,154, filed on Dec. 4, 2003.

(30) Foreign Application Priority Data
May 22, 2003 (WO) PCT/US03/16269

(51) Int. Cl. A61K 48/00; A61K 39/395; A61K 9/127

(52) U.S. Cl. 424/93.2; 424/178.1; 424/450; 514/44

(57) ABSTRACT

The present invention relates to methods and compositions designed for treatment, management, or prevention of a hyperproliferative cell disease, particular cancer. The methods of the invention comprise the administration of an effective amount of a composition that targets cells expressing low molecular weight protein tyrosine kinase (“LMW-PTP”) in particular using moieties that bind an Eph family receptor tyrosine kinase, such as EphA2 or EphA4, and inhibits or reduces LMW-PTP expression and/or activity. In one embodiment, the method of the invention comprises administering to a subject a composition comprising an EphA2 or EphA4 targeting moiety attached to a delivery vehicle, and one or more agents that inhibit LMW-PTP expression and/or activity operatively associated with the delivery vehicle. In another embodiment, the method of the invention comprises administering to a subject a composition comprising a nucleic acid comprising a nucleotide sequence encoding an EphA2 or EphA4 targeting moiety and an agent that inhibits or reduces LMW-PTP expression and/or activity. In yet another embodiment, the method of the invention comprises administering to a subject a composition comprising an EphA2 or EphA4 targeting moiety and a nucleic acid comprising a nucleotide sequence encoding an agent that inhibits or reduces LMW-PTP expression and/or activity, where the nucleic acid is operatively associated with the delivery vehicle. Pharmaceutical compositions are also provided by the present invention.

This application claims the benefit of U.S. Provisional Application Ser. No. 60,527,154, filed Dec. 4, 2003, which is incorporated by reference herein in its enitrety. This application further incorporates by reference in their entireties U.S. Provisional Application Ser. No. 60/382,988, entitled “Low Molecular Weight Protein Tyrosine Phosphatase (LMW-PTP) As a Diagnostic and Therapeutic Target,” filed May 23, 2002, and International Patent Application No. PCT/US03/16269, entitled “Low Molecular Weight Protein Tyrosine Phosphatase (LMW-PTP) As a Diagnostic and Therapeutic Target,” filed May 22, 2003.

### 1. FIELD OF THE INVENTION

The present invention relates to methods and compositions designed for treatment, management, or prevention of a hyperproliferative cell disease, particularly cancer. The methods of the invention comprise the administration of an effective amount of a composition that targets cells expressing low molecular weight protein tyrosine kinase (“LMW-PTP”), in particular, using moieties that bind an Eph family receptor tyrosine kinase, such as EphA2 or EphA4, and inhibits or reduces or reduces LMW-PTP expression and/or activity. In one embodiment, the methods of the invention comprise administering to a subject a composition comprising an EphA2 or EphA4 targeting moiety and one or more agents that inhibit or reduce LMW-PTP expression and/or activity. In another embodiment, the methods of the invention comprise administering to a subject a composition comprising an EphA2 or EphA4 targeting moiety associated with a delivery vehicle, and one or more agents that inhibit LMW-PTP expression and/or activity operatively associated with the delivery vehicle. In another embodiment, the methods of the invention comprise administering to a subject a composition comprising a nucleic acid comprising a nucleotide sequence encoding an EphA2 or EphA4 targeting moiety and an agent that inhibits or reduces or LMW-PTP expression and/or activity. In yet another embodiment, the method of the invention comprises administering to a subject a composition comprising an EphA2 or EphA4 targeting moiety and a nucleic acid comprising a nucleotide sequence encoding an agent that inhibits or reduces or LMW-PTP expression and/or activity. In yet another embodiment, the methods of the invention comprise administering to a subject a composition comprising an EphA2 or EphA4 targeting moiety and a nucleic acid comprising a nucleotide sequence encoding an agent that inhibits or reduces or reduces LMW-PTP expression and/or activity, where the nucleic acid is operatively associated with the delivery vehicle. Pharmaceutical compositions are also provided by the present invention.

### 2. BACKGROUND OF THE INVENTION

2.1. Cancer

A neoplasm, or tumor, is a neoplastic mass resulting from abnormal uncontrolled cell growth which can be benign or malignant. Benign tumors generally remain localized. The term “malignant” generally means that the tumor can invade and destroy neighboring body structures and spread to distant sites to cause death (for review, see Robbins and Angell, 1976, Basic Pathology, 2d Ed., W. B. Saunders Co., Philadelphia, pp. 68-122). Cancer can arise in many sites of the body and behave differently depending upon its origin. Cancerous cells destroy the part of the body in which they originate and then spread to other part(s) of the body where they start new growth and cause more destruction.

More than 1.2 million Americans develop cancer each year. Cancer is the second leading case of death in the United States and, if current trends continue, cancer is expected to be the leading cause of the death by the year 2010. Lung and prostate cancer are the top cancer killers for men in the United States. Lung and breast cancer are the top cancer killers for women in the United States. One in two men in the United States will be diagnosed with cancer at some time during his lifetime. One in three women in the United States will be diagnosed with cancer at some time during her lifetime.

The most life-threatening forms of cancer often arise when a population of tumor cells gains the ability to colonize distant and foreign sites in the body. These metastatic cells survive by overriding restrictions that normally constrain cell colonization into dissimilar tissues. For example, typical mammary epithelial cells will generally not grow or survive if transplanted to the lung, yet lung metastases are a major cause of breast cancer morbidity and mortality. Recent evidence suggests that dissemination of metastatic cells through the body can occur long before clinical presentation of the primary tumor. These micrometastatic cells may remain dormant for many months or years following the detection and removal of the primary tumor. Thus, a better understanding of the mechanisms that allow for the growth and survival of metastatic cells in a foreign microenvironment is critical for the improvement of therapeutics designed to fight metastatic cancer and diagnostics for the early detection and localization of metastases.

2.1.1. Cancer Cell Signaling

Aberrant signal transduction occurs in cancer. Aberrant cell signaling overrides anchorage-dependent constraints on cell growth and survival (Rhim et al., Critical Reviews in Oncogenesis 8: 305, 1997; Patarca, Critical Reviews in Oncogenesis 7: 343, 1996; Malik et al., Biochimica et Biophysica Acta 1287: 73, 1996; Cance et al., Breast Cancer Res Treat 35: 105, 1995). For example, protein tyrosine phosphorylation is understood to initiate powerful signals that govern many different aspects of cell behavior. Tyrosine kinase activity is induced by ECM anchorage and indeed, the expression or function of tyrosine kinases is usually increased in malignant cells (Rhim et al., Critical Reviews in Oncogenesis 8: 305, 1997; Cance et al., Breast Cancer Res Treat 35: 105, 1995; Hunter, Cell 88: 333, 1997). A popular paradigm suggests that a balance between tyrosine kinase and phosphatase activities serves to dictate the cellular levels of protein tyrosine phosphorylation and thereby governs cellular decisions regarding growth, survival and invasiveness. This paradigm generally predicts that tyrosine kinases would be oncogenic whereas tyrosine phosphatases negatively regulate malignant transformation. Although this portioning is generally correct, emerging evidence reveals a more complex interplay between tyrosine kinases and phosphatases. For example, the PTPCAAX tyrosine phosphatase has been recently shown to function as a powerful oncogene. Moreover, the enzymatic activity of Src family kinases is liberated by phosphatase-mediated dephosphorylation of important tyrosine residues. In the latter situation, phosphatases can actually up-regulate protein tyrosine phosphorylation by increasing the enzymatic activity of kinases.

Based on evidence that tyrosine kinase activity is necessary for malignant cell growth, tyrosine kinases have been targeted with new therapeutics (Levitzki et al., Science 267: 1782, 1995; Kondapaka et al., Molecular & Cellular Endocrinology 117: 53, 1996; Fry et al., Current Opinion in BioTechnology 6: 662, 1995). Unfortunately, obstacles associated with specific targeting to tumor cells often limit the application of these drugs. In particular, tyrosine kinase activity is often vital for the function and survival of benign tissues (Levitzki et al., Science 267: 1782, 1995). To minimize collateral toxicity, it is critical to identify and then target tyrosine kinases that are selectively overexpressed in tumor cells.

2.1.2. Cancer Therapy

One barrier to the development of anti-cancer agents has been the assay systems that are used to design and evaluate these drugs. Most conventional cancer therapies target rapidly growing cells. However, cancer cells do not necessarily grow more rapidly but instead survive and grow under conditions that are non-permissive to normal cells (Lawrence and Steeg, 1996, World J. Urol. 14: 124-130). These fundamental differences between the behaviors of normal and malignant cells provide opportunities for therapeutic targeting. The paradigm that micrometastatic tumors have already disseminated throughout the body emphasizes the need to evaluate potential chemotherapeutic drugs in the context of a foreign and three-dimensional microenvironment. Many standard cancer drug assays measure tumor cell growth or survival under typical cell culture conditions (i.e., monolayer growth). However, cell behavior in two-dimensional assays often does not reliably predict tumor cell behavior in vivo.

Currently, cancer therapy may involve surgery, chemotherapy, hormonal therapy and/or radiation treatment to eradicate neoplastic cells in a patient (see, for example, Stockdale, 1998, “Principles of Cancer Patient Management,” in Scientific American: Medicine, vol. 3, Rubenstein and Federman, eds., Chapter 12, Section IV). Recently, cancer therapy may also involve biological therapy or immunotherapy. All of these approaches can pose significant drawbacks for the patient. Surgery, for example, may be contraindicated due to the health of the patient or may be unacceptable to the patient. Additionally, surgery may not completely remove the neoplastic tissue. Radiation therapy is only effective when the neoplastic tissue exhibits a higher sensitivity to radiation than normal tissue, and radiation therapy can also often elicit serious side effects. Hormonal therapy is rarely given as a single agent and, although it can be effective, is often used to prevent or delay recurrence of cancer after other treatments have removed the majority of the cancer cells. Biological therapies/immunotherapies are limited in number and each therapy is generally effective for a very specific type of cancer.

With respect to chemotherapy, there are a variety of chemotherapeutic agents available for treatment of cancer. A significant majority of cancer chemotherapeutics act by inhibiting DNA synthesis, either directly, or indirectly by inhibiting the biosynthesis of the deoxyribonucleotide triphosphate precursors, to prevent DNA replication and concomitant cell division (see, for example, Gilman et al., Goodman and Gilman's: The Pharmacological Basis of Therapeutics, Eighth Ed. (Pergamom Press, New York, 1990)). These agents, which include alkylating agents, such as nitrosourea, anti-metabolites, such as methotrexate and hydroxyurea, and other agents, such as etoposides, campathecins, bleomycin, doxorubicin, daunorubicin, etc., although not necessarily cell cycle specific, kill cells during S phase because of their effect on DNA replication. Other agents, specifically colchicine and the vinca alkaloids, such as vinblastine and vincristine, interfere with microtubule assembly resulting in mitotic arrest. Chemotherapy protocols generally involve administration of a combination of chemotherapeutic agents to increase the efficacy of treatment.

Despite the availability of a variety of chemotherapeutic agents, chemotherapy has many drawbacks (see, for example, Stockdale, 1998, “Principles Of Cancer Patient Management” in Scientific American Medicine, vol. 3, Rubenstein and Federman, eds., ch. 12, sect. 10). Almost all chemotherapeutic agents are toxic, and chemotherapy causes significant, and often dangerous, side effects, including severe nausea, bone marrow depression, immunosuppression, etc. Additionally, even with administration of combinations of chemotherapeutic agents, many tumor cells are resistant or develop resistance to the chemotherapeutic agents. In fact, those cells resistant to the particular chemotherapeutic agents used in the treatment protocol often prove to be resistant to other drugs, even those agents that act by mechanisms different from the mechanisms of action of the drugs used in the specific treatment; this phenomenon is termed pleiotropic drug or multidrug resistance. Thus, because of drug resistance, many cancers prove refractory to standard chemotherapeutic treatment protocols.

There is a significant need for alternative cancer treatments, particularly for treatment of cancer that has proved refractory to standard cancer treatments, such as surgery, radiation therapy, chemotherapy, and hormonal therapy. Further, it is uncommon for cancer to be treated by only one method. Thus, there is a need for development of new therapeutic agents for the treatment of cancer and new, more effective, therapy combinations for the treatment of cancer.

2.2. Asthma

Asthma is a disorder characterized by intermittent airway obstruction. In western countries it affects 15% of the pediatric population and 7.5% of the adult population (Strachan et al., 1994, Arch. Dis. Child 70: 174 178). Most asthma in children and young adults is initiated by IgE mediated allergy (atopy) to inhaled allergens such as house dust mite and cat dander allergens. However, not all asthmatics are atopic, and most atopic individuals do not have asthma. Thus, factors in addition to atopy are necessary to induce the disorder (Fraser et al., eds. (1994) Synopsis of Diseases of the Chest. WB Saunders Company, Philadelphia: 635 53; Djukanovic et al., 1990, Am. Rev. Respir. Dis. 142: 434 457). Asthma is strongly familial, and is due to the interaction between genetic and environmental factors. The genetic factors are thought to be variants of normal genes (“polymorphisms”) which alter their function to predispose to asthma.

Asthma may be identified by recurrent wheeze and intermittent air flow limitation. An asthmatic tendency may be quantified by the measurement of bronchial hyper responsiveness in which an individual's dose response curve to a broncho constrictor such as histamine or methacholine is constructed. The curve is commonly summarized by the dose which results in a 20% fall in air flow (PD20) or the slope of the curve between the initial air flow measurement and the last dose given (slope).

In the atopic response, IgE is produced by B cells in response to allergen stimulation. These antibodies coat mast cells by binding to the high affinity receptor for IgE and initiate a series of cellular events leading to the destabilization of the cell membrane and release of inflammatory mediators. This results in mucosal inflammation, wheezing, coughing, sneezing and nasal blockage.

Atopy can be diagnosed by (i) a positive skin prick test in response to a common allergen; (ii) detecting the presence of specific serum IgE for allergen; or (iii) by detecting elevation of total serum IgE.

2.3. COPD

Chronic obstructive pulmonary disease (COPD) is an umbrella term frequently used to describe two conditions of fixed airways disorders, chronic bronchitis and emphysema. Chronic bronchitis and emphysema are most commonly caused by smoking; approximately 90% of patients with COPD are or were smokers. Although approximately 50% of smokers develop chronic bronchitis, only 15% of smokers develop disabling airflow obstruction. Certain animals, particularly horses, suffer from COPD as well.

The airflow obstruction associated with COPD is progressive, may be accompanied by airway hyperactivity, and may be partially reversible. Non specific airway hyper responsiveness may also play a role in the development of COPD and may be predictive of an accelerated rate of decline in lung function.

COPD is a significant cause of death and disability. It is currently the fourth leading cause of death in the United States and Europe. Treatment guidelines advocate early detection and implementation of smoking cessation programs to help reduce morbidity and mortality due to the disorder. However, early detection and diagnosis has been difficult for a number of reasons. COPD takes years to develop and acute episodes of bronchitis often are not recognized by the general practitioner as early signs of COPD. Many patients exhibit features of more than one disorder (e.g., chronic bronchitis or asthmatic bronchitis) making precise diagnosis a challenge, particularly early in the etiology of the disorder. Also, many patients do not seek medical help until they are experiencing more severe symptoms associated with reduced lung function, such as dyspnea, persistent cough, and sputum production. As a consequence, the vast majority of patients are not diagnosed or treated until they are in a more advanced stage of the disorder.

2.4. IBD

Inflammatory bowel disease (IBD) is an idiopathic and chronic intestinal inflammation. Ulcerative colitis (UC) and Crohn's disease (CD) are the two major types of IBD. See Harrison's Principles of Internal Medicine, pp 1679-1692, 15^{th} Ed., Braunwald et al. ed, McGraw-Hill, 2001. UC is a mucosal disease that usually involves the rectum and extends proximally to involve all or part of the colon. With mild inflammation, the mucosa is erythematous and has a fine granular surface. In more severe UC, the mucosa is hemorrhagic, edematous, and ulcerated. In long-standing disease, inflammatory polyps may be present as a result of epithelial regeneration. Id. at 1681. CD can affect any part of the gastrointestinal tract. Unlike UC, the rectum is often spared in CD. Perirectal fistulas, fissures, abscesses, and anal stenosis are present in one-third of patients with CD. CD may also involve the liver and pancreas. Unlike UC, CD is a transmural process. Aphthous or small superficial ulcerations characterize mild disease. In more active CD, stellate ulcerations fuse longitudinally and transversely to demarcate islands of mucosa that frequently are histologically normal. Active CD is characterized by focal inflammation and formation of fistula tracts, which resolve by fibrosis and structuring of the bowel. The bowel wall thickens and becomes narrowed and fibrotic, leading to chronic, recurrent bowel obstruction. Id. at 1681-1682.

Currently, the mainstay of therapy for mild to moderate UC and CD colitis is sulfasalazine and the other 5-ASA agents. Glucocorticoids, azathioprine, 6-mercaptopurine, methotrexate, cyclosporine, tacrolimus, mycophenolate mofetil, thalidomide, anti-tumor necrosis factor (anti-TNF) antibody (e.g., Inliximab), anti-inflammatory cytokines (e.g., interleukin (IL)-10), surgery, and nutritional therapies are also employed in the treatment and management of IBD currently. Id. at pp 1687-1691.

2.5. Mucin

Mucins are a family of glycoproteins secreted by the epithelial cells including those at the respiratory, gastrointestinal and female reproductive tracts. Mucins are responsible for the viscoelastic properties of mucus (Thornton, et al., 1997, J. Biol. Chem., 272: 9561-9566). Nine mucin genes are known to be expressed in man: MUC 1, MUC 2, MUC 3, MUC 4, MUC 5AC, MUC 5B, MUC 6, MUC 7 and MUC 8 (Bobek et al., 1993, J. Biol. Chem. 268: 20563-9; Dusseyn et al., 1997, J. Biol. Chem. 272: 3168-78; Gendler et al., 1991, Am. Rev. Resp. Dis. 144: S42-S47; Gum et al., 1989, J. Biol. Chem. 264: 6480-6487; Gum et al., 1990, Biochem. Biophys. Res. Comm. 171: 407-415; Lesuffleur et al., 1995, J. Biol. Chem. 270: 13665-13673; Meerzaman et al., 1994, J. Biol. Chem. 269: 12932-12939; Porchet et al., 1991, Biochem. Biophys. Res. Comm. 175: 414-422; Shankar et al., 1994, Biochem. J. 300: 295-298; Toribara et al., 1997, J. Biol. Chem. 272: 16398-403). Many airway disorders such chronic bronchitis, chronic obstructive pulmonary disease, bronchietactis, asthma, cystic fibrosis and bacterial infections are characterized by mucin overproduction (Prescott et al., Eur. Respir. J., 1995, 8: 1333-1338; Kim et al., Eur. Respir. J., 1997, 10: 1438; Steiger et al., 1995, Am. J. Respir. Cell Mol. Biol., 12: 307-314). Mucociliary impairment caused by mucin hypersecretion leads to airway mucus plugging which promotes chronic infection, airflow obstruction and sometimes death. For example, chronic obstructive pulmonary disease (COPD), a disorder characterized by slowly progressive and irreversible airflow limitation is a major cause of death in developed countries. The respiratory degradation consists mainly of decreased luminal diameters due to airway wall thickening and increased mucus caused by goblet cell hyperplasia and hypersecretion. Epidermal growth factor (EGF) is known to upregulate epithelial cell proliferation, and mucin production/secretion (Takeyama et al., 1999, PNAS 96: 3081-6; Burgel et al., 2001, J. Immunol. 167: 5948-54). EGF also causes mucin-secreting cells, such as goblet cells, to proliferate and increase mucin production in airway epithelia (Lee et al., 2000, Am. J. Physiol. Lung Cell. Mol. Physiol. 278: L185-92; Takeyama et al., 2001, Am. J. Respir. Crit. Care. Med. 163: 511-6; Burgel et al., 2000, J. Allergy Clin. Immunol. 106: 705-12). Historically, mucus hypersecretion has been treated in two ways: physical methods to increase clearance and mucolytic agents. Neither approach has yielded significant benefit to the patient or reduced mucus obstruction. Therefore, it would be desirable to have methods for reducing mucin production and treating the disorders associated with mucin hypersecretion.

2.6. Restenosis

Vascular interventions, including angioplasty, stenting, atherectomy and grafting are often complicated by undesirable effects. Exposure to a medical device which is implanted or inserted into the body of a patient can cause the body tissue to exhibit adverse physiological reactions. For instance, the insertion or implantation of certain catheters or stents can lead to the formation of emboli or clots in blood vessels. Other adverse reactions to vascular intervention include endothelial cell proliferation which can lead to hyperplasia, restenosis, ie. the re-occlusion of the artery, occlusion of blood vessels, platelet aggregation, and calcification. Treatment of restenosis often involves a second angioplasty or bypass surgery. In particular, restenosis may be due to endothelial cell injury caused by the vascular intervention in treating a restenosis.

Angioplasty involves insertion of a balloon catheter into an artery at the site of a partially obstructive atherosclerotic lesion. Inflation of the balloon is intended to rupture the intima and dilate the obstruction. About 20 to 30% of obstructions reocclude in just a few days or weeks (Eltchaninoff et al., 1998, J. Am Coll. Cardiol. 32: 980-984). Use of stents reduces the re-occlusion rate, however a significant percentage continues to result in restenosis. The rate of restenosis after angioplasty is dependent upon a number of factors including the length of the plaque. Stenosis rates vary from 10% to 35% depending the risk factors present. Further, repeat angiography one year later reveals an apparently normal lumen in only about 30% of vessels having undergone the procedure.

Restenosis is caused by an accumulation of extracellular matrix containing collagen and proteoglycans in association with smooth muscle cells which is found in both the atheroma and the arterial hyperplastic lesion after balloon injury or clinical angioplasty. Some of the delay in luminal narrowing with respect to smooth muscle cell proliferation may result from the continuing elaboration of matrix materials by neointimal smooth muscle cells. Various mediators may alter matrix synthesis by smooth muscle cells in vivo.

2.7. Neointimal Hyperplasia

Neointimal hyperplasia is the pathological process that underlies graft atherosclerosis, stenosis, and the majority of vascular graft occlusion. Neointimal hyperplasia is commonly seen after various forms of vascular injury and a major component of the vein graft's response to harvest and surgical implantation into high-pressure arterial circulation.

Smooth muscle cells in the middle layer (i.e. media layer) of the vessel wall become activated, divide, proliferate and migrate into the inner layer (i.e. intima layer). The resulting abnormal neointimal cells express pro-inflammatory molecules, including cytokines, chemokines and adhesion molecules that further trigger a cascade of events that lead to occlusive neointimal disease and eventually graft failure.

The proliferation of smooth muscle cells is a critical event in the neointimal hyperplastic response. Using a variety of approaches, studies have clearly demonstrated that blockade of smooth muscle cell proliferation resulted in preservation of normal vessel phenotype and function, causing the reduction of neointimal hyperplasia and graft failure.

Existing treatments for the indications discussed above is inadequate, thus, there exists a need for improved treatments for the above indications.

2.8. EphA2 Receptor Tyrosine Kinase

EphA2, a 130 kD protein, is a member of the largest family of receptor tyrosine kinases (Andres, A. C., Reid, H. H., Zurcher, G., Blaschke, R. J., Albrecht, D., and Ziemiecki, A. (1994), “Expression of Two Novel eph-related Receptor Protein Tyrosine Kinases in Mammary Gland Development and Carcinogenesis,” Oncogene 9, 1461-1467; Lindberg et al., Mol. Cell. Biol. 10: 6316-6324 (1990)). It is expressed primarily in cells of epithelial cell origin such as breast, lung, ovary, colon, etc. This protein, also known as ECK, Myk2, and Sek2, was isolated from an erythropoietin-producing hepatocellular carcinoma cell line (Hirai, H., Maru, Y., Hagiwara, K., Nishida, J., and Takaku, F. (1987), “A Novel Putative Tyrosine Kinase Receptor Encoded by the Eph Gene,” Science 238, 1717-1720). Due to multiple names and a growing family of different but related Eph proteins, a nomenclature committee met to officially name the proteins (Eph Nomenclature Committee (Flanaga, J. G., Gale, N. W., Hunter, T., Pasquale, E. B., and Tessier-Lavgne, M.) (1997), “Unified Nomenclature for Eph Family Receptors and Their Ligands, the Ephrins,” Cell 90, 403-404). The proteins were named either EphA or EphB, depending on whether they bind ligands that are GPI-linked or transmembrane, respectively. EphA proteins bind ephrin-A ligands, whereas EphB proteins bind ephrin-B ligands. The number represents the order in which they were discovered.

Different methods have been used to isolate EphA2. First, hybridization techniques were used to isolate EphA2 from DNA libraries (Lindberg et al., Mol. Cell. Biol. 10: 6316-6324 (1990); Hirai, H., Maru, Y., Hagiwara, K., Nishida, J., and Takaku, F. (1987), “A Novel Putative Tyrosine Kinase Receptor Encoded by the Eph Gene,” Science 238, 1717-1720). Secondly, the polymerase chain reaction (PCR) was employed using primers for the kinase domain (Andres, A. C., Reid, H. H., Zurcher, G., Blaschke, R. J., Albrecht, D., and Ziemiecki, A. (1994), “Expression of Two Novel eph-related Receptor Protein Tyrosine Kinases in Mammary Gland Development and Carcinogenesis,” Oncogene 9, 1461-1467; Gilardi-Hebenstreit, P., Nieto, M. A., Frain, M., Mattei, M. G., Chestier, A., Wilkinson, D. G., and Charnay, P. (1992), “An Eph-related Receptor Protein Tyrosine Kinase Gene Segmentally Expressed in the Developing Mouse Hindbrain,” Oncogene 7, 1499-2506). Next, cDNA expression libraries were probed with antibodies specific for phosphotyrosine (Zhou, R., Copeland, T. D., Kromer, L. F., and Schulz, N. T. (1994), “Isolation and Characterization of Bsk, a Growth Receptor-like Tyrosine Kinase Associated with the Limbic System,” J. Neuro. Res. 37, 129-143). Lastly, monoclonal antibodies were screened against proteins that are tyrosine phosphorylated in oncogenic transforming cells (Zantek, N. D. (1999), Ph.D. Thesis, Purdue University).

EphA2 binds ligands known as ephrinA, with the physiological ligand identified as EphrinA1. Ligand binding induces tyrosine phosphorylation of the Eph protein. EphA2, in particular, is able to bind five different ephrin ligands.

EphA2 has characteristic differences in normal and transformed breast epithelia (Zantek, N. D. (1999), Ph.D. Thesis, Purdue University). In normal breast epithelia, EphA2 is present in low protein levels, it is tyrosine phosphorylated, and, finally, it is localized in the sites of cell-cell adhesion. In transformed breast epithelia, high protein levels of EphA2 exist, it is no longer tyrosine phosphorylated, and it is localized in the membrane ruffles.

EphA2 has been found to have a functional role in cancer. When overexpressed, EphA2 is a powerful oncoprotein (Zelinski, D. P., Zantek, N. D., Stewart, J., Irizarry, A. & Kinch, M. S. (2001) Cancer Res 61, 2301-2306). Overexpression of EphA2 in MCF-10A cells causes malignant transformation. Also, injection of these overexpressing cells into nude mice causes tumors. Interestingly, the EphA2 in cancer cells and in EphA2-overexpressing cells is not tyrosine phosphorylated, whereas EphA2 in nontransformed cells is tyrosine phosphorylated.

LMW-PTP, shown herein to regulate EphA2, has also been shown to interact with another member of the Eph family, EphB1 (Stein, E., Lane, A. A., Cerretti, D. P., Schoecklmann, H. O., Schroff, A. D., Van Etten, R. L., and Daniel, T. O. (1998), “Eph Receptors Discriminate Specific Ligand Oligomers to Determine Alternative Signaling Complexes, Attachment, and Assembly Responses,” Genes & Dev. 12, 667-678).

2.9. EphA4 Receptor Tyrosine Kinase

EphA4 is a receptor tyrosine kinase that is expressed in brain, heart, lung, muscle, kidney, placenta, pancreas (Fox, et al, Oncogene 10: 897, 1995) and melanocytes (Easty, et al., Int. J. Cancer 71: 1061, 1997). EphA4 binds cell membrane-anchored ligands (Ephrins A1, A2, A3, A4, A5, B2, and B3; Pasquale, Curr. Opin. in Cell Biology, 1997, 9: 608; also ligands B61, AL1/RAGS, LERK4, Htk-L, and Elk-L3; Martone, et al., Brain Research 771: 238, 1997), and ligand binding leads to EphA4 autophosphorylation on tyrosine residues (Ellis, et al., Oncogene 12: 1727, 1996). EphA4 tyrosine phosphorylation creates a binding region for proteins with Src Homology 2/3 (SH2/SH3) domains, such as the cytoplasmic tyrosine kinase p59fyn (Ellis, et al., supra; Cheng, et al., Cytokine and Growth Factor Reviews 13: 75, 2002). Activation of EphA4 in Xenopus embryos leads to loss of cadherin-dependent cell adhesion (Winning, et al., Differentiation 70: 46, 2002; Cheng, et al., supra), suggesting a role for EphA4 in tumor angiogenesis; however, the role of EphA4 in cancer progression is unclear. EphA4 appears to be upregulated in breast cancer, esophageal cancer, and pancreatic cancer (Kuang, et al., Nucleic Acids Res. 26: 1116, 1998; Meric, et al, Clinical Cancer Res. 8: 361, 2002; Nemoto, et al., Pathobiology 65: 195, 1997; Logsdon, et al., Cancer Res. 63: 2649, 2003), yet it is downregulated in melanoma tissue (Easty, et al., supra).

2.10. Low Molecular Weight Protein Tyrosine Phosphatase (LMW-PTP)

Protein tyrosine phosphatases (sometimes also referred to phosphotyrosine phosphatases), known as PTPases, catalyze the hydrolysis of phosphomonoesters, specifically, the dephosphorylation of protein phosphotyrosyl residues. There are three major classes of PTPases: dual-specificity PTPases, high molecular weight PTPases and low molecular weight PTPases (Zhang, M., Stauffacher, C., and Van Etten, R. L. (1995), “The Three Dimensional Structure, Chemical Mechanism and Function of the Low Molecular Weight Protein Tyrosine Phosphatase,” Adv. Prot. Phosphatases 9, 1-23). Several different acronyms are used interchangeably for low molecular weight (LMW) PTPase and include LMW-PTP, LMW PTP, LMW-PTPase and LMW PTPase.

LMW-PTPs represent a family of PTPases that includes members isolated from many different organisms. They typically have a relative molecular mass of about 18 kD. Members of the LMW-PTP family found in higher organisms include bovine (Heinrikson, R. L. (1969), “Purification and Characterization of a Low Molecular Weight Acid Phosphatase from Bovine Liver,” J. Biol. Chem. 244, 299-307), Erwinia Burgert, P. and Geider, K. (1997), “Characterization of the ams I Gene Product as a Low Molecular Weight Acid Phosphatase Controlling Exopolysaccharide Synthesis of Erwinia Amylovora,” FEBS Lett. 400, 252-256), budding yeast (Ltp1) (Ostanin, K., Pokalsky, C., Wang, S., and Van Etten, R. L. (1995), “Cloning and Characterization of a Saccharomyces cerevisiae Gene Encoding the Low Molecular Weight Protein-Tyrosine Phosphatase,” J. Biol. Chem. 270, 18491-18499), fission yeast (Stp1) Mondesert, O., Moreno, S., and Russell, P. (1994), “Low Molecular Weight Protein Tyrosine Phosphatases are Highly Conserved Between Fission Yeast and Man,” J. Biol. Chem. 269, 27996-27999), rat ACP1 and ACP2 isozymes (Manao, G., Pazzagli, L., Cirri, P., Caselli, A., Camici, G., Cappugi, G., Saeed, A., and Ramponi, G. (1992), “Rat Liver Low Mᵣ Phosphotyrosine Protein Phosphatase Isoenzymes: Purification and Amino Acid Sequences,” J. Prot. Chem. 11, 333-345), human (HPTP) (Wo, Y.-Y. P., Zhou, M.-M., Stevis, P., Davis, J. P., Zhang, Z.-Y., and Van Etten, R. L. (1992), “Cloning, Expression, and Catalytic Mechanism of the Low Molecular Phosphotyrosyl Protein Phosphatase From Bovine Heart,” Biochemistry 31, 1712-1721; Dissing, J. and Svensmark, O. (1990), “Human Red Cell Acid Phosphatase: Purification and Properties of the A, B, and C Isozymes,” Biochem. Biophys. Acta. 1041, 232-242; Waheed, A., Laidler, P. M., Wo, Y.-Y. P., and Van Etten, R. L. (1988), “Purification and Physiochemical Characterization of a Human Placental Acid Phosphatase Possessing Phosphotyrosyl Protein Phosphatase Activity,” Biochemistry 27, 4265-4273; Boivin, P. and Galand, C. (1986), “The Human Red Cell Acid Phosphatase Is a Phosphotyrosine Protein Phosphatase Which Dephosphorylates the Membrane Protein Band 3,” Biochem. Biophys. Res. Commun. 134, 557-564), and BPTP (Zhang, Z-Y. and Van Etten, R. L. (1990), “Purification and Characterization of a Low-Molecular Weight Acid Phosphatase—A Phosphotyrosyl Protein Phosphatase from Bovine Heart,” Arch. Biochem. Biophys. 282, 39-49; Chemoff, J. and L1, H.-C. (1985), “A Major Phosphotyrosyl-Protein Phosphatase From Bovine Heart is Associated with a Low-Molecular-Weight Acid Phosphatase,” Arch. Biochem. Biophys. 240, 135-145). These proteins, as well as other PTPases, share a common active site sequence motif, Cys-(Xaa)₅-Arg. Some proteins that share a high degree of sequence identity with the higher vertebrate enzymes include the low molecular weight PTPases from Escherichia coli (Stevenson, G. Andrianopopoulos, K. Hobbs, M., and Reeves, P. R. (1996), “Organization of the Escherichia coli K-12 Gene Cluster Responsible for Production of the Extracellular Polysaccharide Colanic Acid,” J. Bact. 178, 4885-4893), Klebsiella (Arakawa, Y., Washarotayankun, R., Nagatsuka, T., Ito, H., Kato, N., and Ohta, M. (1995), “Genomic Organization of the Klebsiella pneumoniae CPS Region Responsible for Serotype K2 Capsular Polysaccharide Synthesis in the Virulent Strain Chedid,” J. Bacteriol. 177, 1788-1796), Synechococcus (Wilbanks, S. M. and Glazer, A. N. (1993), “Rod Structure of a Phycoerythrin II-containing Phycobilisdome. I. Organization and Sequence of the Gene Cluster Encoding the Major Phycobilirotein Rod Components in the Genome of Marine Synechococcus sp. WH8020,” J. Biol. Chem. 268, 1226-1234), and Tritrichomonas foetus (gb U66070).

Some mammalian low molecular weight PTPases exist as isozymes. Within specific species, the amino acid sequence identity between the isozymes is greater than 95%. One such species is human, where the human red cell protein tyrosine phosphatase (HPTP) is expressed. The two forms of this protein, A (fast) and B (slow), differ in their electrophoretic mobility when resolved during starch gel electrophoresis. Except for the variable region, residues 40-73, the isozymes have an identical amino acid sequence.

The human isozymes (A and B) have a high level of amino acid sequence identity when compared to BPTP, 81% and 94%, respectively. The crystal structure of BPTP, the prototype of low molecular weight PTPases, has been solved (Zhang, M., Van Etten, R. L., and Stauffacher, C. V. (1994), “Crystal Structure of Bovine Heart Phosphotyrosyl Phosphatase at 2.2-A Resolution,” Biochemistry 33, 11097-11105). The structure consists of α-helices on both sides of a four-stranded central parallel β-sheet. This structure incorporates a portion of a Rossman fold, the classic nucleotide-binding fold consisting in part of two right-handed βαβ motifs. The crystal structure of HPTP-A and yeast LTP1 have been solved (Wang, S., Stauffacher, C. and Van Etten, R. L. (2000), “Structural and Mechanistic Basis for the Activation of a Low Molecular Weight Protein Tyrosine Phosphatase by Adenine,” Biochemistry 39, 1234-1242; Zhang, M. (1995), Ph.D. Thesis, Purdue University), and resemble BPTP. Low molecular weight PTPases have eight conserved cysteines (all in free thiol form), seven conserved arginines, and two conserved histidines (Davis, J. P., Zhou, M. M., and Van Etten, R. L. (1994), “Kinetic and Site-Directed Mutagenesis Studies of the Cystein Residues of Bovine Low Molecular Weight Phosphotyrosyl Protein Phosphatase,” J. Biol. Chem. 269, 8734-8740).

Tyrosine-phosphorylated proteins and peptides, as well as simpler molecules such as phosphotyrosine and pNPP, are all candidates for substrates of the low molecular weight PTPases.

Natural and synthetic inhibitors of these enzymes also exist. Among the strongest inhibitors of low molecular weight PTPases are the ions vanadate, tungstate, and molybdate.

Citation or discussion of a reference herein shall not be construed as an admission that such is prior art to the present invention.

### 3. SUMMARY OF THE INVENTION

The present invention relates to methods and compositions designed for treatment, management, or prevention of a hyperproliferative cell disease, particularly cancer. The methods of the invention comprise the administration of an effective amount of a composition that targets cells expressing low molecular weight protein tyrosine kinase (“LMW-PTP”), in particular, using moieties that bind an Eph family receptor tyrosine kinase, such as EphA2 or EphA4, and inhibits or reduces or reduces LMW-PTP expression and/or activity.

The present invention provides methods of treating, preventing or managing a hyperproliferative cell disease associated with overexpression of LMW-PTP and/or unphosphorylated EphA2 or EphA4 in a subject in need thereof, said method comprising administering to the subject a therapeutically or prophylactically effective amount of a composition comprising (a) a delivery vehicle conjugated to, contained within, or otherwise associated a moiety that binds EphA2 or EphA4, in a configuration in which the moiety binds EphA2 or EphA4 expressed on a cell; (b) one or more agents that inhibit LMW-PTP expression and/or activity; and (c) a pharmaceutically acceptable carrier. Preferably, the agent that inhibits or reduces or reduces LMW-PTP expression and/or activity is conjugated to, contained within, or otherwise associated with the delivery vehicle, so that the delivery vehicle delivers the agents to cells expressing EphA2 and/or EphA4. In a specific embodiment, the invention provides methods of treating, preventing or managing a hyperproliferative cell disease associated with overexpression of LMW-PTP and/or unphosphorylated EphA2 or EphA4 in a subject in need thereof, said method comprising administering to the subject a therapeutically or prophylactically effective amount of a composition comprising (a) a moiety that binds EphA2 or EphA4; (b) one or more agents that inhibit LMW-PTP expression and/or activity; and (c) a pharmaceutically acceptable carrier. In a preferred embodiment, the moiety and agents are associated such that the agents are targeted to the EphA2 or EphA4 expressing cells.

The present invention also provides compositions for treating, preventing or managing a hyperproliferative cell disease, said composition comprising (a) a delivery vehicle conjugated to, contained within or otherwise associated with a moiety that binds EphA2 or EphA4, in a configuration in which the moiety binds EphA2 or EphA4 expressed on a cell; (b) one or more agents that inhibit LMW-PTP expression and/or activity; and (c) a pharmaceutically acceptable carrier. Preferably, the agent that inhibits or reduces or reduces LMW-PTP expression and/or activity is conjugated to, contained within, or otherwise associated with the delivery vehicle, so that the delivery vehicle delivers the agent specifically to cells expressing EphA2 and/or EphA4. In a specific embodiment, the present invention provides compositions for treating, preventing or managing a hyperproliferative cell disease, said composition comprising (a) a moiety that binds EphA2 or EphA4; (b) one or more agents that inhibit LMW-PTP expression and/or activity; and (c) a pharmaceutically acceptable carrier. In a preferred embodiment, the moiety and agents are associated such that the agents are targeted to the EphA2 or EphA4 expressing cells and agonize EphA2 and/or EphA4 phosphorylation in combination with an LMW-PTP inhibitor or an agent that reduces LMW-PTP expression and/or activity.

In some embodiments, the delivery vehicle is a viral vector, a polycation vector, a peptide vector, a liposome or a hybrid vector.

In some embodiments, the moiety that binds EphA2 is an anti-EphA2 antibody or an EphA2 binding fragment thereof, particularly an antibody or a fragment thereof that binds EphA2 epitopes exposed on cancer cells, or an EphA2 ligand, such as Ephrin-A1, or an EphA2 binding fragment thereof (see, e.g., Table 1). In a specific embodiment, the moiety that binds EphA2 in accordance with the present invention is Ephrin-A1 Fc. In some embodiments, the moiety that binds EphA4 is an anti-EphA4 antibody or an EphA4-binding fragment thereof, particularly an antibody or a fragment thereof that binds EphA4 epitopes exposed on cancer cells (see, e.g., Table 1), or an EphA4 ligand, such as Ephrins A1, A2, A3, A4, A5, B2, and B3; B61, AL1/RAGS, LERK4, Htk-L, and Elk-L3, or an EphA4-binding fragment thereof. In a specific embodiment, the moiety that binds EphA4 in accordance with the present invention is Ephrin-A1 Fc.

In some embodiments, the moiety that binds EphA4 is an anti-EphA4 antibody or an EphA4 binding fragment thereof, particularly an antibody or a fragment thereof that binds EphA2 epitopes exposed on cancer cells, or an EphA4 ligand, such as Ephrin-A1, or an EphA4 binding fragment thereof. In a specific embodiment, the moiety that binds EphA4 in accordance with the present invention is Ephrin-A1 Fc. In some embodiments, the moiety that binds EphA4 is an anti-EphA4 antibody or an EphA4-binding fragment thereof, particularly an antibody or a fragment thereof that binds EphA4 epitopes exposed on cancer cells, or an EphA4 ligand, such as Ephrins A1, A2, A3, A4, A5, B2, and B3; B61, AL1/RAGS, LERK4, Htk-L, and Elk-L3, or an EphA4-binding fragment thereof (Pasquale, Curr. Opin. in Cell Biology, 1997, 9: 608 and Martone, et al., Brain Research 771: 238, 1997). In a specific embodiment, the moiety that binds EphA4 in accordance with the present invention is Ephrin-A1 Fc. In another specific embodiment, the moiety that binds EphA4 is an anti-EphA4 antibody, such as EA44 EA44, an anti-EphA4 scFV antibody which is disclosed in U.S. Non-Provisional application Ser. No. 10/863,729, filed Jun. 7, 2004 and is incorporated by reference herein in its entirety. Cells that express the anti-EphA4 scFv EA44 have been deposited with the American Type Culture Collection (P.O. Box 1549, Manassas, Va. 20108) on Jun. 4, 2004 under the provisions of the Budapest Treaty on the International Recognition of the Deposit of Microorganisms for the Purposes of Patent Procedures, and assigned accession number PTA-6044.

In some embodiments, the agent that inhibits or reduces or reduces LMW-PTP expression and/or activity is an anti-LMW-PTP antibody or a fragment thereof (e.g., an intrabody or a BiTE molecule), a small phosphatase inhibitor, a RNA interference (RNAi) molecule, an antisense oligonucleotide, a ribozyme or an aptamer. In a specific embodiment, the agent that inhibits or reduces or reduces LMW-PTP expression or activity in accordance with the present invention is a nucleic acid molecule comprising a nucleotide sequence encoding an agent that inhibits or reduces or reduces LMW-PTP expression and/or activity. In a specific embodiment, the nucleic acid molecule further comprises a nucleotide sequence that inhibits or reduces or reduces EphA2 or EphA4 expression and/or activity.

In some embodiments, the compositions of the invention further comprise an agent that inhibits or reduces or reduces EphA2 expression or function. In particular, such an agent can be, but is not limited to, an EphA2 agonistic molecule (preferably a peptide, an anti-EphA2 antibody, or an EphA2 binding fragment thereof), a polypeptide (preferably an antibody or a fragment thereof) that preferentially binds EphA2 epitopes exposed on cancer cells, a cancer cell phenotype inhibiting polypeptide (preferably an antibody or a fragment thereof), a polypeptide (preferably an antibody or a fragment thereof) that binds to EphA2 with low K_{off} rate, an antisense oligonucleotide, a ribozyme, a RNA interference (RNAi) molecule or an aptamer.

In some embodiments, the compositions of the invention further comprise an agent that inhibits or reduces or reduces EphA4 expression or function. In particular, such an agent can be, but is not limited to, an EphA4 agonistic molecule (preferably a peptide, an anti-EphA4 antibody (e.g., EA44), or an EphA4 binding fragment thereof), a polypeptide (preferably an antibody or a fragment thereof) that preferentially binds EphA4 epitopes exposed on cancer cells, a cancer cell phenotype inhibiting polypeptide (preferably an antibody or a fragment thereof), a polypeptide (preferably an antibody or a fragment thereof) that binds to EphA4 with low K_{off} rate, an antisense oligonucleotide, a ribozyme, a RNA interference (RNAi) molecule or an aptamer.

In some other embodiments, the compositions of the invention further comprise an agent that stimulates an immune response against the cells associated with the hyperproliferative cell disease to be treated or prevented in the subject. In a specific embodiment, an agent that stimulates an immune response against a hyperproliferative cell is a LMW-PTP, EphA2 or EphA4 vaccine that elicits or mediates an immune response against cells that over express LMW-PTP, EphA2 or EphA4.

In some embodiment, the compositions of the invention are used in combination with one or more hyperproliferative cell disease therapies, such as surgery, radiotherapy, chemotherapy, or other immunotherapies.

The methods and compositions of the invention can be used to treat, prevent or manage a hyperproliferative cell disease, such as cancer. In specific embodiments, the methods and compositions of the invention are used to treat, prevent or manage a metastatic cancer, a cancer that is of an epithelial cell origin, a cancer comprising cells that overexpress EphA2 and/or EphA4 relative to non-cancer cells having the tissue type of said cancer cells, or a cancer of the skin, lung, colon, breast, prostate, bladder, pancreas origin, a renal cell carcinoma or melanoma, a leukemia or a lymphoma.

The method and compositions of the invention can also be used to treat, prevent or manage a non-cancer hyperproliferative disease, e.g., asthma, chronic obstructive pulmonary disease (COPD), psoriasis, lung fibrosis, bronchial hyper responsiveness, seborrheic dermatitis, and cystic fibrosis, inflammatory bowel disease, smooth muscle restenosis, endothelial restenosis, hyperproliferative vascular disease, Behcet's Syndrome, atherosclerosis, or macular degeneration.

3.1. Definitions

As used herein, the term “agonist” refers to any compound including a protein, polypeptide, peptide, antibody, antibody fragment, large molecule, or small molecule (less than 10 kD), that increases the activity, activation or function of another molecule. EphA2 or EphA4 agonists cause increased phosphorylation and degradation of EphA2 or EphA4 protein. EphA2 or EphA4 antibodies that agonize EphA2 or EphA4 may or may not also inhibit cancer cell phenotype (e.g., colony formation in soft agar or tubular network formation in a three-dimensional basement membrane or extracellular matrix preparation) and may or may not preferentially bind an EphA2 or EphA4 epitope that is exposed in a cancer cell relative to a non-cancer cell and may or may not have a low K_{off} rate.

The term “antibodies or fragments thereof that immunospecifically bind to EphA2 or EphA4” as used herein refers to antibodies or fragments thereof that specifically bind to an EphA2 or EphA4 polypeptide or a fragment of an EphA2 or EphA4 polypeptide and do not specifically bind to other non-EphA2 or non-EphA4 polypeptides. Preferably, antibodies or fragments that immunospecifically bind to an EphA2 or EphA4 polypeptide or fragment thereof do not non-specifically cross-react with other antigens (e.g., binding cannot be competed away with a non-EphA2 or non-EphA4 protein, e.g., BSA, in an appropriate immunoassay). Antibodies or fragments that immunospecifically bind to an EphA2 or EphA4 polypeptide can be identified, for example, by immunoassays or other techniques known to those of skill in the art. EphA2 antibodies (e.g., EA2 and EA4) are disclosed, for example, in U.S. Nonprovisional application Ser. No. 10/436,782, filed May 12, 2003 entitled “EphA2 Monoclonal Antibodies and Methods of Use Thereof,” and U.S. Nonprovisional application Ser. No. 10/436,783, filed May 12, 2003 entitled “EphA2 Agonistic Monoclonal Antibodies and Methods of Use Thereof,” each of which is incorporated by reference herein in its entirety. EphA4 antibodies (e.g., EA44) are disclosed, for example, in U.S. Nonprovisional application Ser. No. 10/863,729, filed Jun. 7, 2004 entitled “Use of EphA4 and Modulators of EphA4 For Diagnosis, Treatment and Prevention of Cancer,” which is incorporated by reference herein in its entirety. Antibodies of the invention include, but are not limited to, synthetic antibodies, monoclonal antibodies, recombinantly produced antibodies, intrabodies, multispecific antibodies (including bi-specific antibodies), human antibodies, humanized antibodies, chimeric antibodies, synthetic antibodies, single-chain Fvs (scFv) (including bi-specific scFvs), single chain antibodies Fab fragments, F(ab′) fragments, disulfide-linked Fvs (sdFv), and anti-idiotypic (anti-Id) antibodies, and epitope-binding fragments of any of the above. In particular, antibodies of the present invention include immunoglobulin molecules and immunologically active portions of immunoglobulin molecules, i.e., molecules that contain an antigen binding site that immunospecifically binds to an EphA2 or EphA4 antigen (e.g., one or more complementarity determining regions (CDRs) of an anti-EphA2 or anti-EphA4 antibody). Preferably agonistic antibodies or fragments thereof that immunospecifically bind to an EphA2 or EphA4 polypeptide or fragment thereof preferentially agonize EphA2 or EphA4 and do not significantly agonize other activities.

The antibodies used in the methods of the present invention may be monospecific, bispecific, trispecific or of greater multispecificity. Multispecific antibodies may immunospecifically bind to different epitopes of an EphA2 or EphA4 polypeptide or may immunospecifically bind to both an EphA2 or EphA4 polypeptide as well a heterologous epitope, such as a heterologous polypeptide or solid support material. See, e.g., International Publication Nos. WO 93/17715, WO 92/08802, WO 91/00360, and WO 92/05793; Tutt, et al., 1991, J. Immunol. 147: 60-69; U.S. Pat. Nos. 4,474,893, 4,714,681, 4,925,648, 5,573,920, and 5,601,819; and Kostelny et al., 1992, J. Immunol. 148: 1547-1553.

As used herein, the term “cancer” refers to a disease involving cells that have the potential to metastasize to distal sites and exhibit phenotypic traits that differ from those of non-cancer cells, for example, formation of colonies in a three-dimensional substrate such as soft agar or the formation of tubular networks or weblike matrices in a three-dimensional basement membrane or extracellular matrix preparation, such as MATRIGEL™. Non-cancer cells do not form colonies in soft agar and form distinct sphere-like structures in three-dimensional basement membrane or extracellular matrix preparations. Cancer cells acquire a characteristic set of functional capabilities during their development, albeit through various mechanisms. Such capabilities include evading apoptosis, self-sufficiency in growth signals, insensitivity to anti-growth signals, tissue invasion/metastasis, limitless replicative potential, and sustained angiogenesis. The term “cancer cell” is meant to encompass both pre-malignant and malignant cancer cells.

As used herein, the phrase “cancer cell phenotype inhibiting” refers to the ability of a compound to prevent or reduce cancer cell colony formation in soft agar or tubular network formation in a three-dimensional basement membrane or extracellular matrix preparation or any other method that detects a reduction in a cancer cell phenotype, for example, assays that detect an increase in contact inhibition of cell proliferation (e.g., reduction of colony formation in a monolayer cell culture). Cancer cell phenotype inhibiting compounds may also cause a reduction or elimination of colonies when added to established colonies of cancer cells in soft agar or the extent of tubular network formation in a three-dimensional basement membrane or extracellular matrix preparation. EphA2 or EphA4 antibodies that inhibit cancer cell phenotype may or may not also agonize EphA2 or EphA4 and may or may not have a low K_{off} rate.

As used herein, the term “delivery vehicle” refers to a substance that can be used to administer a therapeutic or prophylactic agent to a subject, particular a human. A delivery vehicle may preferentially deliver the therapeutic/prophylactic agent(s) to a particular subset of cells. A delivery vehicle may target certain types of cells, e.g., by virtue of an innate feature of the vehicle or by a moiety conjugated to, contained within (or otherwise associated with such that the moiety and the delivery vehicle stay together sufficiently for the moiety to target the delivery vehicle) the vehicle, which moiety specifically binds a particular subset of cells, e.g., by binding to a cell surface molecule characteristic of the subset of cells to be targeted. A delivery vehicle may also increase the in vivo half-life of the agent to be delivered and/or the bioavailability of the agent to be delivered. Non-limiting examples of a delivery vehicle are a viral vector, a virus-like particle, a polycation vector, a peptide vector, a liposome, and a hybrid vector. In specific embodiments, the delivery vehicle is not directly conjugated to the moiety that binds EphA2 and/or EphA4. In other embodiments, the delivery vehicle is not an antibody that binds EphA2 and/or EphA4.

The term “derivative” in the context of a polypeptide as used herein refers to a polypeptide that comprises an amino acid sequence of a LMW-PTP, EphA2 or EphA4 polypeptide, a fragment of a LMW-PTP, EphA2 or EphA4 polypeptide, an antibody that immunospecifically binds to a LMW-PTP, EphA2 or EphA4 polypeptide, or an antibody fragment that immunospecifically binds to a LMW-PTP, EphA2 or EphA4 polypeptide, that has been altered by the introduction of amino acid residue substitutions, deletions or additions (i.e., mutations). In some embodiments, an antibody derivative or fragment thereof comprises amino acid residue substitutions, deletions or additions in one or more CDRs. The antibody derivative may have substantially the same binding, better binding, or worse binding to its antigen when compared to a non-derivative antibody. In specific embodiments, one, two, three, four, or five amino acid residues of the CDR have been substituted, deleted or added (i.e., mutated). The term “derivative” as used herein also refers to a LMW-PTP, EphA2 or EphA4 polypeptide, a fragment of a LMW-PTP, EphA2 or EphA4 polypeptide, an antibody that immunospecifically binds to a LMW-PTP, EphA2 or EphA4 polypeptide, or an antibody fragment that immunospecifically binds to a LMW-PTP, EphA2 or EphA4 polypeptide which has been modified, i.e, by the covalent attachment of any type of molecule to the polypeptide. For example, but not by way of limitation, a LMW-PTP, EphA2 or EphA4 polypeptide, a fragment of a LMW-PTP, EphA2 or EphA4 polypeptide, an antibody, or antibody fragment may be modified, e.g., by glycosylation, acetylation, pegylation, phosphorylation, amidation, derivatization by known protecting/blocking groups, proteolytic cleavage, linkage to a cellular ligand or other protein, etc. A derivative of a LMW-PTP, EphA2 or EphA4 polypeptide, a fragment of a LMW-PTP, EphA2 or EphA4 polypeptide, an antibody, or antibody fragment may be modified by chemical modifications using techniques known to those of skill in the art, including, but not limited to, specific chemical cleavage, acetylation, formylation, metabolic synthesis of tunicamycin, etc. Further, a derivative of a LMW-PTP, EphA2 or EphA4 polypeptide, a fragment of a LMW-PTP, EphA2 or EphA4 polypeptide, an antibody, or antibody fragment may contain one or more non-classical amino acids. In one embodiment, a polypeptide derivative possesses a similar or identical function as a LMW-PTP, EphA2 or EphA4 polypeptide, a fragment of a LMW-PTP, EphA2 or EphA4 polypeptide, an antibody, or antibody fragment described herein. In another embodiment, a derivative of LMW-PTP, EphA2 or EphA4 polypeptide, a fragment of a LMW-PTP, EphA2 or EphA4 polypeptide, an antibody, or antibody fragment has an altered activity when compared to an unaltered polypeptide. For example, a derivative antibody or fragment thereof can bind to its epitope more tightly or be more resistant to proteolysis.

The term “epitope” as used herein refers to a portion of a LMW-PTP, EphA2 or EphA4 polypeptide having antigenic or immunogenic activity in an animal, preferably in a mammal, and most preferably in a mouse or a human. An epitope having immunogenic activity is a portion of a LMW-PTP, EphA2 or EphA4 polypeptide that elicits an antibody response in an animal. An epitope having antigenic activity is a portion of a LMW-PTP, EphA2 or EphA4 polypeptide to which an antibody immunospecifically binds as determined by any method well known in the art, for example, by immunoassays. Antigenic epitopes need not necessarily be immunogenic.

As used herein, the term “EphA2” or “EphA4” refer to any Eph receptor polypeptide that has been identified and recognized by the Eph Nomenclature Committee (Eph Nomenclature Committee, 1997, Cell 90: 403-404). In a specific embodiment, an EphA2 or EphA4 receptor polypeptide or fragment thereof is from any species. In a preferred embodiment, an EphA2 or EphA4 receptor polypeptide or fragment thereof is human. The nucleotide and/or amino acid sequences of Eph receptor polypeptides can be found in the literature or public databases (e.g., GenBank), or the nucleotide and/or amino acid sequences can be determined using cloning and sequencing techniques known to one of skill in the art. For example, the GenBank Accession Nos. for the nucleotide and amino acid sequences of the human EphA2 are NM_{—}004431.2 and NP_{—}004422.2, respectively. The GenBank Accession Nos. for the nucleotide and amino acid sequences of the human EphA4 are NM_{—}004438.3 and NP_{—}004429.1, respectively.

As used herein, the term “Ephrin” or “Ephrin ligand” refers to any Ephrin ligand that has or will be identified and recognized by the Eph Nomenclature Committee (Eph Nomenclature Committee, 1997, Cell 90: 403-404). Ephrins of the present invention include, but are not limited to, EphrinA1, EphrinA2, EphrinA3, EphrinA4, EphrinA5, EphrinB1, EphrinB2 and EphrinB3. In a specific embodiment, an Ephrin polypeptide, particularly EphrinA1, is from any species. In a preferred embodiment, an Ephrin polypeptide, particularly Ephrin A1, is human. The nucleotide and/or amino acid sequences of Ephrin polypeptides can be found in the literature or public databases (e.g., GenBank), or the nucleotide and/or amino acid sequences can be determined using cloning and sequencing techniques known to one of skill in the art. For example, GenBank Accession Nos. for the nucleotide and amino acid sequences of human Ephrin A1 variant 1 are NM_{—}004428.2 and NP_{—}004419.2, respectively. The GenBank Accession Nos. for the nucleotide and amino acid sequences of human Ephrin A1 variant 2 are NM_{—}182685.1 and NP_{—}872626.1 for variant 2, respectively.

The “fragments” described herein include a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a LMW-PTP, EphA2 or EphA4 polypeptide or an antibody that immunospecifically binds to a LMW-PTP, EphA2 or EphA4 polypeptide. Preferably, antibody fragments are epitope-binding fragments.

As used herein, the term “humanized antibody” refers to forms of non-human (e.g., murine) antibodies that are chimeric antibodies which contain minimal sequence derived from non-human immunoglobulins. For the most part, humanized antibodies are human immunoglobulins (recipient antibody) in which hypervariable region (as defined below) residues of the recipient are replaced by hypervariable region residues from a non-human species (donor antibody) such as mouse, rat, rabbit or non-human primate having the desired specificity, affinity, and capacity. In some instances, Framework Region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin that immunospecifically binds to a LMW-PTP, EphA2 or EphA4 polypeptide, that has been altered by the introduction of amino acid residue substitutions, deletions or additions (i.e., mutations). In some embodiments, a humanized antibody is a derivative. Such a humanized antibody comprises amino acid residue substitutions, deletions or additions in one or more non-human CDRs. The humanized antibody derivative may have substantially the same binding, better binding, or worse binding to antigen when compared to a non-derivative humanized antibody. In specific embodiments, one, two, three, four, or five amino acid residues of the CDR have been substituted, deleted or added (i.e., mutated). For further details in humanizing antibodies, see European Patent Nos. EP 239,400, EP 592,106, and EP 519,596; International Publication Nos. WO 91/09967 and WO 93/17105; U.S. Pat. Nos. 5,225,539, 5,530,101, 5,565,332, 5,585,089, 5,766,886, and 6,407,213; and Padlan, 1991, Molecular Immunology 28(4/5): 489 498; Studnicka et al., 1994, Protein Engineering 7(6): 805 814; Roguska et al., 1994, PNAS 91: 969 973; Tan et al., 2002, J. Immunol. 169: 1119 25; Caldas et al., 2000, Protein Eng. 13: 353 60; Morea et al., 2000, Methods 20: 267 79; Baca et al., 1997, J. Biol. Chem. 272: 10678 84; Roguska et al., 1996, Protein Eng. 9: 895 904; Couto et al., 1995, Cancer Res. 55 (23 Supp): 5973s 5977s; Couto et al., 1995, Cancer Res. 55: 1717 22; Sandhu, 1994, Gene 150: 409 10; Pedersen et al., 1994, J. Mol. Biol. 235: 959 73; Jones et al., 1986, Nature 321: 522-525; Reichmann et al., 1988, Nature 332: 323-329; and Presta, 1992, Curr. Op. Struct. Biol. 2: 593-596.

As used herein, the term “hypervariable region” refers to the amino acid residues of an antibody which are responsible for antigen binding. The hypervariable region comprises amino acid residues from a “Complementarity Determining Region” or “CDR” (i.e., residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196: 901-917). “Framework Region” or “FR” residues are those variable domain residues other than the hypervariable region residues as herein defined.

As used herein, the term “in combination” refers to the use of more than one therapy (e.g., prophylactic and/or therapeutic agents). The use of the term “in combination” does not restrict the order in which prophylactic and/or therapeutic agents are administered to a subject with a hyperproliferative cell disorder, especially cancer. A first therapy (e.g., prophylactic or therapeutic agent) can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., prophylactic or therapeutic agent) to a subject which had, has, or is susceptible to a hyperproliferative cell disorder, especially cancer. The therapies (e.g., prophylactic or therapeutic agents) are administered to a subject in a sequence and within a time interval such that the therapy of the invention can act together with the other agent to provide an increased benefit than if they were administered otherwise. Any additional therapy (e.g., prophylactic or therapeutic agent) can be administered in any order with the other additional therapies (e.g., prophylactic or therapeutic agents).

As used herein, the phrase “low tolerance” refers to a state in which the patient suffers from side effects from treatment so that the patient does not benefit from and/or will not continue therapy because of the adverse effects and/or the harm from the side effects outweighs the benefit of the treatment.

As used herein, the terms “manage,” “managing” and “management” refer to the beneficial effects that a subject derives from administration of a therapy (e.g., prophylactic or therapeutic agent), which does not result in a cure of the disease. In certain embodiments, a subject is administered one or more therapies (e.g., prophylactic or therapeutic agents) to “manage” a disease so as to prevent the progression or worsening of the disease.

As used herein, the phrase “non-responsive/refractory” is used to describe patients treated with one or more currently available therapies (e.g., cancer therapies) such as chemotherapy, radiation therapy, surgery, hormonal therapy and/or biological therapy/immunotherapy, particularly a standard therapeutic regimen for the particular cancer, wherein the therapy is not clinically adequate to treat the patients such that these patients need additional effective therapy, e.g., remain unsusceptible to therapy. The phrase can also describe patients who respond to therapy yet suffer from side effects, relapse, develop resistance, etc. In various embodiments, “non-responsive/refractory” means that at least some significant portion of the cancer cells are not killed or their cell division arrested. The determination of whether the cancer cells are “non-responsive/refractory” can be made either in vivo or in vitro by any method known in the art for assaying the effectiveness of treatment on cancer cells, using the art-accepted meanings of “refractory” in such a context. In various embodiments, a cancer is “non-responsive/refractory” where the number of cancer cells has not been significantly reduced, or has increased during the treatment.

As used herein, the term “potentiate” refers to an improvement in the efficacy of a therapeutic agent at its common or approved dose.

As used herein, the terms “prevent,” “preventing” and “prevention” refer to the prevention of the onset, recurrence, or spread of a disease in a subject resulting from the administration of a therapy (e.g., prophylactic or therapeutic agent).

As used herein, the term “prophylactic agent” refers to any agent that can be used in the prevention of the onset, recurrence or spread of a disease or disorder associated with LMW-PTP, EphA2 or EphA4 overexpression and/or cell hyperproliferative cell disease, particularly cancer.

As used herein, a “prophylactically effective amount” refers to that amount of the prophylactic agent sufficient to result in the prevention of the onset, recurrence or spread of cell hyperproliferative cell disease, preferably, cancer. A prophylactically effective amount may refer to the amount of prophylactic agent sufficient to prevent the onset, recurrence or spread of hyperproliferative cell disease, particularly cancer, including but not limited to those predisposed to hyperproliferative cell disease, for example, those genetically predisposed to cancer or previously exposed to carcinogens. A prophylactically effective amount may also refer to the amount of the prophylactic agent that provides a prophylactic benefit in the prevention of hyperproliferative cell disease. Further, a prophylactically effective amount with respect to a prophylactic agent of the invention means that amount of prophylactic agent alone, or in combination with other agents, that provides a prophylactic benefit in the prevention of hyperproliferative cell disease. Used in connection with an amount of a LMW-PTP, EphA2 or EphA4 targeting moiety or inhibitory agent of the invention, the term can encompass an amount that improves overall prophylaxis or enhances the prophylactic efficacy of or synergies with another prophylactic agent.

A used herein, a “protocol” includes dosing schedules and dosing regimens.

As used herein, the phrase “side effects” encompasses unwanted and adverse effects of a prophylactic or therapeutic agent. Adverse effects are always unwanted, but unwanted effects are not necessarily adverse. An adverse effect from a prophylactic or therapeutic agent might be harmful or uncomfortable or risky. Side effects from chemotherapy include, but are not limited to, gastrointestinal toxicity such as, but not limited to, early and late forming diarrhea and flatulence, nausea, vomiting, anorexia, leukopenia, anemia, neutropenia, asthenia, abdominal cramping, fever, pain, loss of body weight, dehydration, alopecia, dyspnea, insomnia, dizziness, mucositis, xerostomia, and kidney failure, as well as constipation, nerve and muscle effects, temporary or permanent damage to kidneys and bladder, flu-like symptoms, fluid retention, and temporary or permanent infertility. Side effects from radiation therapy include but are not limited to fatigue, dry mouth, and loss of appetite. Side effects from biological therapies/immunotherapies include but are not limited to rashes or swellings at the site of administration, flu-like symptoms such as fever, chills and fatigue, digestive tract problems and allergic reactions. Side effects from hormonal therapies include but are not limited to nausea, fertility problems, depression, loss of appetite, eye problems, headache, and weight fluctuation. Additional undesired effects typically experienced by patients are numerous and known in the art. Many are described in the Physicians' Desk Reference (56th ed., 2002).

As used herein, the terms “single-chain Fv” or “scFv” refer to antibody fragments comprise the VH and VL domains of antibody, wherein these domains are present in a single polypeptide chain. Generally, the Fv polypeptide further comprises a polypeptide linker between the VH and VL domains which enables the scFv to form the desired structure for antigen binding. For a review of scFv see Pluckthun in The Pharmacology of Monoclonal Antibodies, vol. 113, Rosenburg and Moore eds. Springer-Verlag, New York, pp. 269-315 (1994). In specific embodiments, scFvs include bi-specific scFvs and humanized scFvs.

As used herein, the terms “subject” and “patient” are used interchangeably. As used herein, a subject is preferably a mammal such as a non-primate (e.g., cows, pigs, horses, cats, dogs, rats etc.) and a primate (e.g., monkey and human), most preferably a human.

As used herein, the term “targeting moiety” refers to any moiety that, when linked to another agent (such as a delivery vehicle or another compound), enhances the transport of that agent to a target tissue or a subset of cells with a common characteristic, thereby increasing the local concentration of the agent in and around the targeted tissue or subset of cells. For example, a targeting moiety may bind to a molecule on the surface of some or all of the cells in the target tissue or cell subset. In specific embodiments, a targeting moiety binds to EphA2 or EphA4. In a preferred embodiment, a targeting moiety binds to EphA2 on cancer cells (e.g., EphA2 not bound to a ligand) rather than EphA2 on non-cancer cells (e.g., EphA2 bound to a ligand). In another preferred embodiment, a targeting moiety binds to EphA4 on cancer cells (e.g., EphA4 not bound to a ligand) rather than EphA4 on non-cancer cells (e.g., EphA4 bound to a ligand). In a specific embodiment, a targeting moiety of the invention is not directly conjugated to a therapeutic or prophylactic agent.

As used herein, the terms “treat,” “treating” and “treatment” refer to the eradication, reduction or amelioration of symptoms of a disease or disorder, particularly, the eradication, removal, modification, or control of primary, regional, or metastatic cancer tissue that results from the administration of one or more therapeutic agents. In certain embodiments, such terms refer to the minimizing or delaying the spread of cancer resulting from the administration of one or more therapies (e.g., prophylactic or therapeutic agents) to a subject with such a disease.

As used herein, the term “therapeutic agent” refers to any agent that can be used in the prevention, treatment, or management of a disease or disorder associated with overexpression of LMW-PTP, EphA2 and/or EphA4 and/or cell hyperproliferative cell diseases or disorders, particularly, cancer.

As used herein, a “therapeutically effective amount” refers to that amount of the therapeutic agent sufficient to treat or manage a disease or disorder associated with EphA2 or EphA4 overexpression, LMW-PTP overexpression, and/or cell hyperproliferative cell disease and, preferably, the amount sufficient to destroy, modify, control or remove primary, regional or metastatic cancer tissue. A therapeutically effective amount may refer to the amount of therapeutic agent sufficient to delay or minimize the onset of the hyperproliferative cell disease, e.g., delay or minimize the spread of cancer. A therapeutically effective amount may also refer to the amount of the therapeutic agent that provides a therapeutic benefit in the treatment or management of cancer. Further, a therapeutically effective amount with respect to a therapeutic agent of the invention means that amount of therapeutic agent alone, or in combination with other therapies, that provides a therapeutic benefit in the treatment or management of hyperproliferative cell disease or cancer. Used in connection with an amount of a LMW-PTP, EphA2 or EphA4 antibody of the invention, the term can encompass an amount that improves overall therapy, reduces or avoids unwanted effects, or enhances the therapeutic efficacy of or synergies with another therapeutic agent.

As used herein, the term “therapy” refers to any protocol, method and/or agent that can be used in the prevention, treatment, management or amelioration of a hyperproliferative disorder. In certain embodiments, the terms “therapies” and “therapy” refer to a biological therapy, supportive therapy, and/or other therapies useful in treatment, management, prevention, or amelioration of a hyperproliferative disorder or one or more symptoms thereof known to one of skill in the art such as medical personnel.

### 4. DESCRIPTION OF THE FIGURES

FIG. 1 shows a schematic map of the eukaryotic expression vector pcDNA3, a 5.4 kb mammalian expression vector. Unique restriction sites are indicated. The human protein tyrosine phosphatase (HPTP) gene was cloned into the Hind III/BamH I sites of this vector. Expression of the gene was driven by the CMV promoter.

FIGS. 2A-C show that EphA2 is regulated by an associated phosphatase. (A) Monolayers of MCF-10A human mammary epithelial cells were incubated in the presence or absence (denoted as “C” for control) of 4 mM EGTA for 20 minutes before detergent extraction. The samples were resolved by SDS-PAGE and probed with phosphotyrosine-specific antibodies (PY20 and 4G10; top). The membranes were stripped and reprobed with EphA2 specific antibodies to confirm equal sample loading (below). (B) MCF-10A cells were treated with EGTA, as detailed above, in the presence of absence of NaVO₄ to inhibit phosphatase activity. (C) EphA2 was immunoprecipitated from MDA-MB-231 cells that had been incubated in the presence of the indicated concentrations of NaVO₄ for 10 minutes at 37° C.

FIG. 3 shows that LMW-PTP protein levels are elevated in malignant cell lines. Detergent lysates (lanes 2-7) were harvested from non-transformed (MCF-10Aneo), oncogene transformed (MCF-10AneoST), and tumor derived (MCF-7, SK-BR-3, MDA-MB-435, MDA-MB-231) mammary epithelial cells. The samples were resolved by SDS-PAGE and subjected to Western Blot analysis using LMW-PTP specific antibodies (top). Purified LMW-PTP (lane 1) provided a positive control for western blot analyses. The membranes were then stripped and reprobed with antibodies specific to vinculin to evaluate sample loading (bottom). Note that LMW-PTP is overexpressed in tumor-derived cells despite the relative over-loading of the non-transformed (MCF-10Aneo) samples.

FIGS. 4A-B show that EphA2 and LMW-PTP form a molecular complex in vivo. (A) Complexes of EphA2 were immunoprecipitated from 5×10⁶ MCF-10A or MDA-MB-231 cells, resolved by SDS-PAGE and subjected to Western blot analyses with antibodies specific for LMW-PTP. (B) To confirm complex formation, complexes of LMW-PTP were similarly isolated by immunoprecipitation and probed with EphA2 specific antibodies.

FIG. 5 shows that EphA2 can serve as a substrate for LMW-PTP in vitro. EphA2 was immunoprecipitated from 5×10⁶ MCF-10A cells before incubation with the indicated amounts of LMW-PTP protein for 0-30 minutes at 37° C. The samples were then resolved by SDS-PAGE and subjected to Western blot analysis with phosphotyrosine-specific antibodies. The membranes were stripped and reprobed with EphA2 specific antibodies to confirm equal sample loading.

FIGS. 6A-D show that LMW-PTP dephosphorylates EphA2 in vivo. (A) MCF-10A cells were stably transfected with expression vectors that encode for wild-type LMW-PTP. Detergent lysates were resolved by SDS-PAGE and subjected to Western blot analyses with LMW-PTP antibodies to confirm LMW-PTP overexpression, with purified LMW-PTP providing a positive control. Parallel samples were then probed with antibodies specific for β-catenin as a loading control. (B) EphA2 was immunoprecipitated and Western blot were performed using EphA2 (top) and P-Tyr (bottom)-specific antibodies. (C) The overall levels of phosphotyrosine in control and LMW-PTP-transfected cells were compared using specific antibodies. Note that equal amounts of EphA2 were utilized for these results to overcome differences in endogenous EphA2 expression (in contrast to part B). (D) The protein levels (top) and phosphotyrosine content of EphA2 in MDA-MB-231 cells that had been transfected with a dominant negative form of LMW-PTP (D129A) or a matched vector control were evaluated by Western blot analyses. Note the consistent findings that LMW-PTP activity relates to decreased EphA2 phosphotyrosine content and increased EphA2 protein levels.

FIGS. 7A-B show that LMW-PTP enhances malignant character. (A) To evaluate anchorage-dependent cells growth, 1×10⁵ control or LMW-PTP transfected MCF-10A cells were seeded into monolayer culture and cell numbers were evaluated microscopically at the intervals shown. (B) In parallel studies, the control and LMW-PTP transfected cells were suspended in soft agar. Shown is colony formation (per high powered field) after five days of incubation at 37° C. These results were representative of at least three separate experiments. * Indicates p_{—}0.01.

FIGS. 8A-C show that EphA2 retains enzymatic activity in LMW-PTP transformed cells. Equal amounts of EphA2 were immunoprecipitated from control or LMW-PTP transformed MCF-10A cells and subjected to in vitro kinase assays. (A) Autophosphorylation with ³²P-labeled ATP was evaluated by autoradiography. To confirm equal sample loading, a portion of the immunoprecipitated materials was evaluated by Western blot analyses with (B) EphA2 or (C) phosphotyrosine antibodies. Whereas EphA2 is not tyrosine phosphorylated in LMW-PTP transformed cells, it retains enzymatic activity. Note that equal amounts of EphA2 were utilized for these results to overcome differences in endogenous EphA2 expression (for example, see FIG. 5B).

FIGS. 9A-B shows that malignant transformation by LMW-PTP is related to EphA2 overexpression. MCF-10A cells were treated with EphA2 antisense (AS) oligonucleotides, with inverted antisense (IAS) oligonucleotides or transfection reagents alone providing negative controls. (A) Western blot analysis using EphA2 specific antibodies confirmed that the antisense treatment decreased EphA2 protein levels (top). The membrane was then stripped and reprobed for β-catenin to confirm equal sample loading (bottom). (B) Parallel samples were suspended and incubated in soft agar for 5 days. Shown are the average number colonies per high-powered microscopic field (HPF). Indicates p_{—}0.01.

FIG. 10 shows that LMW-PTP overexpression alters two-dimensional morphology.

FIG. 11 shows LMW-PTP overexpressing cells form foci at high cell density.

FIG. 12 shows LMW-PTP inactivation in transformed cells results in decreased soft agar colonization.

FIG. 13 shows that inactivation of LMW-PTP alters two-dimensional morphology and EphA2 distribution in transformed cells.

FIG. 14 shows EGTA treatment of MDA-MB-231 cells transfected with D129A.

FIG. 15 shows a summary of immunofluorescence findings.

FIGS. 16A-B show co-localization of EphA2 and LMW-PTP transfected MCF-10A cells and co-localization of EphA2 and MDA-MB-231 cells transfected with D129A.

FIG. 17 shows that altered organization of actin cytoskeleton relates to LMW-PTP expression and function.

FIG. 18 shows that altered focal adhesion formation relates to LMW-PTP expression and function.

FIG. 19 shows cytokeratin expression altered by LMW-PTP expression.

FIG. 20 shows vimentin expression altered by LMW-PTP expression.

FIG. 21 shows data relating to tumor development in mice injected with 5×10⁶ cells, implanted subcutaneously for 20 days.

FIG. 22 shows equences of VL and VH of EphA2 antibodies. Amino acid and nucleic acid sequences of Eph099B-208.261 (A) VL (SEQ ID NOs:1 and 9, respectively) and (B) VH (SEQ ID NOs:5 and 13, respectively); Eph099B-233.152 (C) VL (SEQ ID NOs.:17 and 25, respectively) and (D) VH (SEQ ID NOs:21 and 29, respectively). Sequences of the CDRs are indicated.

FIG. 23 shows sequences of VL and VH of EA2 and EA5 antibodies. (A) Amino acid and nucleic acid sequences of EA2 VL (SEQ ID NOs:33 and 41, respectively); (B) amino acid and nucleic acid sequences of EA2 VH (SEQ ID NOs:37 and 45, respectively); (C) amino acid and nucleic acid sequences of EA5 VL; and (D) amino acid and nucleic acid sequences of EA5 VH. Sequences of the CDRs are indicated.

FIG. 24 shows Sequences of the EphA4 scFV clone EA44. The CDR, VH, and VL domains are indicated.

### 5. DETAILED DESCRIPTION OF THE INVENTION

Tyrosine phosphorylation is controlled by cell membrane tyrosine kinases (i.e., enzymes that phosphorylate other proteins or peptides), and increased expression of tyrosine kinases is known to occur in metastatic cancer cells. In addition, increased levels of unphosphorylated EphA2, EphA4, EphB 1 and some other Eph family kinases have been implicated in oncogenesis and, in particular metastasis. Phosphorylation of EphA2 or EphA4 leads to degradation of EphA2 or EphA4, which results in inhibition of oncogenesis, in particular, inhibition of metastasis. The present invention is based, in part, on the inventors' discovery that an enzyme that catalyzes dephosphorylation of EphA2 and EphA4 is a powerful oncoprotein and that this enzyme and EphA2/EphA4 are overexpressed in cancer cells. This enzyme is low molecular weight protein tyrosine phosphatase (LMW-PTP). In particular, the link between EphA2/EphA4 and LMW-PTP expression or activity can be exploited by targeting the cell surface expressing EphA2 and/or EphA4 for delivery of agents that inhibit LMW-PTP expression and/or activity in cells expressing LMW-PTP and EphA2 and/or EphA4.

LMW-PTP is overexpressed in a large number of tumor cells. The Examples in Section 6 demonstrate that the phosphotyrosine content of EphA2 is negatively regulated by LMW-PTP, establishing a role for this phosphatase in oncogenesis. The overexpression of LMW-PTP induces a concomitant increase in EphA2/EphA4 levels and is sufficient to confer malignant transformation upon non-transformed epithelial cells. Cancer or other non-cancer hyperproliferative cell diseases that are associated with increased activation or expression of LMW-PTP (whether or not the cancer cells express EphA2/EphA4) can be treated or prevented by inhibiting the activity of LMW-PTP in accordance with the invention.

Thus, by inhibiting the activity of LMW-PTP, dephosphorylation of EphA2 and/or EphA4 can be slowed or prevented, thereby favorably altering the activity of EphA2 and as a result, preventing or reversing the progression of cancer or other non-cancer hyperproliferative cell diseases.

Treatments that result in an inhibition in the activity of LMW-PTP are therefore expected to be accompanied by a favorable change in the disease state of a cancer patient. Favorable changes in the disease state of a cancer patient include, for example, a reduction in the tumor burden (ie., tumor regression), a slowing of tumor growth, prevention or deferral of disease stage progression and prevention or deferral of metastasis. Favorable changes in the disease state of a patient can be detected using any convenient method including radiography, sonography, biochemical assay, and the like. Moreover, in view of the link between LMW-PTP and EphA2/EphA4 expression, m certain embodiments, EphA2-binding moieties or EphA4-binding moieties can be used to target and deliver LMW-PTP agents that inhibit LMW-PTP expression and/or activity to hyperproliferative cells expressing LMW-PTP, EphA2, and/or EphA4. In certain embodiments, EphA2 or EphA4 targeting moieties may also be inhibitors of EphA2 or EphA4 expression or activity, and when they are used to direct the delivery of LMW-PTP inhibitor, a synergistic effect of inhibiting LMW-PTP, EphA2, and/or EphA4 expression or activity may be observed.

The invention thus provides methods for treating, preventing or managing a hyperproliferative cell disease, particularly cancer, in a subject (preferably an animal, more preferably a mammal, and most preferably a human), wherein the subject is suffering from the hyperproliferative cell disease or is otherwise in need of such treatment, prevention or management. The methods are effective to treat, prevent or manage a disease characterized by cells that overexpress LMW-PTP, EphA2, and/or EphA4, particularly metastatic carcinoma cells of the breast, prostate, colon, lung, bladder, ovary, pancreas and skin (melanoma) that additionally possess overexpressed or functionally altered EphA2 or EphA4 tyrosine kinase receptor (see, e.g., Kinch et al., Clin. Cancer Res., 2003, 9(2): 613-618; Kinch et al., Clin. Exp. Metastasis, 2003, 20(1): 59-68; Walker-Daniels et al., Am J. Pathol., 2003, 162(4): 1037-1042; Zelinski, D. P., Zantek, N. D., Stewart, J., Irizarry, A. & Kinch, M. S. (2001) Cancer Res 61, 2301-2306; Zantek, N. D. (1999), Ph.D. Thesis, Purdue University).

The present invention provides methods of specifically targeting one or more therapeutic or prophylactic agents to cells overexpressing EphA2 or EphA4, thereby making the agents more effective and reduces the chances of adverse side effects. A therapeutic or prophylactic agent that inhibits or reduces or reduces the biological activity of LMW-PTP can be introduced into a subject, either systemically or at the site of a cancer, in an amount effective to inhibit the biological activity of LMW-PTP, e.g., inhibiting dephosphorylation of EphA2, EphA4, EphB 1, or other Eph family tyrosine kinases. Optionally, the agent that inhibits or reduces or reduces LMW-PTP expression or activity can be linked to another drug, preferably a cytotoxic drug, either directly or through a delivery vehicle, thereby possessing the dual activities of inhibiting LMW-PTP and serving as a carrier molecule for the cytotoxic drug. In a specific embodiment, another agent is also delivered to the subject to effect cleavage when a cleavable therapeutic or prophylactic agent is used. In preferred embodiments, the agents that inhibit LMW-PTP expression and/or activity or such agents that are conjugated to other therapeutic or prophylactic agents are delivered to cells that express EphA2/EphA4 and LMW-PTP by a delivery vehicle targeting cells expressing EphA2 and/or EphA4. In specific embodiments, an EphA2-binding moiety or an EphA4-binding moiety is attached to a delivery vehicle so that the delivery vehicle is directed or targeted to cells that express EphA2 and/or EphA4.

In one embodiment, the present invention provides a treatment, prevention or management method comprising co-administration to a subject of a first therapeutic or prophylactic agent that inhibits or reduces or reduces LMW-PTP expression and/or activity, and a second therapeutic or prophylactic agent that inhibits or reduces or reduces EphA2 or EphA4 expression and/or activity. In a specific embodiment, LMW-PTP expression and/or activity is decreased is decreased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, more preferably at least 95%, and most preferably, at least 98%. The therapeutic or prophylactic agent that inhibits or reduces or reduces EphA2 or EphA4 expression and/or activity can be, for example, an antibody, a small molecule, a peptide, a ligand or ligand mimetic, an antisense nucleic acid, an aptamer or a small interfering RNA (siRNA). In a specific embodiment, the second therapeutic or prophylactic agent agonizes EphA2 or EphA4 by binding to an extracellular epitope on the receptor molecule and thereby eliciting EphA2 or EphA4 tyrosine phosphorylation and signaling. In such an embodiment, the EphA2 or EphA4 agonist may also function as the EphA2 or EphA4 targeting moiety. Ligand-mediated activation is characterized by increased EphA2 or EphA4 phosphotyrosine content and is accompanied by decreased EphA2/EphA4 levels and/or activity. A “decreased EphA2/EphA4 activity” refers to a reduction in the activity, number (i.e., protein level) and/or function of EphA2 receptors or EphA4 receptors in cancer cells so as to arrest or reverse cell growth or proliferation, or to initiate or cause killing of the cancer cell. Arrest or reversal of cell growth or proliferation can be evidenced by various phenotypic changes in the cancer cells such as increased differentiation, decreased affinity for ECM proteins, increased cell-cell adhesion, slower growth rate, reduced numbers of EphA2/EphA4 and/or increased localization of EphA2/EphA4, decreased cell migration or invasion, and can be caused either directly or indirectly. In a specific embodiment, EphA2 and/or EphA4 activity is decreased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, more preferably at least 95%, and most preferably, at least 98%. Optionally, the second treatment agent causes EphA2 or EphA4 crosslinking, and/or acceleration in the degradation of EphA2 or EphA4. In another aspect of the invention, the second treatment agent reduces expression of EphA2 or EphA4 in a target cancer or precancerous cell at the DNA/RNA level, for example via the binding of an antisense oligonucleotide (see International Application Nos. PCT/US03/15044 and PCT/US03/15046, each of which is incorporated herein in its entirety) or RNAi. In preferred embodiments, the agents that inhibit LMW-PTP expression and/or activity and/or the second agent that inhibit EphA2 or EphA4 expression and/or activity are delivered to cells that express LMW-PTP, EphA2, and/or EphA4 by a delivery vehicle targeting cells expressing EphA2 or EphA4. In a specific embodiment, EphA2 and/or EphA4 expression and/or activity is decreased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, more preferably at least 95%, and most preferably, at least 98%. In specific embodiments, an EphA2-binding moiety or an EphA4-binding moiety is attached to a delivery vehicle so that the delivery vehicle is directed or targeted to cells that express EphA2 or EphA4. In a specific embodiment, the EphA2-binding moiety or EphA4-binding moiety also inhibits or reduces EphA2 expression and/or activity.

The present invention provides methods of treating, preventing or managing cancer or other non-cancer hyperproliferative cell diseases by inhibiting LMW-PTP expression and/or activity. LMW-PTP can be inhibited either alone or in combination with treatments that inhibit EphA2/EphA4 expression and/or activity. LMW-PTP levels can also serve as a marker in disease detection, or as a surrogate marker to analyze the impact of treatments that target EphA2, EphA4, or other tyrosine kinases associated with the development or progression of cancer.

In preferred embodiments, an EphA2-targeting moiety or an EphA4-targeting moiety is used to deliver one or more agents that inhibit LMW-PTP expression and/or function to hyperproliferative cells expressing LMW-PTP, EphA2, and/or EphA4. In one embodiment, the present invention provides a method of treating, preventing or managing a hyperproliferative cell disease comprising administering to a subject in need thereof a composition comprising an EphA2-targeting moiety or an EphA4-targeting moiety attached to a delivery vehicle, and one or more agents that inhibit LMW-PTP expression and/or activity, wherein the agents are contained within, expressed by, conjugated to, or otherwise associated with the delivery vehicle. In another embodiment, the present invention provides a method of treating, preventing or managing a hyperproliferative cell disease comprising administering to a subject in need thereof a composition comprising a nucleic acid comprising a nucleotide sequence encoding an EphA2-targeting moiety or an EphA4-targeting moiety and one or more agents that inhibit LMW-PTP expression and/or activity. Pharmaceutical compositions and kits are also provided in the present invention.

5.1. Agents that Inhibit LMW-PTP, EphA2 and/or EphA4 Expression or Function

Inhibition in LMW-PTP, EphA2, and/or EphA4 expression and/or activity can be assessed in comparison to LMW-PTP, EphA2, and/or EphA4 expression and/or activity prior to treatment. Typically this is assessed in a laboratory setting using appropriate cell lines (for example, see Section 6, infra). Administration to a patient of a therapeutic or prophylactic agent that causes inhibition of LMW-PTP, EphA2 and/or EphA4 expression and/or activity in a laboratory setting in model systems routinely used for human cancer research (e.g., as disclosed in Section 6) is fully expected to cause inhibition of LMW-PTP, EphA2 and/or EphA4 expression and/or activity in the patent's cells in vivo. It should be understood that the method of the invention is not limited by the way in which, or the extent to which, LMW-PTP, EphA2 and/or EphA4 expression and/or activity is inhibited in the target cells.

Methods for inhibiting LMW-PTP, EphA2 and/or EphA4 expression and/or activity include, but are not limited to, those that act directly on the gene encoding one or more of the LMW-PTP enzymes (such as HPTP-A and HPTP-B), those that inhibit LMW-PTP, EphA2 or EphA4 expression or activity, e.g., agents that agonize EphpA2 or EphA4, agents that lead to increased phosphorylation of EphA2 or EphA4, agents that lead to degradation of EphA2 or EphA4 (specifically, those that bind to epitopes of EphA2 or EphA4 exposed on cancer cells and those agonize EphA2 or EphA4), those that can be used as vaccines, those that act on the mRNA transcript produced by the gene encoding LMW-PTP, EphA2 or EphA4, those that interfere with the translation of the mRNA transcript into the protein, and those that directly impair the activity of the translated protein.

Transcription of a gene can be impeded by delivering to the cell an antisense DNA or RNA molecule, a double stranded RNA molecule. Another way the activity of an enzyme can be inhibited is by interfering with the mRNA transcription product of the gene. For example, a ribozyme (or a DNA vector operably encoding a ribozyme) can be delivered to the cell to cleave the target mRNA. Antisense nucleic acids and double stranded RNAs may also be used to interfere with translation.

Peptides, polypeptides (including antibodies, antibody fragments, fusion proteins), ligands, ligand mimics, peptidomimetic compounds and other small molecules are examples of those that can be used to directly compromise the activity of the translated protein. Any known phosphatase inhibitors can be used to inhibit LMW-PTP expression or activity. Non-limiting examples of phosphatase inhibitors are sodium orthovanadate, and pyrrole compunds (see U.S. Patent Application Publication No. 20030144338). Optionally, these agents can be introduced using a delivery vehicle described in Section 5.3, infra. Alternatively, a proteinaceous intracellular agent that inhibits or reduces the expression and/or activity of LMW-PTP, EphA2 or EphA4 can be delivered as a nucleic acid, for example as RNA, DNA, or analogs or combinations thereof, using conventional methods, wherein the therapeutic polypeptide is encoded by the nucleic acid and operably linked to regulatory elements such that it is expressed in the target mammalian cell.

Preferred therapeutic or prophylactic agents for use in inhibiting LMW-PTP expression and/or activity include, but are not limited to, small molecules, peptides, antisense oligonucleotides, aptamers, substrate mimics (e.g., non-hydrolyzable or substrate trapping inhibitors) and agents that can be used as vaccines to generate antibodies against LMW-PTP. Treatment agents can include antagonists that resemble substrate or that interfere with the binding of LMW-PTP to its substrate, particularly those that interfere with Eph-LMW-PTP interactions, such as EphA2-LMW-PTP interactions or EphA4-LMW-PTP interactions. Small molecules that resemble pyridoxyl phosphate are particularly preferred, such as those that substitute phosphonic acid or sulfonic acid for the phosphate group in pyridoxal phosphate. The active site of LMW-PTP can be targeted, particular Tyr131, Tyr132 and Asp 129. Because the BPTP x-ray crystal structure has been solved, rational drug design can be used to identify or design highly specific inhibitors of LMW-PTP which are expected to be especially useful therapeutically. In one embodiment, an agent that inhibit LMW-PTP, EphA2 or EphA4 activity is an agent that prevents LMW-PTP from binding phosporylated EphA2 or EphA4. In another embodiment, an agent that inhibits or reduces LMW-PTP, EphA2 or EphA4 activity is an agent that prevents LMW-PTP from binding EphA2 or EphA4, regardless whether EphA2 or EphA4 is phosphorylated. In a specific embodiment, an agent that inhibits or reduces LMW-PTP, EphA2, or EphA4 activity is an agent that prevent LMW-PTP from binding to the substrate binding site of EphA2 or EphA4 (i.e., LMW-PTP may bind to a domain on EphA2 or EphA4 other than the substrate binding site).

Preferred therapeutic or prophylactic agents for use in inhibiting EphA2 or EphA4 expression and/or activity include agents that agonize EphA2 or EphA4, agents that lead to increased phosphorylation of EphA2 or EphA4, agents that lead to degradation of EphA2 or EphA4 (specifically, those that bind to epitopes of EphA2 or EphA4 exposed on cancer cells), and agents that can be used as vaccines to generate antibodies against EphA2 or EphA4. Non-limiting examples of such agents are small molecules, Ephrin peptides (particularly Ephrin A1 that binds EphA2 or EphA4), EphA2 or EphA4 binding antibodies and fragments thereof, antisense oligonucleotides, RNA interference (RNAi) molecules and aptamers.

5.1.1. Antibodies

In accordance with the present invention, an anti-EphA2 or anti-EphA4 antibody can be used as an EphA2 or EphA4 targeting moiety, and/or an agent that inhibits EphA2 or EphA4 expression or activity. Antibodies that can inhibit EphA2 or EphA4 expression or activity include, but are not limited to, antibodies (preferably monoclonal antibodies) or fragments thereof that immunospecifically bind to and agonize EphA2 or EphA4 signaling (“EphA2 agonistic antibodies” and “EphA4 agonistic antibodies”); inhibit a cancer cell phenotype, e.g., inhibit colony formation in soft agar or tubular network formation in a three-dimensional basement membrane or extracellular matrix preparation, such as MATRIGEL™ (“cancer cell phenotype inhibiting antibodies”); preferentially bind epitopes on EphA2 or EphA4 that are selectively exposed or increased on cancer cells but not non-cancer cells (“exposed EphA2 epitope antibodies” and “exposed EphA4 epitope antibodies”); and/or bind EphA2 or EphA4 with a K_{off} of less than 3×10⁻³ s⁻¹. In one embodiment, the antibody binds to the extracellular domain of EphA2 or EphA4 and, preferably, also agonizes EphA2 or EphA4, e.g., increases EphA2 or EphA4 phosphorylation and, preferably, causes EphA2 or EphA4 degradation. In another embodiment, the antibody binds to the extracellular domain of EphA2 or EphA4 and, preferably, also inhibits and, even more preferably, reduces the extent of (e.g., by cell killing mechanisms such as necrosis and apoptosis) colony formation in soft agar or tubular network formation in a three-dimensional basement membrane or extracellular matrix preparation. In other embodiments, the antibodies inhibit or reduce a cancer cell phenotype in the presence of another anti-cancer agent, such as a hormonal, biologic, chemotherapeutic or other agent. In another embodiment, the antibody binds to the extracellular domain of EphA2 or EphA4 at an epitope that is exposed in a cancer cell but occluded in a non-cancer cell. In a specific embodiment, the antibody is not EA2 or EA5 (or humanized version thereof). In another specific embodiment, the antibody is not EA44 (or humanized version thereof). In another embodiment, the antibody binds to the extracellular domain of EphA2 or EphA4, preferably with a K_{off} of less than 3×10⁻³ s⁻¹, more preferably less than 1×10⁻³ s⁻¹. In other embodiments, the antibody binds to EphA2 or EphA4 with a K_{off} of less than 5×10⁻³ s⁻¹, less than 10⁻³ s⁻¹, less than 8×10⁻⁴ s⁻¹, less than 5×10⁻⁴ s⁻¹, less than 10⁻⁴ s⁻¹, less than 9×10⁻⁵ s⁻¹, less than 5×10⁻⁵ s⁻¹, less than 10⁻⁵ s⁻¹, less than 5×10⁻⁶ s⁻¹, less than 10⁻⁶ s⁻¹, less than 5×10⁻⁷ s⁻¹, less than 10⁻⁷ s⁻¹, less than 5×10⁻⁸ s⁻¹, less than 10⁻⁸ s⁻¹, less than 5×10⁻⁹ s⁻¹, less than 10⁻⁹ s⁻¹, or less than 10⁻¹⁰ s⁻¹.

In a more preferred embodiment, the antibody is Eph099B-102.147, Eph099B-208.261, Eph099B-210.248, Eph099B-233.152, EA44 or any of the antibodies listed in Table 1. In another embodiment, the antibody binds to an epitope bound by Eph099B-102.147, Eph099B-208.261, Eph099B-210.248, Eph099B-233.152, EA44 or any of the antibodies listed in Table 1 and/or competes for EphA2 or EphA4 binding with Eph099B-102.147, Eph099B-208.261, Eph099B-210.248, Eph099B-233.152, EA44 or any of the antibodies listed in Table 1, e.g. as assayed by ELISA or any other appropriate immunoassay (e.g., ELISA).

In another more preferred embodiment, the antibody is EA2, EA3, EA4, or EA5. In another embodiment, the antibody binds to an epitope bound by EA2, EA3, EA4, or EA5 and/or competes for EphA2 binding with EA2, EA3, EA4, or EA5, e.g. as assayed by ELISA. In other embodiments, the antibody of the invention immunospecifically binds to and agonizes EphA2 signaling and/or preferentially binds an epitope on EphA2 that is selectively exposed or increased on cancer cells but not non-cancer cells and may or may not compete for binding with an EphA2 ligand, e.g., Ephrin A1.

In another more preferred embodiment, the antibody is EA44. In another embodiment, the antibody binds to an epitope bound by EA44 and/or competes for EphA4 binding with EA44, e.g. as assayed by ELISA. In other embodiments, the antibody of the invention immunospecifically binds to and agonizes EphA4 signaling and/or preferentially binds an epitope on EphA4 that is selectively exposed or increased on cancer cells but not non-cancer cells and may or may not compete for binding with an EphA4 ligand, e.g., Ephrin A1, Ephrin A2, Ephrin A3, Ephrin A4, Ephrin A5, Ephrin B2 or Ephrin B3.

In other embodiments, the antibody of the invention immunospecifically binds to and agonizes EphA2 signaling, inhibits a cancer cell phenotype, preferentially binds an epitope on EphA2 that is selectively exposed or increased on cancer cells but not non-cancer cells, and/or has a K_{off} of less than 3×10⁻³ s⁻¹ and may or may not compete for binding with an EphA2 ligand, e.g., Ephrin A1.

In other embodiments, the antibody of the invention immunospecifically binds to and agonizes EphA4 signaling, inhibits a cancer cell phenotype, preferentially binds an epitope on EphA4 that is selectively exposed or increased on cancer cells but not non-cancer cells, and/or has a K_{off} of less than 3×10⁻³ s⁻¹ and may or may not compete for binding with an EphA4 ligand, e.g., Ephrin A1, Ephrin A2, Ephrin A3, Ephrin A4, Ephrin A5, Ephrin B2 or Ephrin B3.

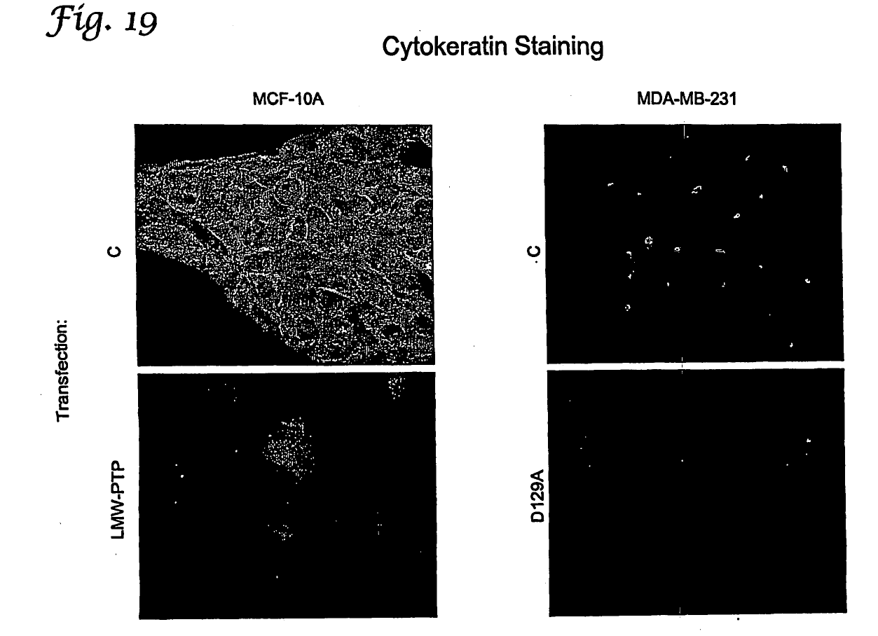

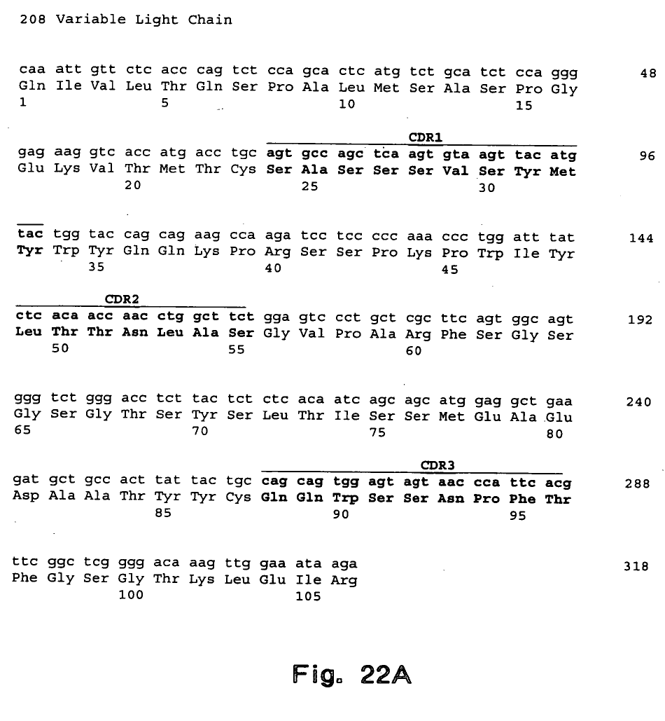

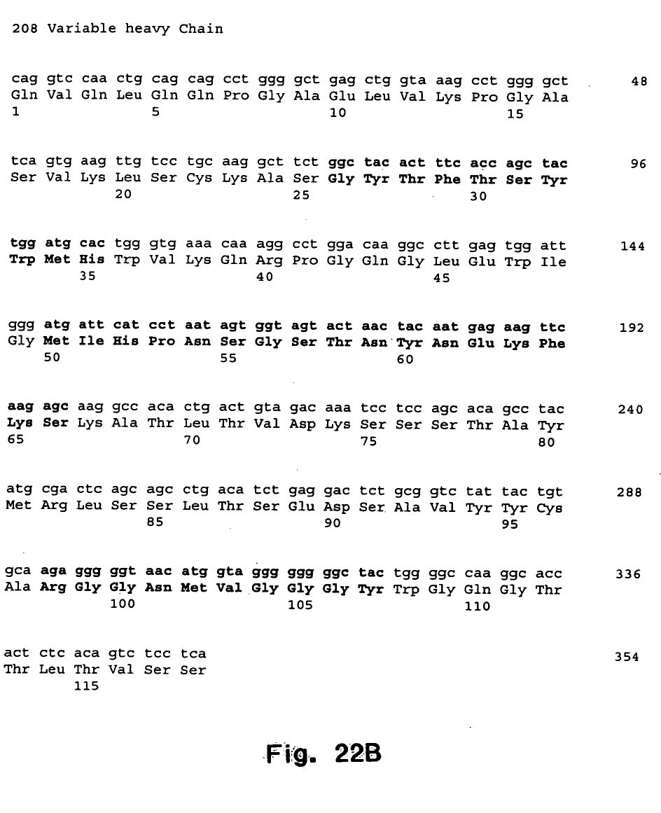

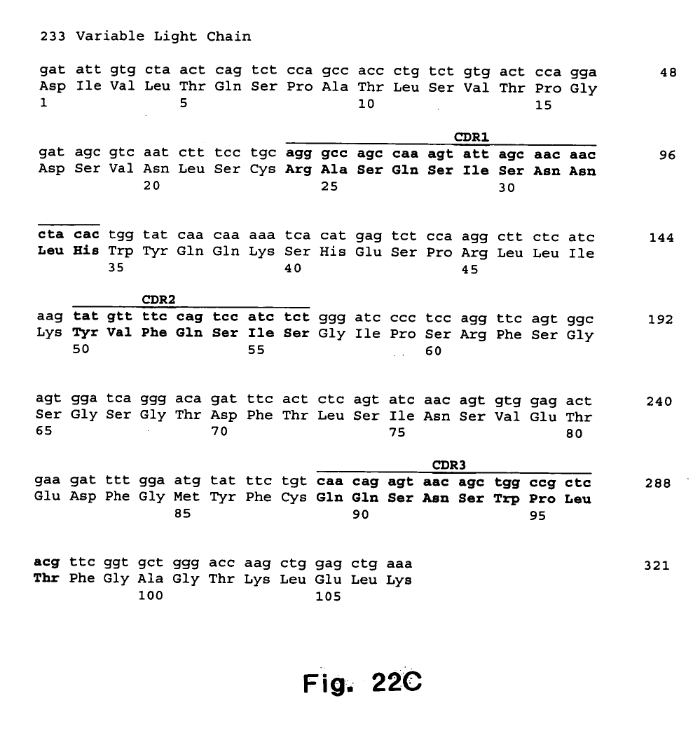

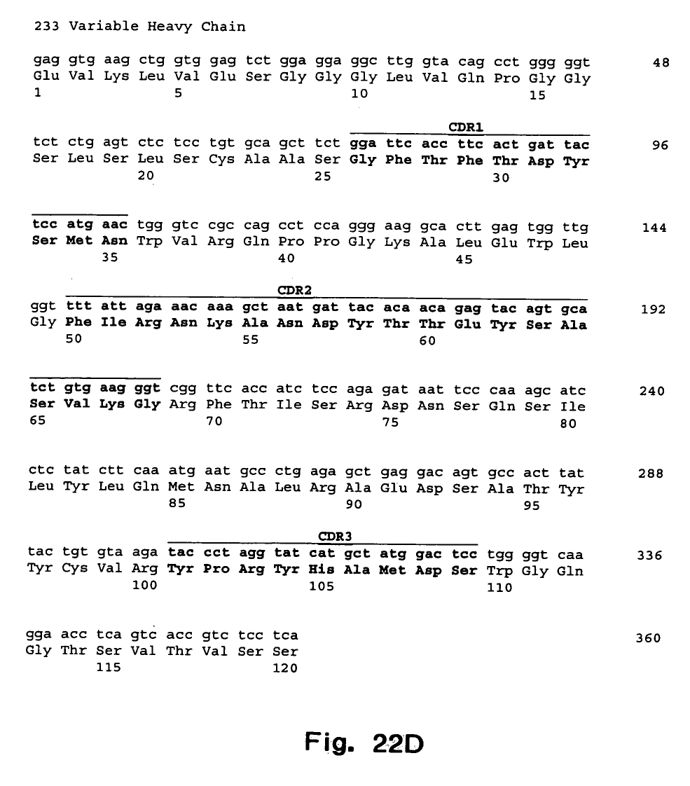

Hybridomas producing Eph099B-102.147, Eph099B-208.261, and Eph099B-210.248 have been deposited with the American Type Culture Collection (ATCC, P.O. Box 1549, Manassas, Va. 20108) on Aug. 7, 2002 under the provisions of the Budapest Treaty on the International Recognition of the Deposit of Microorganisms for the Purposes of Patent Procedures, and assigned accession numbers PTA-4572, PTA-4573, and PTA-4574, respectively, and incorporated by reference. A hybridoma producing Eph099B-233.152 has been deposited with the American Type Culture Collection (ATCC, P.O. Box 1549, Manassas, Va. 20108) on May 12, 2003 under the provisions of the Budapest Treaty on the International Recognition of the Deposit of Microorganisms for the Purposes of Patent Procedures, and assigned accession number PTA-5194, and incorporated by reference. The amino acid and nucleic acid sequences of VL and VH of Eph099B-208.261 and Eph099B-233.152 are shown in FIGS. 22A-19D. The sequences of the Eph099B-208.261 and Eph099B-233.152 CDRs are indicated in Table 1. In a most preferred embodiment, the antibody is human or has been humanized.

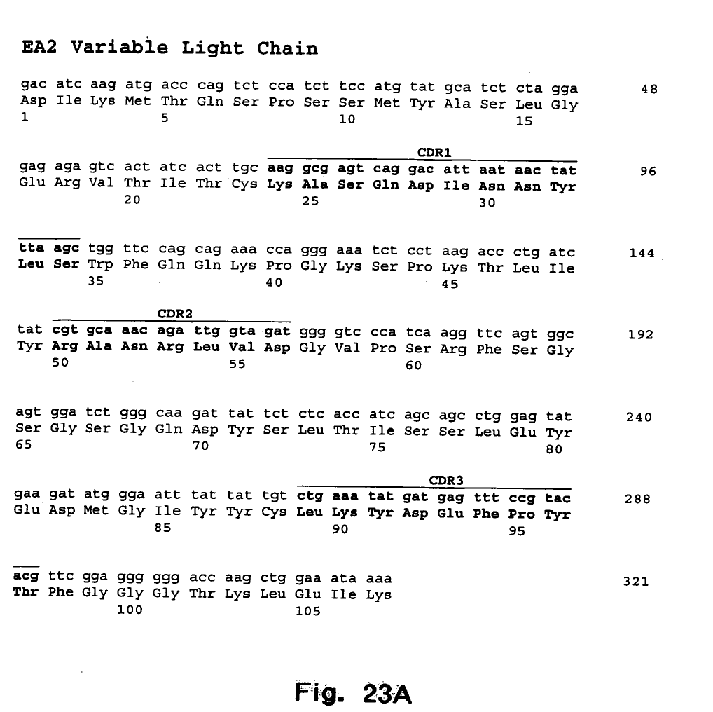

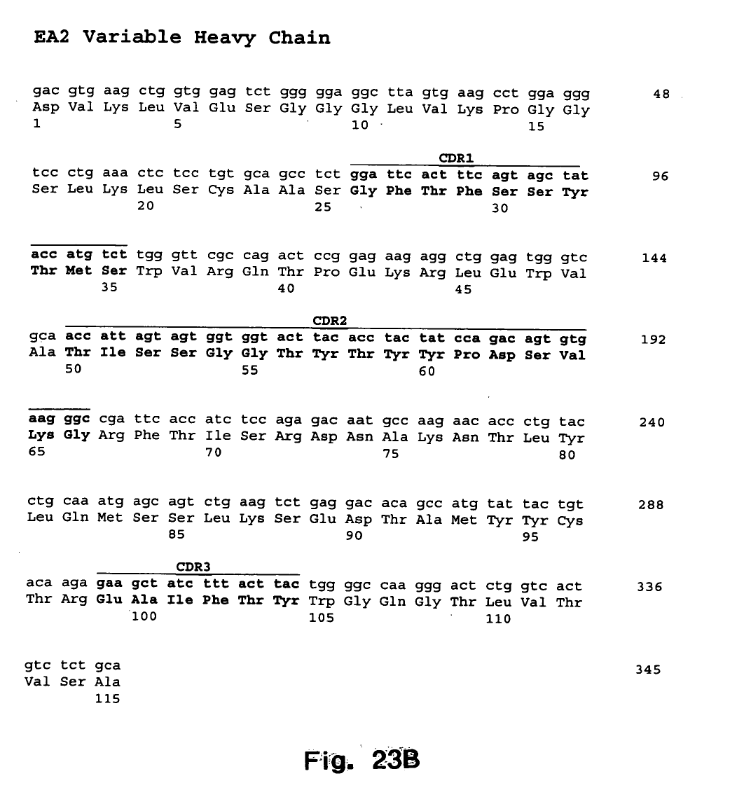

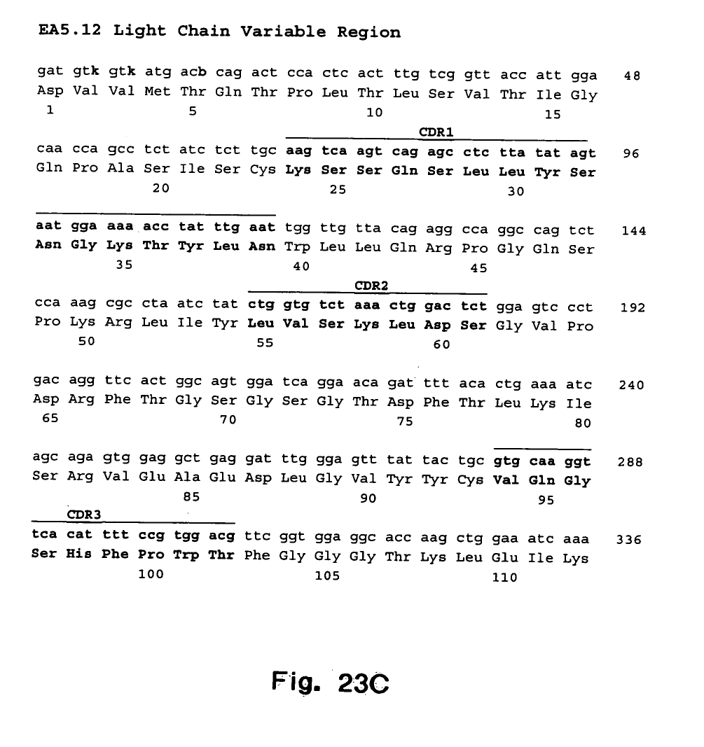

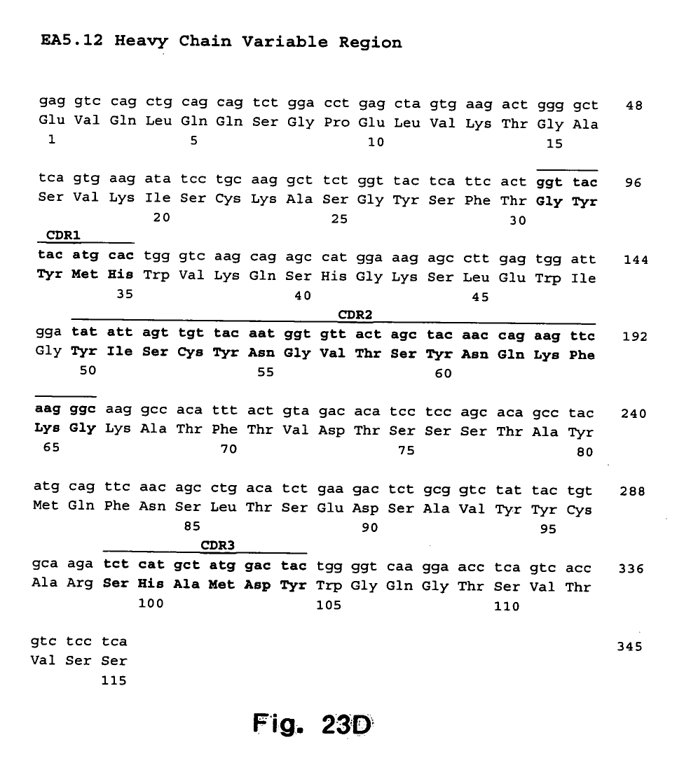

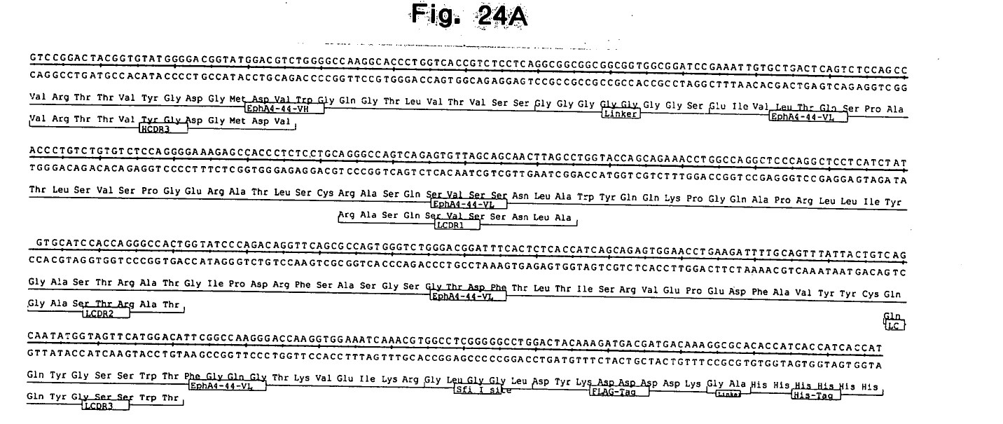

Hybridomas producing antibodies EA2 (strain EA2.31) and EA5 (strain EA5.12) of the invention have been deposited with the American Type Culture Collection (ATCC, P.O. Box 1549, Manassas, Va. 20108) on May 22, 2002 under the provisions of the Budapest Treaty on the International Recognition of the Deposit of Microorganisms for the Purposes of Patent Procedures, and assigned accession numbers PTA-4380 and PTA-4381, respectively and incorporated by reference. The amino acid and nucleic acid sequences of EA2 and EA5 are shown in FIGS. 23A-D. The sequences of the EA2 and EA5 CDRs are indicated in Table 1. In a most preferred embodiment, the antibody is human or has been humanized.

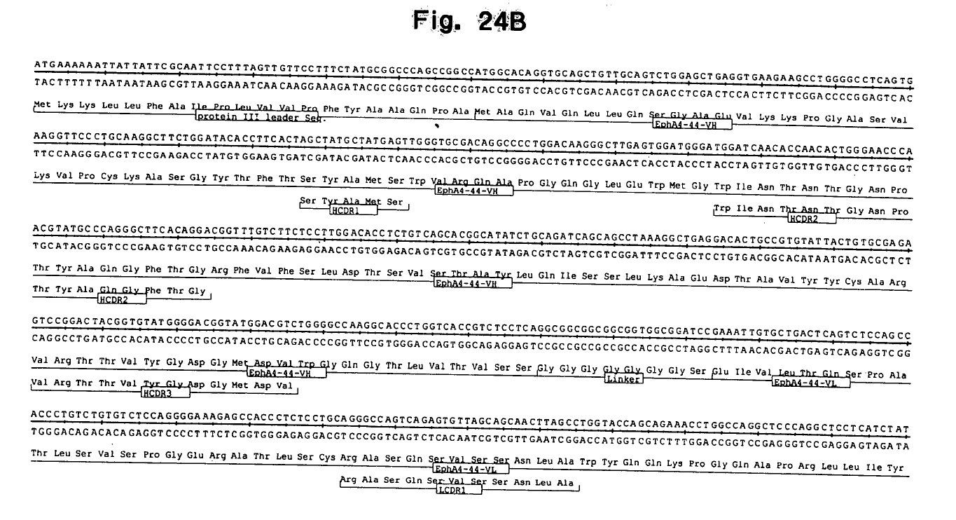

Cells that express the anti-EphA4 scFv EA44 have been deposited with the American Type Culture Collection (P.O. Box 1549, Manassas, Va. 20108) on Jun. 4, 2004 under the provisions of the Budapest Treaty on the International Recognition of the Deposit of Microorganisms for the Purposes of Patent Procedures, and assigned accession number PTA-6044 (see U.S. application Ser. No. 10/863,729, filed Jun. 7, 2004, which is incorporated by reference herein in its entirety). The amino acid and nucleic acid sequences of EA44 are shown in FIGS. 24A-B. The sequences of the EA44 CDRs are indicated in Table 1. In a most preferred embodiment, the antibody is human or has been humanized.

Antibodies of the invention include, but are not limited to, monoclonal antibodies, synthetic antibodies, recombinantly produced antibodies, intrabodies, BiTE molecules, multispecific antibodies (including bi-specific antibodies), human antibodies, humanized antibodies, chimeric antibodies, single-chain Fvs (scFv) (including bi-specific scFvs), single chain antibodies, Fab fragments, F(ab′) fragments, disulfide-linked Fvs (sdFv), and epitope-binding fragments of any of the above. In particular, antibodies used in the methods of the present invention include immunoglobulin molecules and immunologically active portions of immunoglobulin molecules, i.e., molecules that contain an antigen binding site that immunospecifically binds to EphA2 or EphA4 and is an agonist of EphA2 or EphA4, inhibits or reduces a cancer cell phenotype, preferentially binds an EphA2 or EphA4 epitope exposed on cancer cells but not non-cancer cells, and/or binds EphA2 or EphA4 with a K_{off} of less than 3×10⁻³ s⁻¹. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

The antibodies used in the methods of the invention may be from any animal origin including birds and mammals (e.g., human, murine, donkey, sheep, rabbit, goat, guinea pig, camel, horse, or chicken). Preferably, the antibodies are human or humanized monoclonal antibodies. As used herein, “human” antibodies include antibodies having the amino acid sequence of a human immunoglobulin and include antibodies isolated from human immunoglobulin libraries or from mice or other animals that express antibodies from human genes.

The antibodies used in the methods of the present invention may be monospecific, bispecific, trispecific or of greater multispecificity. Multispecific antibodies may immunospecifically bind to different epitopes of an EphA2 or EphA4 polypeptide or may immunospecifically bind to both an EphA2 or EphA4 polypeptide as well as a heterologous epitope, such as a heterologous polypeptide or solid support material. See, e.g., International Publication Nos. WO 93/17715, WO 92/08802, WO 91/00360, and WO 92/05793; Tutt, et al., 1991, J. Immunol. 147: 60-69; U.S. Pat. Nos. 4,474,893, 4,714,681, 4,925,648, 5,573,920, and 5,601,819; and Kostelny et al., 1992, J. Immunol. 148: 1547-1553.

In a specific embodiment, an antibody used in the methods of the present invention is EA2-EA5, Eph099B-102.147, Eph099B-208.261, Eph099B-210.248, Eph099B-233.152, EA44 or any of the antibodies listed in Table 1, or an antigen-binding fragment thereof (e.g., comprising a variable domain or one or more complementarity determining regions (CDRs) of the afore-mentioned antibodies of the invention; e.g., see Table 1). In another embodiment, an agonistic antibody used in the methods of the present invention binds to the same epitope as EA2-5, Eph099B-102.147, Eph099B-208.261, Eph099B-210.248, Eph099B-233.152, EA44 or any of the antibodies listed in Table 1 or competes with EA2-5, Eph099B-102.147, Eph099B-208.261, Eph099B-210.248, Eph099B-233.152 or any of the antibodies listed in Table 1 for binding to EphA2, e.g., in an ELISA assay. In another embodiment, an agonistic antibody used in the methods of the present invention binds to the same epitope as EA44 or competes with EA44 or any of the antibodies listed in Table 1 for binding to EphA4, e.g., in an ELISA assay.

The present invention also encompasses antibodies or fragments thereof that immunospecifically bind to EphA2 and agonize EphA2, inhibit a cancer cell phenotype, preferentially bind an EphA2 epitope exposed in cancer cells, and/or bind EphA2 with a K_{off} of less than 3×10⁻³ s⁻¹, said antibodies comprising a VH CDR having an amino acid sequence of any one of the VH CDRs of EA2-5, Eph099B-102.147, Eph099B-208.261, Eph099B-210.248, Eph099B-233.152, or any of the antibodies listed in Table 1. The present invention also encompasses the use of antibodies that immunospecifically bind to EphA2 and agonize EphA2, inhibit a cancer cell phenotype, preferentially bind an EphA2 epitope exposed in cancer cells, and/or bind EphA2 with a K_{off} of less than 3×10⁻³ s⁻¹, said antibodies comprising a VL CDR having an amino acid sequence of any one of the VL CDRs of EA2-5, Eph099B-102.147, Eph099B-208.261, Eph099B-210.248, Eph099B-233.152, or any of the antibodies listed in Table 1. The present invention also encompasses the use of antibodies that immunospecifically bind to EphA2 and agonize EphA2, inhibit a cancer cell phenotype, preferentially bind an EphA2 epitope exposed in cancer cells, and/or bind EphA2 with a K_{off} of less than 3×10⁻³ s⁻¹, said antibodies comprising one or more VH CDRs and one or more VL CDRs of EA2-5, Eph099B-102.147, Eph099B-208.261, Eph099B-210.248, Eph099B-233.152, or any of the antibodies listed in Table 1. In particular, the invention encompasses the use of antibodies that immunospecifically bind to EphA2 and agonize EphA2, inhibit a cancer cell phenotype, preferentially bind an EphA2 epitope exposed in cancer cells, and/or bind EphA2 with a K_{off} of less than 3×10⁻³ s⁻¹, said antibodies comprising a VH CDR1 and a VL CDR1; a VH CDR1 and a VL CDR2; a VH CDR1 and a VL CDR3; a VH CDR2 and a VL CDR1; VH CDR2 and VL CDR2; a VH CDR2 and a VL CDR3; a VH CDR3 and a VL CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1, a VH CDR2, a VH CDR3 and a VL CDR2; a VH CDR2, a VH CDR3 and a VL CDR3; a VH1 CDR1, a VH CDR3 and a VL CDR1; a VH CDR1, a VH CDR3 and a VL CDR2; a VH CDR1, a VH CDR3 and a VL CDR3; a VH CDR1, a VL CDR1 and a VL CDR2; a VH CDR1, a VL CDR1 and a VL CDR3; a VH CDR1, a VL CDR2 and a VL CDR3; a VH CDR2, a VL CDR1 and a VL CDR2; a VH CDR2, a VL CDR1 and a VL CDR3; a VH CDR2, a VL CDR2 and a VL CDR3; a VH CDR3, a VL CDR1 and a VL CDR2; a VH CDR3, a VL CDR1 and a VL CDR3; a VH CDR3, a VL CDR2 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VL CDR1, a VL CDR2 and a VL CDR3; a VH CDR2, a VL CDR1, a VL CDR2 and a VL CDR3; a VH CDR3, a VL CDR1, a VL CDR2 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR2, a VL CDR2 and a VL CDR3; a VH CDR1, a VH CDR3, a VL CDR1 and a VL CDR2; a VH CDR1, a VH CDR3, a VL CDR1 and a VL CDR3; a VH CDR1, a VH CDR3, a VL CDR2 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VH CDR3, a VL CDR2 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; a VH CDR1, a VH CDR2, a VH CDR3, a VL CDR1, a VL CDR2, and a VL CDR3 or any combination thereof of the VH CDRs and VL CDRs of EA2-5, Eph099B-102.147, Eph099B-208.261, Eph099B-210.248, Eph099B-233.152, or any of the antibodies listed in Table 1. In specific embodiments, the VH CDR1 is SEQ ID NO:6 or 22; the VH CDR2 is SEQ ID NO:7 or 23; the VH CDR3 is SEQ ID NO:8 or 24; the VL CDR1 is SEQ ID NO:2 or 18; the VL CDR2 is SEQ ID NO:3 or 19; and the VL CDR3 is SEQ ID NO:4 or 20 (see, e.g., Table 1). In a more specific embodiment, the VH CDR1 is SEQ ID NO:6; the VH CDR2 is SEQ ID NO:7; the VH CDR3 is SEQ ID NO:8; the VL CDR1 is SEQ ID NO:2; the VL CDR2 is SEQ ID NO:3; and the VL CDR3 is SEQ ID NO:4. In another more specific embodiment, the VH CDR1 is SEQ ID NO:22; the VH CDR2 is SEQ ID NO:23; the VH CDR3 is SEQ ID NO:24; the VL CDR1 is SEQ ID NO:18; the VL CDR2 is SEQ ID NO:19; and the VL CDR3 is SEQ ID NO:20. The invention also encompasses any of the foregoing with one, two, three, four, or five amino acid substitutions, additions, or deletions that bind EphA2.

The present invention also encompasses antibodies or fragments thereof that immunospecifically bind to EphA4 and agonize EphA5, inhibit a cancer cell phenotype, preferentially bind an EphA5 epitope exposed in cancer cells, and/or bind EphA5 with a K_{off} of less than 3×10⁻³ s⁻¹, said antibodies comprising a VH CDR having an amino acid sequence of any one of the VH CDRs of EA44 as listed in Table 1. The present invention also encompasses the use of antibodies that immunospecifically bind to EphA4 and agonize EphA5, inhibit a cancer cell phenotype, preferentially bind an EphA5 epitope exposed in cancer cells, and/or bind EphA4 with a K_{off} of less than 3×10⁻³ s⁻¹, said antibodies comprising a VL CDR having an amino acid sequence of any one of the VL CDRs of EA44 as listed in Table 1. The present invention also encompasses the use of antibodies that immunospecifically bind to EphA4 and agonize EphA5, inhibit a cancer cell phenotype, preferentially bind an EphA5 epitope exposed in cancer cells, and/or bind EphA5 with a K_{off} of less than 3×10⁻³ s⁻¹, said antibodies comprising one or more VH CDRs and one or more VL CDRs of EA44 as listed in Table 1. In particular, the invention encompasses the use of antibodies that immunospecifically bind to EphA4 and agonize EphA4, inhibit a cancer cell phenotype, preferentially bind an EphA4 epitope exposed in cancer cells, and/or bind EphA4 with a K_{off} of less than 3×10⁻³ s⁻¹, said antibodies comprising a VH CDR1 and a VL CDR1; a VH CDR1 and a VL CDR2; a VH CDR1 and a VL CDR3; a VH CDR2 and a VL CDR1; VH CDR2 and VL CDR2; a VH CDR2 and a VL CDR3; a VH CDR3 and a VL CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1, a VH CDR2, a VH CDR3 and a VL CDR2; a VH CDR2, a VH CDR3 and a VL CDR3; a VH1 CDR1, a VH CDR3 and a VL CDR1; a VH CDR1, a VH CDR3 and a VL CDR2; a VH CDR1, a VH CDR3 and a VL CDR3; a VH CDR1, a VL CDR1 and a VL CDR2; a VH CDR1, a VL CDR1 and a VL CDR3; a VH CDR1, a VL CDR2 and a VL CDR3; a VH CDR2, a VL CDR1 and a VL CDR2; a VH CDR2, a VL CDR1 and a VL CDR3; a VH CDR2, a VL CDR2 and a VL CDR3; a VH CDR3, a VL CDR1 and a VL CDR2; a VH CDR3, a VL CDR1 and a VL CDR3; a VH CDR3, a VL CDR2 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VL CDR1, a VL CDR2 and a VL CDR3; a VH CDR2, a VL CDR1, a VL CDR2 and a VL CDR3; a VH CDR3, a VL CDR1, a VL CDR2 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR2, a VL CDR2 and a VL CDR3; a VH CDR1, a VH CDR3, a VL CDR1 and a VL CDR2; a VH CDR1, a VH CDR3, a VL CDR1 and a VL CDR3; a VH CDR1, a VH CDR3, a VL CDR2 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VH CDR3, a VL CDR2 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; a VH CDR1, a VH CDR2, a VH CDR3, a VL CDR1, a VL CDR2, and a VL CDR3 or any combination thereof of the VH CDRs and VL CDRs of EA44 as listed in Table 1. In specific embodiments, the VH CDR1 is SEQ ID NO:70; the VH CDR2 is SEQ ID NO:71; the VH CDR3 is SEQ ID NO:72; the VL CDR1 is SEQ ID NO:66; the VL CDR2 is SEQ ID NO:67; and the VL CDR3 is SEQ ID NO:68 (see, e.g., Table 1). The invention also encompasses any of the foregoing with one, two, three, four, or five amino acid substitutions, additions, or deletions that bind EphA4.

In one embodiment, an antibody that immunospecifically binds to EphA2 and agonizes EphA2, inhibits a cancer cell phenotype, preferentially binds an EphA2 epitope exposed in cancer cells, and/or binds EphA2 with a K_{off} of less than 3×10⁻³ s⁻¹ comprises a VH CDR1 having the amino acid sequence of SEQ ID NO:6 and a VL CDR1 having the amino acid sequence of SEQ ID NO:2. In another embodiment, an antibody that immunospecifically binds to EphA2 and agonizes EphA2, inhibits a cancer cell phenotype, preferentially binds an EphA2 epitope exposed in cancer cells, and/or binds EphA2 with a K_{off} of less than 3×10⁻³ s⁻¹ comprises a VH CDR1 having the amino acid sequence of SEQ ID NO:6 and a VL CDR2 having the amino acid sequence of SEQ ID NO:3. In another embodiment, an antibody that immunospecifically binds to EphA2 and agonizes EphA2, inhibits a cancer cell phenotype, preferentially binds an EphA2 epitope exposed in cancer cells, and/or binds EphA2 with a K_{off} of less than 3×10⁻³ s⁻¹ comprises a VH CDR1 having the amino acid sequence of SEQ ID NO:6 and a VL CDR3 having the amino acid sequence of SEQ ID NO:4.

In one embodiment, an antibody that immunospecifically binds to EphA4 and agonizes EphA4, inhibits a cancer cell phenotype, preferentially binds an EphA4 epitope exposed in cancer cells, and/or binds EphA2 with a K_{off} of less than 3×10⁻³ s⁻¹ comprises a VH CDR1 having the amino acid sequence of SEQ ID NO:70 and a VL CDR1 having the amino acid sequence of SEQ ID NO:66. In another embodiment, an antibody that immunospecifically binds to EphA4 and agonizes EphA4, inhibits a cancer cell phenotype, preferentially binds an EphA4 epitope exposed in cancer cells, and/or binds EphA4 with a K_{off} of less than 3×10⁻³ s⁻¹ comprises a VH CDR1 having the amino acid sequence of SEQ ID NO:70 and a VL CDR2 having the amino acid sequence of SEQ ID NO:67. In another embodiment, an antibody that immunospecifically binds to EphA4 and agonizes EphA4, inhibits a cancer cell phenotype, preferentially binds an EphA4 epitope exposed in cancer cells, and/or binds EphA4 with a K_{off} of less than 3×10⁻³ s⁻¹ comprises a VH CDR1 having the amino acid sequence of SEQ ID NO:70 and a VL CDR3 having the amino acid sequence of SEQ ID NO:68.

In another embodiment, an antibody that immunospecifically binds to EphA2 and agonizes EphA2, inhibits a cancer cell phenotype, preferentially binds an EphA2 epitope exposed in cancer cells, and/or binds EphA2 with a K_{off} of less than 3×10⁻³ s⁻¹ comprises a VH CDR1 having the amino acid sequence of SEQ ID NO:22 and a VL CDR1 having the amino acid sequence of SEQ ID NO:18. In another embodiment, an antibody that immunospecifically binds to EphA2 and agonizes EphA2, inhibits a cancer cell phenotype, preferentially binds an EphA2 epitope exposed in cancer cells, and/or binds EphA2 with a K_{off} of less than 3×10⁻³ s⁻¹ comprises a VH CDR1 having the amino acid sequence of SEQ ID NO:22 and a VL CDR2 having the amino acid sequence of SEQ ID NO:19. In another embodiment, an antibody that immunospecifically binds to EphA2 and agonizes EphA2, inhibits a cancer cell phenotype, preferentially binds an EphA2 epitope exposed in cancer cells, and/or binds EphA2 with a K_{off} of less than 3×10⁻³ s⁻¹ comprises a VH CDR1 having the amino acid sequence of SEQ ID NO:22 and a VL CDR3 having the amino acid sequence of SEQ ID NO:20.

In another embodiment, an antibody that immunospecifically binds to EphA4 and agonizes EphA4, inhibits a cancer cell phenotype, preferentially binds an EphA4 epitope exposed in cancer cells, and/or binds EphA4 with a K_{off} of less than 3×10⁻³ s⁻¹ comprises a VH CDR1 having the amino acid sequence of SEQ ID NO:70 and a VL CDR1 having the amino acid sequence of SEQ ID NO:66. In another embodiment, an antibody that immunospecifically binds to EphA4 and agonizes EphA4, inhibits a cancer cell phenotype, preferentially binds an EphA4 epitope exposed in cancer cells, and/or binds EphA4 with a K_{off} of less than 3×10⁻³ s⁻¹ comprises a VH CDR1 having the amino acid sequence of SEQ ID NO:70 and a VL CDR2 having the amino acid sequence of SEQ ID NO:67. In another embodiment, an antibody that immunospecifically binds to EphA4 and agonizes EphA4, inhibits a cancer cell phenotype, preferentially binds an EphA4 epitope exposed in cancer cells, and/or binds EphA4 with a K_{off} of less than 3×10⁻³ s⁻¹ comprises a VH CDR1 having the amino acid sequence of SEQ ID NO:70 and a VL CDR3 having the amino acid sequence of SEQ ID NO:68.

In another embodiment, an antibody that immunospecifically binds to EphA2 and agonizes EphA2, inhibits a cancer cell phenotype, preferentially binds an EphA2 epitope exposed in cancer cells, and/or binds EphA2 with a K_{off} of less than 3×10⁻³ s⁻¹ comprises a VH CDR2 having the amino acid sequence of SEQ ID NO:7 and a VL CDR1 having the amino acid sequence of SEQ ID NO:2. In another embodiment, an antibody that immunospecifically binds to EphA2 and agonizes EphA2, inhibits a cancer cell phenotype, preferentially binds an EphA2 epitope exposed in cancer cells, and/or binds EphA2 with a K_{off} of less than 3×10⁻³ s⁻¹ comprises a VH CDR2 having the amino acid sequence of SEQ ID NO:7 and a VL CDR2 having the amino acid sequence of SEQ ID NO:3. In another embodiment, an antibody that immunospecifically binds to EphA2 and agonizes EphA2, inhibits a cancer cell phenotype, preferentially binds an EphA2 epitope exposed in cancer cells, and/or binds EphA2 with a K_{off} of less than 3×10⁻³ s⁻¹ comprises a VH CDR2 having the amino acid sequence of SEQ ID NO:7 and a VL CDR3 having the amino acid sequence of SEQ ID NO:4.

In another embodiment, an antibody that immunospecifically binds to EphA4 and agonizes EphA4, inhibits a cancer cell phenotype, preferentially binds an EphA4 epitope exposed in cancer cells, and/or binds EphA4 with a K_{off} of less than 3×10⁻³ s⁻¹ comprises a VH CDR2 having the amino acid sequence of SEQ ID NO:71 and a VL CDR1 having the amino acid sequence of SEQ ID NO:66. In another embodiment, an antibody that immunospecifically binds to EphA4 and agonizes EphA4, inhibits a cancer cell phenotype, preferentially binds an EphA4 epitope exposed in cancer cells, and/or binds EphA4 with a K_{off} of less than 3×10⁻³ s⁻¹ comprises a VH CDR2 having the amino acid sequence of SEQ ID NO:71 and a VL CDR2 having the amino acid sequence of SEQ ID NO:67. In another embodiment, an antibody that immunospecifically binds to EphA4 and agonizes EphA4, inhibits a cancer cell phenotype, preferentially binds an EphA4 epitope exposed in cancer cells, and/or binds EphA4 with a K_{off} of less than 3×10⁻³ s⁻¹ comprises a VH CDR2 having the amino acid sequence of SEQ ID NO:71 and a VL CDR3 having the amino acid sequence of SEQ ID NO:68.

In another embodiment, an antibody that immunospecifically binds to EphA2 and agonizes EphA2, inhibits a cancer cell phenotype, preferentially binds an EphA2 epitope exposed in cancer cells, and/or binds EphA2 with a K_{off} of less than 3×10⁻³ s⁻¹ comprises a VH CDR2 having the amino acid sequence of SEQ ID NO:23 and a VL CDR1 having the amino acid sequence of SEQ ID NO:18. In another embodiment, an antibody that immunospecifically binds to EphA2 and agonizes EphA2, inhibits a cancer cell phenotype, preferentially binds an EphA2 epitope exposed in cancer cells, and/or binds EphA2 with a K_{off} of less than 3×10⁻³ s⁻¹ comprises a VH CDR2 having the amino acid sequence of SEQ ID NO:23 and a VL CDR2 having the amino acid sequence of SEQ ID NO:19. In another embodiment, an antibody that immunospecifically binds to EphA2 and agonizes EphA2, inhibits a cancer cell phenotype, preferentially binds an EphA2 epitope exposed in cancer cells, and/or binds EphA2 with a K_{off} of less than 3×10⁻³ s⁻¹ comprises a VH CDR2 having the amino acid sequence of SEQ ID NO:23 and a VL CDR3 having the amino acid sequence of SEQ ID NO:20.

In another embodiment, an antibody that immunospecifically binds to EphA4 and agonizes EphA4, inhibits a cancer cell phenotype, preferentially binds an EphA4 epitope exposed in cancer cells, and/or binds EphA4 with a K_{off} of less than 3×10⁻³ s⁻¹ comprises a VH CDR2 having the amino acid sequence of SEQ ID NO:71 and a VL CDR1 having the amino acid sequence of SEQ ID NO:66. In another embodiment, an antibody that immunospecifically binds to EphA4 and agonizes EphA4, inhibits a cancer cell phenotype, preferentially binds an EphA4 epitope exposed in cancer cells, and/or binds EphA4 with a K_{off} of less than 3×10⁻³ s⁻¹ comprises a VH CDR2 having the amino acid sequence of SEQ ID NO:71 and a VL CDR2 having the amino acid sequence of SEQ ID NO:67. In another embodiment, an antibody that immunospecifically binds to EphA4 and agonizes EphA4, inhibits a cancer cell phenotype, preferentially binds an EphA4 epitope exposed in cancer cells, and/or binds EphA4 with a K_{off} of less than 3×10⁻³ s⁻¹ comprises a VH CDR2 having the amino acid sequence of SEQ ID NO:71 and a VL CDR3 having the amino acid sequence of SEQ ID NO:68.

In another embodiment, an antibody that immunospecifically binds to EphA2 and agonizes EphA2, inhibits a cancer cell phenotype, preferentially binds an EphA2 epitope exposed in cancer cells, and/or binds EphA2 with a K_{off} of less than 3×10⁻³ s⁻¹ comprises a VH CDR3 having the amino acid sequence of SEQ ID NO:8 and a VL CDR1 having the amino acid sequence of SEQ ID NO:2. In another embodiment, an antibody that immunospecifically binds to EphA2 and agonizes EphA2, inhibits a cancer cell phenotype, preferentially binds an EphA2 epitope exposed in cancer cells, and/or binds EphA2 with a K_{off} of less than 3×10⁻³ s⁻¹ comprises a VH CDR3 having the amino acid sequence of SEQ ID NO:8 and a VL CDR2 having the amino acid sequence of SEQ ID NO:3. In another embodiment, an antibody that immunospecifically binds to EphA2 and agonizes EphA2, inhibits a cancer cell phenotype, preferentially binds an EphA2 epitope exposed in cancer cells, and/or binds EphA2 with a K_{off} of less than 3×10⁻³ s⁻¹ comprises a VH CDR3 having the amino acid sequence of SEQ ID NO:8 and a VL CDR3 having the amino acid sequence of SEQ ID NO:4.

In another embodiment, an antibody that immunospecifically binds to EphA4 and agonizes EphA4, inhibits a cancer cell phenotype, preferentially binds an EphA4 epitope exposed in cancer cells, and/or binds EphA4 with a K_{off} of less than 3×10⁻³ s⁻¹ comprises a VH CDR3 having the amino acid sequence of SEQ ID NO:72 and a VL CDR1 having the amino acid sequence of SEQ ID NO:66. In another embodiment, an antibody that immunospecifically binds to EphA4 and agonizes EphA4, inhibits a cancer cell phenotype, preferentially binds an EphA4 epitope exposed in cancer cells, and/or binds EphA4 with a K_{off} of less than 3×10⁻³ s⁻¹ comprises a VH CDR3 having the amino acid sequence of SEQ ID NO:72 and a VL CDR2 having the amino acid sequence of SEQ ID NO:67. In another embodiment, an antibody that immunospecifically binds to EphA4 and agonizes EphA4, inhibits a cancer cell phenotype, preferentially binds an EphA4 epitope exposed in cancer cells, and/or binds EphA4 with a K_{off} of less than 3×10⁻³ s⁻¹ comprises a VH CDR3 having the amino acid sequence of SEQ ID NO:72 and a VL CDR3 having the amino acid sequence of SEQ ID NO:68.

In another embodiment, an antibody that immunospecifically binds to EphA2 and agonizes EphA2, inhibits a cancer cell phenotype, preferentially binds an EphA2 epitope exposed in cancer cells, and/or binds EphA2 with a K_{off} of less than 3×10⁻³ s⁻¹ comprises a VH CDR3 having the amino acid sequence of SEQ ID NO:24 and a VL CDR1 having the amino acid sequence of SEQ ID NO:18. In another embodiment, an antibody that immunospecifically binds to EphA2 and agonizes EphA2, inhibits a cancer cell phenotype, preferentially binds an EphA2 epitope exposed in cancer cells, and/or binds EphA2 with a K_{off} of less than 3×10⁻³ s⁻¹ comprises a VH CDR3 having the amino acid sequence of SEQ ID NO:24 and a VL CDR2 having the amino acid sequence of SEQ ID NO:19. In another embodiment, an antibody that immunospecifically binds to EphA2 and agonizes EphA2, inhibits a cancer cell phenotype, preferentially binds an EphA2 epitope exposed in cancer cells, and/or binds EphA2 with a K_{off} of less than 3×10⁻³ s⁻¹ comprises a VH CDR3 having the amino acid sequence of SEQ ID NO:24 and a VL CDR3 having the amino acid sequence of SEQ ID NO:20.

In another embodiment, an antibody that immunospecifically binds to EphA4 and agonizes EphA4, inhibits a cancer cell phenotype, preferentially binds an EphA4 epitope exposed in cancer cells, and/or binds EphA4 with a K_{off} of less than 3×10⁻³ s⁻¹ comprises a VH CDR3 having the amino acid sequence of SEQ ID NO:72 and a VL CDR1 having the amino acid sequence of SEQ ID NO:66. In another embodiment, an antibody that immunospecifically binds to EphA4 and agonizes EphA4, inhibits a cancer cell phenotype, preferentially binds an EphA4 epitope exposed in cancer cells, and/or binds EphA4 with a K_{off} of less than 3×10⁻³ s⁻¹ comprises a VH CDR3 having the amino acid sequence of SEQ ID NO:72 and a VL CDR2 having the amino acid sequence of SEQ ID NO:67. In another embodiment, an antibody that immunospecifically binds to EphA4 and agonizes EphA4, inhibits a cancer cell phenotype, preferentially binds an EphA4 epitope exposed in cancer cells, and/or binds EphA4 with a K_{off} of less than 3×10⁻³ s⁻¹ comprises a VH CDR3 having the amino acid sequence of SEQ ID NO:72 and a VL CDR3 having the amino acid sequence of SEQ ID NO:68.

The antibodies used in the methods of the invention include derivatives that are modified, i.e, by the covalent attachment of any type of molecule to the antibody. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The present invention also provides antibodies of the invention or fragments thereof that comprise a framework region known to those of skill in the art. Preferably, the antibody of the invention or fragment thereof is human or humanized. In a specific embodiment, the antibody of the invention or fragment thereof comprises one or more CDRs from EA2-5, Eph099B-102.147, Eph099B-208.261, Eph099B-210.248, Eph099B-233.152, or any of the antibodies listed in Table 1 (or any other EphA2 agonistic antibody or EphA2 cancer cell phenotype inhibiting antibody or an EphA2 antibody that binds EphA2 with a K_{off} of less than 3×10⁻³ s⁻¹), binds EphA2, and, preferably, agonizes EphA2 and/or inhibits a cancer cell phenotype and/or binds EphA2 with a K_{off} of less than 3×10⁻³ s⁻¹. In another specific embodiment, the antibody of the invention or fragment thereof comprises one or more CDRs from EA44 as listed in Table 1 (or any other EphA4 agonistic antibody or EphA4 cancer cell phenotype inhibiting antibody or an EphA4 antibody that binds EphA2 with a K_{off} of less than 3×10⁻³ s⁻¹), binds EphA4, and, preferably, agonizes EphA4 and/or inhibits a cancer cell phenotype and/or binds EphA4 with a K_{off} of less than 3×10⁻³ s⁻¹.

The present invention encompasses single domain antibodies, including camelized single domain antibodies (see e.g., Muyldermans et al., 2001, Trends Biochem. Sci. 26: 230; Nuttall et al., 2000, Cur. Pharm. Biotech. 1: 253; Reichmann and Muyldermans, 1999, J. Immunol. Meth. 231: 25; International Publication Nos. WO 94/04678 and WO 94/25591; U.S. Pat. No. 6,005,079; which are incorporated herein by reference in their entireties). In one embodiment, the present invention provides single domain antibodies comprising two VH domains having the amino acid sequence of any of the VH domains of EA2-5, Eph099B-102.147, Eph099B-208.261, Eph099B-210.248, Eph099B-233.152, EA44 or any of the antibodies listed in Table 1 (or any other EphA2 or EphA4 agonistic antibody, EphA2 or EphA4 cancer cell phenotype inhibiting antibody, exposed EphA2 or EphA4 epitope antibody, or an EphA2 or EphA4 antibody that binds EphA2 or EphA4 with a K_{off} of less than 3×10⁻³ s⁻¹) with modifications such that single domain antibodies are formed. In another embodiment, the present invention also provides single domain antibodies comprising two VH domains comprising one or more of the VH CDRs of EA2-5, Eph099B-102.147, Eph099B-208.261, Eph099B-210.248, Eph099B-233.152, EA44 any of the antibodies listed in Table 1 (or any other EphA2 or EphA4 agonistic antibody, EphA2 or EphA4 cancer cell phenotype inhibiting antibody, exposed EphA2 or EphA4 epitope antibody, or an EphA2 or EphA4 antibody that binds EphA2 or EphA4 with a K_{off} of less than 3×10⁻³ s⁻¹).

The methods of the present invention also encompass the use of antibodies or fragments thereof that have half-lives (e.g., serum half-lives) in a mammal, preferably a human, of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present invention or fragments thereof in a mammal, preferably a human, result in a higher serum titer of said antibodies or antibody fragments in the mammal, and thus, reduce the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor (see, e.g., International Publication Nos. WO 97/34631 and WO 02/060919, which are incorporated herein by reference in their entireties). Antibodies or fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

The present invention also encompasses the use of antibodies or antibody fragments comprising the amino acid sequence of one or both variable domains of EA2-5, Eph099B-102.147, Eph099B-208.261, Eph099B-210.248, Eph099B-233.152, EA44 any of the antibodies listed in Table 1 (e.g., one or more amino acid substitutions) in the variable regions. Preferably, mutations in these antibodies maintain or enhance the avidity and/or affinity of the antibodies for the particular antigen(s) to which they immunospecifically bind. Standard techniques known to those skilled in the art (e.g., immunoassays) can be used to assay the affinity of an antibody for a particular antigen.

Standard techniques known to those skilled in the art can be used to introduce mutations in the nucleotide sequence encoding an antibody, or fragment thereof, including, e.g., site-directed mutagenesis and PCR-mediated mutagenesis, which results in amino acid substitutions. Preferably, the derivatives include less than 15 amino acid substitutions, less than 10 amino acid substitutions, less than 5 amino acid substitutions, less than 4 amino acid substitutions, less than 3 amino acid substitutions, or less than 2 amino acid substitutions relative to the original antibody or fragment thereof. In a preferred embodiment, the derivatives have conservative amino acid substitutions made at one or more predicted non-essential amino acid residues.

The present invention also encompasses antibodies or fragments thereof that immunospecifically bind to EphA2 and agonize EphA2 and/or inhibit a cancer cell phenotype, preferentially bind an EphA2 epitope exposed in cancer cells, and/or bind EphA2 with a K_{off} of less than 3×10⁻³ s⁻¹, said antibodies or antibody fragments comprising an amino acid sequence of a variable light chain and/or variable heavy chain that is at least 45%, at least 50%, at least 55%, at least 60%, at least 65%, at least 70%, at least 75%, at least 80%, at least 85%, at least 90%, at least 95%, or at least 99% identical to the amino acid sequence of the variable light chain and/or heavy chain of EA2-5, Eph099B-102.147, Eph099B-208.261, Eph099B-210.248, Eph099B-233.152, or any of the antibodies listed in Table 1. In some embodiments, antibodies or antibody fragments of the invention immunospecifically bind to EphA2 and comprise an amino acid sequence of a variable light chain that is at least 45%, at least 50%, at least 55%, at least 60%, at least 65%, at least 70%, at least 75%, at least 80%, at least 85%, at least 90%, at least 95%, or at least 99% identical to SEQ ID NO:1 or 17. In other embodiments, antibodies or antibody fragments of the invention immunospecifically bind to EphA2 and comprise an amino acid sequence of a variable heavy chain that is at least 45%, at least 50%, at least 55%, at least 60%, at least 65%, at least 70%, at least 75%, at least 80%, at least 85%, at least 90%, at least 95%, or at least 99% identical to SEQ ID NO:5 or 21. In other embodiments, antibodies or antibody fragments of the invention immunospecifically bind to EphA2 and comprise an amino acid sequence of a variable light chain that is at least 45%, at least 50%, at least 55%, at least 60%, at least 65%, at least 70%, at least 75%, at least 80%, at least 85%, at least 90%, at least 95%, or at least 99% identical to SEQ ID NO:1 or 17 and a variable heavy chain that is at least 45%, at least 50%, at least 55%, at least 60%, at least 65%, at least 70%, at least 75%, at least 80%, at least 85%, at least 90%, at least 95%, or at least 99% identical to SEQ ID NO:5 or 21.

The present invention also encompasses antibodies or fragments thereof that immunospecifically bind to EphA4 and agonize EphA4 and/or inhibit a cancer cell phenotype, preferentially bind an EphA4 epitope exposed in cancer cells, and/or bind EphA4 with a K_{off} of less than 3×10⁻³ s⁻¹, said antibodies or antibody fragments comprising an amino acid sequence of a variable light chain and/or variable heavy chain that is at least 45%, at least 50%, at least 55%, at least 60%, at least 65%, at least 70%, at least 75%, at least 80%, at least 85%, at least 90%, at least 95%, or at least 99% identical to the amino acid sequence of the variable light chain and/or heavy chain of EA44 listed in Table 1. In some embodiments, antibodies or antibody fragments of the invention immunospecifically bind to EphA4 and comprise an amino acid sequence of a variable light chain that is at least 45%, at least 50%, at least 55%, at least 60%, at least 65%, at least 70%, at least 75%, at least 80%, at least 85%, at least 90%, at least 95%, or at least 99% identical to SEQ ID NO:65. In other embodiments, antibodies or antibody fragments of the invention immunospecifically bind to EphA4 and comprise an amino acid sequence of a variable heavy chain that is at least 45%, at least 50%, at least 55%, at least 60%, at least 65%, at least 70%, at least 75%, at least 80%, at least 85%, at least 90%, at least 95%, or at least 99% identical to SEQ ID NO:69. In other embodiments, antibodies or antibody fragments of the invention immunospecifically bind to EphA4 and comprise an amino acid sequence of a variable light chain that is at least 45%, at least 50%, at least 55%, at least 60%, at least 65%, at least 70%, at least 75%, at least 80%, at least 85%, at least 90%, at least 95%, or at least 99% identical to SEQ ID NO:65 and a variable heavy chain that is at least 45%, at least 50%, at least 55%, at least 60%, at least 65%, at least 70%, at least 75%, at least 80%, at least 85%, at least 90%, at least 95%, or at least 99% identical to SEQ ID NO:69.

The present invention further encompasses antibodies or fragments thereof that immunospecifically bind to EphA2 and agonize EphA2 and/or inhibit a cancer cell phenotype, preferentially bind an EphA2 epitope exposed in cancer cells, and/or bind EphA2 with a K_{off} of less than 3×10⁻³ s⁻¹, said antibodies or antibody fragments comprising an amino acid sequence of one or more CDRs that is at least 45%, at least 50%, at least 55%, at least 60%, at least 65%, at least 70%, at least 75%, at least 80%, at least 85%, at least 90%, at least 95%, or at least 99% identical to the amino acid sequence of one or more CDRs of EA2-5, Eph099B-102.147, Eph099B-208.261, Eph099B-210.248, Eph099B-233.152, or any of the antibodies listed in Table 1. In one embodiment, antibodies or antibody fragments of the invention immunospecifically bind to EphA2 and comprise an amino acid sequence of a CDR that is at least 45%, at least 50%, at least 55%, at least 60%, at least 65%, at least 70%, at least 75%, at least 80%, at least 85%, at least 90%, at least 95%, or at least 99% identical to SEQ ID NO:2, 3, or 4. In another embodiment, antibodies or antibody fragments of the invention immunospecifically bind to EphA2 and comprise an amino acid sequence of a CDR that is at least 45%, at least 50%, at least 55%, at least 60%, at least 65%, at least 70%, at least 75%, at least 80%, at least 85%, at least 90%, at least 95%, or at least 99% identical to SEQ ID NO:18, 19, or 20. In another embodiment, antibodies or antibody fragments of the invention immunospecifically bind to EphA2 and comprise an amino acid sequence of a CDR that is at least 45%, at least 50%, at least 55%, at least 60%, at least 65%, at least 70%, at least 75%, at least 80%, at least 85%, at least 90%, at least 95%, or at least 99% identical to SEQ ID NO:6, 7, or 8. In another embodiment, antibodies or antibody fragments of the invention immunospecifically bind to EphA2 and comprise an amino acid sequence of a CDR that is at least 45%, at least 50%, at least 55%, at least 60%, at least 65%, at least 70%, at least 75%, at least 80%, at least 85%, at least 90%, at least 95%, or at least 99% identical to SEQ ID NO:22, 23, or 24.

The present invention further encompasses antibodies or fragments thereof that immunospecifically bind to EphA4 and agonize EphA4 and/or inhibit a cancer cell phenotype, preferentially bind an EphA4 epitope exposed in cancer cells, and/or bind EphA4 with a K_{off} of less than 3×10⁻³ s⁻¹, said antibodies or antibody fragments comprising an amino acid sequence of one or more CDRs that is at least 45%, at least 50%, at least 55%, at least 60%, at least 65%, at least 70%, at least 75%, at least 80%, at least 85%, at least 90%, at least 95%, or at least 99% identical to the amino acid sequence of one or more CDRs of EA44 listed in Table 1. In one embodiment, antibodies or antibody fragments of the invention immunospecifically bind to EphA4 and comprise an amino acid sequence of a CDR that is at least 45%, at least 50%, at least 55%, at least 60%, at least 65%, at least 70%, at least 75%, at least 80%, at least 85%, at least 90%, at least 95%, or at least 99% identical to SEQ ID NO:66, 67, or 68. In another embodiment, antibodies or antibody fragments of the invention immunospecifically bind to EphA4 and comprise an amino acid sequence of a CDR that is at least 45%, at least 50%, at least 55%, at least 60%, at least 65%, at least 70%, at least 75%, at least 80%, at least 85%, at least 90%, at least 95%, or at least 99% identical to SEQ ID NO:70, 71 or 72.

The determination of percent identity of two amino acid sequences can be determined by any method known to one skilled in the art, including BLAST protein searches.

The present invention further encompasses antibodies or fragments thereof that immunospecifically bind to EphA2 and agonize EphA2 and/or inhibit a cancer cell phenotype, preferentially bind an EphA2 epitope exposed in cancer cells, and/or bind EphA2 with a K_{off} of less than 3×10⁻³ s⁻¹, said antibodies or antibody fragments comprising an amino acid sequence of one or more CDRs comprising amino acid residue substitutions, deletions or additions as compared to SEQ ID NO: 2, 3, 4, 6, 7, 8, 18, 19, 20, 22, 23, or 24. The antibody comprising the one or more CDRs comprising amino acid residue substitutions, deletions or additions may have substantially the same binding, better binding, or worse binding when compared to an antibody comprising one or more CDRs without amino acid residue substitutions, deletions or additions. In specific embodiments, one, two, three, four, or five amino acid residues of the CDR have been substituted, deleted or added (i.e., mutated).

The present invention further encompasses antibodies or fragments thereof that immunospecifically bind to EphA4 and agonize EphA4 and/or inhibit a cancer cell phenotype, preferentially bind an EphA4 epitope exposed in cancer cells, and/or bind EphA4 with a K_{off} of less than 3×10⁻³ s⁻¹, said antibodies or antibody fragments comprising an amino acid sequence of one or more CDRs comprising amino acid residue substitutions, deletions or additions as compared to SEQ ID NO: 66, 67, 68, 70, 71 or 72. The antibody comprising the one or more CDRs comprising amino acid residue substitutions, deletions or additions may have substantially the same binding, better binding, or worse binding when compared to an antibody comprising one or more CDRs without amino acid residue substitutions, deletions or additions. In specific embodiments, one, two, three, four, or five amino acid residues of the CDR have been substituted, deleted or added (i.e., mutated).

The present invention also encompasses the use of antibodies or antibody fragments that immunospecifically bind to EphA2 or EphA4 and agonize EphA2 or EphA4 and/or inhibit a cancer cell phenotype, preferentially bind epitopes on EphA2 or EphA4 that are selectively exposed or increased on cancer cells but not non-cancer cells and/or bind EphA2 or EphA4 with a K_{off} less than 3×10⁻³ s⁻¹, where said antibodies or antibody fragments are encoded by a nucleotide sequence that hybridizes to the nucleotide sequence of EA2-5, Eph099B-102.147, Eph099B-208.261, Eph099B-210.248, Eph099B-233.152, EA44 any of the antibodies listed in Table 1 under stringent conditions. In one embodiment, the invention provides antibodies or fragments thereof that immunospecifically bind to EphA2 or EphA4 and agonize EphA2 or EphA4 and/or inhibit a cancer cell phenotype, preferentially bind an epitope on EphA2 that is selectively exposed or increased on cancer cells but not non-cancer cells and/or bind EphA2 or EphA4 with a K_{off} less than 3×10⁻³ s⁻¹, said antibodies or antibody fragments comprising a variable light chain encoded by a nucleotide sequence that hybridizes under stringent conditions to the nucleotide sequence of the variable light chain of EA2-5, Eph099B-102.147, Eph099B-208.261, Eph099B-210.248, Eph099B-233.152, EA44 any of the antibodies listed in Table 1. In a preferred embodiment, the invention provides antibodies or fragments that immunospecifically bind to EphA2 and comprise a variable light chain encoded by a nucleotide sequence that hybridizes under stringent conditions to the nucleotide sequence of SEQ ID NO:9 or 25. In another embodiment, the invention provides antibodies or fragments thereof that immunospecifically bind to EphA2 and agonize EphA2 and/or inhibit a cancer cell phenotype, preferentially bind an epitope on EphA2 that is selectively exposed or increased on cancer cells but not non-cancer cells and/or bind EphA2 with a K_{off} less than 3×10⁻³ s⁻¹, said antibodies or antibody fragments comprising a variable heavy chain encoded by a nucleotide sequence that hybridizes under stringent conditions to the nucleotide sequence of the variable heavy chain of EA2-5, Eph099B-102.147, Eph099B-208.261, Eph099B-210.248, Eph099B-233.152, or any of the antibodies listed in Table 1. In a preferred embodiment, the invention provides antibodies or fragments thereof that immunospecifically bind to EphA2 and comprise a variable heavy chain encoded by a nucleotide sequence that hybridizes under stringent conditions to the nucleotide sequence of SEQ ID NO:13 or 29. In other embodiments, antibodies or antibody fragments of the invention immunospecifically bind to EphA2 and comprise a variable light chain encoded by a nucleotide sequence that hybridizes under stringent conditions to the nucleotide sequence of SEQ ID NO:9 or 25 and a variable heavy chain encoded by a nucleotide sequence that hybridizes under stringent conditions to the nucleotide sequence of SEQ ID NO:13 or 29. In another preferred embodiment, the invention provides antibodies or fragments that immunospecifically bind to EphA and comprise a variable light chain encoded by a nucleotide sequence that hybridizes under stringent conditions to the nucleotide sequence of SEQ ID NO:73. In another embodiment, the invention provides antibodies or fragments thereof that immunospecifically bind to EphA4 and agonize EphA4 and/or inhibit a cancer cell phenotype, preferentially bind an epitope on EphA4 that is selectively exposed or increased on cancer cells but not non-cancer cells and/or bind EphA4 with a K_{off} less than 3×10⁻³ s⁻¹, said antibodies or antibody fragments comprising a variable heavy chain encoded by a nucleotide sequence that hybridizes under stringent conditions to the nucleotide sequence of the variable heavy chain of EA44 listed in Table 1. In a preferred embodiment, the invention provides antibodies or fragments thereof that immunospecifically bind to EphA4 and comprise a variable heavy chain encoded by a nucleotide sequence that hybridizes under stringent conditions to the nucleotide sequence of SEQ ID NO:77. In other embodiments, antibodies or antibody fragments of the invention immunospecifically bind to EphA4 and comprise a variable light chain encoded by a nucleotide sequence that hybridizes under stringent conditions to the nucleotide sequence of SEQ ID NO:73 and a variable heavy chain encoded by a nucleotide sequence that hybridizes under stringent conditions to the nucleotide sequence of SEQ ID NO:77.

In another embodiment, the invention provides antibodies or fragments thereof that immunospecifically bind to EphA2 and agonize EphA2 and/or inhibit a cancer cell phenotype, preferentially bind an EphA2 epitope exposed on cancer cells but not non-cancer cells and/or bind EphA2 with a K_{off} less than 3×10⁻³ s⁻¹, said antibodies or antibody fragments comprising one or more CDRs encoded by a nucleotide sequence that hybridizes under stringent conditions to the nucleotide sequence of one or more CDRs of EA2-5, Eph099B-102.147, Eph099B-208.261, Eph099B-210.248, Eph099B-233.152, or any of the antibodies listed in Table 1. In a preferred embodiment, the antibodies or fragments of the invention immunospecifically bind to EphA2 and comprise a CDR encoded by a nucleotide sequence that hybridizes under stringent conditions the nucleotide sequence of SEQ ID NO:10, 11, or 12. In another preferred embodiment, the antibodies or fragments of the invention immunospecifically bind to EphA2 and comprise a CDR encoded by a nucleotide sequence that hybridizes under stringent conditions the nucleotide sequence of SEQ ID NO:26, 27, or 28. In another preferred embodiment, the antibodies or fragments of the invention immunospecifically bind to EphA2 and comprise a CDR encoded by a nucleotide sequence that hybridizes under stringent conditions the nucleotide sequence of SEQ ID NO:14, 15, or 16. In another preferred embodiment, the antibodies or fragments of the invention immunospecifically bind to EphA2 and comprise a CDR encoded by a nucleotide sequence that hybridizes under stringent conditions the nucleotide sequence of SEQ ID NO:30, 31, or 32.

In another embodiment, the invention provides antibodies or fragments thereof that immunospecifically bind to EphA4 and agonize EphA4 and/or inhibit a cancer cell phenotype, preferentially bind an EphA4 epitope exposed on cancer cells but not non-cancer cells and/or bind EphA4 with a K_{off} less than 3×10⁻³ s⁻¹, said antibodies or antibody fragments comprising one or more CDRs encoded by a nucleotide sequence that hybridizes under stringent conditions to the nucleotide sequence of one or more CDRs of EA44 listed in Table 1. In a preferred embodiment, the antibodies or fragments of the invention immunospecifically bind to EphA4 and comprise a CDR encoded by a nucleotide sequence that hybridizes under stringent conditions the nucleotide sequence of SEQ ID NO:74, 75 or 76. In another preferred embodiment, the antibodies or fragments of the invention immunospecifically bind to EphA2 and comprise a CDR encoded by a nucleotide sequence that hybridizes under stringent conditions the nucleotide sequence of SEQ ID NO:78, 79 or 80.

Stringent hybridization conditions include, but are not limited to, hybridization to filter-bound DNA in 6× sodium chloride/sodium citrate (SSC) at about 45° C. followed by one or more washes in 0.2×SSC/0.1% SDS at about 50-65° C., highly stringent conditions such as hybridization to filter-bound DNA in 6×SSC at about 45° C. followed by one or more washes in 0.1×SSC/0.2% SDS at about 60° C., or any other stringent hybridization conditions known to those skilled in the art (see, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3).

The present invention further encompasses antibodies or fragments thereof that immunospecifically bind to EphA2 and agonize EphA2 and/or inhibit a cancer cell phenotype, preferentially bind an EphA2 epitope exposed in cancer cells, and/or bind EphA2 with a K_{off} of less than 3×10⁻³ s⁻¹, said antibodies or antibody fragments said antibodies or antibody fragments comprising one or more CDRs encoded by a nucleotide sequence of one or more CDRs comprising nucleic acid residue substitutions, deletions or additions as compared to SEQ ID NO:10, 11, 12, 14, 15, 16, 26, 27, 28, 30, 31, or 32. The antibody comprising the one or more CDRs comprising nucleic acid residue substitutions, deletions or additions may have substantially the same binding, better binding, or worse binding when compared to an antibody comprising one or more CDRs without nucleic acid residue substitutions, deletions or additions. In specific embodiments, one, two, three, four, five, six, seven, eight, nine, ten, eleven, twelve, thirteen, fourteen, or fifteen nucleic acid residues of the CDR have been substituted, deleted or added (i.e., mutated). The nucleic acid substitutions may or may not change the amino acid sequence of the mutated CDR.

The present invention further encompasses antibodies or fragments thereof that immunospecifically bind to EphA4 and agonize EphA42 and/or inhibit a cancer cell phenotype, preferentially bind an EphA4 epitope exposed in cancer cells, and/or bind EphA4 with a K_{off} of less than 3×10⁻³ s⁻¹, said antibodies or antibody fragments said antibodies or antibody fragments comprising one or more CDRs encoded by a nucleotide sequence of one or more CDRs comprising nucleic acid residue substitutions, deletions or additions as compared to SEQ ID NO: 66, 67, 68, 70, 71 or 72. The antibody comprising the one or more CDRs comprising nucleic acid residue substitutions, deletions or additions may have substantially the same binding, better binding, or worse binding when compared to an antibody comprising one or more CDRs without nucleic acid residue substitutions, deletions or additions. In specific embodiments, one, two, three, four, five, six, seven, eight, nine, ten, eleven, twelve, thirteen, fourteen, or fifteen nucleic acid residues of the CDR have been substituted, deleted or added (i.e., mutated). The nucleic acid substitutions may or may not change the amino acid sequence of the mutated CDR.

TABLE 1

| | | | | SEQ | |
|---|---|---|---|---|---|
| | | | | ID NO. | |
| | | | SEQ ID NO. | (nucleic | ATCC |
| Antibody | V chain | CDR | (amino acid) | acid) | Deposit No. |
| Eph099B-208.261 | | | | | PTA-4573 |
| | VL | | 1 | 9 | |
| | | VL1 | 2 | 10 | |
| | | VL2 | 3 | 11 | |
| | | VL3 | 4 | 12 | |
| | VH | | 5 | 13 | |
| | | VH1 | 6 | 14 | |
| | | VH2 | 7 | 15 | |
| | | VH3 | 8 | 16 | |
| Eph099B-233.152 | | | | | PTA-5194 |
| | VL | | 17 | 25 | |
| | | VL1 | 18 | 26 | |
| | | VL2 | 19 | 27 | |
| | | VL3 | 20 | 28 | |
| | VH | | 21 | 29 | |
| | | VH1 | 22 | 30 | |
| | | VH2 | 23 | 31 | |
| | | VH3 | 24 | 32 | |
| EA2 | | | | | PTA-4380 |
| | VL | | 33 | 41 | |
| | | VL1 | 34 | 42 | |
| | | VL2 | 35 | 43 | |
| | | VL3 | 36 | 44 | |
| | VH | | 37 | 45 | |
| | | VH1 | 38 | 46 | |
| | | VH2 | 39 | 47 | |
| | | VH3 | 40 | 48 | |
| EA5 | | | | | PTA-4381 |
| | VL | | 49 | 57 | |
| | | VL1 | 50 | 58 | |
| | | VL2 | 51 | 59 | |
| | | VL3 | 52 | 60 | |
| | VH | | 53 | 61 | |
| | | VH1 | 54 | 62 | |
| | | VH2 | 55 | 63 | |
| | | VH3 | 56 | 64 | |
| EA44 | | | | | PTA-6044 |
| | VL | | 65 | 73 | |
| | | VL1 | 66 | 74 | |
| | | VL2 | 67 | 75 | |
| | | VL3 | 68 | 76 | |
| | VH | | 69 | 77 | |
| | | VH1 | 70 | 78 | |
| | | VH2 | 71 | 79 | |
| | | VH3 | 72 | 80 | |

5.1.1.1 Anti-LMW-PTP Intrabodies

An intrabody which inhibits or reduces LMW-PTP, EphA2 or EphA4 activity or expression can be used in accordance with the present invention. Intrabodies are antibodies, often scFvs, that expressed from a recombinant nucleic acid molecule and engineered to be retained intracellularly (e.g., retained in the cytoplasm, endoplasmic reticulum, or periplasm). Intrabodies may be used, for example, to ablate the function of a protein to which the intrabody binds. The expression of intrabodies may also be regulated through the use of inducible promoters in the nucleic acid expression vector comprising the intrabody. Intrabodies of the invention can be produced using methods known in the art, such as those disclosed and reviewed in Chen et al., Hum. Gene Ther. 5: 595-601 (1994); Marasco, W. A., Gene Ther. 4: 11-15 (1997); Rondon and Marasco, Annu. Rev. Microbiol. 51: 257-283 (1997); Proba et al., J. Mol. Biol. 275: 245-253 (1998); Cohen et al., Oncogene 17: 2445-2456 (1998); Ohage and Steipe, J. Mol. Biol. 291: 1119-1128 (1999); Ohage et al., J. Mol. Biol. 291: 1129-1134 (1999); Wirtz and Steipe, Protein Sci. 8: 2245-2250 (1999); Zhu et al., J. Immunol. Methods 231: 207-222 (1999); Steinberger et al., Proc. Natl. Acad. Sci. USA 97: 805-810 (2000); and references cited therein. Each of the references is incorporated herein by reference in its entirety. In a specific embodiment, an agent that inhibit LMW-PTP expression or activity is an anti-LMW-PTP, EphA2 or EphA4 intrabody.

An intrabody comprises at least a portion of an antibody that is capable of immunospecifically binding an antigen and preferably does not contain sequences coding for its secretion. Such antibodies will bind antigen intracellularly. In one embodiment, the intrabody comprises a single-chain Fv (“scFv”). scFvs are antibody fragments comprising the V_{H} and V_{L} domains of antibody, wherein these domains are present in a single polypeptide chain. Generally, the scFv polypeptide further comprises a polypeptide linker between the V_{H} and V_{L} domains which enables the scFv to form the desired structure for antigen binding. For a review of sFvs see Pluckthun in The Pharmacology of Monoclonal Antibodies, vol. 113, Rosenburg and Moore eds. Springer-Verlag, New York, pp. 269-315 (1994). In a further embodiment, the intrabody preferably does not encode an operable secretory sequence and thus remains within the cell (see generally Marasco, W A, 1998, “Intrabodies: Basic Research and Clinical Gene Therapy Applications” Springer: New York).

Generation of intrabodies is well-known to the skilled artisan and is described, for example, in U.S. Pat. Nos. 6,004,940; 6,072,036; 5,965,371, which are incorporated by reference in their entireties herein. Further, the construction of intrabodies is discussed in Ohage and Steipe, 1999, J. Mol. Biol. 291: 1119-1128; Ohage et al., 1999, J. Mol. Biol. 291: 1129-1134; and Wirtz and Steipe, 1999, Protein Science 8: 2245-2250, which references are incorporated herein by reference in their entireties. Recombinant molecular biological techniques may also be used in the generation of intrabodies.

In one embodiment, intrabodies of the invention retain at least about 75% of the binding effectiveness of the complete antibody (ie., having the entire constant domain as well as the variable regions) to the antigen. More preferably, the intrabody retains at least 85% of the binding effectiveness of the complete antibody. Still more preferably, the intrabody retains at least 90% of the binding effectiveness of the complete antibody. Even more preferably, the intrabody retains at least 95% of the binding effectiveness of the complete antibody.

In producing intrabodies, polynucleotides encoding variable region for both the V_{H} and V_{L} chains of interest can be cloned by using, for example, hybridoma mRNA or splenic mRNA as a template for PCR amplification of such domains (Huse et al., 1989, Science 246: 1276). In one preferred embodiment, the polynucleotides encoding the V_{H} and V_{L} domains are joined by a polynucleotide sequence encoding a linker to make a single chain antibody (sFv). The sFv typically comprises a single peptide with the sequence V_{H}-linker-V_{L} or V_{L}-linker-V_{H}. The linker is chosen to permit the heavy chain and light chain to bind together in their proper conformational orientation (see for example, Huston, et al., 1991, Methods in Enzym. 203: 46-121, which is incorporated herein by reference). In a further embodiment, the linker can span the distance between its points of fusion to each of the variable domains (e.g., 3.5 nm) to minimize distortion of the native Fv conformation. In such an embodiment, the linker is a polypeptide of at least 5 amino acid residues, at least 10 amino acid residues, at least 15 amino acid residues, or greater. In a further embodiment, the linker should not cause a steric interference with the V_{H} and V_{L} domains of the combining site. In such an embodiment, the linker is 35 amino acids or less, 30 amino acids or less, or 25 amino acids or less. Thus, in a most preferred embodiment, the linker is between 15-25 amino acid residues in length. In a further embodiment, the linker is hydrophilic and sufficiently flexible such that the V_{H} and V_{L} domains can adopt the conformation necessary to detect antigen. Intrabodies can be generated with different linker sequences inserted between identical V_{H} and V_{L} domains. A linker with the appropriate properties for a particular pair of V_{H} and V_{L} domains can be determined empirically by assessing the degree of antigen binding for each. Examples of linkers include, but are not limited to, those sequences disclosed in Table 2.

TABLE 2

| | |
|---|---|
| Sequence | SEQ ID NO. |

| | |
|---|---|
| (Gly Gly Gly Gly Ser)₃ | SEQ ID NO:81 |
| Glu Ser Gly Arg Ser Gly Gly Gly Gly | SEQ ID NO:82 |
| Ser Gly Gly Gly Gly Ser | |
| Glu Gly Lys Ser Ser Gly Ser Gly Ser | SEQ ID NO:83 |
| Glu Ser Lys Ser Thr | |
| Glu Gly Lys Ser Ser Gly Ser Gly Ser | SEQ ID NO:84 |
| Glu Ser Lys Ser Thr Gln | |
| Glu Gly Lys Ser Ser Gly Ser Gly Ser | SEQ ID NO:85 |
| Glu Ser Lys Val Asp | |
| Gly Ser Thr Ser Gly Ser Gly Lys Ser | SEQ ID NO:86 |
| Ser Glu Gly Lys Gly | |
| Lys Glu Ser Gly Ser Val Ser Ser Glu | SEQ ID NO:87 |
| Gln Leu Ala Gln Phe Arg Ser Leu Asp | |
| Glu Ser Gly Ser Val Ser Ser Glu Glu | SEQ ID NO:88 |
| Leu Ala Phe Arg Ser Leu Asp | |

In one embodiment, intrabodies are expressed in the cytoplasm. In other embodiments, the intrabodies are localized to various intracellular locations. In such embodiments, specific localization sequences can be attached to the intrabody polypeptide to direct the intrabody to a specific location. Intrabodies can be localized, for example, to the following intracellular locations: endoplasmic reticulum (Munro et al., 1987, Cell 48: 899-907; Hangejorden et al., 1991, J. Biol. Chem. 266: 6015); nucleus (Lanford et al., 1986, Cell 46: 575; Stanton et al., 1986, PNAS 83: 1772; Harlow et al., 1985, Mol. Cell Biol. 5: 1605; Pap et al., 2002, Exp. Cell Res. 265: 288-93); nucleolar region (Seomi et al., 1990, J. Virology 64: 1803; Kubota et al., 1989, Biochem. Biophys. Res. Comm. 162: 963; Siomi et al., 1998, Cell 55: 197); endosomal compartment (Bakke et al., 1990, Cell 63: 707-716); mitochondrial matrix (Pugsley, A. P., 1989, “Protein Targeting”, Academic Press, Inc.); Golgi apparatus (Tang et al., 1992, J. Bio. Chem. 267: 10122-6); liposomes (Letourneur et al., 1992, Cell 69: 1183); peroxisome (Pap et al., 2002, Exp. Cell Res. 265: 288-93); trans Golgi network (Pap et al., 2002, Exp. Cell Res. 265: 288-93); and plasma membrane (Marchildon et al., 1984, PNAS 81: 7679-82; Henderson et al., 1987, PNAS 89: 339-43; Rhee et al., 1987, J. Virol. 61: 1045-53; Schultz et al., 1984, J. Virol. 133: 431-7; Ootsuyama et al., 1985, Jpn. J. Can. Res. 76: 1132-5; Ratner et al., 1985, Nature 313: 277-84). Examples of localization signals include, but are not limited to, those sequences disclosed in Table 3.

TABLE 3

| | | |
|---|---|---|
| Localization | Sequence | SEQ ID NO. |

| | | |
|---|---|---|
| endoplasmic reticulum | Lys Asp Glu Leu | SEQ ID NO:89 |
| endoplasmic reticulum | Asp Asp Glu Leu | SEQ ID NO:90 |
| endoplasmic reticulum | Asp Glu Glu Leu | SEQ ID NO:91 |
| endoplasmic reticulum | Gln Glu Asp Leu | SEQ ID NO:92 |
| endoplasmic reticulum | Arg Asp Glu Leu | SEQ ID NO:93 |
| nucleus | Pro Lys Lys Lys Arg Lys Val | SEQ ID NO:94 |
| nucleus | Pro Gln Lys Lys Ile Lys Ser | SEQ ID NO:95 |
| nucleus | Gln Pro Lys Lys Pro | SEQ ID NO:96 |
| nucleus | Arg Lys Lys Arg | SEQ ID NO:97 |
| nucleus | Lys Lys Lys Arg Lys | SEQ ID NO:98 |
| nucleolar region | Arg Lys Lys Arg Arg Gln Arg Arg Arg Ala | SEQ ID NO:99 |
| | His Gln | |
| nucleolar region | Arg Gln Ala Arg Arg Asn Arg Arg Arg Arg | SEQ ID NO:100 |
| | Trp Arg Glu Arg Gln Arg | |
| nucleolar region | Met Pro Leu Thr Arg Arg Arg Pro Ala Ala Ser | SEQ ID NO:101 |
| | Gln Ala Leu Ala Pro Pro Thr Pro | |
| endosomal compartment | Met Asp Asp Gln Arg Asp Leu Ile Ser Asn | SEQ ID NO:102 |
| | Asn Glu Gln Leu Pro | |
| mitochondrial matrix | Met Leu Phe Asn Leu Arg Xaa Xaa Leu Asn | SEQ ID NO:103 |
| | Asn Ala Ala Phe Arg His Gly His Asn Phe | |
| | Met Val Arg Asn Phe Arg Cys Gly Gln Pro | |
| | Leu Xaa | |
| peroxisome | Ala Lys Leu | SEQ ID NO:104 |
| trans Golgi network | Ser Asp Tyr Gln Arg Leu | SEQ ID NO:105 |
| plasma membrane | Gly Cys Val Cys Ser Ser Asn Pro | SEQ ID NO:106 |
| plasma membrane | Gly Gln Thr Val Thr Thr Pro Leu | SEQ ID NO:107 |
| plasma membrane | Gly Gln Glu Leu Ser Gln His Glu | SEQ ID NO:108 |
| plasma membrane | Gly Asn Ser Pro Ser Tyr Asn Pro | SEQ ID NO:109 |
| plasma membrane | Gly Val Ser Gly Ser Lys Gly Gln | SEQ ID NO:110 |
| plasma membrane | Gly Gln Thr Ile Thr Thr Pro Leu | SEQ ID NO:111 |
| plasma membrane | Gly Gln Thr Leu Thr Thr Pro Leu | SEQ ID NO:112 |
| plasma membrane | Gly Gln Ile Phe Ser Arg Ser Ala | SEQ ID NO:113 |
| plasma membrane | Gly Gln Ile His Gly Leu Ser Pro | SEQ ID NO:114 |
| plasma membrane | Gly Ala Arg Ala Ser Val Leu Ser | SEQ ID NO:115 |
| plasma membrane | Gly Cys Thr Leu Ser Ala Glu Glu | SEQ ID NO:116 |

V_{H} and V_{L} domains are made up of the immunoglobulin domains that generally have a conserved structural disulfide bond. In embodiments where the intrabodies are expressed in a reducing environment (e.g., the cytoplasm), such a structural feature cannot exist. Mutations can be made to the intrabody polypeptide sequence to compensate for the decreased stability of the immunoglobulin structure resulting from the absence of disulfide bond formation. In one embodiment, the V_{H} and/or V_{L} domains of the intrabodies contain one or more point mutations such that their expression is stabilized in reducing environments (see Steipe et al., 1994, J. Mol. Biol. 240: 188-92; Wirtz and Steipe, 1999, Protein Science 8: 2245-50; Ohage and Steipe, 1999, J. Mol. Biol. 291: 1119-28; Ohage et al., 1999, J. Mol. Biol. 291: 1129-34).

Intrabody Proteins as Therapeutics

In one embodiment, the recombinantly expressed intrabody protein is administered to a patient. Such an intrabody polypeptide must be intracellular to mediate a prophylactic or therapeutic effect. In this embodiment of the invention, the intrabody polypeptide is associated with a “membrane permeable sequence”. Membrane permeable sequences are polypeptides capable of penetrating through the cell membrane from outside of the cell to the interior of the cell. When linked to another polypeptide, membrane permeable sequences can also direct the translocation of that polypeptide across the cell membrane as well.

In one embodiment, the membrane permeable sequence is the hydrophobic region of a signal peptide (see, e.g., Hawiger, 1999, Curr. Opin. Chem. Biol. 3: 89-94; Hawiger, 1997, Curr. Opin. Immunol. 9: 189-94; U.S. Pat. Nos. 5,807,746 and 6,043,339, which are incorporated herein by reference in their entireties). The sequence of a membrane permeable sequence can be based on the hydrophobic region of any signal peptide. The signal peptides can be selected, e.g., from the SIGPEP database (see e.g., von Heijne, 1987, Prot. Seq. Data Anal. 1: 41-2; von Heijne and Abrahmsen, 1989, FEBS Lett. 224: 439-46). When a specific cell type is to be targeted for insertion of an intrabody polypeptide, the membrane permeable sequence is preferably based on a signal peptide endogenous to that cell type. In another embodiment, the membrane permeable sequence is a viral protein (e.g., Herpes Virus Protein VP22) or fragment thereof (see e.g., Phelan et al., 1998, Nat. Biotechnol. 16: 440-3). A membrane permeable sequence with the appropriate properties for a particular intrabody and/or a particular target cell type can be determined empirically by assessing the ability of each membrane permeable sequence to direct the translocation of the intrabody across the cell membrane. Examples of membrane permeable sequences include, but are not limited to, those sequences disclosed in Table 4.

TABLE 4

| | |
|---|---|
| Sequence | SEQ ID NO. |

| | |
|---|---|
| Ala Ala Val Ala Leu Leu Pro Ala Val | SEQ ID NO:117 |
| Leu Leu Ala Leu Leu Ala Pro | |
| Ala Ala Val Leu Leu Pro Val Leu Leu | SEQ ID NO:118 |
| Ala Ala Pro | |
| Val Thr Val Leu Ala Leu Gly Ala Leu | SEQ ID NO:119 |
| Ala Gly Val Gly Val Gly | |

In another embodiment, the membrane permeable sequence can be a derivative. In this embodiment, the amino acid sequence of a membrane permeable sequence has been altered by the introduction of amino acid residue substitutions, deletions, additions, and/or modifications. For example, but not by way of limitation, a polypeptide may be modified, e.g., by glycosylation, acetylation, pegylation, phosphorylation, amidation, derivatization by known protecting/blocking groups, proteolytic cleavage, linkage to a cellular ligand or other protein, etc. A derivative of a membrane permeable sequence polypeptide may be modified by chemical modifications using techniques known to those of skill in the art, including, but not limited to specific chemical cleavage, acetylation, formylation, metabolic synthesis of tunicamycin, etc. Further, a derivative of a membrane permeable sequence polypeptide may contain one or more non-classical amino acids. In one embodiment, a polypeptide derivative possesses a similar or identical function as an unaltered polypeptide. In another embodiment, a derivative of a membrane permeable sequence polypeptide has an altered activity when compared to an unaltered polypeptide. For example, a derivative membrane permeable sequence polypeptide can translocate through the cell membrane more efficiently or be more resistant to proteolysis.

The membrane permeable sequence can be attached to the intrabody in a number of ways. In one embodiment, the membrane permeable sequence and the intrabody are expressed as a fusion protein. In this embodiment, the nucleic acid encoding the membrane permeable sequence is attached to the nucleic acid encoding the intrabody using standard recombinant DNA techniques (see e.g., Rojas et al., 1998, Nat. Biotechnol. 16: 370-5). In a further embodiment, there is a nucleic acid sequence encoding a spacer peptide placed in between the nucleic acids encoding the membrane permeable sequence and the intrabody. In another embodiment, the membrane permeable sequence polypeptide is attached to the intrabody polypeptide after each is separately expressed recombinantly (see e.g., Zhang et al., 1998, PNAS 95: 9184-9). In this embodiment, the polypeptides can be linked by a peptide bond or a non-peptide bond (e.g. with a crosslinking reagent such as glutaraldehyde or a thiazolidino linkage see e.g., Hawiger, 1999, Curr. Opin. Chem. Biol. 3: 89-94) by methods standard in the art.

The administration of the membrane permeable sequence-intrabody polypeptide can be by parenteral administration, e.g., by intravenous injection including regional perfusion through a blood vessel supplying the tissues(s) or organ(s) having the target cell(s), or by inhalation of an aerosol, subcutaneous or intramuscular injection, topical administration such as to skin wounds and lesions, direct transfection into, e.g., bone marrow cells prepared for transplantation and subsequent transplantation into the subject, and direct transfection into an organ that is subsequently transplanted into the subject. Further administration methods include oral administration, particularly when the complex is encapsulated, or rectal administration, particularly when the complex is in suppository form. A pharmaceutically acceptable carrier includes any material that is not biologically or otherwise undesirable, ie., the material may be administered to an individual along with the selected complex without causing any undesirable biological effects or interacting in a deleterious manner with any of the other components of the pharmaceutical composition in which it is contained.

Conditions for the administration of the membrane permeable sequence-intrabody polypeptide can be readily be determined, given the teachings in the art (see e.g., Remington's Pharmaceutical Sciences, 18^{th} Ed., E. W. Martin (ed.), Mack Publishing Co., Easton, Pa. (1990)). If a particular cell type in vivo is to be targeted, for example, by regional perfusion of an organ or section of artery/blood vessel, cells from the target tissue can be biopsied and optimal dosages for import of the complex into that tissue can be determined in vitro to optimize the in vivo dosage, including concentration and time length. Alternatively, culture cells of the same cell type can also be used to optimize the dosage for the target cells in vivo.

Intrabody Gene Therapy as Therapeutic

In another embodiment, a polynucleotide encoding an intrabody is administered to a patient (e.g., as in gene therapy). In this embodiment, methods as described in Section 5.3. can be used to administer the polynucleotide of the invention.

5.1.1.2. BiTE Molecules

In a specific embodiment, antibodies for use in the methods of the invention are bispecific T cell engagers (BiTEs). Bispecific T cell engagers (BiTE) are bispecific antibodies that can redirect T cells for antigen-specific elimination of targets. A BiTE molecule has an antigen-binding domain that binds to a T cell antigen (e.g. CD3) at one end of the molecule and an antigen binding domain that will bind to an antigen on the target cell. A BiTE molecule was described in International Publication No. WO 99/54440, which is herein incorporated by reference. This publication describes a novel single-chain multifunctional polypeptide that comprises binding sites for the CD19 and CD3 antigens (CD19×CD3). This molecule was derived from two antibodies, one that binds to CD19 on the B cell and an antibody that binds to CD3 on the T cells. The variable regions of these different antibodies are linked by a polypeptide sequence, thus creating a single molecule. Also described, is the linking of the heavy chain (V_{H}) and light chain (V_{L}) variable domains with a flexible linker to create a single chain, bispecific antibody.

In an embodiment of this invention, an antibody or ligand that immunospecifically binds a polypeptide of interest (e.g., EphA2 and/or EphA4) will comprise a portion of the BiTE molecule. For example, the V_{H} and/or V_{L} (preferably a scFV) of an antibody that binds a polypeptide of interest (e.g., EphA2 and/or EphA4) can be fused to an anti-CD3 binding portion such as that of the molecule described above, thus creating a BiTE molecule that targets the polypeptide of interest (e.g., EphA2 and/or EphA4). In addition to the heavy and/or light chain variable domains of antibody against a polypeptide of interest (e.g., EphA2 and/or EphA4), other molecules that bind the polypeptide of interest (e.g., EphA2 and/or EphA4) can comprise the BiTE molecule, for example receptors (e.g., EphA2 and/or EphA4). In another embodiment, the BiTE molecule can comprise a molecule that binds to other T cell antigens (other than CD3). For example, ligands and/or antibodies that immunospecifically bind to T-cell antigens like CD2, CD4, CD8, CD11a, TCR, and CD28 are contemplated to be part of this invention. This list is not meant to be exhaustive but only to illustrate that other molecules that can immunospecifically bind to a T cell antigen can be used as part of a BiTE molecule. These molecules can include the VH and/or VL portions of the antibody or natural ligands (for example LFA3 whose natural ligand is CD3).

5.1.1.3 Agonistic Molecules

Any molecule that agonizing EphA2 or EphA4 (i.e., elicit EphA2 or EphA4 phosphorylation) can be used in accordance with the present invention. In one embodiment, EphA2 or EphA4 ligands, e.g., Ephrin-A1 is used. Ligand binding leads to EphA2/EphA4 receptor dimerization, activation of the kinase domain, and autophosphorylation. In a preferred embodiment, Ephrin-A1 Fc domain or Ephrin-A1 Fc fused to another peptide is used. In another embodiment, proteins (including peptides and polypeptides) that preferably agonize (i.e., elicit EphA2 phosphorylation) as well as immunospecifically bind to the EphA2/EphA4 receptor are used in accordance with the present invention. When agonized, EphA2 or EphA4 becomes phosphorylated and then subsequently degraded. Any method known in the art to assay either the level of EphA2/EphA4 phosphorylation, activity, or expression can be used to assay candidate EphA2 agonistic molecules or candidate EphA4 agonistic molecules to determine their agonistic activity.

In a specific embodiment, an agonistic molecule is an anti-EphA2 antibody EA2-EA5 (see U.S. patent application Ser. No. 10/436,783, entitled “EphA2 Agonistic Monoclonal Antibodies and Methods of Use,” filed May 12, 2003, which is incorporated by reference herein in its entirety).

In another specific embodiment, an agonistic molecule is an anti-EphA4 antibody such as EA44 (see U.S. patent application Ser. No. 10/863,729, entitled “Use of EphA4 and Modulators of EphA4 For Diagnosis, Treatment and Prevention of Cancer,” filed Jun. 7, 2004, which is incoporated by reference herein in its entirety).

5.1.2 Proteins that Preferentially Bind EphA2 or EphA4 Epitopes Exposed on Cancer Cells

Proteins (e.g., antibodies or fragments thereof) that preferably bind to EphA2 or EphA4 epitopes exposed on cancer cells (e.g., cells overexpressing EphA2 or EphA4 and/or cells with substantial EphA2 or EphA4 that is not bound to ligand) but not to non-cancer cells or cells where EphA2 or EphA4 is bound to a ligand can also be used in accordance with the present invention. In this embodiment, proteins of the invention are proteins directed to an EphA2 or EphA4 epitope not exposed on non-cancer cells but exposed on cancer cells. Differences in EphA2 or EphA4 membrane distribution between non-cancer cells and cancer cells expose certain epitopes on cancer cells that are not exposed on non-cancer cells. For example, normally EphA2 or EphA4 is bound to its ligand, e.g., EphrinA1, and localizes at areas of cell-cell contacts. However, cancer cells generally display decreased cell-cell contacts as well as overexpress EphA2 or EphA4 in excess of its ligand. Thus, in cancer cells, there is an increased amount of unbound EphA2 or EphA4 that is not localized to cell-cell contacts. As such, in one embodiment, a protein that preferentially binds unbound, unlocalized EphA2 or EphA4 can be used in accordance with the present invention.

Any method known in the art to determine candidate EphA2-binding protein or EphA4-binding protein binding/localization on a cell can be used to screen candidate proteins for desirable binding properties. In a one embodiment, immunofluorescence microscopy is used to determine the binding characteristics of an EphA2-binding protein or an EphA4-binding protein. Standard techniques can be used to compare the binding of an EphA2 protein or an EphA4 protein binding to cells grown in vitro. In a specific embodiment, protein binding to cancer cells is compared to protein binding to non-cancer cells. An exposed EphA2/EphA4 epitope peptide binds poorly to non-cancer cells but binds well to cancer cells. In another specific embodiment, protein binding to non-cancer dissociated cells (e.g., treated with a calcium chelator such as EGTA) is compared to protein binding to non-cancer cells that have not been dissociated. An exposed EphA2/EphA4 epitope peptide binds poorly non-cancer cells that have not been dissociated but binds well to dissociated non-cancer cells. In one embodiment, a protein that preferentially bind EphA2 or EphA4 epitopes exposed on cancer cells prevents LMW-PTP from binding phosphorylated EphA2 or EphA4. In another embodiment, a protein that preferentially bind EphA2 or EphA4 epitopes exposed on cancer cells prevents LMW-PTP from binding EphA2 or EphA4, regardless whether EphA2 or EphA4 is phosphorylated. In a specific embodiment, a protein that preferentially bind EphA2 or EphA4 prevents LMW-PTP from binding the substrate-binding site on EphA2 or EphA4, even though LMW-PTP may be able to bind to a non-substrate binding site on EphA2 or EphA4.

In another embodiment, flow cytometry is used to determine the binding characteristics of an EphA2-binding protein or an EphA4-binding protein. In this embodiment, EphA2 or EphA4 may or may not be crosslinked to its ligand, e.g., Ephrin A1. An exposed EphA2 or EphA4 epitope peptide binds poorly crosslinked EphA2/EphA4 but binds well to uncrosslinked EphA2/EphA4.

In another embodiment, cell-based or immunoassays are used to determine the binding characteristics of an EphA2-binding protein or EphA4-binding protein. In this embodiment, for example, candidates can be assayed for activity to compete for binding to EphA2 or EphA4 to a known EphA2/EphA4 binding protein, e.g., an EphA2 or EphA4 ligand (e.g., Ephrin A1) or an anti-EphA2 antibody (e.g., EA2 or EA5) or an anti-EphA4 antibody (e.g., EA44). In a specific embodiment, candidates are assayed for activity to compete for binding to EphA2 to EA2, EA5 or B2D6. The EphA2/EphA4 binding protein used in this assay can be soluble protein (e.g., recombinantly expressed) or expressed on a cell so that it is anchored to the cell.

5.1.3 Cancer Cell Phenotype Inhibiting Agents

Agents that preferably inhibit (and preferably reduce) cancer cell colony formation in, for example, soft agar, or tubular network formation in a three-dimensional basement membrane or extracellular matrix preparation as well as immunospecifically bind to the EphA2 or EphA4 receptor can be used in accordance with the present invention. One of skill in the art can assay candidate EphA2/EphA4 agents for their ability to inhibit such behavior (see, e.g., Section 6.2 infra). Metastatic tumor cells suspended in soft agar form colonies while benign tumors cells do not. Colony formation in soft agar can be assayed as described in Zelinski et al. (2001, Cancer Res. 61: 2301-6, incorporated herein by reference in its entirety). Agents to be assayed for agonistic activity can be included in bottom and top agar solutions. Metastatic tumor cells can be suspended in soft agar and allowed to grow. EphA2 or EphA4 cancer cell phenotype inhibiting peptides will inhibit colony formation.

Another behavior specific to metastatic cells that can be used to identify cancer cell phenotype inhibiting agents is tubular network formation within a three-dimensional microenvironment, such as MATRIGEL™. Normally, cancer cells quickly assemble into tubular networks that progressively invade all throughout the MATRIGEL™. In the presence of an EphA2/EphA4 cancer cell phenotype inhibiting agent, cancer cells assemble into spherical structures that resemble the behavior of differentiated, non-cancerous cells. Accordingly, EphA2/EphA4 cancer cell phenotype inhibiting agents can be identified by their ability to inhibit tubular network formation of cancer cells.

Any other method that detects an increase in contact inhibition of cell proliferation (e.g., reduction of colony formation in a monolayer cell culture) may also be used to identify cancer cell phenotype inhibiting agents.

In addition to inhibiting cancer cell colony formation, cancer cell phenotype inhibiting agents may also cause a reduction or elimination of colonies when added to already established colonies of cancer cells by cell killing, e.g., by necrosis or apoptosis. Methods for assaying for necrosis and apoptosis are well known in the art.

5.1.4 Antibodies with Low K_{off} Rates

The binding affinity of a monoclonal antibody to EphA2 or EphA4 or a fragment thereof and the off-rate of a monoclonal antibody-EphA2 or a monoclonal antibody-EphA4 interaction can be determined by competitive binding assays. One example of a competitive binding assay is a radioimmunoassay comprising the incubation of labeled EphA2 or EphA4 (e.g., ³H or ¹²⁵I) with the monoclonal antibody of interest in the presence of increasing amounts of unlabeled EphA2 or EphA4, and the detection of the monoclonal antibody bound to the labeled EphA2 or EphA4. The affinity of a monoclonal antibody for an EphA2 or EphA4 and the binding off-rates can be determined from the data by scatchard plot analysis. Competition with a second monoclonal antibody can also be determined using radioimmunoassays. In this case, EphA2 is incubated with a monoclonal antibody conjugated to a labeled compound (e.g., ³H or ¹²⁵I) in the presence of increasing amounts of a second unlabeled monoclonal antibody.

In a preferred embodiment, a candidate EphA2 or EphA4 antibody may be assayed using any surface plasmon resonance based assays known in the art for characterizing the kinetic parameters of the EphA2-EphA2 antibody interaction or the EphA4-EphA4 antibody interaction. Any SPR instrument commercially available including, but not limited to, BIACORE Instruments, available from Biacore AB (Uppsala, Sweden); IAsys instruments available form Affinity Sensors (Franklin, Mass.); IBIS system available from Windsor Scientific Limited (Berks, UK), SPR-CELLIA systems available from Nippon Laser and Electronics Lab (Hokkaido, Japan), and SPR Detector Spreeta available from Texas Instruments (Dallas, Tex.) can be used in the instant invention. For a review of SPR-based technology see Mullet et al., 2000, Methods 22: 77-91; Dong et al., 2002, Review in Mol. Biotech., 82: 303-23; Fivash et al., 1998, Current Opinion in Biotechnology 9: 97-101; Rich et al., 2000, Current Opinion in Biotechnology 11: 54-61; all of which are incorporated herein by reference in their entirety. Additionally, any of the SPR instruments and SPR based methods for measuring protein-protein interactions described in U.S. Pat. Nos. 6,373,577; 6,289,286; 5,322,798; 5,341,215; 6,268,125 are contemplated in the methods of the invention, all of which are incorporated herein by reference in their entirety.

Briefly, SPR based assays involve immobilizing a member of a binding pair on a surface, and monitoring its interaction with the other member of the binding pair in solution. SPR is based on measuring the change in refractive index of the solvent near the surface that occurs upon complex formation or dissociation. The surface onto which the immobilization occur is the sensor chip, which is at the heart of the SPR technology; it consists of a glass surface coated with a thin layer of gold and forms the basis for a range of specialized surfaces designed to optimize the binding of a molecule to the surface. A variety of sensor chips are commercially available especially from the companies listed supra, all of which may be used in the methods of the invention. Examples of sensor chips include those available from BIAcore AB, Inc., e.g., Sensor Chip CM5, SA, NTA, and HPA. A molecule of the invention may be immobilized onto the surface of a sensor chip using any of the immobilization methods and chemistries known in the art, including but not limited to direct covalent coupling via amine groups, direct covalent coupling via sulfhydryl groups, biotin attachment to avidin coated surface, aldehyde coupling to carbohydrate groups and attachment through the histidine tag with NTA chips.

In a more preferred embodiment, BIACORE™ kinetic analysis is used to determine the binding on and off rates of monoclonal antibodies to EphA2 or EphA4 (see, e.g., Section 6.7 infra). BIACORE™ kinetic analysis comprises analyzing the binding and dissociation of a monoclonal antibody from chips with immobilized EphA2/EphA4 or fragment thereof on their surface.

Once an entire data set is collected, the resulting binding curves are globally fitted using computer algorithms supplied by the manufacturer, BIAcore, Inc. (Piscataway, N.J.). These algorithms calculate both the Kₒₙ and K_{off}, from which the apparent equilibrium binding constant, K_{D} is deduced as the ratio of the two rate constants (i.e., K_{off}/Kₒₙ). More detailed treatments of how the individual rate constants are derived can be found in the BIAevaluaion Software Handbook (BIAcore, Inc., Piscataway, N.J.). The analysis of the generated data may be done using any method known in the art. For a review of the various methods of interpretation of the kinetic data generated see Myszka, 1997, Current Opinion in Biotechnology 8: 50-7; Fisher et al., 1994, Current Opinion in Biotechnology 5: 389-95; O'Shannessy, 1994, Current Opinion in Biotechnology, 5: 65-71; Chaiken et al., 1992, Analytical Biochemistry, 201: 197-210; Morton et al., 1995, Analytical Biochemistry 227: 176-85; O'Shannessy et al., 1996, Analytical Biochemistry 236: 275-83; all of which are incorporated herein by reference in their entirety.

The invention encompasses antibodies that immunospecifically bind to EphA2 or and preferably have a K_{off} rate 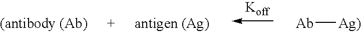
off less than 3×10⁻³ s⁻¹, more preferably less than 1×10⁻³ s⁻¹. In other embodiments, the antibodies of the invention immunospecifically bind to EphA2 or EphA4 and have a K_{off} of less than 5×10⁻³ s⁻¹, less than 10⁻³ s⁻¹, less than 8×10⁻⁴ s⁻¹, less than 5×10⁻⁴ s⁻¹, less than 10⁻⁴ s⁻¹, less than 9×10⁻⁵ s⁻¹, less than 5×10⁻⁵ s⁻¹, less than 10⁻⁵ s⁻¹, less than 5×10⁻⁶ s⁻¹, less than 10⁻⁶ s⁻¹, less than 5×10⁻⁷ s⁻¹, less than 10⁻⁷ s⁻¹, less than 5×10⁻⁸ s⁻¹, less than 10⁻⁸ s⁻¹, less than 5×10⁻⁹ s⁻¹, less than 10⁻⁹ s⁻¹, or less than 10⁻¹⁰ s⁻¹.

Thus, the invention encompasses methods of assaying and screening for EphA2 or EphA4 antibodies of the invention by incubating antibodies that specifically bind EphA2 or EphA4, particularly that bind the extracellular domain of EphA2 or EphA4, with cells that express EphA2 or EphA4, particularly cancer cells, preferably metastatic cancer cells, that overexpress EphA2 or EphA4 (relative to non-cancer cells of the same cell type) and then assaying for an increase in EphA2 or EphA4 phosphorylation and/or EphA2 or EphA4 degradation (for agonistic antibodies), or reduction in colony formation in soft agar or tubular network formation in three-dimensional basement membrane or extracellular matrix preparation (for cancer cell phenotype inhibiting antibodies), or increased peptide binding to cancer cells as compared to non-cancer cells by e.g., immunofluorescence (for exposed EphA2 or EphA4 epitope peptides) thereby identifying an EphA2 or EphA4 peptide of the invention.

5.1.5 Nucleic Acid Molecules

Nucleic acid molecules specific for LMW-PTP, EphA2 or EphA4, particularly those that inhibit or encode one or more moieties that inhibit LMW-PTP, EphA2 or EphA4 expression, can also be used in methods of the invention.

5.1.5.1 Antisense

The present invention encompasses antisense nucleic acid molecules, i.e., molecules which are complementary to all or part of a sense nucleic acid encoding LMW-PTP, EphA2 or EphA4, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand, or to only a portion thereof, e.g., all or part of the protein coding region (or open reading frame). An antisense nucleic acid molecule can be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids. Antisense nucleic acid molecules may be determined by any method known in the art, using the nucleotide sequences in publicly available databases such as GenBank. For example, using the nucleotide sequence of human EphA2 (GenBank accession no. NM_{—}004431.2) or the nucleotide sequence of human EphA4 (GenBank accession no. NM_{—}004438.3). In one embodiment, the antisense nucleic acid molecule is 5′-CCAGCAGTACCGCTTCCTTGCCCTGCGGCCG-3′ (SEQ ID NO:120). In a specific embodiment, an EphA2 antisense nucleic acid molecule is not 5′-CCAGCAGTACCACTTCCTTGCCCTGCGCCG-3′ (SEQ ID NO:121) and/or 5′-GCCGCGTCCCGTTCCTTCACCATGACGACC-3′ (SEQ ID NO:122). In another specific embodiment, an EphA2 antisense nucleic acid moleucle is not 5′-CCAGCAGTACCGCTTCCTTGCCCTGCGGCCG-3′ (SEQ ID NO:123) and/or 5′-GCCGCGTCCCGTTCCTTCACCATGACGACC-3′ (SEQ ID NO:124). In certain embodiments, an EphA2 or EphA4 binding moiety of the invention is not an EphA2 antisense nucleic acid molecule.

An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, β-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, β-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, e.g., LMW-PTP, EphA2 or EphA4).

The antisense nucleic acid molecules of the invention are typically administered to a subject or generated in situ such that they hybridize with or bind to cellular mRNA and/or genomic DNA encoding a selected polypeptide of the invention to thereby inhibit expression, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or peptides which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al., 1987, Nucleic Acids Res. 15: 6625). The antisense nucleic acid molecule can also comprise a 2′-o-methylribonucleotide (Inoue et al., 1987, Nucleic Acids Res. 15: 6131) or a chimeric RNA-DNA analogue (Inoue et al., 1987, FEBS Lett. 215: 327).

5.1.5.2 Ribozymes

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes; described in Haselhoff and Gerlach, 1988, Nature 334: 585-591) can be used to catalytically cleave mRNA transcripts to thereby inhibit translation of the protein encoded by the mRNA. A ribozyme having specificity for a nucleic acid molecule encoding LMW-PTP, EphA2 or EphA4 can be designed based upon the nucleotide sequence of LMW-PTP, EphA2 and EphA4. For example, a derivative of a Tetrahymena L-19 IVS RNA can be constructed in which the nucleotide sequence of the active site is complementary to the nucleotide sequence to be cleaved in U.S. Pat. Nos. 4,987,071 and 5,116,742. Alternatively, an mRNA encoding a polypeptide of the invention can be used to select a catalytic RNA having a specific ribonuclease activity from a pool of RNA molecules. See, e.g., Bartel and Szostak, 1993, Science 261: 1411.

5.1.5.3 RNA Interference

In certain embodiments, an RNA interference (RNAi) molecule is used to inhibit LMW-PTP, EphA2 and/or EphA4 expression or activity. RNA interference (RNAi) is defined as the ability of double-stranded RNA (dsRNA) to suppress the expression of a gene corresponding to its own sequence. RNAi is also called post-transcriptional gene silencing or PTGS. Since the only RNA molecules normally found in the cytoplasm of a cell are molecules of single-stranded mRNA, the cell has enzymes that recognize and cut dsRNA into fragments containing 21-25 base pairs (approximately two turns of a double helix). The antisense strand of the fragment separates enough from the sense strand so that it hybridizes with the complementary sense sequence on a molecule of endogenous cellular mRNA. This hybridization triggers cutting of the mRNA in the double-stranded region, thus destroying its ability to be translated into a polypeptide. Introducing dsRNA corresponding to a particular gene thus knocks out the cell's own expression of that gene in particular tissues and/or at a chosen time.

Double-stranded (ds) RNA can be used to interfere with gene expression in mammals (Wianny & Zernicka-Goetz, 2000, Nature Cell Biology 2: 70-75; incorporated herein by reference in its entirety). dsRNA is used as inhibitory RNA or RNAi of the function of EphA2 to produce a phenotype that is the same as that of a null mutant of EphA2 (Wianny & Zernicka-Goetz, 2000, Nature Cell Biology 2: 70-75).

5.1.5.4 Aptamers

In specific embodiments, the invention provides aptamers of LMW-PTP, EphA2 and EphA4. As is known in the art, aptamers are macromolecules composed of nucleic acid (e.g., RNA, DNA) that bind tightly to a specific molecular target (e.g., LMW-PTP, EphA2 or EphA4 proteins, LMW-PTP, EphA2 or EphA4 polypeptides and/or LMW-PTP, EphA2 or EphA4 epitopes as described herein). A particular aptamer may be described by a linear nucleotide sequence and is typically about 15-60 nucleotides in length. The chain of nucleotides in an aptamer form intramolecular interactions that fold the molecule into a complex three-dimensional shape, and this three-dimensional shape allows the aptamer to bind tightly to the surface of its target molecule. Given the extraordinary diversity of molecular shapes that exist within the universe of all possible nucleotide sequences, aptamers may be obtained for a wide array of molecular targets, including proteins and small molecules. In addition to high specificity, aptamers have very high affinities for their targets (e.g., affinities in the picomolar to low nanomolar range for proteins). Aptamers are chemically stable and can be boiled or frozen without loss of activity. Because they are synthetic molecules, they are amenable to a variety of modifications, which can optimize their function for particular applications. For in vivo applications, aptamers can be modified to dramatically reduce their sensitivity to degradation by enzymes in the blood. In addition, modification of aptamers can also be used to alter their biodistribution or plasma residence time.

Selection of aptamers that can bind to LMW-PTP, EphA2 or EphA4 or a fragment thereof can be achieved through methods known in the art. For example, aptamers can be selected using the SELEX (Systematic Evolution of Ligands by Exponential Enrichment) method (Tuerk and Gold, 1990, Science 249: 505-510, which is incorporated by reference herein in its entirety). In the SELEX method, a large library of nucleic acid molecules (e.g., 10¹⁵ different molecules) is produced and/or screened with the target molecule (e.g., LMW-PTP, EphA2 or EphA4 proteins, LMW-PTP, EphA2 or EphA4 polypeptides and/or LMW-PTP, EphA2 or EphA4 epitopes or fragments thereof as described herein). The target molecule is allowed to incubate with the library of nucleotide sequences for a period of time. Several methods can then be used to physically isolate the aptamer target molecules from the unbound molecules in the mixture and the unbound molecules can be discarded. The aptamers with the highest affinity for the target molecule can then be purified away from the target molecule and amplified enzymatically to produce a new library of molecules that is substantially enriched for aptamers that can bind the target molecule. The enriched library can then be used to initiate a new cycle of selection, partitioning, and amplification. After 5-15 cycles of this selection, partitioning and amplification process, the library is reduced to a small number of aptamers that bind tightly to the target molecule. Individual molecules in the mixture can then be isolated, their nucleotide sequences determined, and their properties with respect to binding affinity and specificity measured and compared. Isolated aptamers can then be further refined to eliminate any nucleotides that do not contribute to target binding and/or aptamer structure (i.e., aptamers truncated to their core binding domain). See, e.g., Jayasena, 1999, Clin. Chem. 45: 1628-1650 for review of aptamer technology, the entire teachings of which are incorporated herein by reference).

In particular embodiments, the aptamers of the invention have the binding specificity and/or functional activity described herein for the antibodies of the invention. Thus, for example, in certain embodiments, the present invention is drawn to aptamers that have the same or similar binding specificity as described herein for the antibodies of the invention (e.g., binding specificity for LMW-PTP, EphA2 or EphA4 polypeptide, fragments of vertebrate LMW-PTP, EphA2 or EphA4 polypeptides, epitopic regions of vertebrate EphA2 or EphA4 polypeptides (e.g., epitopic regions of LMW-PTP, EphA2 or EphA4 that are bound by the antibodies of the invention). In particular embodiments, the aptamers of the invention can bind to a LMW-PTP, EphA2 or EphA4 polypeptide and inhibit one or more activities of the LMW-PTP, EphA2 or EphA4 polypeptide.

5.1.5.5 Gene Therapy

In a specific embodiment, nucleic acids that reduce LMW-PTP, EphA2 or EphA4 expression (e.g., LMW-PTP, EphA2 or EphA4 antisense nucleic acids or LMW-PTP, EphA2 or EphA4 dsRNA) are administered to treat, prevent or manage a hyperproliferative disease, particular cancer, by way of gene therapy. Gene therapy refers to therapy performed by the administration to a subject of an expressed or expressible nucleic acid. In this embodiment of the invention, the antisense nucleic acids are produce and mediate a prophylactic or therapeutic effect.

Any of the methods for gene therapy available in the art can be used according to the present invention. Exemplary methods are described below.

For general reviews of the methods of gene therapy, see Goldspiel et al., 1993, Clinical Pharmacy 12: 488; Wu and Wu, 1991, Biotherapy 3: 87; Tolstoshev, 1993, Ann. Rev. Pharmacol. Toxicol. 32: 573; Mulligan, 1993, Science 260: 926-932; and Morgan and Anderson, 1993, Ann. Rev. Biochem. 62: 191; May, 1993, TIBTECH 11: 155. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, a composition of the invention comprises LMW-PTP, EphA2 or EphA4 nucleic acids that reduce LMW-PTP, EphA2 or EphA4 expression, said nucleic acids being part of an expression vector that expresses the nucleic acid in a suitable host. In particular, such nucleic acids have promoters, preferably heterologous promoters, said promoter being inducible or constitutive, and, optionally, tissue-specific. In another particular embodiment, nucleic acid molecules are used in which the nucleic acid that reduces LMW-PTP, EphA2 or EphA4 expression and any other desired sequences are flanked by regions that promote homologous recombination at a desired site in the genome, thus providing for intrachromosomal expression of the nucleic acids that reduce LMW-PTP, EphA2 or EphA4 expression (Koller and Smithies, 1989, PNAS 86: 8932; Zijlstra et al., 1989, Nature 342: 435).

Delivery of the nucleic acids into a subject may be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy. For detailed description of delivery methods, see Section 5.3., infra.

5.1.6 Other Kinase Inhibitors

In one embodiment, other kinase inhibitors that are capable of inhibiting or reducing the expression of EphA2 or EphA4 can be used in methods of the invention. Such kinase inhibitors include, but are not limited to, inhibitors of Ras, and inhibitors of certain other oncogenic receptor tyrosine kinases such as EGFR and HER2. Non-limiting examples of such inhibitors are disclosed in U.S. Pat. Nos. 6,462,086; 6,130,229; 6,638,543; 6,562,319; 6,355,678; 6,656,940; 6,653,308; 6,642,232, and 6,635,640, each of which is incorporated herein by reference in its entirety. In a particular embodiment, the the kinase inhibitors inhibit or reduce EphA2 and/or EphA4 expression by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline) in an assay described herein or known in the art (e.g., RT-PCR, a Northern blot or an immunoassay such as an ELISA, Western blot).

5.2. EphA2 or EphA4 Targeting Moieties

In accordance with the present invention, moieties that bind to cells expressing LMW-PTP, EphA2, and/or EphA4 can be used to target agents that inhibit LMW-PTP expression and/or activity to such cells. In some preferred embodiments, the targeting moieties that bind to EphA2 are used. In other preferred embodiments, the targeting moieties that bind to EphA4 are used. Non-limiting examples of EphA2 or EphA4 targeting moieties are all or an EphA2/EphA4 binding portion of its ligand, e.g., Ephrin A1, and an anti-EphA2 or anti-EphA4 antibody (particularly that bind the extracellular domain, i.e., EphA2 or EphA4 on the cell surface and disclosed in Section 5.1.1, supra). Preferably, moieties bind to EphA2 or EphA4 on cancer cells (e.g., EphA2 or EphA4 not bound to ligand) rather than EphA2 or EphA4 on non-cancer cells (e.g., EphA2 or EphA4 bound to ligand) are used in accordance with the present invention. In a preferred embodiment, Ephrin A1 Fc or Ephrin A1 Fc fused to another peptide is used in accordance with the present invention. In a specific embodiment of the invention, the EphA2 or EphA4 targeting moiety is not Ephrin A1 or a fragment thereof, or is not Ephrin A1 Fc. In specific embodiments, the EphA2 and/or EphA4 targeting moieties bind to EphA2 and/or EphA4 on hyperproliferative cells, particularly cancer cells, as opposed to EphA2 and/or EphA4 on non-hyperproliferative (i.e., non-cancer cells) or non-EphA2 and/or non-EphA4 antigens, with at least, 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative higher relative to a control (e.g., phosphate buffered saline or bovine serum albumin) as determined by any assay known to those skilled in the art (e.g., a BIAcore assay).

In a specific embodiment, an EphA2 or EphA4 targeting moiety used in the compositions and methods of the invention is any one of the peptides disclosed in Table 1 of U.S. Patent Publication No. U.S. 2004/0180823 A1 (Sep. 16, 2004) by Pasquale et al or International Publication No. WO 2004/028551 A1 (Apr. 8, 2004) by Pasquale et al. that bind to EphA2 and/or EphA4. In another specific embodiment, a targeting moiety of the invention is not any of the peptides disclosed in U.S. Patent Publication No. U.S. 2004/0180823 A1 (Sep. 16, 2004) by Pasquale et al or International Publication No. WO 2004/028551 A1 (Apr. 8, 2004) by Pasquale et al.

The agents that inhibit EphA2 or EphA4 expression or function as described in Section 5.1 may preferentially bind to EphA2 or EphA4, and thus can also be used as targeting moieties to direct another substance (such as a delivery vehicle or another compound) to cells that expressing LMW-PTP, EphA2, and/or EphA4.

A nucleic acid can be a target moiety and used in vivo for cell specific uptake and expression, by targeting a specific receptor, preferably EphA2 or EphA4.

In addition to those described in Section 5.1, any substance that has preference for cancer cells or non-cancer hyperproliferative cells that express EphA2 or EphA4 can be used to direct a therapeutic or prophylactic agent to such cells in accordance with the present invention.

For example, targeting moieties can be, but are not limited to, antibodies or fragments thereof, receptors, ligands, peptides and other molecules that bind to cells of, or in the vicinity of, the target tissue. An antibody targeting moiety may be an intact (whole) molecule, a fragment thereof, or a functional equivalent thereof. Examples of antibody fragments are F(ab′)2, Fab′, Fab, Fv fragments and single chain Fvs, which may be produced by conventional methods or by genetic or protein engineering. Preferably, a targeting moiety in accordance with the present invention specifically targets EphA2 or EphA4. EphA2 monoclonal antibodes are disclosed in the U.S. patent application Ser. No. 10/436,782 (entitled “EphA2 Monoclonal Antibodies and Methods of Use Thereof,” filed May 12, 2003) and Ser. No. 10/436,783 (entitled “EphA2 Agonistic Monoclonal Antibodies and Methods of Use Thereof,” filed May 12, 2003), each of which is incorporated herein by reference in its entirety. EphA4 monoclonal antibodies are disclosed in the U.S. Non-Provisional application Ser. No. 10/863,729 (entitled “Use of EphA4 and Modulator of EphA4 for Diagnosis, Treatment and Prevention of Cancer,” filed Jun. 7, 2004), which is incorporated by reference herein in its entirety.

In a specific embodiment, a targeting moiety is any polypeptide (or fragment thereof) that is a natural ligand of EphA2 (e.g., Ephrin A1) or EphA4 (e.g., Ephrin A1, -A2, -A3, -A4, -A5, -B2 and -B3). The amino acid sequences for Ephrin A1-B3, may be found, for example, in any publicly available database, such as GenBank.

In a specific embodiment, a targeting moiety of the invention is an EphrinA1 polypeptide. In a specific embodiment, an targeting moiety of the invention is a fragment of EphrinA1 (“EphrinA1 Fragment”). In accordance with this embodiment, the EphrinA1 Fragment preferably retains the ability to bind to EphA2 or EphA4. In a preferred embodiment, an EphrinA1 Fragment of the invention agonizes EphA2 and/or EphA4 signaling and/or degradation, preferably in a hyperproliferative cell and not in a non-hyperproliferative cell.

Various assays known to one of skill in the art may be performed to measure EphA2 or EphA4 signaling. For example, EphA2 or EphA4 phosphorylation may be measured to determine whether EphA2 or EphA4 signaling is activated upon ligand binding by measuring the amount of phosphorylated EphA2 or EphA4 present in EphrinA1-treated cells relative to control cells that are not treated with EphrinA1. EphA2 or EphA4 may be isolated using any protein immunoprecipitation method known to one of skill in the art and an EphA2 or EphA4 antibody of the invention. Phosphorylated EphA2 or EphA4 may then be measured using anti-phosphotyrosine antibodies (Upstate Tiotechnology, Inc., Lake Placid, N.Y.) using any standard immunoblotting method known to one of skill in the art. See, e.g., Cheng et al., 2002, Cytokine & Growth Factor Rev. 13: 75-85. In another embodiment, MAPK phosphorylation may be measured to determine whether EphA2 or EphA4 signaling is activated upon ligand binding by measuring the amount of phosphorylated MAPK present in EphrinA1-treated cells relative to control cells that are not treated with EphrinA1 using standard immunoprecipitation and immunoblotting assays known to one of skill in the art (see, e.g., Miao et al., 2003, J. Cell Biol. 7: 1281-1292, which is incorporated by reference herein in its entirety).

Non-limiting examples of EphrinA1 Fragments include, but are not limited to, any fragment of human EphrinA1 as disclosed in the GenBank database (e.g., GenBank Accession Nos. NP_{—}004419 (variant 1) and NP_{—}872626 (variant 2)). In a specific embodiment, an EphrinA1 Fragment is soluble (i.e., not membrane-bound). In a specific embodiment, an EphrinA1 Fragment of the invention comprises the extracellular domain of human EphrinA1 or a portion thereof. In further embodiments, an EphrinA1 Fragment of the invention comprises the extracellular domain of human EphrinA1 or a fragment thereof and is not membrane-bound. In specific embodiments, an EphrinA1 Fragment of the invention comprises specific fragments of the extracellular domain of human EphrinA1 variant 1 or a fragment thereof and is not membrane bound. In other specific embodiments, an EphrinA1 Fragment of the invention comprises specific fragments of the extracellular domain of human EphrinA1 variant 2 or a fragment thereof and is not membrane-bound.

The EphrinA1 Fragments include polypeptides that are 100%, 98%, 95%, 90%, 85%, 80%, 75%, 70%, 65%, 60%, 55%, 50%, 45%, 40% identical to endogenous EphrinA1 sequences. The determination of percent identity of two amino acid sequences can be determined by any method known to one skilled in the art, including BLAST protein searches. In specific embodiments, EphrinA1 Fragments of the invention can be analogs or derivatives of EphrinA1. For example, EphrinA1 Fragments of the invention include derivatives that are modified, i.e., by covalent attachment of any type of molecule to the polypeptide. For example, but not by way of limitation, the polypeptide derivatives (e.g., EphrinA1 polypeptide derivatives) include polypeptides that have been modified, e.g., by glycosylation, acetylation, pegylation, phosphorylation, amidation, derivatization by known protecting/blocking groups, proteolytic cleavage, linkage to a cellular ligand, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

In a specific embodiment, a targeting moiety of the the invention is an Ephrin A1 fusion protein. In accordance with this embodiment, the Ephrin A1 fusion protein may be soluble (e.g., not membrane-bound). Non-limiting examples of Ephrin A1 fusion proteins include soluble forms of Ephrin A1 such as Ephrin A1 Fc (see, e.g., Duxbury et al., 2004, Biochem. & Biophys. Res. Comm. 320: 1096-1102, which is incorporated by reference herein in its entirety). In a specific embodiment, an Ephrin A1 fusion protein comprises Ephrin A1 fused to an Fc domain of human immunoglobulin IgG. In another embodiment, an Ephrin A1 fusion protein comprises an Ephrin A1 Fragment which retains its ability to bind EphA2 or EphA4 fused to the Fc domain of human immunoglobulin IgG. In yet a further embodiment, an Ephrin A1 fusion protein comprises an Ephrin A1 Fragment which retains its ability to bind EphA2 or EphA4 fused to a heterologous protein (e.g., human serum albumin).

In further embodiments, a targeting moiety of the invention is an Ephrin A2, Ephrin A3, Ephrin A4, Ephrin A5, Ephrin B2 or Ephrin B3 fusion protein. Non-limiting examples of such fusion proteins include soluble forms of Ephrin A2, Ephrin A3, Ephrin A4, Ephrin A5, Ephrin B2 or Ephrin B3 fused to an Fc domain of human immunoglobulin IgG (e.g., Ephrin A2 Fc, Ephrin A3 Fc, Ephrin A4 Fc, Ephrin A5 Fc, Ephrin B2 Fc and Ephrin B3 Fc). In another embodiment, such fusion proteins retain their ability to bind EphA2 and/or EphA4 and agonize EphA2 and/or EphA4 signaling. In a further embodiment, such fusion proteins which retain their ability to bind EphA2 and/or EphA4 are fused to a heterologous protein (e.g., human serum albumin).

Fragments of EphrinA1 can be made and assayed for the ability to bind EphA2 or EphA4, using biochemical, biophysical, genetic, and/or computational techniques for studying protein-protein interactions that are described herein or by any method known in the art. Non-limiting examples of methods for detecting protein binding (e.g., for detecting EphA2 or EphA4 binding to EphrinA1), qualitatively or quantitatively, in vitro or in vivo, include GST-affinity binding assays, far-Western Blot analysis, surface plasmon resonance (SRP), fluorescence resonance energy transfer (FRET), fluorescence polarization (FP), isothermal titration calorimetry (ITC), circular dichroism (CD), protein fragment complementation assays (PCA), various two-hybrid systems, and proteomics and bioinformatics-based approaches, such as the Scansite program for computational analysis (see, e.g., Fu, H., 2004, Protein-Protein Interactions: Methods and Applications (Humana Press, Totowa, N.J.); and Protein-Protein Interactions: A Molecular Cloning Manual, 2002, Golemis, ed. (Cold Spring Harbor Laboratory Press, Cold Spring Harbor, N.Y.) which are incorporated by reference herein in their entireties).

5.2.1 Methods of Producing Antibodies

The antibodies or fragments thereof useful in the invention can be produced by any method known in the art for the production, selection and synthesis of antibodies, in particular, by chemical synthesis or, preferably, by monoclonal antibody technology, including recombinant expression techniques.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with LMW-PTP, EphA2 or EphA4 (either the full length protein or a domain thereof, e.g., the extracellular domain or the ligand binding domain) and once an immune response is detected, e.g., antibodies specific for LMW-PTP, EphA2 or EphA4 are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. Hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, monoclonal antibodies can be generated by culturing a hybridoma cell secreting an antibody of the invention wherein, preferably, the hybridoma is generated by fusing splenocytes isolated from a mouse immunized with LMW-PTP, EphA2 or EphA4 or fragment thereof with myeloma cells and then screening the hybridomas resulting from the fusion for hybridoma clones that secrete an antibody able to bind LMW-PTP, EphA2 or EphA4.

Antibody fragments which recognize specific LMW-PTP, EphA2 or EphA4 epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of lymphoid tissues). The DNA encoding the VH and VL domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to the LMW-PTP, EphA2 or EphA4 epitope of interest can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182: 41-50; Ames et al., 1995, J. Immunol. Methods 184: 177; Kettleborough et al., 1994, Eur. J. Immunol. 24: 952-958; Persic et al., 1997, Gene 187: 9; Burton et al., 1994, Advances in Immunology 57: 191-280; International Application No. PCT/GB91/01134; International Publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

Phage may be screened for LMW-PTP, EphA2 or EphA4 binding, particularly to the extracellular domain of EphA2 or EphA4. Agonizing EphA2 or EphA4 activity (e.g., increasing EphA2 or EphA4 phosphorylation, reducing EphA2 or EphA4 levels) or cancer cell phenotype inhibiting activity (e.g., reducing colony formation in soft agar or tubular network formation in a three-dimensional basement membrane or extracellular matrix preparation, such as MATRIGEL™) or preferentially binding to an EphA2 or EphA4 epitope exposed on cancer cells but not non-cancer cells (e.g., binding poorly to EphA2 or EphA4 that is bound to ligand in cell-cell contacts while binding well to EphA2 that is not bound to ligand or in cell-cell contacts) may also be screened.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in International Publication No. WO 92/22324; Mullinax et al., 1992, BioTechniques 12: 864; Sawai et al., 1995, AJRI 34: 26; and Better et al., 1988, Science 240: 1041 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lambda constant regions. Preferably, the vectors for expressing the VH or VL domains comprise an EF-1α promoter, a secretion signal, a cloning site for the variable domain, constant domains, and a selection marker such as neomycin. The VH and VL domains may also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

Antibodies of the invention, e.g., any EphA2/EphA4 agonistic antibody, a LMW-PTP antibody, or EphA2/EphA4 cancer cell phenotype inhibiting antibody or exposed EphA2/EphA4 epitope antibody or EphA2/EphA4 antibody that binds EphA2 or EphA4 with a K_{off}, may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol. 8: 724-33; Harayama, 1998, Trends Biotechnol. 16: 76; Hansson, et al., 1999, J. Mol. Biol. 287: 265; and Lorenzo and Blasco, 1998, BioTechniques 24: 308 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination. One or more portions of a polynucleotide encoding an antibody or antibody fragment, which portions immunospecifically bind to LMW-PTP, EphA2 or EphA4 may be recombined with one or more components, motifs, sections, parts, domains, fragments, etc. of one or more heterologous agents.

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13: 65-93). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Fremont, Calif.) and Medarex (Princeton, N.J.) can be engaged to provide human antibodies directed against a selected antigen using technology similar to that described above.

A chimeric antibody is a molecule in which different portions of the antibody are derived from different immunoglobulin molecules such as antibodies having a variable region derived from a non-human antibody and a human immunoglobulin constant region. Methods for producing chimeric antibodies are known in the art. See e.g., Morrison, 1985, Science 229: 1202; Oi et al., 1986, BioTechniques 4: 214; Gillies et al., 1989, J. Immunol. Methods 125: 191-202; and U.S. Pat. Nos. 6,311,415, 5,807,715, 4,816,567, and 4,816,397, which are incorporated herein by reference in their entirety. Chimeric antibodies comprising one or more CDRs from a non-human species and framework regions from a human immunoglobulin molecule can be produced using a variety of techniques known in the art including, for example, CDR-grafting (EP 239,400; International Publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(4/5): 489-498; Studnicka et al., 1994, Protein Engineering 7: 805; and Roguska et al., 1994, PNAS 91: 969), and chain shuffling (U.S. Pat. No. 5,565,332.

Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., U.S. Pat. No. 5,585,089; and Riechmann et al., 1988, Nature 332: 323, which are incorporated herein by reference in their entireties.)

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immunoglobulin. A humanized antibody comprises substantially all of at least one, and typically two, variable domains in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG₁, IgG₂, IgG₃ and IgG₄. Usually the constant domain is a complement fixing constant domain where it is desired that the humanized antibody exhibit cytotoxic activity, and the class is typically IgG₁. Where such cytotoxic activity is not desirable, the constant domain may be of the IgG₂ class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework region (FR) and CDR sequences, more often 90%, and most preferably greater than 95%. Humanized antibodies can be produced using variety of techniques known in the art, including but not limited to, CDR-grafting (European Patent No. EP 239,400; International Publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5): 489-498; Studnicka et al., 1994, Protein Engineering 7(6): 805-814; and Roguska et al., 1994, PNAS 91: 969-973), chain shuffling (U.S. Pat. No. 5,565,332), and techniques disclosed in, e.g., U.S. Pat. Nos. 6,407,213, 5,766,886, 5,585,089, International Publication No. WO 9317105, Tan et al., 2002, J. Immunol. 169: 1119-25, Caldas et al., 2000, Protein Eng. 13: 353-60, Morea et al., 2000, Methods 20: 267-79, Baca et al., 1997, J. Biol. Chem. 272: 10678-84, Roguska et al., 1996, Protein Eng. 9: 895-904, Couto et al., 1995, Cancer Res. 55 (23 Supp): 5973s-5977s, Couto et al., 1995, Cancer Res. 55: 1717-22, Sandhu, 1994, Gene 150: 409-10, Pedersen et al., 1994, J. Mol. Biol. 235: 959-73, Jones et al., 1986, Nature 321: 522-525, Riechmann et al., 1988, Nature 332: 323, and Presta, 1992, Curr. Op. Struct. Biol. 2: 593-596. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332: 323, which are incorporated herein by reference in their entireties.)

Further, the antibodies of the invention can, in turn, be utilized to generate anti-idiotype antibodies using techniques well known to those skilled in the art. (See, e.g., Greenspan & Bona, 1989, FASEB J. 7: 437-444; and Nissinoff, 1991, J. Immunol. 147: 2429-2438). The invention provides methods employing the use of polynucleotides comprising a nucleotide sequence encoding an antibody of the invention or a fragment thereof.

5.2.2. Polynucleotides Encoding an Antibody

Polynucleotides that encode a particular antibody may be obtained, and the nucleotide sequence of the polynucleotides determined, by any method known in the art. Since the amino acid sequences of the antibodies are known, nucleotide sequences encoding these antibodies can be determined using methods well known in the art, i.e., nucleotide codons known to encode particular amino acids are assembled in such a way to generate a nucleic acid that encodes the antibody or fragment thereof of the invention. Such a polynucleotide encoding the antibody may be assembled from chemically synthesized oligonucleotides (e.g., as described in Kutmeier et al., 1994, BioTechniques 17: 242), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody useful in the invention, e.g., clones deposited in American Type Culture Collection (ATCC, P.O. Box 1549, Manassas, Va. 20108) as PTA-4572, PTA-4573, PTA-4574 and PTA-5194, each of which is incorporated herein by reference) (see U.S. patent application Ser. No. 10/436,782, entitled “EphA2 Monoclonal Antibodies and Methods of Use Thereof,” filed May 12, 2003, which is incorporated herein by reference) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, one or more of the CDRs is inserted within framework regions using routine recombinant DNA techniques. The framework regions may be naturally occurring or consensus framework regions, and preferably human framework regions (see, e.g., Chothia et al., 1998, J. Mol. Biol. 278: 457-479 for a listing of human framework regions). Preferably, the polynucleotide generated by the combination of the framework regions and CDRs encodes an antibody that specifically binds to EphA2 or EphA4. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

5.2.3. Recombinant Expression of an Antibody

Recombinant expression of an antibody, derivative, analog or fragment thereof, (e.g., a heavy or light chain of an antibody or a portion thereof or a single chain antibody), requires construction of an expression vector containing a polynucleotide that encodes the antibody. Once a polynucleotide encoding an antibody molecule or a heavy or light chain of an antibody, or portion thereof (preferably, but not necessarily, containing the heavy or light chain variable domain), has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., International Publication Nos. WO 86/05807 and WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or portion thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45: 101; and Cockett et al., 1990, BioTechnology 8: 2). In a specific embodiment, the expression of nucleotide sequences encoding antibodies or fragments thereof which immunospecifically bind to EphA2 or EphA4 is regulated by a constitutive promoter, inducible promoter or tissue specific promoter.

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12: 1791), in which the antibody coding sequence may be ligated individually into the vector in frame with the lac Z coding region so that a fusion protein is produced; pIN vectors (Inouye & Inouye, 1985, Nucleic Acids Res. 13: 3101-3109; Van Heeke & Schuster, 1989, J. Biol. Chem. 24: 5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, PNAS 8 1: 355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153: 516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, W138, BT483, Hs578T, HTB2, BT2O, NS1 and T47D, NSO (a murine myeloma cell line that does not endogenously produce any immunoglobulin chains), CRL7O3O and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11: 223), glutamine synthetase, hypoxanthine guanine phosphoribosyltransferase (Szybalska & Szybalski, 1992, Proc. Natl. Acad. Sci. USA 48: 202), and adenine phosphoribosyltransferase (Lowy et al., 1980, Cell 22: 8-17) genes can be employed in tk-, gs-, hgprt- or aprt-cells, respectively. Also, antimetabolite resistance can be used as the basis of selection for the following genes: dhfr, which confers resistance to methotrexate (Wigler et al., 1980, PNAS 77: 357; O'Hare et al., 1981, PNAS 78: 1527); gpt, which confers resistance to mycophenolic acid (Mulligan & Berg, 1981, PNAS 78: 2072); neo, which confers resistance to the aminoglycoside G-418 (Wu and Wu, 1991, Biotherapy 3: 87; Tolstoshev, 1993, Ann. Rev. Pharmacol. Toxicol. 32: 573; Mulligan, 1993, Science 260: 926; and Morgan and Anderson, 1993, Ann. Rev. Biochem. 62: 191; May, 1993, TIB TECH 11: 155-); and hygro, which confers resistance to hygromycin (Santerre et al., 1984, Gene 30: 147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150: 1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3: 257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322: 52; and Kohler, 1980, PNAS 77: 2197). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

5.2.4. Antibody Conjugates

The present invention encompasses the use of antibodies or fragments thereof recombinantly fused or chemically conjugated (including both covalent and non-covalent conjugations) to a heterologous agent to generate a fusion protein as both targeting moieties and anti-LMW-PTP, EphA2 and/or EphA4 agents. The heterologous agent may be a polypeptide (or portion thereof, preferably to a polypeptide of at least 10, at least 20, at least 30, at least 40, at least 50, at least 60, at least 70, at least 80, at least 90 or at least 100 amino acids), nucleic acid, small molecule (less than 1000 daltons), or inorganic or organic compound. Preferably, the heterologous agent is an agent that inhibits or reduces LMW-PTP activity or expression. The fusion does not necessarily need to be direct, but may occur through linker sequences. Antibodies fused or conjugated to heterologous agents may be used in vivo to detect, treat, manage, or monitor the progression of a disorder using methods known in the art. See e.g., International Publication WO 93/21232; EP 439,095; Naramura et al., 1994, Immunol. Lett. 39: 91-99; U.S. Pat. No. 5,474,981; Gillies et al., 1992, PNAS 89: 1428-1432; and Fell et al., 1991, J. Immunol. 146: 2446-2452, which are incorporated by reference in their entireties. In some embodiments, the disorder to be detected, treated, managed, or monitored is malignant cancer that overexpresses EphA2 or EphA4. In other embodiments, the disorder to be detected, treated, managed, or monitored is a pre-cancerous condition associated with cells that overexpress EphA2 or EphA4. In a specific embodiments, the pre-cancerous condition is high-grade prostatic intraepithelial neoplasia (PIN), fibroadenoma of the breast, fibrocystic disease, or compound nevi.

The present invention further includes compositions comprising heterologous agents fused or conjugated to antibody fragments. For example, the heterologous polypeptides may be fused or conjugated to a Fab fragment, Fd fragment, Fv fragment, F(ab)₂ fragment, or portion thereof. Methods for fusing or conjugating polypeptides to antibody portions are known in the art. See, e.g., U.S. Pat. Nos. 5,336,603, 5,622,929, 5,359,046, 5,349,053, 5,447,851, and 5,112,946; EP 307,434; EP 367,166; International Publication Nos. WO 96/04388 and WO 91/06570; Ashkenazi et al., 1991, PNAS 88: 10535-10539; Zheng et al., 1995, J. Immunol. 154: 5590-5600; and Vil et al., 1992, PNAS 89: 11337-11341 (said references incorporated by reference in their entireties).

In one embodiment, antibodies of the present invention or fragments or variants thereof are conjugated to a marker sequence, such as a peptide, to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, PNAS 86: 821, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37: 767) and the “flag” tag.

In other embodiments, antibodies of the present invention or fragments or variants thereof are conjugated to a diagnostic or detectable agent. Such antibodies can be useful for monitoring or prognosing the development or progression of a cancer as part of a clinical testing procedure, such as determining the efficacy of a particular therapy. Additionally, such antibodies can be useful for monitoring or prognosing the development or progression of a pre-cancerous condition associated with cells that overexpress EphA2 or EphA4 (e.g., high-grade prostatic intraepithelial neoplasia (PIN), fibroadenoma of the breast, fibrocystic disease, or compound nevi). In one embodiment, an exposed EphA2 or EphA4 epitope antibody is conjugated to a diagnostic or detectable agent.

Such diagnosis and detection can accomplished by coupling the antibody to detectable substances including, but not limited to various enzymes, such as but not limited to horseradish peroxidase, alkaline phosphatase, beta-galactosidase, or acetylcholinesterase; prosthetic groups, such as but not limited to streptavidin/biotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷ Lu), manganese (⁵⁴Mn), molybdenum (⁹⁹Mo), palladium (¹⁰³Pd), phosphorous (³P), praseodymium (¹⁴²Pr), promethium (¹⁴⁹Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

In other embodiments, antibodies of the present invention or fragments or variants thereof are conjugated to a therapeutic agent such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Examples include paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

In other embodiments, antibodies of the present invention or fragments or variants thereof are conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol. 6: 1567), and VEGI (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1”), interleukin-2 (“IL-2”), interleukin-4 (“IL-4”), interleukin-6 (“IL-6”), interleukin-7 (“IL-7”), interleukin-9 (“IL-9”), interleukin-15 (“IL-15”), interleukin-12 (“IL-12”), granulocyte macrophage colony stimulating factor (“GM-CSF”), and granulocyte colony stimulating factor (“G-CSF”)), or a growth factor (e.g., growth hormone (“GH”)).

In other embodiments, antibodies of the present invention or fragments or variants thereof are conjugated to a therapeutic agent such as a radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (DOTA) which can be attached to the antibody via a linker molecule. Such linker molecules are commonly known in the art and described in Denardo et al., 1998, Clin Cancer Res. 4: 2483-90; Peterson et al., 1999, Bioconjug. Chem. 10: 553; and Zimmerman et al., 1999, Nucl. Med. Biol. 26: 943-50 each incorporated by reference in their entireties.

In a preferred embodiment, antibodies of the present invention or fragment or variants thereof are conjugated to an agent that inhibits or reduces LMW-PTP activity or expression. Non-limiting examples of such agents that inhibit LMW-PTP activity or expression are given in Section 5.1, supra.

In a specific embodiment, the conjugated antibody is an EphA2 or EphA4 antibody that preferably binds an EphA2 or EphA4 epitope exposed on cancer cells but not on non-cancer cells (i.e., exposed EphA2 or EphA4 epitope antibody).

Techniques for conjugating therapeutic moieties to antibodies are well known. Moieties can be conjugated to antibodies by any method known in the art, including, but not limited to aldehyde/Schiff linkage, sulphydryl linkage, acid-labile linkage, cis-aconityl linkage, hydrazone linkage, enzymatically degradable linkage (see generally Garnett, 2002, Adv. Drug Deliv. Rev. 53: 171-216). Additional techniques for conjugating therapeutic moieties to antibodies are well known, see, e.g., Amon et al., “Monoclonal Antibodies For Immunotargeting Of Drugs In Cancer Therapy,” in Monoclonal Antibodies And Cancer Therapy, Reisfeld et al. (eds.), pp. 243-56 (Alan R. Liss, Inc. 1985); Hellstrom et al., “Antibodies For Drug Delivery,” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review,” in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy,” in Monoclonal Antibodies For Cancer Detection And Therapy, Baldwin et al. (eds.), pp. 303-16 (Academic Press 1985), and Thorpe et al., 1982, Immunol. Rev. 62: 119-58. Methods for fusing or conjugating antibodies to polypeptide moieties are known in the art. See, e.g., U.S. Pat. Nos. 5,336,603, 5,622,929, 5,359,046, 5,349,053, 5,447,851, and 5,112,946; EP 307,434; EP 367,166; International Publication Nos. WO 96/04388 and WO 91/06570; Ashkenazi et al., 1991, PNAS 88: 10535-10539; Zheng et al., 1995, J. Immunol. 154: 5590-5600; and Vil et al., 1992, PNAS 89: 11337-11341. The fusion of an antibody to a moiety does not necessarily need to be direct, but may occur through linker sequences. Such linker molecules are commonly known in the art and described in Denardo et al., 1998, Clin Cancer Res. 4: 2483-90; Peterson et al., 1999, Bioconjug. Chem. 10: 553; Zimmerman et al., 1999, Nucl. Med. Biol. 26: 943-50; Garnett, 2002, Adv. Drug Deliv. Rev. 53: 171-216, each of which is incorporated herein by reference in its entirety.

Alternatively, an antibody can be conjugated to a second antibody to form an antibody heteroconjugate as described by Segal in U.S. Pat. No. 4,676,980, which is incorporated herein by reference in its entirety.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

5.3 Delivery Methods and Vehicles

The present invention provides methods and compositions designed for treatment, management, or prevention of a hyperproliferative cell disease, particularly cancer. To enhance the therapeutic or prophylactic effects of anti-LMW-PTP agents or other anti-cancer agents, and/or to decrease the unwanted side effects of such agents, the methods and compositions of the invention preferably target certain types of cells or specific tissues, particularly cells overexpressing EphA2 or EphA4.

Any delivery vehicle known in the art can be used in accordance with the present invention. Various delivery systems are known and can be used to administer one or more compositions of the invention, e.g., encapsulation in liposomes, microparticles, microcapsules, recombinant cells capable of expressing the antibody or antibody fragment, receptor-mediated endocytosis (see, e.g., Wu and Wu, 1987, J. Biol. Chem. 262: 4429-4432), construction of a nucleic acid as part of a retroviral or other vector, etc. For example, nucleic acid molecules can be delivered by use of microparticle bombardment (e.g., a gene gun; Biolistic, Dupont), or coating with lipids or transfecting agents that are conjugated to an EphA2 or EphA4 targeting moiety, encapsulation in liposomes, microparticles, or microcapsules, or by administering them in linkage to a peptide which is known to enter the nucleus, or by administering it in linkage to a ligand subject to receptor-mediated endocytosis (see, e.g., Wu and Wu, 1987, J. Biol. Chem. 262: 4429) (which can be used to target cell types specifically expressing the receptors), etc.

In a specific embodiment, the nucleic acid sequences are directly administered in vivo. This can be accomplished by any of numerous methods known in the art, e.g., by constructing them as part of an appropriate nucleic acid expression vector (e.g., vectors as described above and target to EphA2 or EphA4) and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or using any delivery vehicles known in the art and targeting EphA2 or EphA4 by conjugated to an appropriated targeting moiety (see Section 5.2, supra), or by administering them in linkage to a peptide which is known to enter the nucleus, by administering it in linkage to a ligand subject to receptor-mediated endocytosis (see, e.g., Wu and Wu, 1987, J. Biol. Chem. 262: 4429) (which can be used to target cell types specifically expressing the receptors, e.g., EphA2 or EphA4), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor, preferably EphA2 or EphA4 (see Section 5.2, supra). Alternatively, the nucleic acid can be introduced intracellularly and incorporated within host cell DNA for expression, by homologous recombination (Koller and Smithies, 1989, PNAS USA 86: 8932; and Zijlstra et al., 1989, Nature 342: 435).

In one embodiment, the nucleic acid is introduced into a cell prior to administration in vivo of the resulting recombinant cell. Such introduction can be carried out by any method known in the art, including but not limited to, transfection, electroporation, microinjection, infection with a viral or bacteriophage vector containing the nucleic acid sequences, cell fusion, chromosome-mediated gene transfer, microcell mediated gene transfer, spheroplast fusion, etc. Numerous techniques are known in the art for the introduction of foreign genes into cells (see, e.g., Loeffler and Behr, 1993, Meth. Enzymol. 217: 599; Cohen et al., 1993, Meth. Enzymol. 217: 618) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. The amount of cells envisioned for use depends on the desired effect, patient state, etc., and can be determined by one skilled in the art.

A delivery vehicle may target certain type of cells, e.g., by virtue of an innate feature of the vehicle, or by a moiety conjugated to the vehicle, which moiety specifically binds a particular subset of cells, e.g., by binding to a cell surface molecule characteristic of the subset of cells to be targeted. In a preferred embodiment, a delivery vehicle of the invention targets cells expressing EphA2, and may preferably target cells expressing EphA2 or EphA4 not bound to a ligand over EphA2 or EphA4 bound to a ligand. In a specific embodiment, an EphA2 targeting moiety is attached to a delivery vehicle of the invention.

The delivery vehicle can be, for example, a peptide vector, a peptide-DNA aggregate, a liposome, a gas-filled microsome, an encapsulated macromolecule, a nanosuspension, and the like (see e.g., Torchilin, Drug Targeting. Eur. J. Phamaceutical Sciences: v. 11, pp. S81-S91 (2000); Gerasimov, Boomer, Qualls, Thompson, Cytosolic drug delivery using pH- and light-sensitive liposomes, Adv. Drug Deliv. Reviews: v. 38, pp. 317-338 (1999); Hafez, Cullis, Roles of lipid polymorphism in intracellular delivery, Adv. Drug Deliv. Reviews: v. 47, pp. 139-148 (2001); Hashida, Akamatsu, Nishikawa, Fumiyoshi, Takakura, Design of polymeric prodrugs of prostaglandin E1 having galactose residue for hepatocyte targeting, J. Controlled Release: v. 62, pp. 253-262 (1999); Shah, Sadhale, Chilukuri, Cubic phase gels as drug delivery systems, Adv. Drug Deliv. Reviews: v. 47, pp. 229-250 (2001); Muller, Jacobs, Kayser, Nanosuspensions as particulate drug formulations in therapy: Rationale for development and what we can expect for the future, Adv. Drug Delivery Reviews: v. 47, pp. 3-19 (2001)). In some embodiments, the delivery vehicle is a viral vector. In a specific embodiment, a delivery vehicle can be, for example, an HVJ (Sendai virus)-liposome gene delivery system (see e.g., Kaneda et al., Ann. N.Y. Acad. Sci. 811: 299-308 (1997)); a “peptide vector” (see e.g., Vidal et al., CR Acad. Sci III 32: 279-287 (1997)); a peptide-DNA aggregate (see e.g., Niidome et al., J. Biol. Chem. 272: 15307-15312 (1997)); lipidic vector systems (see e.g., Lee et al., Crit Rev Ther Drug Carrier Syst. 14: 173-206 (1997)); polymer coated liposomes (Marin et al., U.S. Pat. No. 5,213,804; Woodle et al., U.S. Pat. No. 5,013,556); cationic liposomes (Epand et al., U.S. Pat. No. 5,283,185; Jessee, J. A., U.S. Pat. No. 5,578,475; Rose et al, U.S. Pat. No. 5,279,833; Gebeyehu et al., U.S. Pat. No. 5,334,761); gas filled microspheres (Unger et al., U.S. Pat. No. 5,542,935), or encapsulated macromolecules (Low et al., U.S. Pat. No. 5,108,921; Curiel et al., U.S. Pat. No. 5,521,291; Groman et al., U.S. Pat. No. 5,554,386; Wu et al., U.S. Pat. No. 5,166,320) (all references are incorporated herein by reference in their entireties).

Methods of packaging the therapeutic or prophylactic agent(s) into a delivery vehicle depend on various factors, such as the type of the delivery vehicle being used, or the hydrophobic or hydrophilic nature of the agent(s). Any packaging method known in the art can be used in the present invention.

5.3.1 Viruses

Viruses are attractive delivery vehicles for their natural ability to infect host cells and introduce foreign nucleic acids.

Viral vector systems useful in the practice of the instant invention include, for example, naturally occurring or recombinant viral vector systems. For example, viral vectors can be derived from the genome of human or bovine adenoviruses, vaccinia virus, herpes virus, adeno-associated virus (see e.g., Xiao et al., Brain Res. 756: 76-83 (1997), minute virus of mice (MVM), HIV, HPV and HPV-like particles, sindbis virus, and retroviruses (including but not limited to Rous sarcoma virus), and MoMLV, hepatitis B virus (see e.g., Ji et al., J. Viral Hepat. 4: 167-173 (1997)). Typically, genes of interest are inserted into such vectors to allow packaging of the gene construct, typically with accompanying viral DNA, followed by infection of a sensitive host cell and expression of the gene of interest. One example of a preferred recombinant viral vector is the adenoviral vector delivery system which has a deletion of the protein IX gene (see, International Patent Application WO 95/11984, which is herein incorporated by reference in its entirety). Another example of a preferred recombinant viral vector is the recombinant parainfluenza virus vector (recombinant PIV vectors, disclosed in e.g., Internation Patent Application Publication No. WO 03/072720, MedImmune Vaccines, Inc., incorporated herein by reference in its entirety) or a recombinant metapneumovirus vector (recombinant MPV vectors, disclosed in e.g., International Patent Application Publication No. WO 03/072719, MedImmune Vaccines, Inc., incorporated herein by reference in its entirety).

In some instances it may be advantageous to use vectors derived from a different species from that which is to be treated in order to avoid the preexisting immune response. For example, equine herpes virus vectors for human gene therapy are described in WO 98/27216 published Aug. 5, 1998. The vectors are described as useful for the treatment of humans as the equine virus is not pathogenic to humans. Similarly, ovine adenoviral vectors may be used in human gene therapy as they are claimed to avoid the antibodies against the human adenoviral vectors. Such vectors are described in WO 97/06826 published Apr. 10, 1997, which is incorporated herein by reference.

The virus can be replication competent (e.g., completely wild-type or essentially wild-type such as Ad d1309 or Ad d1520), conditionally replicating (designed to replicate under certain conditions) or replication deficient (substantially incapable of replication in the absence of a cell line capable of complementing the deleted functions). Alternatively, the viral genome can possess certain modifications to the viral genome to enhance certain desirable properties such as tissue selectivity. For example, deletions in the E1a region of adenovirus result in preferential replication and improved replication in tumor cells. The viral genome can also modified to include therapeutic transgenes. The virus can possess certain modifications to make it “selectively replicating,” i.e. that it replicates preferentially in certain cell types or phenotypic cell states, e.g., cancerous. For example, a tumor or tissue specific promoter element can be used to drive expression of early viral genes resulting in a virus which preferentially replicates only in certain cell types. Alternatively, one can employ a pathway-selective promoter active in a normal cell to drive expression of a repressor of viral replication. Selectively replicating adenoviral vectors that replicate preferentially in rapidly dividing cells are described in International Patent Application Nos. WO 990021451 and WO 990021452, each of which is incorporated herein by reference.

In a specific embodiment, viral vectors that contain nucleic acid sequences that reduce LWM-PTP, EphA2 or EphA4 expression and/or function are used. For example, a retroviral vector can be used (see Miller et al., 1993, Meth. Enzymol. 217: 581). These retroviral vectors contain the components necessary for the correct packaging of the viral genome and integration into the host cell DNA. The nucleic acid sequences to be used in accordance with the present invention are cloned into one or more vectors, which facilitates delivery of the nucleic acid into a subject. More detail about retroviral vectors can be found in Boesen et al., 1994, Biotherapy 6: 291-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93: 644-651; Klein et al., 1994, Blood 83: 1467-1473; Salmons and Gunzberg, 1993, Human Gene Therapy 4: 129-141; and Grossman and Wilson, 1993, Curr. Opin. in Genetics Devel. 3: 110-114.

Adenoviruses are other viral vectors that can be used in delivering nucleic acid molecules of the invention. Adenoviruses are especially attractive vehicles for delivering genes to respiratory epithelia. Adenoviruses naturally infect respiratory epithelia where they cause a mild disease. Adenoviruses have the advantage of being capable of infecting non-dividing cells. Kozarsky and Wilson, 1993, Current Opinion in Genetics Development 3: 499 present a review of adenovirus-based gene therapy. Bout et al., 1994, Human Gene Therapy 5: 3-10 demonstrated the use of adenovirus vectors to transfer genes to the respiratory epithelia of rhesus monkeys. Other instances of the use of adenoviruses as a delivery vehicle can be found in Rosenfeld et al., 1991, Science 252: 431; Rosenfeld et al., 1992, Cell 68: 143; Mastrangeli et al., 1993, J. Clin. Invest. 91: 225; International Publication No. WO94/12649; and Wang et al., 1995, Gene Therapy 2: 775. In a preferred embodiment, adenovirus vectors are used.

Adeno-associated virus (AAV) has also been proposed for use as a delivery vehicle (Walsh et al., 1993, Proc. Soc. Exp. Biol. Med. 204: 289-300; and U.S. Pat. No. 5,436,146).

A variety of approaches to create targeted viruses have been described in the literature. For example, cell targeting has been achieved with adenovirus vectors by selective modification of the viral genome knob and fiber coding sequences to achieve expression of modified knob and fiber domains having specific interaction with unique cell surface receptors, e.g., engineered to contain an EphA2 or EphA4 targeting moiety. Examples of such modifications are described in Wickham et al. (1997) J. Virol. 71(11): 8221-8229 (incorporation of RGD peptides into adenoviral fiber proteins); Arnberg et al. (1997) Virology 227: 239-244 (modification of adenoviral fiber genes to achieve tropism to the eye and genital tract); Harris and Lemoine (1996) TIG 12(10): 400-405; Stevenson et al. (1997) J. Virol. 71(6): 4782-4790; Michael et al. (1995) Gene Therapy 2: 660-668 (incorporation of gastrin releasing peptide fragment into adenovirus fiber protein); and Ohno et al. (1997) Nature Biotechnology 15: 763-767 (incorporation of Protein A-IgG binding domain into Sindbis virus).

Other methods of cell specific targeting rely on the conjugation of antibodies or antibody fragments to the envelope proteins (see e.g. Michael et al. (1993) J. Biol. Chem. 268: 6866-6869, Watkins et al. (1997) Gene Therapy 4: 1004-1012; Douglas et al. (1996) Nature Biotechnology 14: 1574-1578). For example, an antibody or an antibody fragment that binds EphA2 or EphA4 can be chemically conjugated to the surface of the virion by modification of amino acyl side chains in the antibody (particularly through lysine residues). Another non-limiting example of decorating the surface of a virus for targeting purpose is demonstrated in the U.S. Pat. No. 6,635,476, which is incorporated herein by reference. Alternative to the use of antibodies, others have complexed targeting proteins to the surface of the virion. See, e.g. Nilson et al. (1996) Gene Therapy 3: 280-286 (conjugation of EGF to retroviral proteins).

In some embodiments, an EphA2 targeting moiety, e.g., an anti-EphA2 antibody, an EphA2 ligand, a peptide or other targeting moieties known in the art, is attached to the surface of the virus, and thus direct the virus to the cells that expressing EphA2.

In some embodiments, an EphA4 targeting moiety, e.g., an anti-EphA4 antibody, an EphA4 ligand, a peptide or other targeting moieties known in the art, is attached to the surface of the virus, and thus direct the virus to the cells that expressing EphA4.

5.3.2 Synthetic Vectors

Non-viral synthetic vectors can also be used as a delivery vehicle in accordance with the present invention. For examples, a targeting moiety can be attached to a polycation (e.g., lipid or polymer) backbone. The polycation backbone also forms a complex with the therapeutic or prophylactic agent (e.g., a nucleic acid molecule) to be delivered. A non-limiting example of such delivery vehicle is polylysine, which has been conjugated to a diverse set of ligands that selectively target particular receptors on certain cell types. See e.g., Cotton et al., Proc. Natl. Acad. Sci. 87: 4033-4037 (1990); Fur et al., Receptor-mediated targeted gene delivery using asialoglycoprotein-polylysine conjugates, in Gene Therapeutics: Methods and Applications of Direct Gene Transfer, Wolff J A Ed, Birkhauser: Boston, pp 382-390 (1994); McGraw et al., Internalization and sorting of macromolecules: Endocytosis, in Targeted Drug Delivery, Juliano R L ed., Springer: New York, pp 11-41 (1991); and Uike et al., Biosci Biotechnol. Biochem. 62: 1247-1248 (1998). In some preferred embodiments, an EphA2 targeting moiety, e.g., an anti-EphA2 antibody, an EphA2 ligand, a peptide or other targeting moieties known in the art, is attached to the polycation backbone (e.g., polylysine), and thereby directs the therapeutic agent(s) to the cells that express LMW-PTP, EphA2 or EphA4. In some preferred embodiments, an EphA4 targeting moiety, e.g., an anti-EphA4 antibody, an EphA2 ligand, a peptide or other targeting moieties known in the art, is attached to the polycation backbone (e.g., polylysine), and thereby directs the therapeutic agent(s) to the cells that express EphA4 or LMW-PTP.

Chimeric multi-domain peptides can also be used as delivery vehicles in accordance with the present invention. See e.g., Fominaya et al., J. Biol. Chem. 271: 10560-10568 (1996); and Uherek et al., J. Biol. Chem. 273: 8835-8841 (1998). Such carrier incorporates targeting (i.e., EphA2), endosomal escape, and DNA binding motifs into a single synthetic peptide molecule.

5.3.3 Liposomes

In accordance with the present invention, liposomes can be used as a delivery vehicle. Liposomes are closed lipid vesicles used for a variety of therapeutic purposes, and in particular, for carrying therapeutic or prophylactic agents to a target region or cell by systemic administration of liposomes. Liposomes are usually classified as small unilamellar vesicles (SUV), large unilamellar vesicles (LUV), or multi-lamellar vesicles (MLV). SUVs and LUVs, by definition, have only one bilayer, whereas MLVs contain many concentric bilayers. Liposomes may be used to encapsulate various materials, by trapping hydrophilic molecules in the aqueous interior or between bilayers, or by trapping hydrophobic molecules within the bilayer. Gangliosides are believed to inhibit nonspecific adsorption of serum proteins to liposomes, thereby prevent nonspecific recognition of liposomes by macrophages.

In particular, liposomes having a surface grafted with chains of water-soluble, biocompatible polymer, in particular polyethylene glycol, have become important drug carries. These liposomes offer an extended blood circulation lifetime over liposomes lacking the polymer coating. The grafted polymer chains shield or mask the liposome, thus minimizing nonspecific interaction by plasma proteins. This in turn slows the rate at which the liposomes are cleared or eliminated in vivo since the liposome circulate unrecognized by macrophages and other cells of the reticuloendothelial system. Furthermore, due to the so-called enhanced permeability and retention effect, the liposomes tend to accumulate in sites of damaged or expanded vasculature, e.g., tumors, and sites of inflammation.

It would be desirable to formulate a liposome composition having a long blood circulation lifetime and capable of retaining an entrapped drug for a desired time, yet able to release the drug on demand. One approach described in the art for achieving these features has been to formulate a liposome from a non-vesicle-forming lipid, such as dioleoylphosphatidylethanolamine (DOPE), and a lipid bilayer stabilizing lipid, such as methoxy-polyethylene glycol-distearoyl phosphatidylethanolamine (mPEG-DSPE) (Kirpotin et al., FEBS Lett. 388: 115-118 (1996)). In this approach, the mPEG is attached to the DSPE via a cleavable linkage. Cleavage of the linkage destabilizes the liposome for a quick release of the liposome contents.

Labile bonds for linking PEG polymer chains to liposomes have been described (U.S. Pat. Nos. 5,013,556, 5,891,468; WO 98/16201). The labile bond in these liposome compositions releases the PEG polymer chains from the liposomes, for example, to expose a surface attached targeting ligand or to trigger fusion of the liposome with a target cell.

In a liposomal drug delivery system, an anti-LMW-PTP, an anti-EphA2 agent, or an anti-EphA4 agent is entrapped during liposome formation and then administered to the patient to be treated. See e.g., U.S. Pat. Nos. 3,993,754, 4,145,410, 4,224,179, 4,356,167, and 4,377,567. In the present invention, a liposome is preferably modified to have one or more EphA2 targeting moieties or EphA4 targeting moieties (see Section 5.1 and 5.2., supra) on its surface.

5.3.4. Hybrid Vectors

Hybrid vectors exploit endosomal escape capabilities of viruses in combination with the flexibility of non-viral vectors. Hybrid vectors can be divided into two subclasses: (1) membrane disrupting particles, either virus particles or other fusogenic peptides, added as separate entities in conjunction with non-viral vectors; and (2) such particles combined into a single complex with a traditional non-viral vector.

For example, a hybrid vector may use adenovirus in trans with a targeted non-viral vector, for example, adenovirus together with complexes of transferrin/polylysine, antibody/polylysine, or asialoglycoprotein/polylysine. See e.g., Cotton et al., Proc. Natl. Acad. Sci. 89: 6094-6098 (1992); Curiel et al., Receptor-mediated gene delivery empoying adenovirus-polylysine-DNA complexes, in Gene Therapeutics: Methods and Applications of Direct Gene Transfer, Wolff J A ed., Birkhauser: Boston, pp 99-116 (1994); Wagner et al., Proc. Natl. Acad. Sci. 89: 6099-6103 (1992); Christiano et al., Proc. Natl. Acad. Sci. 90: 2122-2126 (1993); each of which is incorporated herein by reference in its entirety. The mechanism of action of such hybrid vectors begins with the specific binding of both targeted complex and virus particle to their respective receptors. Upon binding, targeted complex and virus particle can either be internalized in the same vesicle or into separate endosomes. In a specific embodiment, a viral particle is directly conjugated to a targeted vector. Incorporation of viral particles into targeted complexes can be done, e.g., through streptavidin/biotinylation of adenovirus and polylysine, through antibodies pre-coupled to polylysine, or through direct chemical conjugation. See e.g., Verga et al., Biotechnology and Bioengineering 70(6): 593-605 (2000).

Preferably, the present invention provides hybrid vectors comprising one or more EphA2 targeting moieties and/or one or more EphA4 targeting moieties.

5.4. Prophylactic and/or Therapeutic Methods

The present invention encompasses methods for treating, preventing, or managing a disease or disorder associated with overexpression of EphA2 or EphA4 and/or cell hyperproliferative disorders, particularly cancer, in a subject comprising administering an effective amount of a composition that targets cells expressing LMW-PTP, EphA2, and/or EphA4, and inhibiting LMW-PTP expression or function. In one embodiment, the methods of the invention comprise administering to a subject a composition comprising an EphA2 or EphA4 targeting moiety and one or more agents that inhibit LMW-PTP expression and/or activity. In another embodiment, the methods of the invention comprise administering to a subject a composition comprising an EphA2 or EphA4 targeting moiety attached to a delivery vehicle, and one or more agents that inhibit LMW-PTP expression and/or activity operatively associated with the delivery vehicle. In another embodiment, the methods of the invention comprise administering to a subject a composition comprising a nucleic acid comprising a nucleotide sequence encoding an EphA2 or EphA4 targeting moiety and an agent that inhibits or reduces LMW-PTP expression and/or activity. In yet another embodiment, the method of the invention comprises administering to a subject a composition comprising an EphA2 or EphA4 targeting moiety and a nucleic acid comprising a nucleotide sequence encoding an agent that inhibits or reduces LMW-PTP expression and/or activity. In yet another embodiment, the methods of the invention comprise administering to a subject a composition comprising an EphA2 or EphA4 targeting moiety and a nucleic acid comprising a nucleotide sequence encoding an agent that inhibits or reduces LMW-PTP expression and/or activity, where the nucleic acid is operatively associated with the delivery vehicle.

In one embodiment, the compositions of the invention can be administered in combination with one or more other therapeutic agents useful in the treatment, prevention or management of diseases or disorders associated with EphA2 or EphA4 overexpression, hyperproliferative disorders, and/or cancer. In certain embodiments, one or more compositions of the invention are administered to a mammal, preferably a human, concurrently with one or more other therapeutic agents useful for the treatment of cancer. The term “concurrently” is not limited to the administration of prophylactic or therapeutic agents at exactly the same time, but rather it is meant that the compositions of the invention and the other agent are administered to a subject in a sequence and within a time interval such that the peptides of the invention can act together with the other agent to provide an increased benefit than if they were administered otherwise. For example, each prophylactic or therapeutic agent may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route. In other embodiments, the compositions of the invention are administered before, concurrently with or after surgery. Preferably the surgery completely removes localized tumors or reduces the size of large tumors. Surgery can also be done as a preventive measure or to relieve pain.

In another specific embodiment, the therapeutic and prophylactic methods of the invention comprise administration of an inhibitor of LMW-PTP, EphA2 and/or EphA4 expression, such as but not limited to, antisense nucleic acids specific for LMW-PTP, EphA2 and/or EphA4, double stranded LMW-PTP, EphA2 and/or EphA4 RNA that mediates RNAi, anti-LMW-PTP, anti-EphA2 or antiEphA4 ribozymes, and LMW-PTP, EphA2 or EphA4 aptamers, etc. (see Section 5.1.6., supra), a recombinant nucleic acid molecule encoding an intrabody that inhibits or reduces LMW-PTP activity or expression, or an agonist of EphA2 or EphA4 activity other than an EphA2 or EphA4 peptide, such as small molecule inhibitors or agonists of EphA2 or EphA4 activity.

5.4.1. Patient Population

The invention provides methods for treating, preventing, and managing a disease or disorder associated with EphA2 or EphA4 overexpression, low levels of EphA2 or EphA4 phosphorylation, LMW-PTP overexpression, and/or hyperproliferative cell disease, particularly cancer, by administrating to a subject in need thereof a therapeutically or prophylactically effective amount of one or more compositions of the invention. In another embodiment, the compositions of the invention can be administered in combination with one or more other therapeutic agents. The subject is preferably a mammal such as non-primate (e.g., cows, pigs, horses, cats, dogs, rats, etc.) and a primate (e.g., monkey, such as a cynomolgous monkey and a human). In a preferred embodiment, the subject is a human.

Specific examples of cancers that can be treated by the methods encompassed by the invention include, but are not limited to, cancers that overexpress EphA2 or EphA4. In a further embodiment, the cancer is of an epithelial origin. Examples of such cancers are cancer of the lung, colon, prostate, breast, and skin. Other cancers include cancer of the bladder and pancreas and renal cell carcinoma and melanoma. Additional cancers are listed by example and not by limitation in the following section 5.4.1.1. In particular embodiments, methods of the invention can be used to treat and/or prevent metastasis from primary tumors.

The methods and compositions of the invention comprise the administration of one or more compositions of the invention to subjects/patients suffering from or expected to suffer from cancer, e.g., have a genetic predisposition for a particular type of cancer, have been exposed to a carcinogen, or are in remission from a particular cancer. As used herein, “cancer” refers to primary or metastatic cancers. Such patients may or may not have been previously treated for cancer. The methods and compositions of the invention may be used as a first line or second line cancer treatment. Included in the invention is also the treatment of patients undergoing other cancer therapies and the methods and compositions of the invention can be used before any adverse effects or intolerance of these other cancer therapies occurs. The invention also encompasses methods for administering one or more EphA2 or EphA4 antibodies of the invention to treat or ameliorate symptoms in refractory patients. In a certain embodiment, that a cancer is refractory to a therapy means that at least some significant portion of the cancer cells are not killed or their cell division arrested by the therapy. The determination of whether the cancer cells are refractory can be made either in vivo or in vitro by any method known in the art for assaying the effectiveness of treatment on cancer cells, using the art-accepted meanings of “refractory” in such a context. In various embodiments, a cancer is refractory where the number of cancer cells has not been significantly reduced, or has increased. The invention also encompasses methods for administering one or more compositions to prevent the onset or recurrence of cancer in patients predisposed to having cancer.

In particular embodiments, the compositions of the invention, or other therapeutics that reduce EphA2 or EphA4 expression, are administered to reverse resistance or reduced sensitivity of cancer cells to certain hormonal, radiation and chemotherapeutic agents thereby resensitizing the cancer cells to one or more of these agents, which can then be administered (or continue to be administered) to treat or manage cancer, including to prevent metastasis. In a specific embodiment, compositions of the invention are administered to patients with increased levels of the cytokine IL-6, which has been associated with the development of cancer cell resistance to different treatment regimens, such as chemotherapy and hormonal therapy. In another specific embodiment, compositions of the invention are administered to patients suffering from breast cancer that have a decreased responsiveness or are refractory to tamoxifen treatment. In another specific embodiment, compositions of the invention are administered to patients with increased levels of the cytokine IL-6, which has been associated with the development of cancer cell resistance to different treatment regimens, such as chemotherapy and hormonal therapy.

In alternate embodiments, the invention provides methods for treating patients' cancer by administering one or more compositions of the invention in combination with any other treatment or to patients who have proven refractory to other treatments but are no longer on these treatments. In certain embodiments, the patients being treated by the methods of the invention are patients already being treated with chemotherapy, radiation therapy, hormonal therapy, or biological therapy/immunotherapy. Among these patients are refractory patients and those with cancer despite treatment with existing cancer therapies. In other embodiments, the patients have been treated and have no disease activity and one or more agonistic antibodies of the invention are administered to prevent the recurrence of cancer.

In preferred embodiments, the existing treatment is chemotherapy. In particular embodiments, the existing treatment includes administration of chemotherapies including, but not limited to, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, docetaxel, etc. Among these patients are patients treated with radiation therapy, hormonal therapy and/or biological therapy/immunotherapy. Also among these patients are those who have undergone surgery for the treatment of cancer.

Alternatively, the invention also encompasses methods for treating patients undergoing or having undergone radiation therapy. Among these are patients being treated or previously treated with chemotherapy, hormonal therapy and/or biological therapy/immunotherapy. Also among these patients are those who have undergone surgery for the treatment of cancer.

In other embodiments, the invention encompasses methods for treating patients undergoing or having undergone hormonal therapy and/or biological therapy/immunotherapy. Among these are patients being treated or having been treated with chemotherapy and/or radiation therapy. Also among these patients are those who have undergone surgery for the treatment of cancer.

Additionally, the invention also provides methods of treatment of cancer as an alternative to chemotherapy, radiation therapy, hormonal therapy, and/or biological therapy/immunotherapy where the therapy has proven or may prove too toxic, i.e., results in unacceptable or unbearable side effects, for the subject being treated. The subject being treated with the methods of the invention may, optionally, be treated with other cancer treatments such as surgery, chemotherapy, radiation therapy, hormonal therapy or biological therapy, depending on which treatment was found to be unacceptable or unbearable.

In other embodiments, the invention provides administration of one or more agonistic monoclonal antibodies of the invention without any other cancer therapies for the treatment of cancer, but who have proved refractory to such treatments. In specific embodiments, patients refractory to other cancer therapies are administered one or more agonistic monoclonal antibodies in the absence of cancer therapies.

In other embodiments, patients with a pre-cancerous condition associated with cells that overexpress EphA2 can be administered antibodies of the invention to treat the disorder and decrease the likelihood that it will progress to malignant cancer. In a specific embodiments, the pre-cancerous condition is high-grade prostatic intraepithelial neoplasia (PIN), fibroadenoma of the breast, fibrocystic disease, or compound nevi.

In yet other embodiments, the invention provides methods of treating, preventing and managing non-cancer hyperproliferative cell disorders, particularly those associated with overexpression of EphA2, including but not limited to, asthma, chromic obstructive pulmonary disorder (COPD), restenosis (smooth muscle and/or endothelial), psoriasis, etc. These methods include methods analogous to those described above for treating, preventing and managing cancer, for example, by administering the EphA2 or EphA4 antibodies of the invention, as well as agents that inhibit EphA2 or EphA4 expression, combination therapy, administration to patients refractory to particular treatments, etc.

5.4.1.1 Cancers

Cancers and related disorders that can be treated, prevented, or managed by methods and compositions of the present invention include but are not limited to cancers of an epithelial cell origin. Examples of such cancers include the following: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

Accordingly, the methods and compositions of the invention are also useful in the treatment or prevention of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burkitt's lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented in the skin, lung, colon, breast, prostate, bladder, kidney, pancreas, ovary, or uterus. In other specific embodiments, sarcoma, melanoma, or leukemia is treated or prevented.

In some embodiments, the cancer is malignant and overexpresses EphA2. In other embodiments, the disorder to be treated is a pre-cancerous condition associated with cells that overexpress EphA2. In a specific embodiments, the pre-cancerous condition is high-grade prostatic intraepithelial neoplasia (PIN), fibroadenoma of the breast, fibrocystic disease, or compound nevi.

In preferred embodiments, the methods and compositions of the invention are used for the treatment and/or prevention of breast, colon, ovarian, lung, and prostate cancers and melanoma and are provided below by example rather than by limitation.

5.4.2 Other Prophylactic and/or Therapeutic Agents

In some embodiments, therapy by administration of one or more compositions of the invention is combined with the administration of one or more therapies such as, but not limited to, chemotherapies, radiation therapies, hormonal therapies, and/or biological therapies/immunotherapies. Prophylactic/therapeutic agents include, but are not limited to, vaccines, proteinaceous molecules, including, but not limited to, peptides, polypeptides, proteins, including post-translationally modified proteins, antibodies etc.; or small molecules (less than 1000 daltons), inorganic or organic compounds; or nucleic acid molecules including, but not limited to, double-stranded or single-stranded DNA, or double-stranded or single-stranded RNA, triple helix nucleic acid molecules, or aptamers. Prophylavtic/therapeutic agents can be derived from any known organism (including, but not limited to, animals, plants, bacteria, fungi, and protista, or viruses) or from a library of synthetic molecules.

In a specific embodiment, the methods of the invention encompass administration of a composition of the invention in combination with the administration of one or more prophylactic/therapeutic agents that are inhibitors of kinases such as, but not limited to, ABL, ACK, AFK, AKT (e.g., AKT-1, AKT-2, and AKT-3), ALK, AMP-PK, ATM, Aurora1, Aurora2, bARK1, bArk2, BLK, BMX, BTK, CAK, CaM kinase, CDC2, CDK, CK, COT, CTD, DNA-PK, EGF-R, ErbB-1, ErbB-2, ErbB-3, ErbB-4, ERK (e.g., ERK1, ERK2, ERK3, ERK4, ERK5, ERK6, ERK7), ERT-PK, FAK, FGR (e.g., FGF1R, FGF2R), FLT (e.g., FLT-1, FLT-2, FLT-3, FLT-4), FRK, FYN, GSK (e.g., GSK1, GSK2, GSK3-alpha, GSK3-beta, GSK4, GSK5), G-protein coupled receptor kinases (GRKs), HCK, HER2, HKII, JAK (e.g., JAK1, JAK2, JAK3, JAK4), JNK (e.g., JNK1, JNK2, JNK3), KDR, KIT, IGF-1 receptor, IKK-1, IKK-2, INSR (insulin receptor), IRAK1, IRAK2, IRK, ITK, LCK, LOK, LYN, MAPK, MAPKAPK-1, MAPKAPK-2, MEK, MET, MFPK, MHCK, MLCK, MLK3, NEU, NIK, PDGF receptor alpha, PDGF receptor beta, PHK, PI-3 kinase, PKA, PKB, PKC, PKG, PRK1, PYK2, p38 kinases, p135tyk2, p34cdc2, p42cdc2, p42mapk, p44 mpk, RAF, RET, RIP, RIP-2, RK, RON, RS kinase, SRC, SYK, S6K, TAK1, TEC, TIE1, TIE2, TRKA, TXK, TYK2, UL13, VEGFR1, VEGFR2, YES, YRK, ZAP-70, and all subtypes of these kinases (see e.g., Hardie and Hanks (1995) The Protein Kinase Facts Book, I and II, Academic Press, San Diego, Calif.). In preferred embodiments, an antibody of the invention is administered in combination with the administration of one or more prophylactic/therapeutic agents that are inhibitors of Eph receptor kinases (e.g., EphA2, EphA4). In a most preferred embodiment, an antibody of the invention is administered in combination with the administration of one or more prophylactic/therapeutic agents that are inhibitors of EphA2.

In another specific embodiment, the methods of the invention encompass administration of a composition of the invention in combination with the administration of one or more prophylactic/therapeutic agents that are angiogenesis inhibitors such as, but not limited to: Angiostatin (plasminogen fragment); antiangiogenic antithrombin III; Angiozyme; ABT-627; Bay 12-9566; Benefin; Bevacizumab; BMS-275291; cartilage-derived inhibitor (CDI); CAI; CD59 complement fragment; CEP-7055; Col 3; Combretastatin A-4; Endostatin (collagen XVIII fragment); fibronectin fragment; Gro-beta; Halofuginone; Heparinases; Heparin hexasaccharide fragment; HMV833; Human chorionic gonadotropin (hCG); IM-862; Interferon alpha/beta/gamma; Interferon inducible protein (IP-10); Interleukin-12; Kringle 5 (plasminogen fragment); Marimastat; Metalloproteinase inhibitors (TIMPs); 2-Methoxyestradiol; MMI 270 (CGS 27023A); MoAb IMC-1C11; Neovastat; NM-3; Panzem; PI-88; Placental ribonuclease inhibitor; Plasminogen activator inhibitor; Platelet factor-4 (PF4); Prinomastat; Prolactin 16 kD fragment; Proliferin-related protein (PRP); PTK 787/ZK 222594; Retinoids; Solimastat; Squalamine; SS 3304; SU 5416; SU6668; SU11248; Tetrahydrocortisol-S; tetrathiomolybdate; thalidomide; Thrombospondin-1 (TSP-1); TNP-470; Transforming growth factor-beta (TGF-β); Vasculostatin; Vasostatin (calreticulin fragment); ZD6126; ZD6474; farnesyl transferase inhibitors (FTI); and bisphosphonates.

In another specific embodiment, the methods of the invention encompass administration of a composition of the invention in combination with the administration of one or more prophylactic/therapeutic agents that are anti-cancer agents such as, but not limited to: acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cisplatin, cladribine, crisnatol mesylate, cyclophosphamide, cytarabine, dacarbazine, dactinomycin, daunorubicin hydrochloride, decarbazine, decitabine, dexormaplatin, dezaguanine, dezaguanine mesylate, diaziquone, docetaxel, doxorubicin, doxorubicin hydrochloride, droloxifene, droloxifene citrate, dromostanolone propionate, duazomycin, edatrexate, eflornithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, flurocitabine, fosquidone, fostriecin sodium, gemcitabine, gemcitabine hydrochloride, hydroxyurea, idarubicin hydrochloride, ifosfamide, ilmofosine, interleukin 2 (including recombinant interleukin 2, or rIL2), interferon alpha-2a, interferon alpha-2b, interferon alpha-n1, interferon alpha-n3, interferon beta-I a, interferon gamma-I b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nitrosoureas, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazoftirin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, zorubicin hydrochloride. Other anti-cancer drugs include, but are not limited to: 20-epi-1,25 dihydroxyvitamin D3,5-ethynyluracil, abiraterone, aclarubicin, acylfulvene, adecypenol, adozelesin, aldesleukin, ALL-TK antagonists, altretamine, ambamustine, amidox, amifostine, aminolevulinic acid, amrubicin, amsacrine, anagrelide, anastrozole, andrographolide, angiogenesis inhibitors, antagonist D, antagonist G, antarelix, anti-dorsalizing morphogenetic protein-1, antiandrogens, antiestrogens, antineoplaston, aphidicolin glycinate, apoptosis gene modulators, apoptosis regulators, apurinic acid, ara-CDP-DL-PTBA, arginine deaminase, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitor, bicalutamide, bisantrene, bisaziridinylspermine, bisnafide, bistratene A, bizelesin, breflate, bropirimine, budotitane, buthionine sulfoximine, calcipotriol, calphostin C, camptothecin derivatives, canarypox IL-2, capecitabine, carboxamide-amino-triazole, carboxyamidotriazole, CaRest M3, CARN 700, cartilage derived inhibitor, carzelesin, casein kinase inhibitors (ICOS), castanospermine, cecropin B, cetrorelix, chloroquinoxaline sulfonamide, cicaprost, cis-porphyrin, cladribine, clomifene analogues, clotrimazole, collismycin A, collismycin B, combretastatin A4, combretastatin analogue, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, deslorelin, dexamethasone, dexifosfamide, dexrazoxane, dexverapamil, diaziquone, didemnin B, didox, diethylnorspermine, dihydro-5-azacytidine, dihydrotaxol, dioxamycin, diphenyl spiromustine, docetaxel, docosanol, dolasetron, doxifluridine, droloxifene, dronabinol, duocarmycin SA, ebselen, ecomustine, edelfosine, edrecolomab, eflornithine, elemene, emitefur, epirubicin, epristeride, estramustine analogue, estrogen agonists, estrogen antagonists, etanidazole, etoposide phosphate, exemestane, fadrozole, fazarabine, fenretinide, filgrastim, finasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, heregulin, hexamethylene bisacetamide, hypericin, ibandronic acid, idarubicin, idoxifene, idramantone, ilmofosine, ilomastat, imidazoacridones, imiquimod, immunostimulant peptides, insulin-like growth factor-1 receptor inhibitor, interferon agonists, interferons, interleukins, iobenguane, iododoxorubicin, ipomeanol, iroplact, irsogladine, isobengazole, isohomohalicondrin B, itasetron, jasplakinolide, kahalalide F, lamellarin-N triacetate, lanreotide, leinamycin, lenograstim, lentinan sulfate, leptolstatin, letrozole, leukemia inhibiting factor, leukocyte alpha interferon, leuprolide+estrogen+progesterone, leuprorelin, levamisole, liarozole, linear polyamine analogue, lipophilic disaccharide peptide, lipophilic platinum compounds, lissoclinamide 7, lobaplatin, lombricine, lometrexol, lonidamine, losoxantrone, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, menogaril, merbarone, meterelin, methioninase, metoclopramide, MIF inhibitor, mifepristone, miltefosine, mirimostim, mismatched double stranded RNA, mitoguazone, mitolactol, mitomycin analogues, mitonafide, mitotoxin fibroblast growth factor-saporin, mitoxantrone, mofarotene, molgramostim, monoclonal antibody, human chorionic gonadotrophin, monophosphoryl lipid A+myobacterium cell wall sk, mopidamol, multiple drug resistance gene inhibitor, multiple tumor suppressor 1-based therapy, mustard anticancer agent, mycaperoxide B, mycobacterial cell wall extract, myriaporone, N-acetyldinaline, N-substituted benzamides, nafarelin, nagrestip, naloxone+pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, O6-benzylguanine, octreotide, okicenone, oligonucleotides, onapristone, ondansetron, ondansetron, oracin, oral cytokine inducer, ormaplatin, osaterone, oxaliplatin, oxaunomycin, paclitaxel, paclitaxel analogues, paclitaxel derivatives, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, perillyl alcohol, phenazinomycin, phenylacetate, phosphatase inhibitors, picibanil, pilocarpine hydrochloride, pirarubicin, piritrexim, placetin A, placetin B, plasminogen activator inhibitor, platinum complex, platinum compounds, platinum-triamine complex, porfimer sodium, porfiromycin, prednisone, propyl bis-acridone, prostaglandin J2, proteasome inhibitors, protein A-based immune modulator, protein kinase C inhibitor, protein kinase C inhibitors, microalgal, protein tyrosine phosphatase inhibitors, purine nucleoside phosphorylase inhibitors, purpurins, pyrazoloacridine, pyridoxylated hemoglobin polyoxyethylene conjugate, raf antagonists, raltitrexed, ramosetron, ras farnesyl protein transferase inhibitors, ras inhibitors, ras-GAP inhibitor, retelliptine demethylated, rhenium Re 186 etidronate, rhizoxin, ribozymes, RII retinamide, rogletimide, rohitukine, romurtide, roquinimex, rubiginone B1, ruboxyl, safingol, saintopin, SarCNU, sarcophytol A, sargramostim, Sdi 1 mimetics, semustine, senescence derived inhibitor 1, sense oligonucleotides, signal transduction inhibitors, signal transduction modulators, single chain antigen binding protein, sizofiran, sobuzoxane, sodium borocaptate, sodium phenylacetate, solverol, somatomedin binding protein, sonermin, sparfosic acid, spicamycin D, spiromustine, splenopentin, spongistatin 1, squalamine, stem cell inhibitor, stem-cell division inhibitors, stipiamide, stromelysin inhibitors, sulfinosine, superactive vasoactive intestinal peptide antagonist, suradista, suramin, swainsonine, synthetic glycosaminoglycans, tallimustine, tamoxifen methiodide, tauromustine, taxol, tazarotene, tecogalan sodium, tegafur, tellurapyrylium, telomerase inhibitors, temoporfin, temozolomide, teniposide, tetrachlorodecaoxide, tetrazomine, thaliblastine, thalidomide, thiocoraline, thioguanine, thrombopoietin, thrombopoietin mimetic, thymalfasin, thymopoietin receptor agonist, thymotrinan, thyroid stimulating hormone, tin ethyl etiopurpurin, tirapazamine, titanocene bichloride, topsentin, toremifene, totipotent stem cell factor, translation inhibitors, tretinoin, triacetyluridine, triciribine, trimetrexate, triptorelin, tropisetron, turosteride, tyrosine kinase inhibitors, tyrphostins, UBC inhibitors, ubenimex, urogenital sinus-derived growth inhibitory factor, urokinase receptor antagonists, vapreotide, variolin B, vector system, erythrocyte gene therapy, velaresol, veramine, verdins, verteporfin, vinorelbine, vinxaltine, vitaxin, vorozole, zanoterone, zeniplatin, zilascorb, and zinostatin stimalamer. Preferred additional anti-cancer drugs are 5-fluorouracil and leucovorin.

In more particular embodiments, the present invention also comprises the administration of one or more compositions of the invention in combination with the administration of one or more therapies such as, but not limited to anti-cancer agents such as those disclosed in Table 5, preferably for the treatment of breast, ovary, melanoma, prostate, colon and lung cancers as described above. In specific embodiments, the present invention comprises the administration of additional anti-cancer agents that are not the moieties that bind EphA2 or EphA4 of the invention and are not the anti-LMW-PTP agents of the invention. Such additional anti-cancer therapies include, but are not limited to, chemotherapy, biological therapy, hormonal therapy, radiation and surgery.

TABLE 5

| Therapeutic Agent | Administration | Dose | Intervals |
|---|---|---|---|
| doxorubicin | Intravenous | 60-75 mg/m² on Day 1 | 21 day intervals |
| hydrochloride | | | |
| (Adriamycin RDF ® | | | |
| and Adriamycin | | | |
| PFS ®) | | | |
| epirubicin | Intravenous | 100-120 mg/m² on Day 1 of | 3-4 week cycles |
| hydrochloride | | each cycle or divided equally | |
| (Ellence ™) | | and given on Days 1-8 of the | |
| | | cycle | |
| fluorousacil | Intravenous | How supplied: | |
| | | 5 ml and 10 ml vials | |
| | | (containing 250 and 500 mg | |
| | | flourouracil respectively) | |
| docetaxel | Intravenous | 60-100 mg/m² over 1 hour | Once every 3 weeks |
| (Taxotere ®) | | | |
| paclitaxel | Intravenous | 175 mg/m² over 3 hours | Every 3 weeks for 4 courses |
| (Taxol ®) | | | (administered sequentially to |
| | | | doxorubicin-containing |
| | | | combination chemotherapy) |
| tamoxifen citrate | Oral | 20-40 mg | Daily |
| (Nolvadex ®) | (tablet) | Dosages greater than 20 mg | |
| | | should be given in divided | |
| | | doses (morning and evening) | |
| leucovorin calcium | Intravenous or | How supplied: | Dosage is unclear from text. |
| for injection | intramuscular | 350 mg vial | PDR 3610 |
| | injection | | |
| luprolide acetate | Single | 1 mg (0.2 ml or 20 unit mark) | Once a day |
| (Lupron ®) | subcutaneous | | |
| | injection | | |
| flutamide | Oral (capsule) | 250 mg | 3 times a day at 8 hour |
| (Eulexin ®) | | (capsules contain 125 mg | intervals (total daily dosage |
| | | flutamide each) | 750 mg) |
| nilutamide | Oral | 300 mg or 150 mg | 300 mg once a day for 30 |
| (Nilandron ®) | (tablet) | (tablets contain 50 or 150 mg | days followed by 150 mg |
| | | nilutamide each) | once a day |
| bicalutamide | Oral | 50 mg | Once a day |
| (Casodex ®) | (tablet) | (tablets contain 50 mg | |
| | | bicalutamide each) | |
| progesterone | Injection | USP in sesame oil 50 mg/ml | |
| ketoconazole | Cream | 2% cream applied once or | |
| (Nizoral ®) | | twice daily depending on | |
| | | symptoms | |
| prednisone | Oral | Initial dosage may vary from | |
| | (tablet) | 5 mg to 60 mg per day | |
| | | depending on the specific | |
| | | disease entity being treated. | |
| estramustine | Oral | 14 mg/kg of body weight | Daily given in 3 or 4 divided |
| phosphate sodium | (capsule) | (i.e. one 140 mg capsule for | doses |
| (Emcyt ®) | | each 10 kg or 22 lb of body | |
| | | weight) | |
| etoposide or VP-16 | Intravenous | 5 ml of 20 mg/ml solution | |
| | | (100 mg) | |
| dacarbazine | Intravenous | 2-4.5 mg/kg | Once a day for 10 days. |
| (DTIC-Dome ®) | | | May be repeated at 4 week |
| | | | intervals |
| polifeprosan 20 with | wafer placed in | 8 wafers, each containing 7.7 mg | |
| carmustine implant | resection cavity | of carmustine, for a total | |
| (BCNU) (nitrosourea) | | of 61.6 mg, if size and shape | |
| (Gliadel ®) | | of resection cavity allows | |
| cisplatin | Injection | How supplied: | |
| | | solution of 1 mg/ml in multi- | |
| | | dose vials of 50 mL and | |
| | | 100 mL | |
| mitomycin | Injection | supplied in 5 mg and 20 mg | |
| | | vials (containing 5 mg and 20 mg | |
| | | mitomycin) | |
| gemcitabine HCl | Intravenous | For NSCLC-2 schedules | 4 week schedule- |
| (Gemzar ®) | | have been investigated and | Days 1, 8 and 15 of each 28- |
| | | the optimum schedule has not | day cycle. Cisplatin |
| | | been determined | intravenously at 100 mg/m² |
| | | 4 week schedule- | on day 1 after the infusion of |
| | | administration intravenously | Gemzar. |
| | | at 1000 mg/m² over 30 | 3 week schedule- |
| | | minutes on 3 week schedule- | Days 1 and 8 of each 21 day |
| | | Gemzar administered | cycle. Cisplatin at dosage of |
| | | intravenously at 1250 mg/m² | 100 mg/m² administered |
| | | over 30 minutes | intravenously after |
| | | | administration of Gemzar on |
| | | | day 1. |
| carboplatin | Intravenous | Single agent therapy: | Every 4 weeks |
| (Paraplatin ®) | | 360 mg/m² I.V. on day 1 | |
| | | (infusion lasting 15 minutes | |
| | | or longer) | |
| | | Other dosage calculations: | |
| | | Combination therapy with | |
| | | cyclophosphamide, Dose | |
| | | adjustment recommendations, | |
| | | Formula dosing, etc. | |
| ifosamide | Intravenous | 1.2 g/m² daily | 5 consecutive days |
| (Ifex ®) | | | Repeat every 3 weeks or |
| | | | after recovery from |
| | | | hematologic toxicity |
| topotecan | Intravenous | 1.5 mg/m² by intravenous | 5 consecutive days, starting |
| hydrochloride | | infusion over 30 minutes | on day 1 of 21 day course |
| (Hycamtin ®) | | daily | |

The invention also encompasses administration of the compositions of the invention in combination with radiation therapy comprising the use of x-rays, gamma rays and other sources of radiation to destroy the cancer cells. In preferred embodiments, the radiation treatment is administered as external beam radiation or teletherapy wherein the radiation is directed from a remote source. In other preferred embodiments, the radiation treatment is administered as internal therapy or brachytherapy wherein a radioactive source is placed inside the body close to cancer cells or a tumor mass.

Cancer therapies and their dosages, routes of administration and recommended usage are known in the art and have been described in such literature as the Physician's Desk Reference (56th ed., 2002).

5.4.2.1 EphA2 and EphA4 Vaccines

In a specific embodiment, a therapeutic or prophylactic agent of the invention is an EphA2 and/or an EphA4 vaccine. As used herein, the term “EphA2 vaccine” refers to any reagent that elicits or mediates an immune response against cells that overexpress EphA2, preferably associated with a hyperproliferative cell disorder. In certain embodiments, an EphA2 vaccine is an EphA2 antigenic peptide, an expression vehicle (e.g., a naked nucleic acid or a viral or bacterial vector or a cell) for an EphA2 antigenic peptide (e.g., which delivers the EphA2 antigenic peptide), or T cells or antigen presenting cells (e.g., dendritic cells or macrophages) that have been primed with the EphA2 antigenic peptide of the invention. As used herein, the terms “EphA2 antigenic peptide” and “EphA2 antigenic polypeptide” refer to an EphA2 polypeptide, or a fragment, analog, or derivative thereof comprising one or more B cell epitopes or T cell epitopes of EphA2. The EphA2 polypeptide may be from any species. The EphA2 polypeptide may be from any species. For example, the human EphA2 sequence may be found in any publicly available data base, such as GenBank (Accession Nos. NM_{—}004431.2 for the nucleotide sequence and NP_{—}004422.2 for the amino acid sequence). In certain embodiments, an EphA2 polypeptide refers to the mature, processed form of EphA2. In other embodiments, an EphA2 polypeptide refers to an immature form of EphA2. For a description of EphA2 vaccines, see, e.g., U.S. Provisional Application Ser. No. 60/556,601, entitled “EphA2 Vaccines,” filed Mar. 26, 2004; U.S. Provisional Application Ser. No. ______, filed Aug. 18, 2004, entitled “EphA2 Vaccines” (Attorney Docket No. 10271-136-888); U.S. Provisional Application Ser. No. ______, filed Oct. 1, 2004, entitled “EphA2 Vaccines” (Attorney Docket No. 10271-143-888); U.S. Provisional Application Ser. No. ______, filed Oct. 7, 2004, entitled “EphA2 Vaccines” (Attorney Docket No. 10271-148-888), and International Application No. ______, filed Oct. 15, 2004 entitled “EphA2 Vaccines” (Attorney Docket No. 10271-148-228) each of which is incorporated by reference herein in its entirety.

In a specific embodiment, therapeutic or prophylactic agent of the invention is an EphA4 Vaccine. As used herein, the term “EphA4 vaccine” refers to any reagent that elicits or mediates an immune response against EphA41 on EphA4-expressing cells. In certain embodiments, an EphA4 vaccine is an EphA4 antigenic peptide of the invention, an expression vehicle (e.g., a naked nucleic acid or a viral or bacterial vector or a cell) for an EphA4 antigenic peptide (e.g., which delivers the EphA4 antigenic peptide), or T cells or antigen presenting cells (e.g., dendritic cells or macrophages) that have been primed with the EphA4, antigenic peptide of the invention. As used herein, the terms “EphA4 antigenic peptide” and “EphA4 antigenic polypeptide” refer to an EphA4 polypeptide, or a fragment, analog, or derivative thereof comprising one or more B cell epitopes or T cell epitopes of EphA4. The EphA4 polypeptide may be from any species. For example, the human EphA4 sequence may be found in any publicly available data base, such as GenBank (Accession Nos. NM_{—}004438.3 for the nucleotide sequence and NP_{—}004429.1 for the amino acid sequence). In certain embodiments, an EphA4 polypeptide refers to the mature, processed form of EphA4. In other embodiments, an EphA4 polypeptide refers to an immature form of EphA4.

The present invention thus provides therapeutic and/or prophylactic agents that are EphA2 or EphA4 vaccines. In a specific embodiment, a therapeutic and/or prophylactic agent is an EphA2- and/or EphA4 antigenic peptide expression vehicle expressing an EphA4 or an EphA4 antigenic peptide that can elicit or mediate a cellular immune response, a humoral response, or both, against cells that overexpress EphA2 or EphA4. Where the immune response is a cellular immune response, it can be a Tc, Th1 or a Th2 immune response. In a preferred embodiment, the immune response is a Th2 cellular immune response. In another preferred embodiment, an EphA2 or an EphA4 antigenic peptide expressed by an EphA2- or EphA4-antigenic peptide expression vehicle is an EphA2 or EphA4 antigenic peptide that is capable of eliciting an immune response against EphA2- and/or EphA4-expressing cells involved in an infection.

In a specific embodiment, the EphA2- and/or EphA4 antigenic expression vehicle is a microorganism expressing an EphA2 and/or an EphA4 antigenic peptide. In another specific embodiment, the EphA2- and/or EphA4 antigenic expression vehicle is an attenuated bacteria. Non-limiting examples of bacteria that can be utilized in accordance with the invention as an expression vehicle include Listeria monocytogenes, include but are not limited to Borrelia burgdorferi, Brucella melitensis, Escherichia coli, enteroinvasive Escherichia coli, Legionella pneumophila, Salmonella typhi, Salmonella typhimurium, Shigella spp., Streptococcus spp., Treponema pallidum, Yersinia enterocohtica, Listeria monocytogenes, Mycobacterium avium, Mycobacterium bovis, Mycobacterium tuberculosis, BCG, Mycoplasma hominis, Rickettsiae quintana, Cryptococcus neoformans, Histoplasma capsulatum, Pneumocystis carnii, Eimeria acervulina, Neospora caninum, Plasmodium falciparum, Sarcocystis suihominis, Toxoplasma gondii, Leishmania amazonensis, Leishmania major, Leishmania mexacana, Leptomonas karyophilus, Phytomonas spp., Trypanasoma cruzi, Encephahtozoon cuniculi, Nosema helminthorum, Unikaryon legeri. In a specific embodiment, an EphA2/EphA4 vaccine is a Listeria-based vaccine expresses an EphA2 and/or an EphA4 antigenic peptide. In a further embodiment, the Listeria-based vaccine expressing an EphA2- and/or an EphA4 antigenic peptide is attenuated. In a specific embodiment, an EphA2 or EphA4 vaccine is not Listeria-based or is not EphA2-based.

In another embodiment, the EphA2- and/or EphA4 antigenic peptide expression vehicle is a virus expressing an EphA2- and/or an EphA4 antigenic peptide. Non-limiting examples of viruses that can be utilized in accordance with the invention as an expression vehicle include RNA viruses (e.g., single stranded RNA viruses and double stranded RNA viruses), DNA viruses (e.g., double stranded DNA viruses), enveloped viruses, and non-enveloped viruses. Other non-limiting examples of viruses useful as EphA2- and/or EphrinA1 antigenic peptide expression vehicles include retroviruses (including but not limited to lentiviruses), adenoviruses, adeno-associated viruses, or herpes simplex viruses. Preferred viruses for administration to human subjects are attenuated viruses. A virus can be attenuated, for example, by exposing the virus to mutagens, such as ultraviolet irradiation or chemical mutagens, by multiple passages and/or passage in non-permissive hosts, and/or genetically altering the virus to reduce the virulence and pathogenicity of the virus.

Microorganisms can be produced by a number of techniques well known in the art. For example, antibiotic-sensitive strains of microorganisms can be selected, microorganisms can be mutated, and mutants that lack virulence factors can be selected, and new strains of microorganisms with altered cell wall lipopolysaccharides can be constructed. In certain embodiments, the microorganisms can be attenuated by the deletion or disruption of DNA sequences which encode for virulence factors which insure survival of the microorganisms in the host cell, especially macrophages and neutrophils, by, for example, homologous recombination techniques and chemical or transposon mutagenesis. Many, but not all, of these studied virulence factors are associated with survival in macrophages such that these factors are specifically expressed within macrophages due to stress, for example, acidification, or are used to induced specific host cell responses, for example, macropinocytosis, Fields et al., 1986, Proc. Natl. Acad. Sci. USA 83: 5189-5193. Bacterial virulence factors include, for example: cytolysin; defensin resistance loci; DNA K; fimbriae; GroEL; inv loci; lipoprotein; LPS; lysosomal fusion inhibition; macrophage survival loci; oxidative stress response loci; pho loci (e.g., PhoP and PhoQ); pho activated genes (pag; e.g., pagB and pagC); phoP and phoQ regulated genes (prg); porins; serum resistance peptide; virulence plasmids (such as spvB, traT and ty2).

Yet another method for the attenuation of the microorganisms is to modify substituents of the microorganism which are responsible for the toxicity of that microorganism. For example, lipopolysaccharide (LPS) or endotoxin is primarily responsible for the pathological effects of bacterial sepsis. The component of LPS which results in this response is lipid A (LA). Elimination or mitigation of the toxic effects of LA results in an attenuated bacteria since 1) the risk of septic shock in the patient would be reduced and 2) higher levels of the bacterial EphA2 or EphrinA1 antigenic peptide expression vehicle could be tolerated.

Rhodobacter (Rhodopseudomonas) sphaeroides and Rhodobacter capsulatus each possess a monophosphoryl lipid A (MLA) which does not elicit a septic shock response in experimental animals and, further, is an endotoxin antagonist. Loppnow et al., 1990, Infect. Immun. 58: 3743-3750; Takayma et al., 1989, Infect. Immun. 57: 1336-1338. Gram negative bacteria other than Rhodobacter can be genetically altered to produce MLA, thereby reducing its potential of inducing septic shock.

Yet another example for altering the LPS of bacteria involves the introduction of mutations in the LPS biosynthetic pathway. Several enzymatic steps in LPS biosynthesis and the genetic loci controlling them in a number of bacteria have been identified, and several mutant bacterial strains have been isolated with genetic and enzymatic lesions in the LPS pathway. In certain embodiments, the LPS pathway mutant is a firA mutant. firA is the gene that encodes the enzyme UDP-3-O(R-30 hydroxymyristoyl)-glycocyamine N-acyltransferase, which regulates the third step in endotoxin biosynthesis (Kelley et al., 1993, J. Biol. Chem. 268: 19866-19874).

As a method of insuring the attenuated phenotype and to avoid reversion to the non-attenuated phenotype, the bacteria may be engineered such that it is attenuated in more than one manner, e.g., a mutation in the pathway for lipid A production and one or more mutations to auxotrophy for one or more nutrients or metabolites, such as uracil biosynthesis, purine biosynthesis, and arginine biosynthesis.

The EphA2 or EphA4 antigenic peptides are preferably expressed in a microorganism, such as bacteria, using a heterologous gene expression cassette. A heterologous gene expression cassette is typically comprised of the following ordered elements: (1) prokaryotic promoter; (2) Shine-Dalgarno sequence; (3) secretion signal (signal peptide); and, (4) heterologous gene. Optionally, the heterologous gene expression cassette may also contain a transcription termination sequence, in constructs for stable integration within the bacterial chromosome. While not required, inclusion of a transcription termination sequence as the final ordered element in a heterologous gene expression cassette may prevent polar effects on the regulation of expression of adjacent genes, due to read-through transcription.

The expression vectors introduced into the microorganism EphA2 or EphA4 vaccines are preferably designed such that microorganism-produced EphA2 or EphA4 peptides and, optionally, prodrug converting enzymes, are secreted by microorganism. A number of bacterial secretion signals are well known in the art and may be used in the compositions and methods of the present invention. In certain embodiments of the present invention, the bacterial EphA2 or EphA4 antigenic peptide expression vehicles are engineered to be more susceptible to an antibiotic and/or to undergo cell death upon administration of a compound. In other embodiments of the present invention, the bacterial EphA2 or EphA4 antigenic peptide expression vehicles are engineered to deliver suicide genes to the target EphA2- or EphA4-expressing cells. These suicide genes include pro-drug converting enzymes, such as Herpes simplex thymidine kinase (TK) and bacterial cytosine deaminase (CD). TK phosphorylates the non-toxic substrates acyclovir and ganciclovir, rendering them toxic via their incorporation into genomic DNA. CD converts the non-toxic 5-fluorocytosine (5-FC) into 5-fluorouracil (5-FU), which is toxic via its incorporation into RNA. Additional examples of pro-drug converting enzymes encompassed by the present invention include cytochrome p450 NADPH oxidoreductase which acts upon mitomycin C and porfiromycin (Murray et al., 1994, J. Pharmacol. Exp. Therapeut. 270: 645-649). Other exemplary pro-drug converting enzymes that may be used include: carboxypeptidase; beta-glucuronidase; penicillin-V-amidase; penicillin-G-amidase; beta-lactamase; beta.-glucosidase; nitroreductase; and carboxypeptidase A.

Exemplary secretion signals that can be used with gram-positive microorganisms include SecA (Sadaie et al., 1991, Gene 98: 101-105), SecY (Suh et al., 1990, Mol. Microbiol. 4: 305-314), SecE (Jeong et al., 1993, Mol. Microbiol. 10: 133-142), FtsY and FfH (PCT/NL 96/00278), and PrsA (International Publication No. WO 94/19471). Exemplary secretion signals that may be used with gram-negative microorganisms include those of soluble cytoplasmic proteins such as SecB and heat shock proteins; that of the peripheral membrane-associated protein SecA; and those of the integral membrane proteins SecY, SecE, SecD and SecF.

The promoters driving the expression of the EphA2 or EphA4 antigenic peptides and, optionally, pro-drug converting enzymes, may be either constitutive, in which the peptides or enzymes are continually expressed, inducible, in which the peptides or enzymes are expressed only upon the presence of an inducer molecule(s), or cell-type specific control, in which the peptides or enzymes are expressed only in certain cell types. For example, a suitable inducible promoter can be a promoter responsible for the bacterial “SOS” response (Friedberg et al., In: DNA Repair and Mutagenesis, pp. 407-455, Am. Soc. Microbiol. Press, 1995). Such a promoter is inducible by numerous agents including chemotherapeutic alkylating agents such as mitomycin (Oda et al., 1985, Mutation Research 147: 219-229; Nakamura et al., 1987, Mutation Res. 192: 239-246; Shimda et al., 1994, Carcinogenesis 15: 2523-2529) which is approved for use in humans. Promoter elements which belong to this group include umuC, sulA and others (Shinagawa et al., 1983, Gene 23: 167-174; Schnarr et al., 1991, Biochemie 73: 423-431). The sulA promoter includes the ATG of the sulA gene and the following 27 nucleotides as well as 70 nucleotides upstream of the ATG (Cole, 1983, Mol. Gen. Genet. 189: 400-404). Therefore, it is useful both in expressing foreign genes and in creating gene fusions for sequences lacking initiating codons.

In certain embodiments, an EphA2 or EphA4 vaccine does not comprise a microorganism.

5.5. Pharmaceutical Compositions

The compositions of the invention include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. In one embodiment, the composition of the invention further comprises an additional therapeutic, e.g., anti-cancer, agent.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), or MF59C.1 adjuvant available from Chiron, Emeryville, Calif.), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Methods of administering a prophylactic or therapeutic agent of the invention include, but are not limited to, parenteral administration (e.g., intradermal, intramuscular, intraperitoneal, intravenous and subcutaneous), epidural, and mucosal (e.g., intranasal, inhaled, and oral routes). In a specific embodiment, prophylactic or therapeutic agents of the invention are administered intramuscularly, intravenously, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the prophylactic or therapeutic agents of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers.

In yet another embodiment, the prophylactic or therapeutic agent can be delivered in a controlled release or sustained release system. In one embodiment, a pump may be used to achieve controlled or sustained release (see Langer, supra; Sefton, 1987, CRC Crit. Ref. Biomed. Eng. 14: 20; Buchwald et al., 1980, Surgery 88: 507; Saudek et al., 1989, N. Engl. J. Med. 321: 574). In another embodiment, polymeric materials can be used to achieve controlled or sustained release of the antibodies of the invention or fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23: 61; see also Levy et al., 1985, Science 228: 190; During et al., 1989, Ann. Neurol. 25: 351; Howard et al., 1989, J. Neurosurg. 7 1: 105); U.S. Pat. Nos. 5,679,377; 5,916,597; 5,912,015; 5,989,463; 5,128,326; International Publication Nos. WO 99/15154 and WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249: 1527-1533). Any technique known to one of skill in the art can be used to produce sustained release formulations comprising one or more therapeutic agents of the invention. See, e.g., U.S. Pat. No. 4,526,938; International Publication Nos. WO 91/05548 and WO 96/20698; Ning et al., 1996, Radiotherapy & Oncology 39: 179-189; Song et al., 1995, PDA Journal of Pharmaceutical Science & Technology 50: 372-397; Cleek et al., 1997, Pro. Int'l. Symp. Control. Rel. Bioact. Mater. 24: 853-854; and Lam et al., 1997, Proc. Int'l. Symp. Control Rel. Bioact. Mater. 24: 759-760, each of which is incorporated herein by reference in its entirety.

5.5.1. Formulations

Pharmaceutical compositions for use in accordance with the present invention may be formulated in conventional manner using one or more physiologically acceptable carriers or excipients.

Thus, the compositions of the invention may be formulated for administration by inhalation or insufflation (either through the mouth or the nose) or oral, parenteral or mucosal (such as buccal, vaginal, rectal, sublingual) administration. In a preferred embodiment, local or systemic parenteral administration is used.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the prophylactic or therapeutic agents for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The prophylactic or therapeutic agents may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The prophylactic or therapeutic agents may also be formulated in rectal compositions such as suppositories or retention enemas, e.g., containing conventional suppository bases such as cocoa butter or other glycerides.

In addition to the formulations described previously, the prophylactic or therapeutic agents may also be formulated as a depot preparation. Such long acting formulations may be administered by implantation (for example subcutaneously or intramuscularly) or by intramuscular injection. Thus, for example, the prophylactic or therapeutic agents may be formulated with suitable polymeric or hydrophobic materials (for example as an emulsion in an acceptable oil) or ion exchange resins, or as sparingly soluble derivatives, for example, as a sparingly soluble salt.

The invention also provides that a prophylactic or therapeutic agent is packaged in a hermetically sealed container such as an ampoule or sachette indicating the quantity. In one embodiment, the prophylactic or therapeutic agent is supplied as a dry sterilized lyophilized powder or water free concentrate in a hermetically sealed container and can be reconstituted, e.g., with water or saline to the appropriate concentration for administration to a subject.

In a preferred embodiment of the invention, the formulation and administration of various chemotherapeutic, biological/immunotherapeutic and hormonal therapeutic agents are known in the art and often described in the Physician's Desk Reference, 56^{th} ed. (2002). For instance, in certain specific embodiments of the invention, the therapeutic agents of the invention can be formulated and supplied as provided in Table 1.

In other embodiments of the invention, radiation therapy agents such as radioactive isotopes can be given orally as liquids in capsules or as a drink. Radioactive isotopes can also be formulated for intravenous injections. The skilled oncologist can determine the preferred formulation and route of administration.

In certain embodiments the compositions of the invention, are formulated at 1 mg/ml, 5 mg/ml, 10 mg/ml, and 25 mg/ml for intravenous injections and at 5 mg/ml, 10 mg/ml, and 80 mg/ml for repeated subcutaneous administration and intramuscular injection.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

5.5.2. Dosages and Frequency of Administration

The amount of a prophylactic or therapeutic agent or a composition of the invention which will be effective in the prevention, treatment, management, and/or amelioration of a hyperproliferative disease or one or more symptoms thereof can be determined by standard clinical methods. The frequency and dosage will vary also according to factors specific for each patient depending on the specific therapies (e.g., the specific therapeutic or prophylactic agent or agents) administered, the severity of the disorder, disease, or condition, the route of administration, as well as age, body, weight, response, and the past medical history of the patient. For example, the dosage of a prophylactic or therapeutic agent or a composition of the invention which will be effective in the treatment, prevention, management, and/or amelioration of an hyperproliferative disease or one or more symptoms thereof can be determined by administering the composition to an animal model such as, e.g., the animal models disclosed herein or known in to those skilled in the art. In addition, in vitro assays may optionally be employed to help identify optimal dosage ranges. Suitable regimens can be selected by one skilled in the art by considering such factors and by following, for example, dosages are reported in literature and recommended in the Physician's Desk Reference (58th ed., 2004).

In various embodiments, the prophylactic or therapeutic agents are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, two or more components are administered within the same patient visit.

The dosage amounts and frequencies of administration provided herein are encompassed by the terms therapeutically effective and prophylactically effective. The dosage and frequency further will typically vary according to factors specific for each patient depending on the specific therapeutic or prophylactic agents administered, the severity and type of cancer, the route of administration, as well as age, body weight, response, and the past medical history of the patient. Suitable regimens can be selected by one skilled in the art by considering such factors and by following, for example, dosages reported in the literature and recommended in the Physician's Desk Reference (58^{th} ed., 2004).

Exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

For antibodies, proteins, polypeptides, peptides and fusion proteins encompassed by the invention, the dosage administered to a patient is typically 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention or fragments thereof may be reduced by enhancing uptake and tissue penetration of the antibodies by modifications such as, for example, lipidation.

In a specific embodiment, the dosage of EphA2 and/or EphA4 binding moieties (e.g., antibodies, compositions, or combination therapies of the invention) administered to prevent, treat, manage, and/or ameliorate a hyperproliferative disease or one or more symptoms thereof in a patient is 150 μg/kg or less, preferably 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of a patient's body weight. In another embodiment, the dosage of the EphA2 and/or EphA4 binding moieties or combination therapies of the invention administered to prevent, treat, manage, and/or ameliorate a hyperproliferative disease, or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

In other embodiments, a subject is administered one or more doses of an effective amount of one or EphA2/EphrinA1 Modulators of the invention, wherein the dose of an effective amount achieves a serum titer of at least 0.1 μg/ml, at least 0.5 μg/ml, at least 1 μg/ml, at least 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml of the EphA2/EphrinA1 Modulators of the invention. In yet other embodiments, a subject is administered a dose of an effective amount of one or more EphA2/EphrinA1 Modulators of the invention to achieve a serum titer of at least 0.1 μg/ml, at least 0.5 μg/ml, at least 1 μg/ml, at least, 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml of the antibodies and a subsequent dose of an effective amount of one or more EphA2 or EphA4 binding moieties of the invention is administered to maintain a serum titer of at least 0.1 μg/ml, 0.5 μg/ml, 1 μg/ml, at least, 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml. In accordance with these embodiments, a subject may be administered 1, 2, 3, 4, 5, 6, 7, 8, 9, 10, 11, 12 or more subsequent doses.

In a specific embodiment, the invention provides methods of preventing, treating, managing, or ameliorating a hyperproliferative disease or one or more symptoms thereof, said method comprising administering to a subject in need thereof a dose of at least 10 μg, preferably at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, at least 100 μg, at least 105 μg, at least 110 μg, at least 115 μg, or at least 120 μg of one or more EphA2/EphrinA1 Modulators, combination therapies, or compositions of the invention. In another embodiment, the invention provides a method of preventing, treating, managing, and/or ameliorating a hyperproliferative disease or one or more symptoms thereof, said methods comprising administering to a subject in need thereof a dose of at least 10 μg, preferably at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, at least 100 μg, at least 105 μg, at least 110 μg, at least 115 μg, or at least 120 μg of one or more EphA2 and/or EphA4 binding moieties, combination therapies, or compositions of the invention once every 3 days, preferably, once every 4 days, once every 5 days, once every 6 days, once every 7 days, once every 8 days, once every 10 days, once every two weeks, once every three weeks, or once a month.

The present invention provides methods of preventing, treating, managing, or preventing a hyperproliferative disease or one or more symptoms thereof, said method comprising: (a) administering to a subject in need thereof one or more doses of a prophylactically or therapeutically effective amount of one or more EphA2 and/or EphA4 binding moieties, combination therapies, or compositions of the invention; and (b) monitoring the plasma level/concentration of the said administered EphA2 and/or EphA4 binding moieties in said subject after administration of a certain number of doses of the said EphA2/EphrinA1 Modulators. Moreover, preferably, said certain number of doses is 1, 2, 3, 4, 5, 6, 7, 8, 9, 10, 11, or 12 doses of a prophylactically or therapeutically effective amount one or more EphA2 and/or binding moieties, compositions, or combination therapies of the invention.

In a specific embodiment, the invention provides a method of preventing, treating, managing, and/or ameliorating a hyperproliferative disease or one or more symptoms thereof, said method comprising: (a) administering to a subject in need thereof a dose of at least 10 μg (preferably at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, or at least 100 μg) of one or more EphA2/EphrinA1 Modulators of the invention; and (b) administering one or more subsequent doses to said subject when the plasma level of the EphA2 and/or EphA4 binding moiety administered in said subject is less than 0.1 μg/ml, preferably less than 0.25 μg/ml, less than 0.5 μg/ml, less than 0.75 μg/ml, or less than 1 μg/ml. In another embodiment, the invention provides a method of preventing, treating, managing, and/or ameliorating a hyperproliferative disease or one or more symptoms thereof, said method comprising: (a) administering to a subject in need thereof one or more doses of at least 10 μg (preferably at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, or at least 100 μg) of one or more antibodies of the invention; (b) monitoring the plasma level of the administered EphA2 and/or EphA4 binding moieties of the invention in said subject after the administration of a certain number of doses; and (c) administering a subsequent dose of EphA2 and/or EphA4 binding moieties of the invention when the plasma level of the administered EphA2/EphrinA1 Modulator in said subject is less than 0.1 μg/ml, preferably less than 0.25 μg/ml, less than 0.5 μg/ml, less than 0.75 μg/ml, or less than 1 μg/ml. Preferably, said certain number of doses is 1, 2, 3, 4, 5, 6, 7, 8, 9, 10, 11, or 12 doses of an effective amount of one or more EphA2 and/or EphA4 binding moieties of the invention.

Therapies (e.g., prophylactic or therapeutic agents), other than the EphA2 and/or EphA4 binding moieties of the invention, which have been or are currently being used to prevent, treat, manage, and/or ameliorate a hyperproliferative disease or one or more symptoms thereof can be administered in combination with one or more EphA2 and/or EphA4 binding moieties according to the methods of the invention to treat, manage, prevent, and/or ameliorate a hyperproliferative disease or one or more symptoms thereof. Preferably, the dosages of prophylactic or therapeutic agents used in combination therapies of the invention are lower than those which have been or are currently being used to prevent, treat, manage, and/or ameliorate a hyperproliferative disease or one or more symptoms thereof. The recommended dosages of agents currently used for the prevention, treatment, management, or amelioration of a hyperproliferative disease or one or more symptoms thereof can be obtained from any reference in the art including, but not limited to, Hardman et al., eds., 2001, Goodman & Gilman's The Pharmacological Basis Of Basis Of Therapeutics, 10th ed., Mc-Graw-Hill, New York; Physician's Desk Reference (PDR) 58th ed., 2004, Medical Economics Co., Inc., Montvale, N.J., which are incorporated herein by reference in its entirety.

In various embodiments, the therapies (e.g., prophylactic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In preferred embodiments, two or more therapies are administered within the same patient visit.

In certain embodiments, one or more antibodies of the invention and one or more other therapies (e.g., prophylactic or therapeutic agents) are cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

In certain embodiments, the administration of the same EphA2 and/or EphA4 binding moiety of the invention may be repeated and the administrations may be separated by at least 1 day, 2 days, 3 days, 5 days, 10 days, 15 days, 30 days, 45 days, 2 months, 75 days, 3 months, or at least 6 months. In other embodiments, the administration of the same therapy (e.g., prophylactic or therapeutic agent) other than an EphA2 and/or EphA4 binding moieties of the invention may be repeated and the administration may be separated by at least at least 1 day, 2 days, 3 days, 5 days, 10 days, 15 days, 30 days, 45 days, 2 months, 75 days, 3 months, or at least 6 months.

In certain embodiments, the EphA2 or EphA4 antigenic peptides and anti-idiotypic antibodies of the invention are formulated at 1 mg/ml, 5 mg/ml, 10 mg/ml, and 25 mg/ml for intravenous injections and at 5 mg/ml, 10 mg/ml, and 80 mg/ml for repeated subcutaneous administration and intramuscular injection.

Where the EphA2 or EphA4 vaccine is a bacterial vaccine, the vaccine can be formulated at amounts ranging between approximately 1×10² CFU/ml to approximately 1×10¹² CFU/ml, for example at 1×10² CFU/ml, 5×10² CFU/ml, 1×10³ CFU/ml, 5×10³ CFU/ml, 1×10⁴ CFU/ml, 5×10⁴ CFU/ml, 1×10⁵ CFU/ml, 5×10⁵ CFU/ml, 1×10⁶ CFU/ml, 5×10⁶ CFU/ml, 1×10⁷ CFU/ml, 5×10⁷ CFU/ml, 1×10⁸ CFU/ml, 5×10⁸ CFU/ml, 1×10⁹ CFU/ml, 5×10⁹ CFU/ml, 1×10¹⁰ CFU/ml, 5×10¹⁰ CFU/ml, 1×10¹¹ CFU/ml, 5×10¹¹ CFU/ml, or 1×10¹² CFU/ml.

For EphA2 and EphA4 antigenic peptides or anti-idiotypic antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight.

With respect to the dosage of bacterial EphA2 and EphA4 vaccines of the invention, the dosage is based on the amount colony forming units (c.f.u.). Generally, in various embodiments, the dosage ranges are from about 1.0 c.f.u./kg to about 1×10¹⁰ c.f.u./kg; from about 1.0 c.f.u./kg to about 1×10⁸ c.f.u./kg; from about 1×10² c.f.u./kg to about 1×10⁸ c.f.u./kg; and from about 1×10⁴ c.f.u./kg to about 1×10⁸ c.f.u./kg. Effective doses may be extrapolated from dose-response curves derived animal model test systems. In certain exemplary embodiments, the dosage ranges are 0.001-fold to 10,000-fold of the murine LD₅₀, 0.01-fold to 1,000-fold of the murine LD₅₀, 0.1-fold to 500-fold of the murine LD₅₀, 0.5-fold to 250-fold of the murine LD₅₀, 1-fold to 100-fold of the murine LD₅₀, and 5-fold to 50-fold of the murine LD₅₀. In certain specific embodiments, the dosage ranges are 0.00.1-fold, 0.01-fold, 0.1-fold, 0.5-fold, 1-fold, 5-fold, 10-fold, 50-fold, 100-fold, 200-fold, 500-fold, 1,000-fold, 5,000-fold or 10,000-fold of the murine LD₅₀.

5.6. Detection of Hyperproliferative Conditions

LMW-PTP, EphA2 or EphA4 can also serve as markers for cancer or precancerous conditions. The invention therefore also includes a method for diagnosing a cancerous or precancerous condition, or staging a cancer, by detecting and, optionally, quantifying the amount or activity of LMW-PTP, EphA2 or EphA4 in a biological sample. The diagnostic method of the invention can be used to obtain or confirm an initial diagnosis of cancer, or to provide information on cancer localization, cancer metastasis, or cancer prognosis.

In one embodiment of the diagnostic method, a biological sample such as a tissue, organ or fluid is removed from the mammal, cells are lysed, and the lysate is contacted with a polyclonal or monoclonal LMW-PTP, EphA2 or EphA4 antibody. The resulting antibody/LMW-PTP, antibody/EphA2 or antibody/EphA4 bound complex is either itself detectable or capable of associating with another compound to form a detectable complex. Bound antibody can be detected directly in an ELISA or similar assay; alternatively, the diagnostic agent can comprise a detectable label, and the detectable label can be detected using methods known in the art.

In embodiments of the diagnostic method wherein LMW-PTP, EphA2 or EphA4 is detected via the binding of a detectably labeled diagnostic agent such as an antibody, preferred labels include chromogenic dyes, fluorescent labels and radioactive labels. Among the most commonly used chromagens are 3-amino-9-ethylcarbazole (AEC) and 3,3′-diaminobenzidine tetrahydrocholoride (DAB). These can be detected using light microscopy.

The most commonly used fluorescent labeling compounds are fluorescein isothiocyanate, rhodamine, phycoerythrin, phycocyanin, allophycocyanin, o-phthaldehyde and fluorescamine. Chemiluminescent and bioluminescent compounds such as luminol, isoluminol, theromatic acridinium ester, imidazole, acridinium salt, oxalate ester, luciferin, luciferase, and aequorin also may be used. When the fluorescent-labeled antibody is exposed to light of the proper wavelength, its presence can be detected due to its fluorescence.

Radioactive isotopes which are particularly useful for labeling the antibodies of the present invention include ³H, ¹²⁵I, ¹³¹I, ³⁵S, ³²P, and ¹⁴C. The radioactive isotope can be detected by such means as the use of a gamma counter, a scintillation counter, or by autoradiography.

Antibody-antigen complexes can be detected using western blotting, dot blotting, precipitation, agglutination, enzyme immunoassay (EIA) or enzyme-linked immunosorbent assay (ELISA), immunohistochemistry, in situ hybridization, flow cytometry on a variety of tissues or bodily fluids, and a variety of sandwich assays. These techniques are well known in the art. See, for example, U.S. Pat. No. 5,876,949, hereby incorporated by reference. In an enzyme immunoassay (EIA) or enzyme-linked immunosorbent assay (ELISA), the enzyme, when subsequently exposed to its substrate, reacts with the substrate and generates a chemical moiety which can be detected, for example, by spectrophotometric, fluorometric, or visual means. Enzymes which can be used to detectably label antibodies include, but are not limited to malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase, and acetylcholinesterase. Other methods of labeling and detecting antibodies are known in the art and are within the scope of this invention.

In another embodiment of the diagnostic method, a biological sample is subjected to a biochemical assay for LMW-PTP phosphatase activity or EphA2/EphA4 kinase activity. Detection can also be accomplished by employing a detectable reagent that binds to DNA or RNA coding for the LMW-PTP, EphA2 or EphA4 protein.

LMW-PTP, EphA2 and/or EphA4 can be used as a marker for cancer, precancerous or metastatic disease in a wide variety of tissue samples, including biopsied tumor tissue and a variety of body fluid samples, such as blood, plasma, spinal fluid, saliva, and urine.

Other antibodies may be used in combination with antibodies that bind to LMW-PTP, EphA2 or EphA4 to provide further information concerning the presence or absence of cancer and the state of the disease. For example, the use of phosphotyrosine-specific antibodies provides additional data for determining detecting or evaluating malignancies.

5.7. Characterization and Demonstration of Therapeutic Utility

Toxicity and efficacy of the prophylactic and/or therapeutic protocols of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The anti-cancer activity of the therapies used in accordance with the present invention also can be determined by using various experimental animal models for the study of cancer such as the SCID mouse model or transgenic mice where a mouse EphA2 or EphA4 is replaced with the human EphA2 or EphA4, nude mice with human xenografts, animal models described in Section 6 infra, or any animal model (including hamsters, rabbits, etc.) known in the art and described in Relevance of Tumor Models for Anticancer Drug Development (1999, eds. Fiebig and Burger); Contributions to Oncology (1999, Karger); The Nude Mouse in Oncology Research (1991, eds. Boven and Winograd); and Anticancer Drug Development Guide (1997 ed. Teicher), herein incorporated by reference in their entireties.

LMW-PTP can serve as a surrogate marker to evaluate the efficacy of cancer therapeutic agents, particularly those that target EphA2 or EphA4. The amount or activity of LMW-PTP in a cancer cell that overexpresses LMW-PTP (the control) is compared to the amount or activity of LMW-PTP in an analogous cancer cell that has been treated with a candidate therapeutic agent. Reduction in the amount or activity of LMW-PTP in the treated cell is indicative of an efficacious cancer treatment.

5.7.1. Demonstration of Therapeutic or Prophylactic Utility

The protocols and compositions of the invention are preferably tested in vitro, and then in vivo, for the desired therapeutic or prophylactic activity, prior to use in humans. For example, in vitro assays which can be used to determine whether administration of a specific therapeutic protocol is indicated, include in vitro cell culture assays in which a patient tissue sample is grown in culture, and exposed to or otherwise administered a protocol, and the effect of such protocol upon the tissue sample is observed, e.g., increased phosphorylation/degradation of EphA2 or EphA4, inhibition of or decrease in growth and/or colony formation in soft agar or tubular network formation in three-dimensional basement membrane or extracellular matrix preparations. A lower level of proliferation or survival of the contacted cells indicates that the therapeutic agent is effective to treat the condition in the patient. Alternatively, instead of culturing cells from a patient, therapeutic agents and methods may be screened using cells of a tumor or malignant cell line. Many assays standard in the art can be used to assess such survival and/or growth; for example, cell proliferation can be assayed by measuring ³H-thymidine incorporation, by direct cell count, by detecting changes in transcriptional activity of known genes such as proto-oncogenes (e.g., fos, myc) or cell cycle markers; cell viability can be assessed by trypan blue staining, differentiation can be assessed visually based on changes in morphology, increased phosphorylation/degradation of EphA2 or EphA4, decreased growth and/or colony formation in soft agar or tubular network formation in three-dimensional basement membrane or extracellular matrix preparation, etc.

Compounds for use in therapy can be tested in suitable animal model systems prior to testing in humans, including but not limited to in rats, mice, chicken, cows, monkeys, rabbits, hamsters, etc., for example, the animal models described above. The compounds can then be used in the appropriate clinical trials.

Further, any assays known to those skilled in the art can be used to evaluate the prophylactic and/or therapeutic utility of the combinatorial therapies disclosed herein for treatment or prevention of cancer.

5.8. Kits

The invention provides a pharmaceutical pack or kit comprising one or more containers filled with a composition of the invention. Additionally, one or more other prophylactic or therapeutic agents useful for the treatment of a cancer can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### 6. EXAMPLES

6.1. Protein-Protein Interaction Involving LMW-PTP and EphA2

6.1.1. Materials and Methods

Protein Production. Ampicillin, N-Z-amine A (casein hydrolysate), IPTG, and SP-Sephadex C-50 all were obtained from Sigma. The SP-Sephadex G-50 was purchased from Pharmacia. The YM3 membranes were from Amicon. All other materials were purchased either from Sigma or BioRad.

Cell lines. The cell models used for this study were breast epithelia. A commonly used cell line in this research laboratory is MCF-10A. This cell line is a part of the family of MCF-10 cells, an established immortal human mammary epithelial cell line. MCF-10 cells were isolated from the mammary tissue of an adult woman who had fibrocystic disease. MCF-10A cells grow as attached cells. The MCF-10A (Neo) cell line is the parent cell line, MCF-10A, with a neomycin resistance gene. The MDA-MB-231 cell line is a highly invasive and metastatic mammary cell line. These cells were isolated from an adult woman with breast cancer.

Care for these cells consist of handling them every two days, either by refreshing the media or splitting them. To split the cells, the media was first removed by aspiration. The cells were washed in 2-3 ml PBS, then trypsin solution (2-3 ml diluted 1:50 in PBS) was added and the plates were placed in the incubator at 37° C. for 10-30 minutes. Next, 2-3 ml of media was added to each plate. The cells in the PBS/trypsin solution and media were spun to a pellet in a tabletop centrifuge for 5 minutes. The PBS/trypsin solution and media were aspirated, and the cells were resuspended in media. The cells were then plated in the tissue culture dishes.

The growth medium for the MCF-10A (Neo) cells consists of DMEM/F12, 5.6% horse serum, 20 ng/ml epidermal growth factor, all from Upstate Biotechnology, Inc., 100 μg/ml streptomycin, 100 units/ml penicillin, 10 μg/ml insulin from Sigma, 0.25 μg/ml fungizone, and 2 nM L-Glutamine. The growth medium for the MDA-MB-231 cells consists of RPMI, 2 nM L-Glutamine, 100 μg/ml streptomycin, and 100 units/ml penicillin.

Antibodies. An antibody that recognizes the intracellular domain of EphA2 is D7 (Upstate Biochemicals, New York). This monoclonal antibody (MAb) was produced from a bulk culture as stated in Zantek, Ph.D. Thesis, Purdue University, 1999. For immunoprecipitations with this antibody, 30 μl were used. For immunoblotting, a dilution of 1:1 in TBSTB (30 ml 5M NaCl, 50 ml 1 M Tris, pH 7.6, 1 ml Tween-20, 1 g BSA, and 920 ml ddH₂O) was used. For immunofluorescence microscopy, the antibody was used without dilution.

The monoclonal antibodies directed against HPTP (10.1 and 7.1) were developed as stated in Alfred Schroff, Ph.D. Thesis, Purdue University, 1997. For immunoprecipitations, with 10.1 (α-HPTP-B) 10 μl were used. For immunoblotting, the antibody was diluted in TBSTB at 1:100. For immunofluorescence microscopy, the antibody was diluted at 1:10 in PBS. The same conditions were used for the other MAb directed against HPTP, 7.1 (α-HPTP-A/B). For the polyclonal antibodies against HPTP, 10 μl of antibody were used for immunoprecipitation. For immunoblotting, the antibody was diluted in TBSTB at 1:2000. For immunofluorescence microscopy, the antibody was diluted in PBS at 1:100.

To detect phosphotyrosine, the antibody known as 4G10 was used. This antibody was produced from a bulk culture. For immunoblotting, a dilution of 1:1 in TBSTB was used. The secondary antibodies used for immunofluorescence microscopy were DAR-Fl at a 1:40 dilution and/or DAM-Rh at a 1:100 dilution in PBS. For immunoblotting experiments, either Goat Anti-Mouse (for MAb) or Goat Anti-Rabbit (for PAb) was used at a 1:10,000 dilution in TBSTB.

Affinity matrices. Protein-A Sepharose was purchased from Sigma. The Affi-gel 10 was purchased from BioRad.

Other materials. Other materials were purchased from Fisher, Pierce, Malinckrodt, New England Biolabs, QIAGEN, and Roche Diagnostics.

LMW-PTP expression and purification. The growth medium (M9ZB) was prepared as follows: in a 4 L flask, 20 g N-Z-amine A (casein hydrolysate), 10 g NaCl, 2 g NH₄Cl, 6 g KH₂PO₄, and 12 g Na₂HPO₄H₂O were dissolved in 2 L of ddH₂O. The pH of the medium was then adjusted to 7.4 with NaOH pellets. To a 500-ml flask, 200 ml of M9ZB solution were poured. The two containers of media were then autoclaved for 20 minutes. After cooling to room temperature, filter sterilized solutions of 20 mL 40% glucose and 2 mL 1M MgSO₄ were added per 2 L of medium. Just prior to inoculation 200 μl of 50 mg/mL Amp was added to the flask containing 200 ml (M9ZB) medium. A 200-ml culture of the BL21 strain of E. coli containing the recombinant plasmid with the gene of interest was grown overnight on a gyratory shaker set at 250-300 rpm at 37° C.

The next day, 1.8 ml of 50 mg/mL Amp was added to the remaining 1.8 L of fresh medium. The overnight culture was then diluted 1:10 in the fresh (M9ZB) medium, and the cells were allowed to grow an additional 3 hours. When the optical density at 600 nm (OD₆₀₀) reached between 0.6 and 1.0, 2 ml of 4 mM IPTG were added to induce protein expression. The culture was incubated at 37° C. for an additional 3 hours for WT-PTPase or 6 hours for mutant PTPases. The cells were harvested by refrigerated centrifugation for 15 minutes at 5000 rpm. The supernatant was poured back into the 4-L flask, then autoclaved for 20 minutes before discarding. The cell pellet was resuspended and washed in 10 mL 0.85% NaCl, spun to a pellet again at 500 rpm, then resuspended in 2 mL 0.85% NaCl. The mixture was placed in a small centrifuge tube then spun at 5000 rpm for 10 minutes. The supernatant was poured off, and the pellet was either stored at −20° C. overnight or lysed immediately.

The cell pellet was thawed (if applicable) then resuspended in 100 mM CH₃COONa buffer, pH 5.0, containing 1 mM EDTA and 1 mM DTT. The DTT was added just prior to use. The cells were disrupted by passing them twice through a pre-chilled French pressure cell set at a pressure gauge of 1000 psi. The lysates were spun to a pellet in the refrigerated centrifuge at 16,000 rpm for 15 minutes. The supernatant was poured into a new centrifuge tube, then loaded onto an SP-Sephadex C-50 cation-exchange column (1.5×30 cm) that was pre-equilibrated with 10 mM CH₃COONa buffer, pH 4.8, containing 30 mM NaH₂PO₄, 1 mM EDTA and 60 mM NaCl.

The C-50 column was washed with 10 bed-volumes of 10 mM CH₃COONa buffer until the A₂₈₀ was roughly zero. The protein was then eluted with a high salt solution, 300 mM NaH₂PO₄ and 1 mM EDTA at pH 5.1. The flow-rate was set at 30-40 mL/hr. Each fraction collected contained approximately 6 ml. The fractions with the highest A₂₈₀ were resolved on a 15% SDS-polyacrylamide gel to access protein purity. The purest fractions were combined, then concentrated to roughly 5 ml using an Amicon ultrafiltration apparatus. The concentrate was loaded on a Sephadex G-50 size exclusion column that was pre-equilibrated with 10 mM CH₃COONa buffer at pH 4.8, containing 30 mM NaH₂PO₄, 1 mM EDTA and 60 mM NaCl. The flow-rate was set at 15-25 ml/hr and fractions of approximately 6 mL were collected. The fractions with the highest A₂₈₀ were tested on a 15% SDS-polyacrylamide gel to assess protein purity. The purest fractions were combined and stored at 4° C. in G-50 buffer.

Immunofluorescence microscopy. Up to five glass coverslips were placed in a 3.5 cm dish. The cell line(s) appropriate for the particular study was plated into those dishes 24 hours prior to use. The cells usually reached a confluence of 60-70% by this time. The cells were fixed in a 3.7% formaldehyde solution for 2 minutes, then permeabilized in 1% Triton for 5 minutes, and washed in Universal Buffer (UB) for 5 minutes. The cells were then incubated at room temperature with the primary antibody for 30 minutes. Next, the cells were washed in UB (12 ml 5 M NaCl, 20 ml 1 M Tris, pH 7.6, 4 ml 10% Azide) for 5 minutes. The cells were then incubated with a secondary antibody for 30 minutes. After a brief wash for 5 seconds in ddH₂O, the coverslips were placed face down on approximately 5 μl of Fluor Save (Calbiochem) on a glass slide. The cells were allowed to dry at room temperature for approximately 15 minutes; then they were placed under a hair dryer set on “low” for an additional 15 minutes or until dry. The cells were viewed under an oil immersion lens (60×) of a fluorescence microscope.

Immunoprecipitation. For immunoprecipitations (IPs) with monoclonal antibodies, Rabbit Anti-Mouse Protein-A Sepharose (RAMPAS) was used. For those with polyclonal antibodies, Protein-A Sepharose (PAS) was used. The beads were prepared by, first, adding Protein-A Sepharose to the 100 μl-mark of a 1.5 mL microfuge tube. Next, 1 ml of UB was added to swell the beads. For RAMPAS, 50 μl/ml Rabbit Anti-Mouse (RAM) IgG was also added to the tube of beads and UB. The mixture(s) were allowed to rotate on a rotary stirrer overnight at 4° C. The next day, the beads were washed three times in 1 ml of UB. The beads were then brought to a 50% slurry in UB.

The plate of cells was placed on ice. The cells were washed once with 2-3 ml of PBS. Afterwards, the cells lysed in a 1% Triton lysis buffer (5 ml 1 M Tris, pH 7.6, 3 ml 5 M NaCl, 1 ml 10% NaN₃, 1 ml 200 mM EDTA, 10 ml 10% Triton X-100, 80 ml ddH₂O) or RIPA lysis buffer (5 ml 1M Tris, pH 7.6, 3 ml 5 M NaCl, 1 ml 10%, NaN₃, 1 ml 200 mM EDTA, 10 ml 10% Triton X-100, 5 ml 10% deoxycholate, 500 μl 20% SDS, 74.5 ml ddH₂O) lysis buffer containing 1 mM Na₃VO₄, 10 μg/ml leupeptin, and 10 μg/ml aprotinin for 5 minutes on ice. The lysates were collected, and each set of lysates was normalized for equal protein content using Coomassie Protein Assay Reagent. A plate-reader was used to measure the absorbance of 590 nm. After equalizing the lysates with the appropriate lysis buffer, the samples were prepared.

For each sample, 30 μl PAS (or RAMPAS) was added to each sample tube. Next, the appropriate primary antibody was added. Finally, 150-200 μl portions of the lysates were added. The samples were allowed to rotate at 4° C. for either 1.5 hours or overnight. The samples were then washed three times in 1 ml of the same lysis buffer that had been used to lyse the cells. After the final wash, 15 μl Laemmli buffer was added to the pelleted beads, and the samples were boiled for 10 minutes. Afterwards, the samples were loaded and resolved on a 15% SDS-polyacrylamide gel set at 220 V for 1.75 hours. After protein resolution, the proteins were transferred to nitrocellulose overnight.

Substrate trapping. Purified, catalytically-inactive LMW-PTP recombinant mutants, D129A-BPTP and C12A-BPTP, were used to create potential substrate trap(s). The affinity support was prepared by, first, washing 1-1.5 ml of Affi-gel 10 in several volumes of cold ddH₂O. Next, the moist gel was added to a 15-ml conical tube, along with a 5 mg/ml of pure PTPase mutant. The tube was rotated at 4° C. for 4 hours to allow the protein to couple to the beads. Afterwards, 100 μl ethanolamine per 1 ml Affi-gel was added to block reactive gel sites that had not been bound by protein, then the tube was rotated for an additional hour. The slurry was poured into a small plastic column. The beads were allowed to settle in the column, they were then washed with 20 ml of ddH₂O. The pH of the wash was measured. If greater than or equal to seven, the pH was adjusted with 10 mM HCl. Next, the A₂₈₀ was measured. If not near zero, the washes were continued until the A₂₈₀ read near zero. The column was stored at 4° C. until used.

Prior to application of the lysates, the column was washed in 10 ml of ddH₂O three times, then equilibrated in the appropriate lysis buffer. The cells were lysed in the appropriate lysis buffer for 5 minutes on ice. The lysates were collected and added to the column to incubate for various times at 4° C. The beads were then washed in the appropriate lysis buffer three times. Laemmli buffer was added to the beads, which were boiled for 10 minutes. The samples were resolved on a 15% SDS-polyacrylamide gel, and finally transferred to nitrocellulose overnight.

Dephosphorylation. MCF-10A (Neo) cells were grown to 80% confluence. The cells were lysed in 1% Triton lysis buffer for 5 minutes on ice. The lysates were collected and combined. EphA2 IPs were prepared: 30 μl D7, 30 μl RAMPAS, and 200 μl lysates. The IPs were mixed for 1.5 hours at 4° C. They were washed two times in 500 μl Triton lysis buffer, then twice in 500 μl of ddH₂O. Each pellet was resuspended in 10 mM 50 μl CH₃COONa buffer, and the tubes were placed in a 37° C. waterbath for 5 minutes to adjust the temperature to physiological conditions. Next, 500 μl of PTPase solution at the chosen concentration, was added to the tubes to react with EphA2 for the chosen times. At the end of the reaction, the beads were pelleted and the supernatant was removed by aspiration. Laemmli buffer was added to each sample and they were boiled for 10 minutes. Finally, the proteins were separated on a 10% SDS-polyacrylamide gel, and transferred to nitrocellulose overnight.

Immunoblotting. The nitrocellulose membrane was stained with Ponceau S to identify and mark the location of the molecular weight markers. The membrane was rinsed several times in ddH₂O to remove the dye. Non-specific sites on the membrane were blocked with a solution of Teleostean gelatin (50 ml of TBSTB and enough gelatin to give a “tea” color). The membrane was incubated in the blocking solution at room temperature for 30 minutes. Next, the membrane was incubated with primary antibody for 30 minutes. The membrane was washed three times for 10 minutes each in TBSTB, which was followed by a 30-minute incubation with secondary antibody. Afterwards, the membrane was washed three times for 8 minutes each in TBSTB, then twice for 6 minutes each in TBS (30 ml 5 M NaCl, 50 ml 1 M Tris, pH 7.6, 920 ml ddH₂O). Next, the chemiluminescent reagents were added to the membrane (1:1). Finally, the film was exposed to the membrane, which was wrapped in Saran Wrap, and developed.

Small-scale DNA purification. The plasmid pET-11d containing the gene for HPTP, was purified from the BL21 strain of E. coli using the commercially available QIAprep Miniprep from QIAGEN. E. coli containing HPTP-A and HPTP-B were both, but separately, streaked onto LB/Amp plates. Both plates were placed in a 37° C. incubator overnight. The next day, 3 ml of LB medium and 6 μl Amp were placed into two sterile snap-top tubes. The tubes were then labeled HPTP-A or HPTP-B. One colony from each plate was used to inoculate the respectively labeled tube with a colony containing the HPTP-A gene or the HPTP-B gene. The tubes were placed on a shaker set at 250 rpm overnight (12-16 hours). The next day, the cultures were spun to a pellet, and the supernatant was removed by aspiration.

To purify the DNA from bacterial pellets using the QIAprep Miniprep protocol, the bacterial pellets were resuspended in 250 μl of a buffered RNase A solution (Buffer P1). Next, the cell suspension was placed in a microfuge tube and lysed in 250 μl of an alkaline lysis buffer (Buffer P2) consisting of NaOH and SDS. The tubes were inverted gently five times. Lysis was carried out for 5 minutes. The mixture was then neutralized by adding 350 μl neutralizing buffer (Buffer N3).

After spinning the tubes at 13,000 rpm for 10 minutes, the supernatant was transferred to the QIAprep spin column. The spin column was placed in a 2-ml collection tube. Together, they were placed in a centrifuge and spun at 13,000 rpm for 1 minute. The flow-through was discarded. Next, the spin column was washed with 750 μl of Buffer PE, and spun at 13,000 rpm for 1 minute. After discarding the flow-through, the spin column was spun once more at 13,000 rpm for 1 minute. The spin column was placed in a clean microfuge tube, and the DNA was eluted with 60 μl of Buffer EB and stored at −20° C.

Amplification of the coding regions. Polymerase chain reaction was used to amplify the coding regions of the genes. The primer designed for the forward strand contains a Hind III restriction site: AAT TTA AAG CTT CCA TGG CGG AAC AGG CTA CCA AG (SEQ ID NO:125). The primer designed for the reverse strand contains an EcoR I restriction site: CGT TCT TGG AGA AGG CCC ACT GAG AAT TCT TCG T (SEQ ID NO:126). An additional primer designed for the reverse strand contains a BamH I restriction site: GCG CGC GGA TCC TCA GTG GGC CTT CTC C (SEQ ID NO:127).

Briefly, 50 μl reaction mixtures consisting of 40 μl ddH₂O, 5 μl 10× buffer, 1 μl forward primer, 1 μl reverse primer, 1 μl dNTPs and 1 μl pfu polymerase were prepared. The reaction mixtures were placed in a thermal cycler set for the following cycle: 94° C. for 2 minutes, 94° C. for 1 minute, 55° C. for 1 minute, 65° C. for 1 minute, 65° C. for 10 minutes, then hold at 4° C. Steps two through four were repeated 30 times prior to proceeding to the next step, 65° C. for 10 minutes.

At the end of the cycle, the PCR products were analyzed on a 1% agarose gel (600 mg agarose, 1.2 ml 50×TAE, 58.8 ml ddH₂O). The PCR products were then purified using the commercially available QIAquick PCR purification kit from QIAGEN. Briefly, five volumes of Buffer PB were added to one volume of the PCR product reaction mixture and mixed briefly. The mixture was added to the QIAquick spin column and spun for 1 minute at 13,000 rpm. After discarding the flow-through, 750 μl of PE Buffer was added to the column and spun for 1 minute more at 13,000 rpm. The column was placed in a clean microfuge tube, and 30 μl of Buffer EB was added to the column. The column incubated at room temperature with the buffer for 1 minute before being spun at 13,000 rpm for 1 minute to elute the DNA.

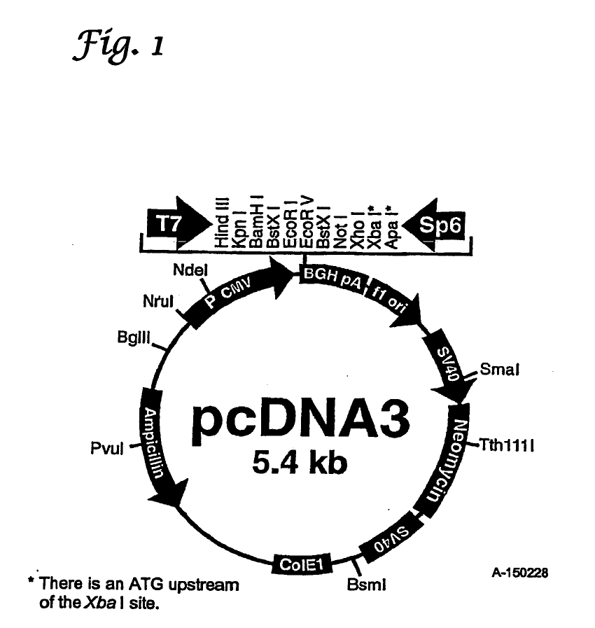

Removal of the extensions. The PCR product reaction mixtures were prepared for digestion: 5 μl PCR product, 1 μl NEB-Buffer 2, 1 μl 10×BSA, and 0.9 μl Hind III/BamH I stock. The Hind III/BamH I stock consisted of 2.4 μl Hind III and 1.2 μl Barn H I. The reaction mixture for digestion for the pcDNA3 vector (FIG. 1) from Invitrogen was prepared: 1 μl pcDNA3, 1.5 μl NEB-Buffer 2, 1.5 μl 10×BSA, 0.5 μl Hind III, and 0.5 μl BamH I. The plasmid pcDNA3 is a 5.4 kb mammalian expression vector. The HPTP gene was cloned into the Hind III/BamH I sites of this vector, and expression of the gene was driven by the CMV promoter. The PCR products and the mammalian expression vector, pcDNA3, were digested at 37° C. for 2.5 hours. The digests were analyzed on a 1% agarose gel. After resolution, a photograph was taken of the gel. Digestion of the PCR products and the pcDNA3 vector were expected to generate fragments that were 491 bp and 5,428 bp, respectively.

Gel pieces containing the digested products were removed from the gel and placed in a microfuge tube. To remove the digested products from the gel, a commercially available QIAquick Gel Extraction kit from QIAGEN was used. Briefly, 210 μl of Buffer QG were added to the tubes. The tubes were placed in a 50° C. waterbath for approximately 10 minutes, with mixing every 2-3 minutes. Next, 70 μl of isopropanol were added to the tubes and mixed. The samples were then placed in a column attached to a collection tube and spun at 13,000 rpm for 1 minute. After discarding the flow-through, 500 μl of Buffer QG were added to the column, and the column was spun for 1 minute at 13,000 rpm. The flow-through was discarded, and the column was washed with 750 μl of Buffer PE then spun at 13,000 rpm for 1 minute. The flow-through was discarded, and the column was spun once more at 13,000 rpm for 1 minute to elute the DNA. The DNA was stored at −20° C.

Ligation and transformation. The amplified HPTP-A and HPTP-B genes were both, but separately, ligated with the pcDNA3 vector. The ligation reaction mixture was prepared: 10 μl insert, 5 μl vector, 2 μl 10× ligation buffer, 2 μl 10×ATP, and 1 μl ligase. The ligation mixtures were placed in a thermal cycler set at 16° C. for 18 hours followed by holding the temperature at 4° C.

The DH5α strain of competent E. coli was transformed with the ligation mixtures. Two microfuge tubes each with 200 μl of competent E. coli (DH5α) were thawed on ice. The ligation mixture was added to each tube of cells, the tubes were vortexed briefly, and then incubated on ice for 20 minutes. The tubes were placed in a 42° C. waterbath for 1.5 minutes, then placed on ice for 2 minutes. The contents of the tubes were placed separately into tubes containing 1 ml of LB. The mixtures were placed on the shaker set at 250 rpm for 45 minutes. Next, 200 μl of each culture were spread onto two LB/Amp plates. The plates were placed in the 37° C. incubator lid side up for 10 minutes, then lid side down overnight (16-18 hours).

Screen of colonies. The QIAprep Miniprep protocol was used to purify the DNA from each of the six bacterial cultures. Tubes containing the purified DNA were labeled appropriately: colony A1, colony A2, colony B1, colony B2, etc. To screen the colonies, purified DNA from each was digested with Hind III and BamH I; Nde I and EcoR I; and Acc I. The Hind III/BamH I digestion reactions were prepared: 5 μl vector/insert, 1 μl NEB-Buffer 2, 1 μl 10×BSA, 1.8 μl Hind III/BamH I stock, 6.2 μl ddH₂O. The Hind III/BamH I stock was prepared as follows: 7.2 μl BamH I added to 9.6 μl Hind III. Next, the Nde I/EcoR I digestion reactions were prepared: 5 μl vector/insert, 0.5 μl EcoR I, 0.3 μl Nde I, 1.5 μl NEB-Buffer 4, 7.7 μl ddH₂O. Finally, the Acc I digestion reactions were prepared: 5 μl vector/insert, 0.5 μl Acc I, 1.5 μl NEB-Buffer 3, and 8 μl ddH₂O. All digests were done overnight at 37° C. The digests were resolved on a 1% agarose gel and a photograph was taken of the gel.

Medium-scale DNA purification. A six-hour 5 ml culture grew at 37° C. on the shaker set at 250 rpm. The 5-ml culture was diluted with 50 ml of LB. The tube was placed on the shaker overnight. On the next day, 40 ml of the overnight culture was transferred to a 5-ml screw-cap centrifuge tube and pelleted by centrifugation for 5 minutes at 5000 rpm. The commercially available QUANTUM MidiPrep from BioRad was used to purify DNA on a medium scale. Briefly, the supernatant was poured off and 5 ml of Cell Resuspension solution were added to the cell pellet. The tube was vortexed to resuspend the cells. Next, 5 ml of Cell Lysis solution were added to the tube, then inverted six to eight times. The mixture was neutralized by adding 5 ml Neutralization solution, followed by inverting the tube six to eight times, neutralized the solution. The mixture was spun for 10 minutes at 8000 rpm. The supernatant was transferred to a new tube along with 1 ml of Quantum-Prep matrix. The mixture was gently swirled for 15 to 30 seconds, then spun for 2 minutes at 8000 rpm. The supernatant was poured off, then 10 ml of wash buffer were added to the matrix and mixed by shaking. The tube was spun for 2 minutes at 8000 rpm. After pouring the wash buffer from the pellet, 600 μl Wash Buffer were added to the tube to resuspend the pellet. The spin column was attached to a microfuge tube and a hole was punctured in the lid of the microfuge tube. After spinning the tube for 30 seconds at 12,000 rpm, the flow-through was discarded. Next, 500 μl of Wash Buffer was added to the tube, and the tube was spun for 30 seconds at 12,000 rpm. The flow-through was discarded, then the column was spun for 2 minutes more at 12,000 rpm to remove residual Wash Buffer. The column was transferred to a clean microfuge tube. The DNA was eluted with 600 μl of TE (pH 8).

Next, the DNA was ethanol-precipitated by adding 1/10 the volume of 5 M NaCl following by two times that total volume (NaCl plus DNA) of 100% ethanol. The microfuge tube was gently inverted a few times and incubated at −20° C. for 20 minutes. The DNA was spun to a pellet for 10 minutes at 13,000 rpm. Under sterile conditions, the ethanol/NaCl was aspirated from the pellet. The pellet was left to air dry in the hood. Afterwards, the DNA was resuspended in 100 μl sterile TE (pH 8). To determine the concentration of the DNA sample, the absorbance at 260 nm (A₂₆₀) was measured. The DNA was stored at −20° C.

Transfection. To overexpress HPTP in the MCF-10A (Neo) cell line, the commercially available FuGENE© transfection kit from Roche Diagnostics was used. The cells were plated 18 hours prior to use in 6-well plates such that their confluence would be approximately 50% on the day of transfection. In a microfuge tube, 97 μl serum-free dilution media was added to 3 μl of the FuGENE reagent. The diluted FuGENE was incubated at room temperature for 5 minutes. Next, 1 μg of DNA was added to a second microfuge tube. Dropwise, the diluted FuGENE reagent was added to the DNA. The tube was gently tapped to mix the contents of the tube. The tube was then incubated for 15 minutes at room temperature. The media on the cells was replaced with 2 ml of fresh media. Dropwise, the FuGENE/media solution was added to the plated cells, then the plate was swirled to distribute the contents around the plate. The cells were incubated at 37° C. for 36 to 48 hours.

On the day of analysis, the cells were lysed in 1% Triton lysis buffer, HPTP and D7 immunoprecipitations were done. The samples were eventually resolved on a 15% SDS-polyacrylamide gel, then transferred to nitrocellulose overnight. The next day, immunoblotting was done with antibodies directed against EphA2, HPTP, and phosphotyrosine.

6.1.2. Results

6.1.2.1 Expression and Purification of the LMW-PTP

LMW-PTPs can be purified using a two-step purification scheme involving cation-exchange chromatography (typically using a SP-Sephadex C-50 column) followed by size exclusion chromatography (typically using a Sephadex G50 column). A minor difference between the recombinant protein (isolated after expression in E. coli) and the native bovine or human protein is that the recombinant protein is not acetylated on the N-terminal alanine residue as in the native tissue protein. In this Example, WT-BPTP, D129A-BPTP, and C12A-BPTP were expressed and purified using the pET-1 expression system. A stock supply of the purified proteins, HPTP-A and HPTP-B, was already on hand.

Expression and purification of WT-BPTP from E. coli occurred without difficulty and generated good quantities of protein (40-50 mg per liter of expression medium). Expression of recombinant, mutant PTPases resulted in less protein (approximately 10-15 mg per liter of expression medium). In the case of the D129A bovine mutant, the induction period was increased to six hours, and the wash buffer was changed to 1 mM EDTA to increase the binding of the mutant proteins to the C50-columns. Once purified, the protein was stable for months at −20° C. in phosphate buffer.

6.1.2.2. Comparison of the LMW-PTP in MCF-10A (Neo) and MDA-MB-231 Cell Lines

Protein levels. EphA2 is tyrosine phosphorylated in the non-transformed MCF-10A (Neo) cell line, but not in the malignant MDA-MB-231 cell line.

Endogenous protein levels of the LMW-PTP in the MCF-10A (Neo) and the MDA-MB-231 cell lines were first compared by immunoblotting analyses. The results revealed lower protein levels of the LMW-PTP in the MCF-10A (Neo) cell line relative to the levels of the protein in the MDA-MB-231 cell line. This suggests that the higher protein levels of the LMW-PTP observed in the MDA-MB-231 cell line might correlate with EphA2 being substantially more dephosphorylated in that cell line compared to MCF-10A (Neo) cell line.

Although EphA2 is tyrosine phosphorylated in MCF-10A (Neo) cells, even higher levels of tyrosine phosphorylation of the cells can be achieved if EphA2 is treated with a soluble form of its ligand or artificially activated at the cell surface (Zantek, N. D. (1999), Ph.D. Thesis, Purdue University). With this in mind, it may also be suggested that the LMW-PTP dephosphorylates EphA2 in MCF-10A (Neo) cells, but not to the same degree as in the MDA-MB-231 cells. There might be a competition between phosphorylation and dephosphorylation of EphA2, and that in MDA-MB-231 cells the balance is tilted toward dephosphorylation. However, in the MCF-10A (Neo) cells, the balance is not tilted substantially in one direction or the other. As a result, EphA2 retains some of its tyrosine phosphorylation in the MCF-10A (Neo) cell line.

Subcellular localization. A panel of polyclonal and monoclonal antibodies, all directed against the LMW-PTP, was used to stain MCF-10A (Neo) and MDA-MB-231 cells. To determine the subcellular localization of the LMW-PTP in the MCF-10A (Neo) and MDA-MB-231, cells were grown on coverslips overnight. After fixation and permeabilization, the cells were stained with a primary antibody to detect the LMW-PTP. A fluorescent tag attached to the secondary antibody facilitated observation of the subcellular location of the LMW-PTP on the fluorescence microscope. The LMW-PTP was found to be diffuse and widely distributed in the MCF-10A (Neo) cells. When MDA-MB-231 cells were stained, the LMW-PTP was found localized in the membrane ruffles. This was an exciting finding because EphA2 was known to localize in the membrane ruffles in the MDA-MB-231 cell line, as well (Zantek, N. D. (1999), Ph.D. Thesis, Purdue University).

6.1.2.3. In Vitro Protein-Protein Interaction Between LMW-PTP and EphA2

Co-immunoprecipitation, Attempts were made to co-immunoprecipitate the two proteins with separate antibodies directed against either protein. Co-immunoprecipitation of the LMW-PTP was readily detectable when immunoprecipitating with D7, an EphA2-specific antibody, followed by immunoblotting analyses with either the 7.1 or 10.0 LMW-PTP antibody. The co-immunoprecipitation was more evident when blotting with the 10.1 antibody. As would be predicted from the relative protein level analysis of the LMW-PTP, more LMW-PTP was co-immunoprecipitated from the MDA-MB-231 cell line than from the MCF-10A (Neo) cell line. A somewhat less dramatic result was obtained when immunoprecipitating with either the 7.1 or 10.1 antibody, followed by immunoblotting with the D7 antibody. Bands appeared in the lanes of the 7.1 and 10.1 IPs that were roughly co-linear with those of the D7 IP control. This suggests that these bands might represent EphA2.

It was somewhat surprising that co-immunoprecipitation of the proteins occurred in both of our cell lines. It was predicted that an interaction would be detected, but this was expected to be more likely in the MCF-10A (Neo) cell line because EphA2 is tyrosine phosphorylated there. However, because the interaction of a phosphatase with its substrate is either so transient or so weak, it was also thought that the interaction might not be easily detected. In our case, an interaction was detected in both cell lines.

6.1.2.4. In Vitro Dephosphorylation

Attempts at substrate trapping to detect direct interaction between EphA2 and LMW-PTP failed, so an alternative in vitro test was conducted. We examined the ability of pure LMW-PTP to dephosphorylate EphA2 isolated by immunoprecipitation. We found that the LMW-PTP dephosphorylated EphA2 in an enzyme concentration-dependent and a time-dependent manner.

As would be expected, the extent of dephosphorylation of EphA2 by the LMW-PTP was found to be greater when larger amounts of phosphatase are used than in cases when smaller amounts are used. The enzyme concentration-dependent dephosphorylation of EphA2 by the LMW-PTP is consistent with the hypothesis that high levels of LMW-PTP suppress tyrosine phosphorylation of EphA2 in MDA-MB-231 cells. It is thought that the higher LMW-PTP levels cause substantial dephosphorylation of EphA2 in those cells. The enzyme concentration-dependent dephosphorylation of EphA2 follows basic kinetic behavior. The rate of the reaction increases with increasing enzyme concentration. As a result, there is greater turnover per unit time. When the progress of the reaction is studied over longer periods of time, it is found that greater enzyme concentrations continue to dephosphorylate EphA2 in comparison with smaller enzyme concentrations. The leveling off of dephosphorylation that is observed may be due to instability of the protein under very dilute conditions.

6.1.2.5. In Vivo Protein-Protein Interaction Between LMW-PTP and EphA2

Vector construction. To explore the effects of overexpressing the LMW-PTP in the MCF-10A (Neo) cell line, a pcDNA3 eukaryotic expression vector containing the coding region of the LMW-PTP was constructed. Microgram amounts of the pET-11d plasmid were isolated without difficulty using a commercially available DNA purification kit. Primers were designed and used to amplify the coding region of the A- and B-isoforms of the LMW-PTP.

The PCR products were purified using a commercially available PCR product purification kit from QIAGEN. The amplified coding regions of the LMW-PTP isoenzymes were digested with BamH I and Hind III to remove the extensions. The “sticky-ends” that were produced allowed for directional cloning of the inserts in the mammalian expression vector, pcDNA3, which was also digested with Hind III and BamH I. Digestion of the isoenzymes generated 491 bp fragment. Digestion of the pcDNA3 vector generated an open vector with 18 fewer base pairs than the circular vector.

After cell transformation, the constructed vectors were isolated from the cells and screened with a panel of restriction enzymes to determine if the coding regions of the human A- and B-isoenzymes of LMW-PTP were present. The coding regions were present in their respective vectors as indicated by the cuts produced by the restriction enzymes.

Overexpression of the LMW-PTP in MCF-10A (Neo) cells. Overexpression of the LMW-PTP in the MCF-10A (Neo) cell line was attempted in order to explore the effects of increased protein levels of the phosphatase on EphA2's tyrosine phosphorylation status. Large quantities of the constructed vectors were isolated in a highly pure form using a commercially available DNA purification kit. Isolation of the vectors using this procedure occurred without great difficulty. The commercially available transfection kit, FuGENE, was used to transfect the MCF-10A (Neo) cell line with “empty” pcDNA3, HPTP-A/pcDNA3 and HPTP-B/pcDNA3, respectively. The “empty” vector served as a control in the experiments such that any changes in EphA2's tyrosine phosphorylation status should be attributable to increased levels of the LMW-PTP and not to the presence of the mammalian expression vector.

Overexpression of the HPTP-B in the MCF-10A (Neo) cell line resulted in decreased tyrosine phosphorylation levels of EphA2. No noticeable difference in EphA2's tyrosine phosphorylation was seen when HPTP-A was overexpressed in the same cell line. From this information, it might be concluded that the interaction of EphA2 the LMW-PTP is isoenzyme specific, which is not an unreasonable possibility. Differences in the amino acid sequence of the isoenzymes could be the underlying reason why only one isoenzyme appears to interact preferentially with EphA2. However, there are many other reasons that explain the difference as well.

6.1.3. Discussion

In transformed breast epithelia such as MDA-MB-231, EphA2 is not tyrosine phosphorylated. However, restoration of tyrosine phosphorylation of EphA2 occurs when these cells are treated with the pervanadate ion (Zantek, N. D. (1999), Ph.D. Thesis, Purdue University). This gives a strong indication that a PTPase is causing the loss of tyrosine phosphorylation of EphA2. Also, treatment of EphA2 with a soluble form of the ephrinA1 ligand and cross-linking of EphA2 at the surface of the cell leads to transient tyrosine phosphorylation of EphA2. The loss of tyrosine phosphorylation of EphA2 that occurs over time with these treatments could be due to a PTPase interacting with EphA2.

6.2. Regulation of EphA2 by LMW-PTP

6.2.1. Materials and Methods

Cell Lines and Antibodies. Human breast (MCF-10A, MCF 10A ST, MCF-7, MDA-MB-231, MDA-MB-435, SK-BR-3) epithelial cells were cultured as described in Example I and previously (Paine, T. M., Soule, H. D., Pauley, R. J. & Dawson, P. J. (1992) Int J Cancer 50, 463-473; Jacob, A. N., Kalapurakal, J., Davidson, W. R., Kandpal, G., Dunson, N., Prashar, Y. & Kandpal, R. P. (1999) Cancer Detection & Prevention 23, 325-332; Shevrin, D. H., Gomy, K. I. & Kukreja, S. C. (1989) Prostate 15, 187-194.). Monoclonal antibodies specific for phospho-tyrosine (PY20) and β-catenin were purchased from Transduction Laboratories (Lexington, Ky.). Monoclonal antibodies specific for phosphotyrosine (4G10) and EphA2 (clone D7) were purchased from Upstate Biotechnology, Inc. (Lake Placid, N.Y.). Monoclonal antibodies against vinculin were purchased from NeoMarkers (Fremont, Calif.).

Cell Lysates. Cell lysates were harvested and normalized for equal loading as described previously (Kinch, M. S., Clark, G. J., Der, C. J. & Burridge, K. (1995) J Cell Biol 130, 461-471). To confirm equal loading, blots were stripped as described previously and reprobed with antibodies specific to β-catenin or vinculin (Kinch, M. S., Clark, G. J., Der, C. J. & Burridge, K. (1995) J Cell Biol 130, 461-471).

Immunoprecipitation and Western Blot Analyses: Immunoprecipitation of EphA2 or LMW-PTP were performed using rabbit anti-mouse (Chemicon, Temecula, Calif.) conjugated Protein A Sepharose (Sigma, St. Louis, Mo.) as described previously (Zantek, N. D., Azimi, M., Fedor-Chaiken, M., Wang, B., Brackenbury, R. & Kinch, M. S. (1999) Cell Growth & Differentiation 10, 629-638.). To confirm equal loading, blots were stripped as described previously (Kinch, M. S., Clark, G. J., Der, C. J. & Burridge, K. (1995) J Cell Biol 130, 461-471) and reprobed with EphA2 or LMW-PTP specific antibodies. Western blot analysis were performed on normalized cells lysates and immunoprecipitations as detailed (Zantek, N. D., Azimi, M., Fedor-Chaiken, M., Wang, B., Brackenbury, R. & Kinch, M. S. (1999) Cell Growth & Differentiation 10, 629-638). Antibody binding was detected by enhanced chemiluminescence, (ECL; Pierce, Rockford, Ill.), and visualized by autoradiography (Kodak X-OMAT; Kodak, Rochester, N.Y.).

EGTA and Pervanadate Treatments. “Calcium Switch” experiments were performed as described previously (Zantek, N. D., Azimi, M., Fedor-Chaiken, M., Wang, B., Brackenbury, R. & Kinch, M. S. (1999) Cell Growth & Differentiation 10, 629-638) using MCF-10A cells grown to 70% confluence and medium containing a final concentration of 4 mM EGTA. Pervanadate was added to MDA-MB-231 in monolayer culture at a final concentration of 0, 1, 10 or 100 mM and the treatment was allowed to incubate for 10 minutes at 37° C., 5% CO₂. For the combined EGTA-Pervanadate Treatment, MDA-MB-231 cells were first treated with 100 mM Pervanadate and were then subjected tot he EGTA treatment.

In Vitro Kinase and Phosphatase Assays. To evaluate LMW-PTP activity against EphA2, EphA2 was immunoprecipitated from MCF-10A cells and incubated with purified LMW-PTP protein at a concentration of 0.45, 7.8, or 26.5 mg/mL for 0, 5, 15, or 30 minutes. The assay was terminated through the addition of Laemmli sample buffer. The phosphotyrosine content of the EphA2 in the treatments was then observed using Western blot analysis with antibodies specific to phosphotyrosine. To determine in vitro autophosphorylation activity, immunoprecipitated EphA2 was evaluated using in vitro kinase assays as detailed previously (Zantek, N. D., Azimi, M., Fedor-Chaiken, M., Wang, B., Brackenbury, R. & Kinch, M. S. (1999) Cell Growth & Differentiation 10, 629-638).

Transfection and Selection. Monolayers of MCF-10A cells were grown to 30-50% confluence and were transfected with pcDNA3.1-LMW-PTP or pcDNA3.1-D129A-LMW-PTP using Lipofectamine PLUS (Life Technologies, Inc., Grand Island, N.Y.). As a control for the transfection procedure, empty pcDNA3.1 vector was transfected into the same cell line in parallel. Transient transfections were allowed to grow for 48 hours post-transfection. For stable lines, neomycin-resistant cells were selected in growth medium containing 16 mg/mL neomycin (Mediatech, Inc., Herndon, Va.). To confirm LMW-PTP overexpression, Western blot analysis was performed using LMW-PTP specific antibodies. Parental cells and cells transfected with empty pcDNA3.1 vector were used as negative controls.

Growth Assay. To evaluate cell growth using monolayer assays, 1×105 cells were seeded into tissue-culture treated multi-well dishes for 1, 2, 4 or 6 days in triplicate experiments. Cell numbers were evaluated by trypsin suspension of the samples followed by microscopic evaluation using a hemacytometer. Soft agar colony formation was performed and quantified as detailed (Zelinski, D. P., Zantek, N. D., Stewart, J., Irizarry, A. & Kinch, M. S. (2001) Cancer Res 61, 2301-2306); Clark, G. J., Kinch, M. S., Gilmer, T. M., Burridge, K. & Der, C. J. (1996) Oncogene 12, 169-176). For experiments with EphA2 antisense, cells were incubated with oligonucleotides prior to suspension in soft agar. The data shown is representative of at least three different experiments.

Antisense Treatment. Monolayers of MCF-10A Neo cells and MCF-10A cells stably overexpressing LMW-PTP were grown to 30% confluence and were transfected with EphA2 antisense oligonucleotides as detailed. Samples that had been transfected with an inverted EphA2 antisense oligonucleotide or with the transfection reagent alone provided negative controls.

6.2.2. Results

6.2.2.1. EphA2 is Regulated by an Associated Tyrosine Phosphatase

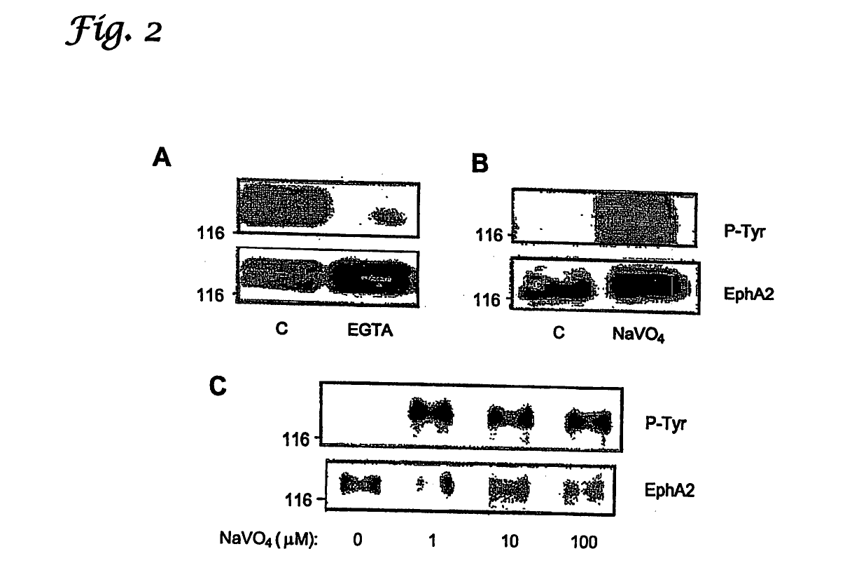

Several independent lines of investigation suggested that EphA2 is regulated by an associated tyrosine phosphatase. First, EphA2 could be rapidly dephosphorylated in non-transformed epithelial cells. Western blot analysis with phosphotyrosine antibodies (PY20 or 4G10) indicated lower levels of EphA2 phosphotyrosine content within 5 minutes following EGTA-mediated disruption of EphA2-ligand binding (FIG. 2A). Similarly, tyrosine phosphorylation of EphA2 decreased following incubation of non-transformed epithelial cells with dominant-negative inhibitors of EphA2-ligand binding (e.g., EphA2-Fc). Identical results were obtained using multiple non-transformed epithelial cell systems, including MCF-12A, MCF10-2, HEK293, MDCK and MDBK cells. Based on these findings, we asked whether tyrosine phosphatase inhibitors could prevent the loss of EphA2 phosphotyrosine content in response to EGTA treatment. Indeed, inhibitors such as sodium orthovanadate prevented the decrease in EphA2 phosphotyrosine following treatment of MCF-10A cells with EGTA (FIG. 2B).

Previous studies by our laboratory have shown that the phosphotyrosine content of EphA2 is greatly reduced in malignant epithelial cells as compared with non-transformed epithelia (Zelinski, D. P., Zantek, N. D., Stewart, J., Irizarry, A. & Kinch, M. S. (2001) Cancer Res 61, 2301-2306; Zantek, N. D., Azimi, M., Fedor-Chaiken, M., Wang, B., Brackenbury, R. & Kinch, M. S. (1999) Cell Growth & Differentiation 10, 629-638). Thus, we asked if tyrosine phosphatase activity could contribute to the reduced phosphotyrosine content of EphA2 in malignant cells. Whereas EphA2 was not tyrosine phosphorylated in malignant breast cancer cells (MDA-MB-231, MDA-435, MCFneoST, or PC-3 cells), incubation with increasing concentrations of sodium orthovanadate induced rapid and vigorous tyrosine phosphorylation of EphA2 (FIG. 2C). As vanadate treatment of cells can often lead to exaggerated phosphorylation of physiologically irrelevant sites, we performed phosphopeptide-mapping studies using EphA2 that had been labeled with 32P-ATP either in vitro or in vivo. These studies revealed identical patterns of tyrosine phosphorylation in non-transformed MCF-10A cells and vanadate treated MDA-MB-231 cells. Although the cytoplasmic domain contains multiple sites that could have been phosphorylated promiscuously, these were not phosphorylated under the conditions utilized here, suggesting that vanadate had not increased the phosphorylation of irrelevant sites. Altogether, these results indicate that EphA2 is regulated by an associated phosphatase that suppresses EphA2 phosphotyrosine content in malignant cells.

6.2.2.2. LMW-PTP Interacts with and Dephosphorylates EphA2

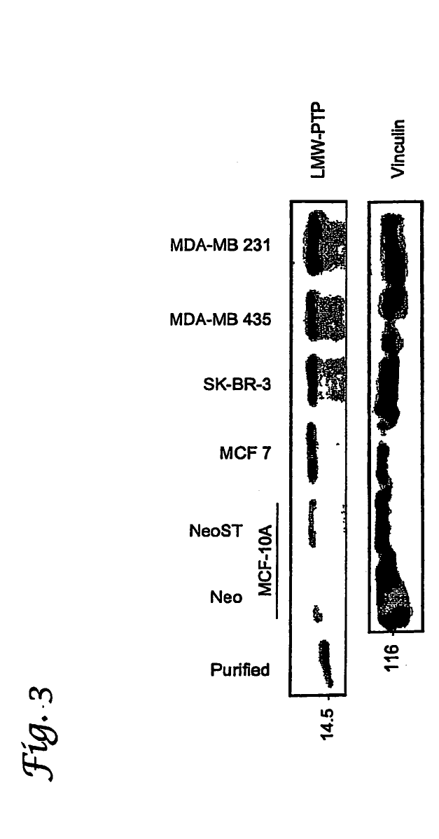

To identify tyrosine phosphatases that might regulate EphA2 in malignant cells, we considered a recent report that LMW-PTP regulates a related molecule, EphB4 (Jacob, A. N., Kalapurakal, J., Davidson, W. R., Kandpal, G., Dunson, N., Prashar, Y., and Kandpal, R. P. (1999) Cancer Detect. Prev. 23, 325-33). Our initial experiments began to catalog the expression and function of LMW-PTP in non-transformed (MCF-10A Neo) and malignant (MCF-7, SK-BR-3, MDA-MB-435, MDA-MB-231) mammary epithelial cells (FIG. 3). Western blot analyses of whole cell lysates revealed relatively high levels of LMW-PTP in tumor-derived breast cancer cells as compared with non-transformed MCF-10A mammary epithelial cells. To confirm equal sample loading, the membranes were stripped and re-probed with antibodies against a control protein (Vinculin), verifying that the high levels of LMW-PTP did not reflect a loading error or a generalized increase in protein levels in the malignant cells. A malignant variant of MCF-10A, MCFneoST, also demonstrated elevated LMW-PTP expression, which was intriguing based on a recent report that EphA2 is not tyrosine phosphorylated in those cells (Zantek, N. D., Walker-Daniels, J., Stewart, J. C., Hansen, R. K., Robinson, D., Miao, H., Wang, B., Kung, H. J., Bissell, M. J. & Kinch, M. S. (2001) Clin Cancer Res 7, 3640-3648). The use of a genetically-matched system also precluded potential differences due to cell origin or culture conditions. Thus, the highest levels of LMW-PTP were consistently found in malignant epithelial cells and inversely related to EphA2 phosphotyrosine content.

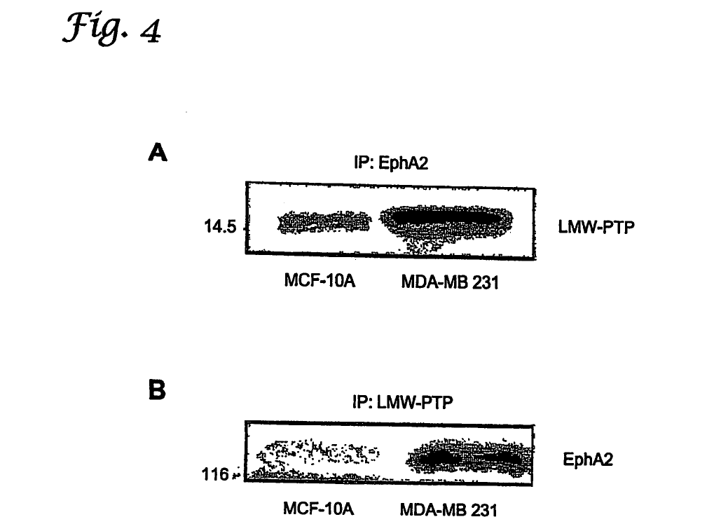

The results above provided suggestive, but indirect, evidence that LMW-PTP might negatively regulate the phosphotyrosine content of EphA2 in tumor cells. To explore this hypothesis further, we first asked if the two molecules interacted in vivo. EphA2 was immunoprecipitated from MDA-MB-231 cells using specific antibodies (clone D7) and these complexes were resolved by SDS-PAGE. Subsequent Western blot analyses revealed that LMW-PTP was prominently found within EphA2 immune complexes (FIG. 4A). The inverse experiment confirmed that EphA2 could similarly be detected in complexes of immunoprecipitated LMW-PTP (FIG. 4B). Control immunoprecipitations with irrelevant antibodies confirmed the specificity of the interactions of the two molecules.

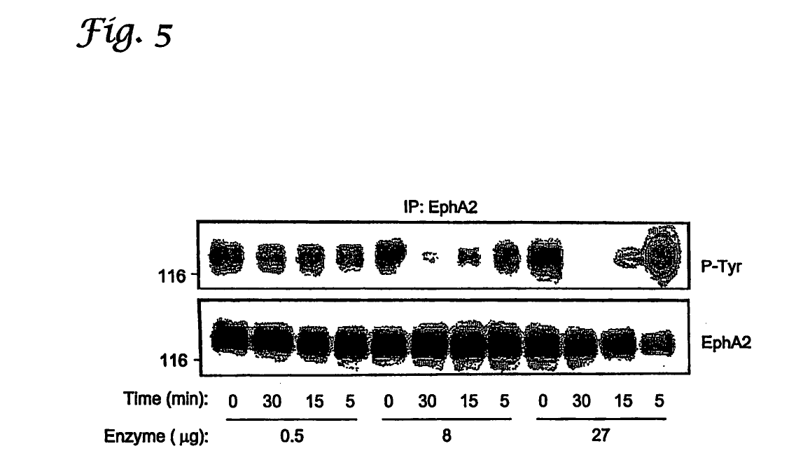

The co-immunoprecipitation studies did not clarify whether EphA2 can serve as a substrate for LMW-PTP. To address this directly, EphA2 was immunoprecipitated from MCF-10A cells, where it is normally tyrosine phosphorylated. The purified EphA2 was then incubated with different concentrations of purified LMW-PTP before Western blot analyses of EphA2 with phosphotyrosine-specific antibodies (PY20 and 4G10). These experiments demonstrated that purified LMW-PTP could dephosphorylate EphA2 in a dose and time-dependent manner (FIG. 5A).

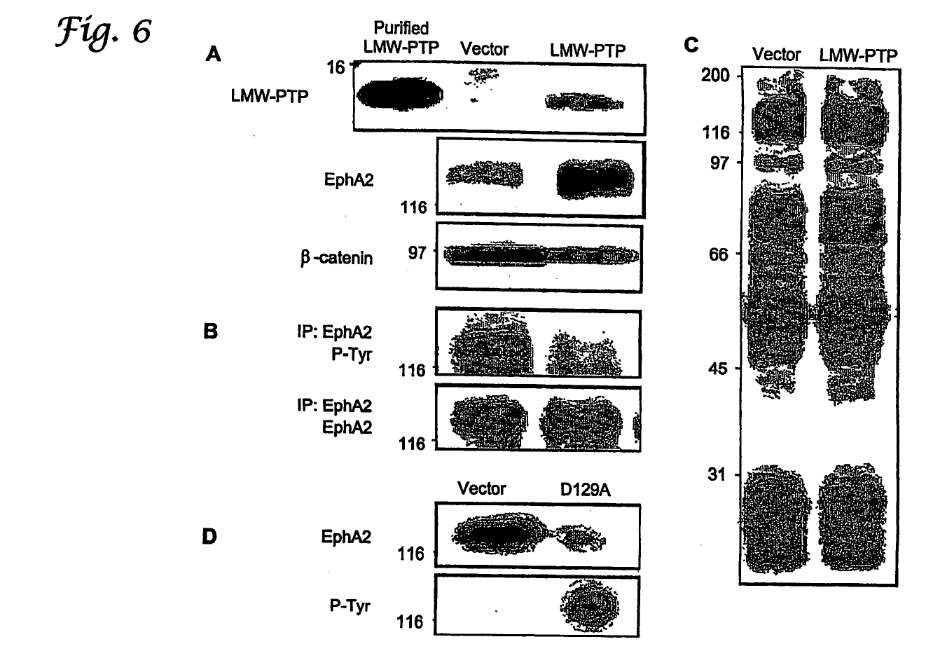

Although in vitro studies indicated that EphA2 could be phosphorylated by LMW-PTP in vitro, we recognized that in vitro studies are not always be representative of the analogous situation in vivo. Thus, LMW-PTP was ectopically overexpressed in MCF-10A cells. This particular cell system was selected because non-transformed MCF-10A cells have low levels of endogenous LMW-PTP and because the EphA2 in these non-transformed epithelial cells is normally tyrosine phosphorylated. Ectopic overexpression of LMW-PTP was achieved by stable transfection, as determined by Western blot analyses with specific antibodies (FIG. 6A). Importantly, overexpression of LMW-PTP was sufficient to reduce the phosphotyrosine content of EphA2 as compared with vector-transfected negative controls (FIG. 6A). Identical results were obtained using different experiments, with different transfectants and in both stably and transiently-transfected samples, thus eliminating potential concerns about clonal variation. Moreover, the decreased phosphotyrosine content was specific for EphA2 as the phosphotyrosine content LMW-PTP overexpressing cells was not generally decreased (FIG. 6B).

6.2.2.3. LMW-PTP Overexperssion Causes Malignant Transformation of Epithelial Cells

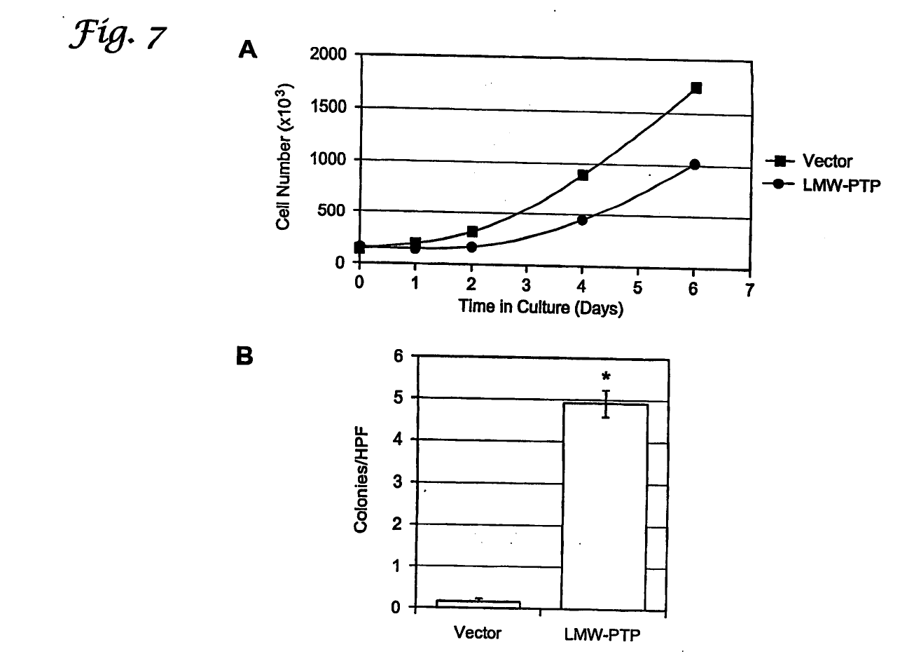

Tyrosine phosphorylated EphA2 negatively regulates tumor cell growth whereas unphosphorylated EphA2 acts as a powerful oncoprotein (Zelinski, D. P., Zantek, N. D., Stewart, J., Irizarry, A. & Kinch, M. S. (2001) Cancer Res 61, 2301-2306; Zantek, N. D., Azimi, M., Fedor-Chaiken, M., Wang, B., Brackenbury, R. & Kinch, M. S. (1999) Cell Growth & Differentiation 10, 629-638). Thus, we asked whether overexpression of LMW-PTP would be sufficient to induce malignant transformation. To address this question, we utilized the MCF-10A cells, described above, which had been transfected with either wild-type LMW-PTP or a vector control. Our initial studies evaluated the growth rates of control and LMW-PTP-overexpressing cells in monolayer culture. When evaluated using standard, two-dimensional culture conditions, the growth rates of LMW-PTP-overexpressing MCF-10A cells were significantly lower than the growth rates of matched controls (P<0.05) (FIG. 7A).

Two-dimensional assessments of growth often do not reflect the malignant character of tumor cells. Instead, three-dimensional analyses of cell behavior using soft agar and reconstituted basement membranes can provide a more relevant way of assessing malignant behavior. Whereas vector-transfected MCF-10A cells were largely incapable of colonizing soft agar, LMW-PTP-overexpressing cells formed an average of 4.9 colonies per high-powered microscope field (P<0.01; FIG. 7B). Based on recent findings with other three-dimensional assay systems, we also evaluated cell behavior using three-dimensional, reconstituted basement membranes. Consistent with a more aggressive phenotype, microscopic assessment of cell behavior in Matrigel confirmed the malignant character of LMW-PTP overexpressing cells. When plated atop or within Matrigel, LMW-PTP-overexpressing cells formed larger colonies than vector-transfected cells. Altogether, consistent results with multiple and different systems suggest that overexpression of LMW-PTP is sufficient to induce malignant transformation.

6.2.2.4. The Oncogenic Phenotype of LMW-PTP-Overexpressing Cells is Related to EphA2 Expression

Tyrosine phosphorylation of EphA2 induces its internalization and degradation. Thus, we postulated that overexpression of LMW-PTP might increase the protein levels of EphA2. Indeed, Western blot analyses of whole cell lysates revealed higher levels of EphA2 in MCF-10A cells that overexpress LMW-PTP as compared with vector-transfected controls (FIG. 6A). Moreover, this EphA2 was not tyrosine phosphorylated (FIG. 6B). However, Western blot analyses revealed that the reduced phosphotyrosine content was selective for EphA2, as the general levels of phosphotyrosine were not altered in LMW-PTP transformed cells (FIG. 6C).

The finding that overexpression of LMW-PTP increased EphA2 expression and decreased its phosphotyrosine content was intriguing since this phenotype was reminiscent of highly aggressive tumor cells (Zelinski, D. P., Zantek, N. D., Stewart, J., Irizarry, A. & Kinch, M. S. (2001) Cancer Res 61, 2301-2306; Zantek, N. D., Azimi, M., Fedor-Chaiken, M., Wang, B., Brackenbury, R. & Kinch, M. S. (1999) Cell Growth & Differentiation 10, 629-638)). Thus, we asked whether selective targeting of LMW-PTP in malignant cells would impact EphA2. To accomplish this, an enzymatic mutant of LMW-PTP (D129A) that is catalytically inactive (Zhang, Z., Harms, E. & Van Etten, R. L. (1994) Journal of Biological Chemistry 269, 25947-25950) was overexpressed in MDA-MB-231 cells, which have high levels of wild-type LMW-PTP (FIG. 3) and overexpress unphosphorylated EphA2. Ectopic overexpression of LMW-PTPD129A was found to decrease the levels of EphA2. Moreover, Western blot analyses of immunoprecipitated material revealed that this EphA2 was tyrosine phosphorylated (FIG. 6C). Thus, consistent results indicate that overexpression of wild type LMW-PTP is necessary and sufficient to confer the overexpression and functional alterations of EphA2 that have been observed in tumor-derived cells.

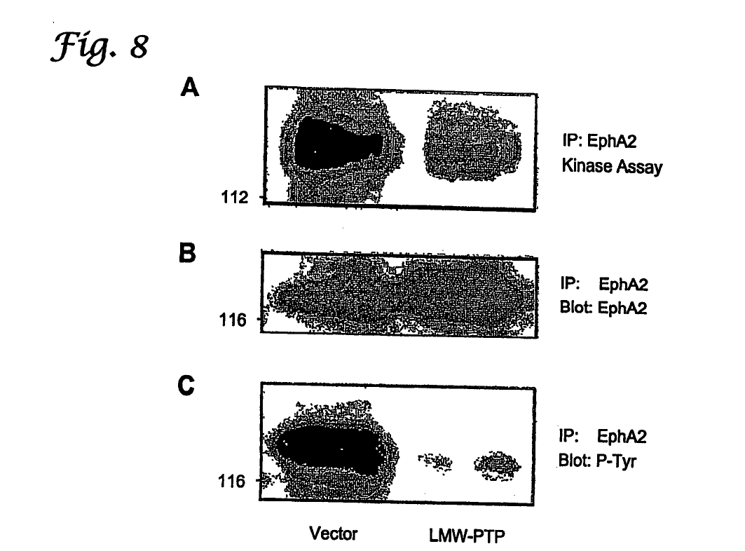

Although the EphA2 in the LMW-PTP overexpressing MCF-10A cells was not tyrosine phosphorylated, it retained enzymatic activity. In vitro kinase assays verified that the EphA2 from LMW-PTP-transformed MCF-10A cells had levels of enzymatic activity that were comparable to vector-transfected controls (FIG. 8A). To verify equal sample loading, two controls were performed. Equal amounts of input lysate were verified by Western blot analyses with β-catenin antibodies. In addition, the immunoprecipitated EphA2 was divided and half of the material was resolved by SDS-PAGE and analyzed by Western blot analyses with EphA2 and phosphotyrosine-specific antibodies (FIG. 8B). Thus phosphorylated and unphosphorylated EphA2 were both capable of enzymatic activity.

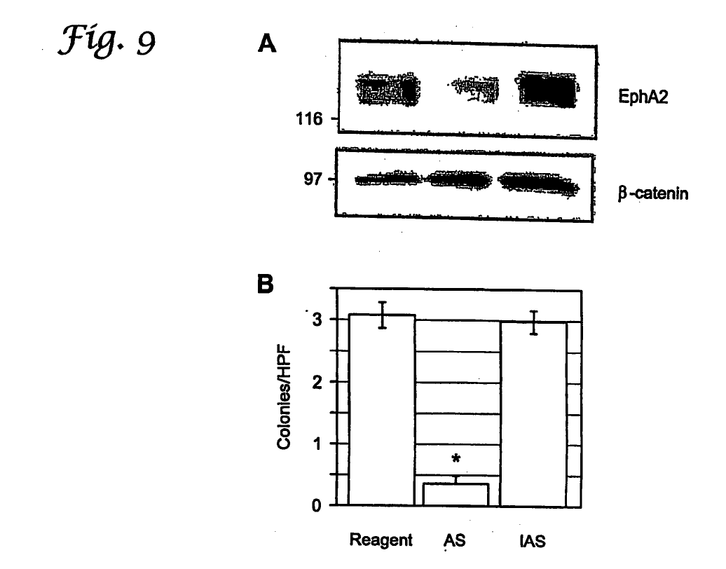

Since the levels of EphA2 were elevated in LMW-PTP transformed cells, we asked whether the oncogenic activity of EphA2 might have contributed to this phenotype. To address this, we utilized our experience with antisense strategies to selectively decrease EphA2 expression in LMW-PTP transformed cells (Hess A. R., Seftor, E. A., Gardner, L. M., Carles-Kinch, K., Schneider, G. B., Seftor, R. E., Kinch, M. S. & Hendrix, M. J. C. (2001) Cancer Res 61, 3250-3255.). We verified the success of these strategies by Western blot analyses (FIG. 9A) and then asked if decreased EphA2 expression would alter soft agar colonization. Indeed, transfection with EphA2 antisense oligonucleotides decreased the soft agar colonization of LMW-PTP-transformed MCF-10A cells by at least 87% (P<0.01; FIG. 9B). In contrast, transfection of these cells with an inverted antisense control nucleotide control did not significantly alter soft agar colonization. Thus, we were able to exclude that the results with the antisense oligonucleotides had resulted from non-specific toxicities caused by the transfection procedure. Altogether, our results indicate that, in cells that express EphA2, the oncogenic actions of overexpressed LMW-PTP require high levels of EphA2.

6.2.3. Discussion

The major finding of our present study is that EphA2 is regulated by an associated tyrosine phosphatase and we identify LMW-PTP as a critical regulator of EphA2 tyrosine phosphorylation. We also demonstrate that LMW-PTP is overexpressed in metastatic cancer cells and that LMW-PTP overexpression is sufficient to confer malignant transformation upon non-transformed epithelial cell models. Finally, we demonstrate that LMW-PTP upregulates the expression of EphA2 and that the oncogenic activities of LMW-PTP require this overexpression of EphA2.

Recent reports from our laboratory and others have shown that many malignant epithelial cells express high levels of EphA2 that is not tyrosine phosphorylated. Previously, we had related these depressed levels of EphA2 tyrosine phosphorylation with decreased ligand binding. Malignant cells often have unstable cell-cell contacts and we postulated that this decreases the ability of EphA2 to stably interact with its ligands, which are anchored to the membrane of adjacent cell. In part, our present data suggests a new paradigm in which the phosphotyrosine content of EphA2 is also negatively regulated by an associated tyrosine phosphatase that is overexpressed in malignant cells. Given the relationship between EphA2 phosphorylation and cell-cell adhesion, we cannot exclude that cell-cell contacts could also regulate the expression or function of LMW-PTP and future investigation should address this possibility.

The fact that high levels of LMW-PTP were observed in several different cell models of metastatic cancer is notable given that LMW-PTP overexpression is sufficient to confer malignant transformation. LMW-PTP overexpressing cells gain the ability to colonize soft agar and acquire a malignant phenotype when cultured in three-dimensional basement membranes, such as Matrigel. Notably, however, LMW-PTP-overexpressing MCF-10A epithelial cells displayed reduced rates of cell growth as measured using two-dimensional assays of cell growth. This latter observation is consistent with recent reports that high levels of LMW-PTP similarly decrease the monolayer growth rates of other cell types (Shimizu, H., Shiota, M., Yamada, N., Miyazaki, K., Ishida, N., Kim, S. & Miyazaki, H. (2001) Biochemical & Biophysical Research Communications 289, 602-607; Fiaschi, T., Chiarugi, P., Buricchi, F., Giannoni, E., Taddei, M. L., Talini, D., Cozzi, G., Zecchi-Orlandini, S., Raugei, G. & Ramponi, G. (2001) Journal of Biological Chemistry 276, 49156-49163). Although such a finding had been interpreted to suggest that LMW-PTP might negatively regulate malignant transformation, our findings support a very different conclusion. Consistent with this, recent studies by our laboratory and others have shown that malignant transformation of MCF-10A cells is often accompanied by decreased monolayer growth rates and that the most aggressive variants of MCF-10A in vivo demonstrate the slowest growth in monolayer culture. These findings have important implications for the design and interpretation of oncogene function when using non-transformed epithelial cell systems.

The biochemical consequences of EphA2 tyrosine phosphorylation remain largely unclear. Unlike other receptor tyrosine kinases, where autophosphorylation is necessary for enzymatic activity, tyrosine phosphorylation of EphA2 is not required for its enzymatic activity. Consistent with our present results, EphA2 retains comparable levels of enzymatic activity in non-transformed and tumor-derived cells, despite dramatic differences in its phosphotyrosine content (Zantek, N. D., Azimi, M., Fedor-Chaiken, M., Wang, B., Brackenbury, R. & Kinch, M. S. (1999) Cell Growth & Differentiation 10, 629-638). Similarly, antibody-mediated stimulation of EphA2 autophosphorylation does not change the levels of EphA2 enzymatic activity. Phosphopeptide analyses of the EphA2 cytoplasmic domain provide one potential explanation. Although EphA2 has a predicted activation loop tyrosine at residue 772 (Lindberg, R. A. & Hunter, T. (1990) Molecular & Cellular Biology 10, 6316-6324), neither in vitro nor in vivo phosphopeptide analyses found that this site is not phosphorylated either in normal cell models or in response to exogenous ligands in malignant cell models. Thus, the lack of a consensus activation loop tyrosine may account for the retention of EphA2 enzymatic activity in cells where it is not tyrosine phosphorylated.

Whereas tyrosine phosphorylation of EphA2 does not appear to be necessary for its intrinsic enzymatic activity, ligand-mediated tyrosine phosphorylation regulates EphA2 protein stability. Specifically, tyrosine phosphorylation fates EphA2 to interact with the c-Cbl adapter protein and to subsequently be internalized and degraded within proteosomes (J. Walker-Daniels et al., Mol. Cancer Res. 2002 November; 1(1): 79-87). Consequently, the phosphatase activity of LMW-PTP would be predicted to increase EphA2 protein stability. Indeed, the highest levels of EphA2 are consistently found in cells with high levels of LMW-PTP. One interesting implication of this finding is that it provides a mechanism, independent of genetic regulation of the EphA2 gene, to explain why high levels of EphA2 are found in many different tumors. An alternative possibility is that LMW-PTP upregulates EphA2 gene expression and our present findings do not formally eliminate this possibility. The fact that EphA2 inhibitors reversed the malignant character of LMW-PTP overexpressing cells suggests that the upregulation of EphA2 is relevant to the cellular behaviors of LMW-PTP-mediated transformation.

In summary, our present studies, as described in this example and in Kikawa et al., J. Biol. Chem. 277 (42): 39274-39279 (2002)) identify LMW-PTP as a new oncogene that is overexpressed in tumor-derived carcinoma cells. We also link the biochemical and biological actions of overexpressed LMW-PTP as with EphA2. These findings have important implications for understanding the biochemical and biological mechanisms that contribute to the metastatic progression of epithelial cells. Moreover, our present studies identify an important signaling system that could ultimately provide an opportunity to target the large number of cancer cells that overexpress EphA2 or LMW-PTP.

6.3. Effects of LMW-PTP

Cell lines and reagents were as described in Example II (Kikawa et al., J. Biol. Chem. 277 (42): 39274-39279 (2002)). Methods for making cell lysates and for performing immunoprecipitation and western blot (immunoblot) analyses, EGTA and pervanadate treatments, transfection and selection, and growth assays were also as described in Example II ((Kikawa et al., J. Biol. Chem. 277 (42): 39274-39279 (2002)).

6.3.1. Morphological Effects of LMW-PTP Overexpression in Non-Transformed Cells

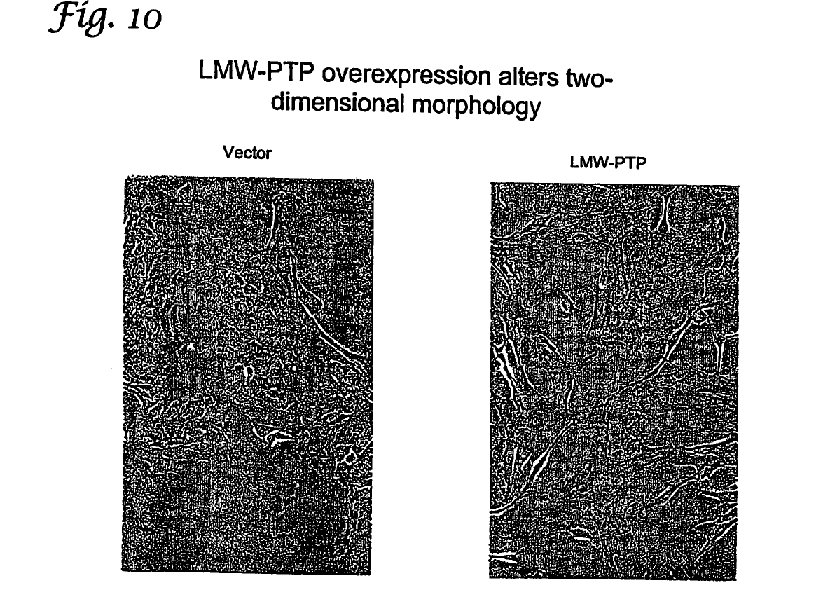

Monolayer cultures of MCF-10A cells that had been stably transfected with either wild-type human LMW-PTP, or a matching vector control, were subjected to microphotography (600×) (FIG. 10). Whereas the non-transformed (vector) cells retained a characteristic epithelial morphology, LMW-PTP-transfected cells adopted a mesenchymal phenotype that is characteristic of malignant epithelial cells. Overexpression of LMW-PTP was thus observed to alter two-dimensional morphology of the cells.

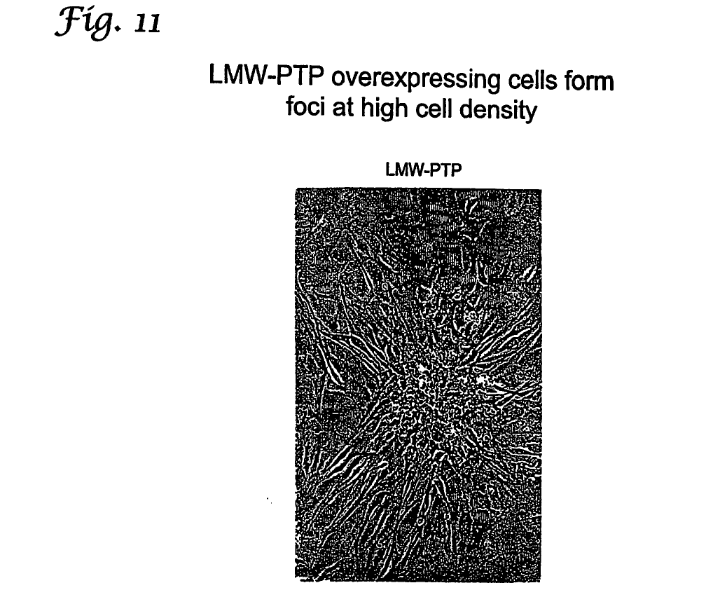

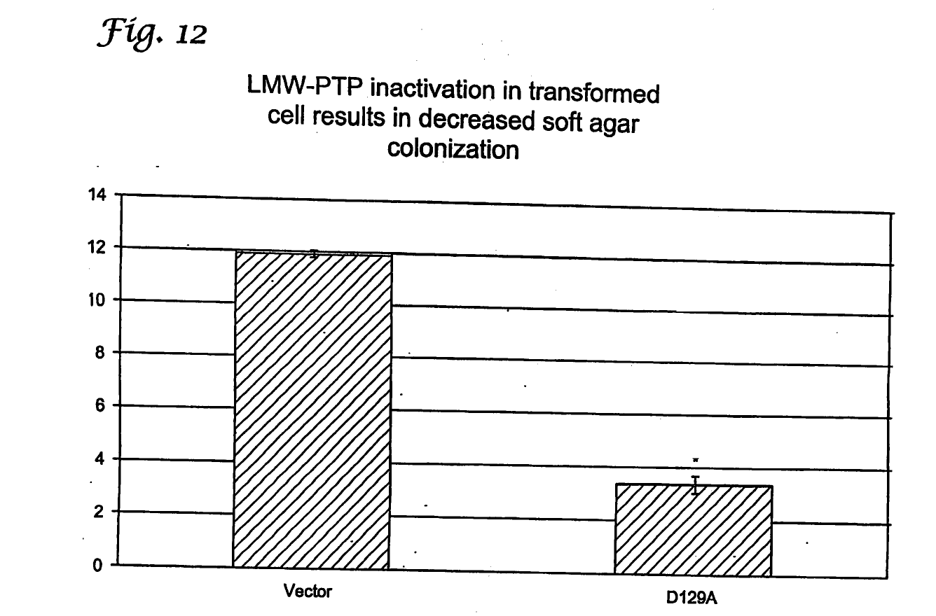

The LMW-PTP transfected MCF-10A cells were further observed to form three-dimensional foci, a hallmark of malignant transformation, when cultured at high cell density (FIG. 11).

6.3.2. Effects of LMW-PTP Inactivation in Transformed Cells

To evaluate the biological outcomes of inhibiting LMW-PTP in tumor cells, highly invasive MDA-MB-231 cells were stably transfected with a mutant of LMW-PTP (D129A). D129A functions as a substrate trapping mutant and thereby competes away the activity of endogenous LMW-PTP in the tumor cells. It effectively inactivates LMW-PTP in the transformed cells.

D129A transfected cells showed reduced colony formation in soft agar relative to matched (vector) controls (FIG. 12). Thus LMW-PTP inactivation in transformed cells results in decreased soft agar colonization. This indicates that LMW-PTP is necessary for anchorage-independent cell growth and/or survival, which are hallmarks of malignant cells.

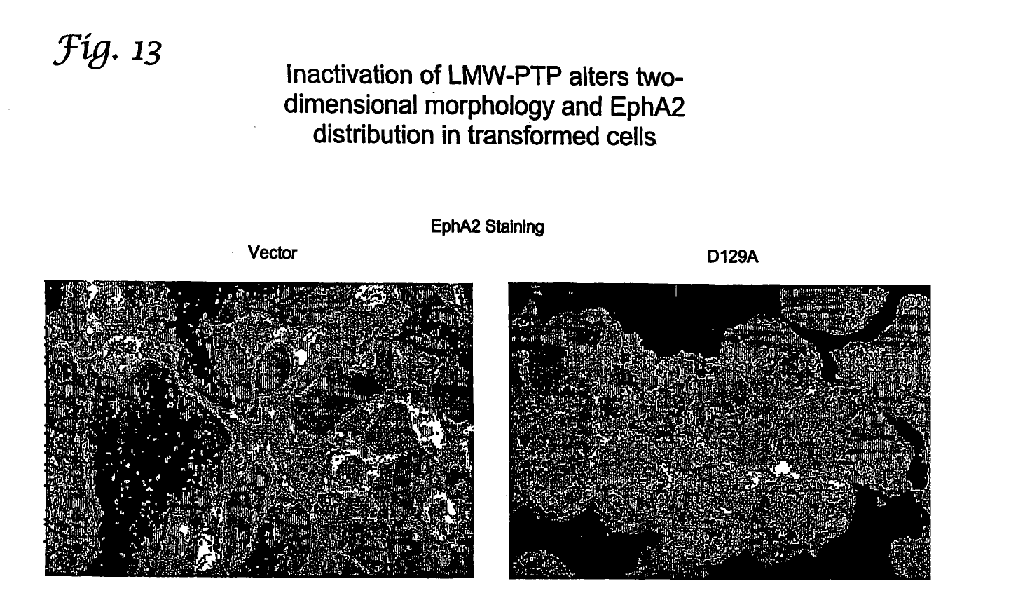

It was also found that inactivation of LMW-PTP alters two-dimensional morphology and EphA2 distribution in transformed cells. The morphology of MDA-MB-231 cells that express dominant-negative LMW-PTP (D129A) or a matched vector control was evaluated by immunofluorescence microphotography of labeled EphA2 (FIG. 13). Control cultures MDA-MB-231 normally adopt a mesenchymal morphology with EphA2 diffusely distributed or enriched with membrane ruffles. In contrast, D129A-transfected cells display a characteristic epithelial morphology, with EphA2 enriched within sites of cell-cell contact.

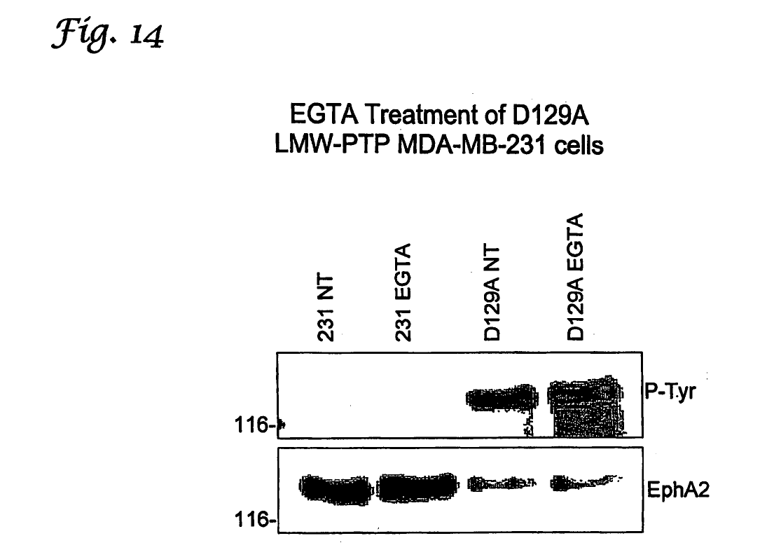

D129A LMW-PTP MDA-MB-231 cells were treated with EGTA to determine its effect on the phosphorylation status of EphA2. Detergent extracts from 5×10⁶ control and D129A-transfected MDA-MB-231 cells were harvested as described in Examples 1 and 2. After immunoprecipitating EphA2 with D7 antibodies, the samples were resolved by SDS-PAGE and subjected to Western blot analyses with phosphotyrosine-specific (4G10) antibodies. The EphA2 in D129A-transfected cells was found to be more highly tyrosine phosphorylated, even following treatment with EGTA (FIG. 14). EGTA destabilizes cell-cell contacts and thereby prevent EphA2 from binding its membrane-anchored ligands. This suggests that D129A prevents EphA2 from being dephosphorylated even after loss of ligand binding.

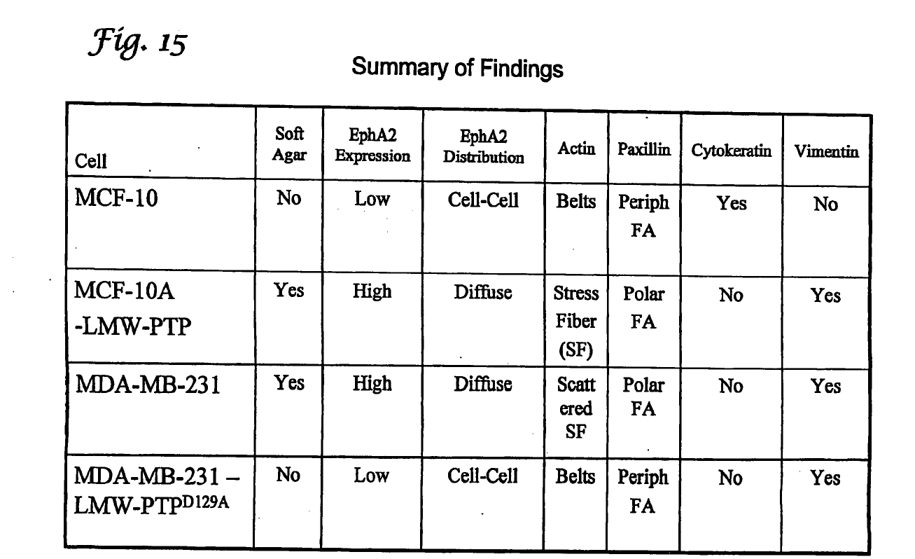

FIG. 15 shows a is a table that summarizes evidence from immunofluorescence microscopic studies using LMW-PTP transfected MCF-10A cells and D129A-transfected MDA-MB-231 cells. The altered morphology and markers of LMW-PTP-transfected MCF-10A cells is consistent with malignant transformation. Moreover, the morphology of D129A overexpressing cells is consistent with a less aggressive (more differentiated) phenotype.

6.3.3. Co-Localization of EphA2 and LMW-PTP in Transformed and Nontransformed Cells

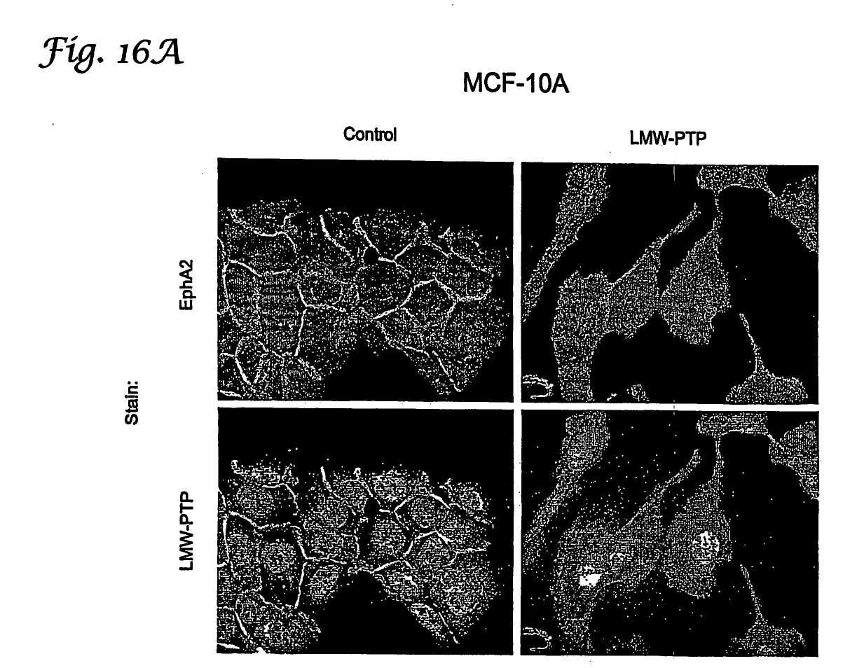

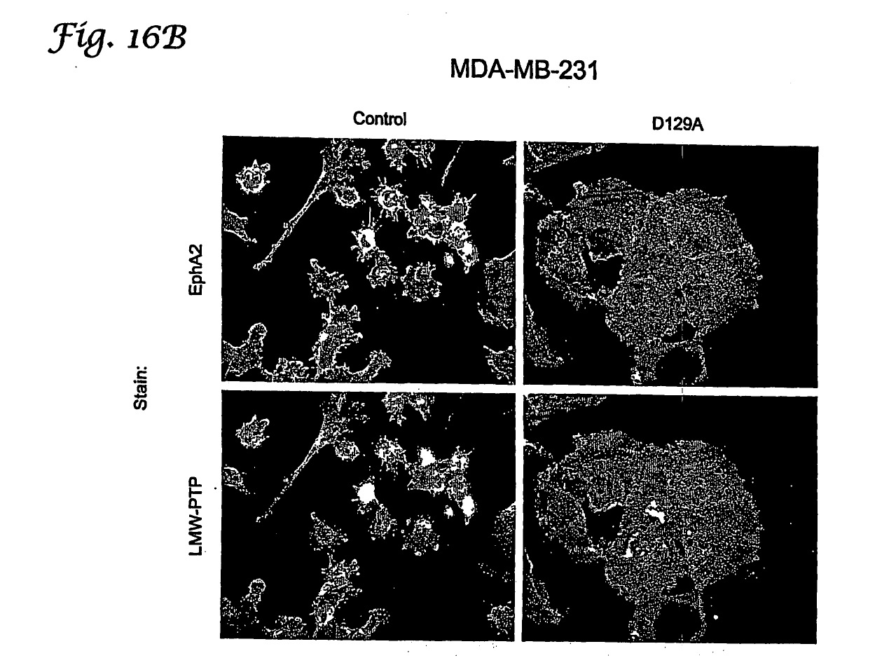

Subcellular localization of EphA2 (using D7 antibodies) and LMW-PTP (using rabbit polyclonal sera) in control and LMW-transfected MCF-10A cells was evaluated in formalin-fixed (3.7%, 2 minutes), detergent permeabilized (PBS containing 0.5% Triton-X-100) monolayers, cultured on glass coverslips. The images (FIG. 16A) were viewed on a Nikon microscope (600×) and images captured using Nikon digital cameras and software.

Subcellular localization of EphA2 (using D7 antibodies) and LMW-PTP (using rabbit polyclonal sera) was likewise evaluated in control and D129A overexpressing MDA-MB-23.1 cells was likewise evaluated (FIG. 16B).

6.3.4. Effects of LMW-PTP Overexpression on Actin Organization in Transformed and Nontransformed Cells

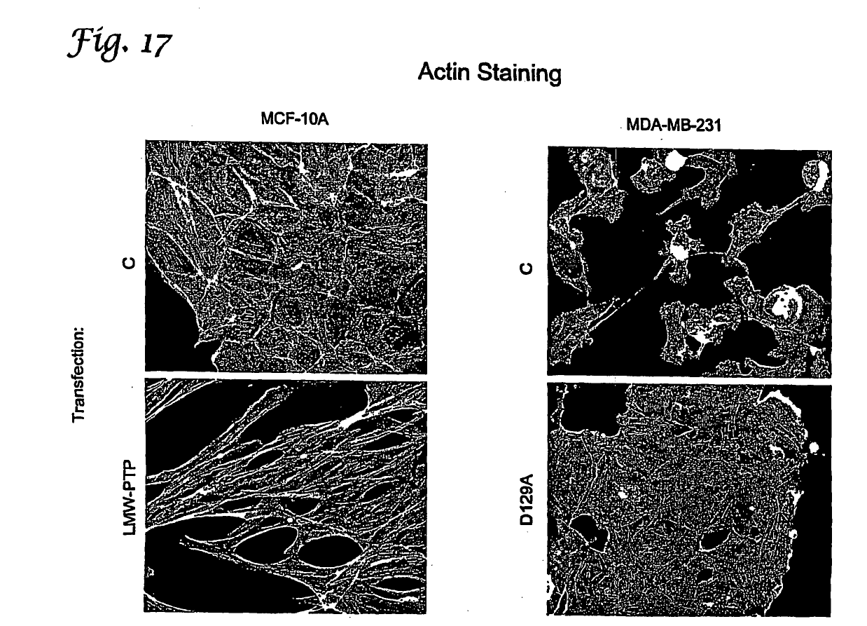

The organization of the actin cytoskeleton was evaluated in a MDA-MB-231 cell line stably expressing the D129A LMW-PTP mutation (B (form), the MCF-10A cell line, and the MCF-10A cell line stably expressing the wild-type (WT) LMW-PTP molecule (B (form) by immunofluorescence localization of fluorescein-conjugated phalloidin (Molecular Probes, Eugene, Oreg.). The subcellular localization of actin (phalloidin staining) was evaluated in formalin-fixed (3.7%, 2 minutes), detergent permeabilized (PBS containing 0.5% Triton-X-100) monolayers, cultured on glass coverslips. The images (FIG. 17) were viewed on a Nikon microscope (600×) and images captured using Nikon digital cameras and software.

Overexpression of wild-type LMW-PTP was found to cause the formation of stress fibers (as opposed to the adhesion belts that predominate in control cells). In the converse situation, dominant-negative inhibitors (D129A) of LMW-PTP decrease the number of stress fibers in MDA-MB-231. These observations are consistent with the hypothesis that wild-type LMW-PTP promotes a malignant (migratory and invasive) phenotype whereas inhibition of LMW-PTP is sufficient to reverse an aggressive phenotype.

6.3.5. Effects of LMW-PTP Overexpression on Focal Adhesion

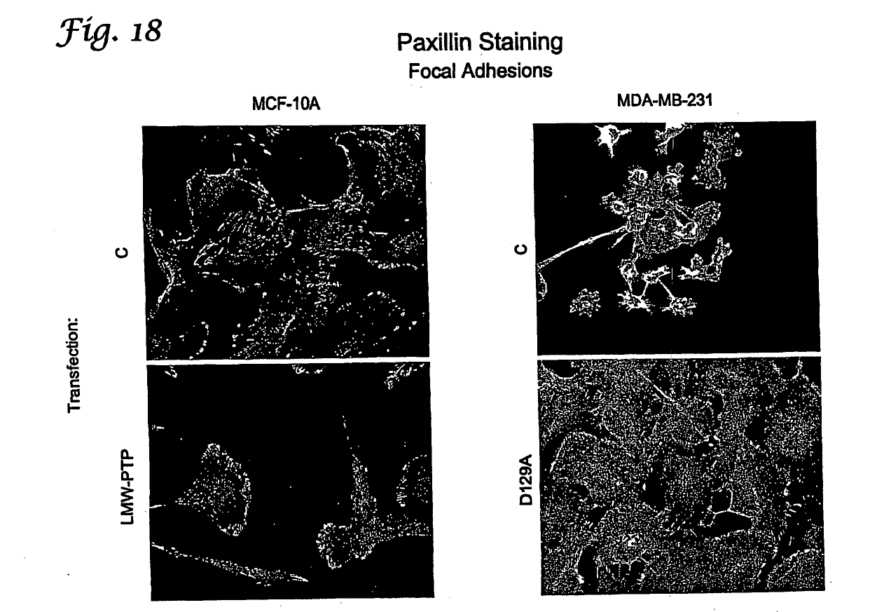

The organization of focal adhesion, as determined using paxillin-specific antibodies, was also evaluated in MDA-MB-231 cells by immunofluorescence microscopy. The subcellular localization of paxillin was evaluated in formalin-fixed (3.7%, 2 minutes), detergent permeabilized (PBS containing 0.5% Triton-X-100) monolayers, cultured on glass coverslips. The images (FIG. 18) were viewed on a Nikon microscope (600×) and images captured using Nikon digital cameras and software.

Overexpression of wild-type LMW-PTP was found to increase the prominence of focal adhesion, particularly at the leading edge of cell migration and invasion, which is consistent with a more aggressive phenotype. In the converse situation, dominant-negative inhibitors (D129A) of LMW-PTP decrease the predominance of focal adhesion in MDA-MB-231 cells, resulting in a diffuse (rather than polarized) distribution of focal adhesions, which is not consistent with cell migration or invasion.

6.3.6. Pathological Markers of Malignant Character

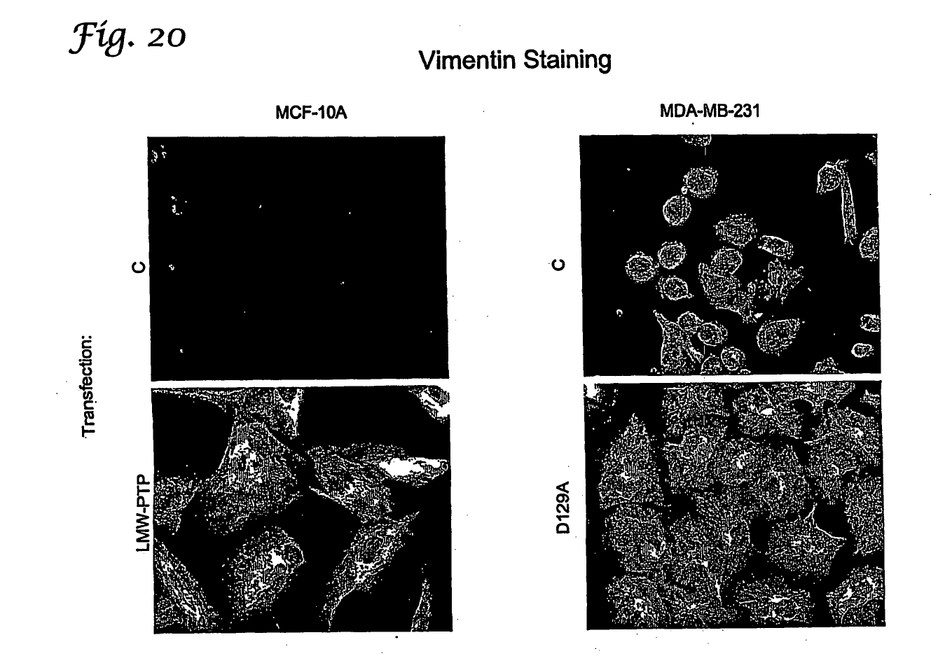

The expression of cytokeratin (FIG. 19) and vimentin (FIG. 20) was evaluated using immunofluorescence microscopy. The staining of cytokeratin and vimentin was evaluated in formalin-fixed (3.7%, 2 minutes), detergent permeabilized (PBS containing 0.5% Triton-X-100) monolayers, cultured on glass coverslips. The images were viewed on a Nikon microscope (600×) and images captured using Nikon digital cameras and software.

Overexpression of wild-type LMW-PTP was found to decrease cytokeratin but increase vimentin expression. These results are notable given that these changes in intermediate filament protein expression are frequently used by pathologists for cancer diagnosis and typing.

6.4. Effects of LMW-PTP Overexpression on Tumorigenic Potential of Non-Transformed Epithelial Cells

Cells (MCF-10A, MCF-10A Neo (control) and MCF-10A cells stably overexpressing wild-type LMW-PTP) were introduced into mice via injection subcutaneously. Two dosage levels were used: approximately 2 million and 5 million cells. Three mice were included in each group. The mice were observed 20 days after injection, and the size of the tumor (if present) was measured.

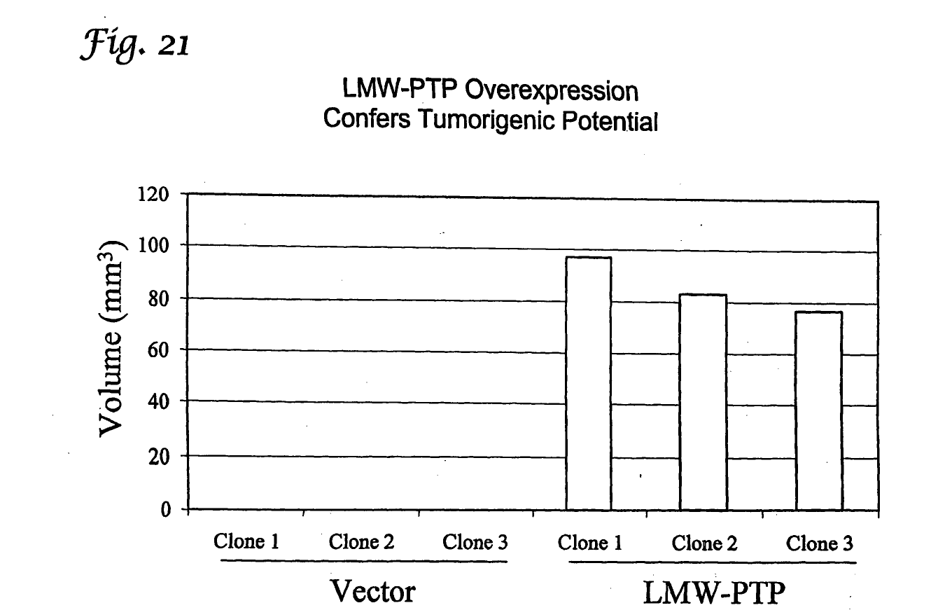

None of the mice injected with the parental MCF-10A cells or the control vector exhibited tumorogenesis at the injection site. Mice injected with the MCF-10A cells stably overexpressing WT LMW-PTP, however, exhibited significant growth in all 3 of the mice injected with 5 million cells and 2 of the 3 mice injected with 1 million cells. These results suggest that LMW-PTP overexpression is sufficient to confer tumorigenic potential upon non-transformed epithelial cells. FIG. 21 shows the data for mice injected with 5×10⁶ cells, implanted subcutaneously for 20 days. EphA2 is the only other oncogene we are aware of that is capable of conferring tumorigenic potential upon non-transformed epithelial cells.

7. Equivalents

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

1. A method of treating, preventing or managing a hyperproliferative cell disease associated with cells that express LMW-PTP, EphA2 or EphA4 in a subject in need thereof, said method comprising administering to said subject a therapeutically or prophylactically effective amount of a composition comprising:
(a) a delivery vehicle associated with a moiety that binds EphA2 or EphA4 expressed on a cell;
(b) an agent that inhibits or reduces LMW-PTP expression or activity contained within or attached to said delivery vehicle; and
(c) a pharmaceutically acceptable carrier.

2. The method of claim 1, wherein said hyperproliferative cell disease is cancer.

3. The method of claim 2, wherein said cancer is a metastatic cancer.

4. The method of claim 2, wherein said cancer is of an epithelial cell origin.

5. The method of claim 2, wherein said cancer comprises cells that overexpress EphA2 or EphA4 relative to non-cancer cells having the tissue type of said cancer cells.

6. The method of claim 2, wherein said cancer is of the skin, lung, colon, breast, prostate, bladder or pancreas, a renal cell carcinoma, a melonoma, a leukemia, or a lymphoma.

7. The method of claim 1, wherein said hyperproliferative cell disease is a non-cancer hyperproliferative cell disease.

8. The method of claim 7, wherein said non-cancer hyperproliferative cell disease is asthma, chronic obstructive pulmonary disease (COPD), psoriasis, lung fibrosis, bronchial hyper responsiveness, seborrheic dermatitis, and cystic fibrosis, inflammatory bowel disease, smooth muscle restenosis, endothelial restenosis, hyperproliferative vascular disease, Behcet's Syndrome, atherosclerosis, or macular degeneration.

9. The method of claim 1, wherein said delivery vehicle is a viral vector, a polycation vector, a peptide vector, a liposome, or a hybrid vector.

10. The method of claim 1, wherein said moiety that binds EphA2 or EphA4 is an anti-EphA2 or anti-EphA4 antibody or an antigen-binding fragment thereof, an antibody that binds EphA2 or EphA4 epitopes exposed on cancer cells, or Ephrin A1 or fragment thereof that binds EphA2 or EphA4.

11. The method of claim 10, wherein said Ephrin A1 or fragment thereof is fused to an Fc domain.

12. The method of claim 1, wherein said agent that inhibits or reduces LMW-PTP expression or activity is an anti-LMW-PTP antibody or an antigen-binding fragment thereof, a small phosphatase inhibitor, a RNA interference (RNAi) molecule, an antisense oligonucleotide, or a ribozyme.

13. The method of claim 1, wherein said composition comprises a second therapeutic or prophylactic agent that inhibits or reduces EphA2 or EphA4 expression or activity, wherein said second therapeutic or prophylactic agent is not attached to or contained within said delivery vehicle.

14. The method of claim 13, wherein said therapeutic or prophylactic agent is an EphA2 or EphA4 agonistic antibody, an antibody that preferentially binds EphA2 or EphA4 epitopes exposed on cancer cells, a cancer cell phenotype inhibiting antibody, an antibody that binds to EphA2 or EphA4 with low K_{off} rate, an EphA2 or EphA4 antisense oligonucleotide, an EphA2 or EphA4 ribozyme, or an EphA2 or EphA4 RNA interference (RNAi) molecule, or an EphA2 or EphA4 aptamer.

15. The method of claim 14, wherein said EphA2 or EphA4 agonistic antibody is Eph099B-208.261, Eph099B-233.152. EA2, EA5 or EA44.

16. The method of claim 15, wherein said EphA2 or EphA4 agonistic antibodies are humanized or chimeric versions of Eph099B-208.261, Eph099B-233.152. EA2, EA5 or EA44.

17. The method of claim 1, wherein said composition comprises an agent that stimulates an immune response against said cells associated with said hyperproliferative cell disease in said subject.

18. The method of claim 1, wherein said administration increases EphA2 or EphA4 phosphorylation in a cancer cell relative to the level of EphA2 or EphA4 phosphorylation in an untreated cancer cell.

19. The method of claim 1, wherein said agent that inhibits or reduces LMW-PTP expression or activity is a nucleic acid molecule comprising a nucleotide sequence encoding an agent that inhibits or reduces LMW-PTP expression or activity.

20. The method of claim 19, wherein said nucleic acid molecule comprises a nucleotide sequence that inhibits or reduces EphA2 expression or activity.

21. The method of claim 1, wherein said subject is an animal.

22. The method of claim 21, wherein said animal is a mammal.

23. The method of claim 21, wherein said animal is a human.

24. A pharmaceutical composition comprising a therapeutically effective amount of:
(a) a delivery vehicle associated with a moiety that binds EphA2 or EphA4 expressed on a cell;
(b) an agent that inhibits or reduces LMW-PTP expression or activity contained within or attached to said delivery vehicle; and
(c) a pharmaceutically acceptable carrier.

25. The pharmaceutical composition of claim 24, wherein said delivery vehicle is a viral vector, a polycation vector, a peptide vector, a liposome, or a hybrid vector.

26. The pharmaceutical composition of claim 24, wherein said moiety that binds EphA2 or EphA4 is an anti-EphA2 or anti-EphA4 antibody or an antigen-binding fragment thereof, an antibody that binds EphA2 or EphA4 epitopes exposed on cancer cells, or Ephrin A1 or fragment thereof that binds EphA2 or EphA4.

27. The pharmaceutical composition of claim 26, wherein said Ephrin A1 or fragment thereof is fused to an Fc domain.

28. The pharmaceutical composition of claim 24, wherein said agent that inhibits or reduces LMW-PTP expression or activity is an anti-LMW-PTP antibody or an antigen-binding fragment thereof, a small phosphatase inhibitor, a RNAi, a antisense oligonucleotide, or a ribozyme.

29. The pharmaceutical composition of claim 24, wherein said composition comprises a second therapeutic or prophylactic agent that inhibits or reduces EphA2 or EphA4 expression or activity, wherein said second therapeutic or prophylactic agent is not attached to or contained within said delivery vehicle.

30. The pharmaceutical composition of claim 29, wherein said therapeutic or prophylactic agent is an EphA2 or EphA4 agonistic antibody, an antibody that preferentially binds EphA2 or EphA4 epitopes exposed on cancer cells, a cancer cell phenotype inhibiting antibody, an antibody that binds to EphA2 or EphA4 with low K_{off} rate, an EphA2 or EphA4 antisense oligonucleotide, an EphA2 or EphA4 ribozyme, or an EphA2 or EphA4 RNA interference (RNAi) molecule, or an EphA2 or EphA4 aptamer.

31. The pharmaceutical coposition of claim 30, wherein said EphA2 or EphA4 agonistic antibody is Eph099B-208.261, Eph099B-233.152. EA2, EA5 or EA44.

32. The pharmaceutical composition of claim 31, wherein said EphA2 or EphA4 agonistic antibodies are humanized or chimeric versions of Eph099B-208.261, Eph099B-233.152. EA2, EA5 or EA44.

33. The pharmaceutical composition of claim 24, wherein said composition comprises an agent that stimulates an immune response against said cells associated with said hyperproliferative cell disease in said subject.

34. The pharmaceutical composition of claim 24, wherein said agent that inhibits or reduces LMW-PTP expression or activity is a nucleic acid molecule comprising a nucleotide sequence encoding an agent that inhibits or reduces LMW-PTP expression or activity.

35. The pharmaceutical composition of claim 34, wherein said nucleic acid molecule comprises a nucleotide sequence that inhibits or reduces EphA2 expression or activity.

36. The method of claim 1, comprising the administration of an additional anti-cancer therapy.

37. The method of claim 36, wherein said additional anti-cancer therapy is not a moiety that binds EphA2 or EphA4.

38. The method of claim 37, wherein said additional anti-cancer therapy is selected from the group consisting of chemotherapy, biological therapy, hormonal therapy, radiation and surgery.

39. The method of making the pharmaceutical composition of claim 24, comprising associating a delivery vehicle with:
(a) a moiety that binds EphA2 or EphA4 expressed on a cell;
(b) an agent that inhibits or reduces LMW-PTP expression or activity contained within or attached to said delivery vehicle; and
(c) a pharmaceutically acceptable carrier.

40. The method of claim 39, wherein said delivery vehicle is a viral vector, a polycation vector, a peptide vector, a liposome, or a hybrid vector.

41. The method of claim 39, wherein said moiety that binds EphA2 or EphA4 is an anti-EphA2 or anti-EphA4 antibody or an antigen-binding fragment thereof, an antibody that binds EphA2 or EphA4 epitopes exposed on cancer cells, or Ephrin A1 or fragment thereof that binds EphA2 or EphA4.

42. The method of claim 41, wherein said Ephrin A1 or fragment thereof is fused to an Fc domain.

43. The method of claim 41, wherein said moiety that binds EphA2 or EphA4 also inhibits or reduces EphA2 or EphA4 expression or activity.

44. The method of claim 41, wherein said anti-EphA2 or anti-EphA4 antibody is Eph099B-208.261, Eph099B-233.152. EA2, EA5 or EA44.

45. The method of claim 44, wherein said anti-EphA2 or anti-EphA4 antibodies are humanized or chimeric versions of Eph099B-208.261, Eph099B-233.152. EA2, EA5 or EA44.


SEQUENCE LISTING (127 sequences)

SEQ ID NO: 1 - protein, 106 aa, Homo Sapiens
QIVLTQSPALMSASPGEKVTMTCSASSSVSYMYWYQQKPRSSPKPWIYLTTNLASGVPARFSGSGSGTSYSLTISSMEAEDAATYYCQQWSSNPFTFGSGTKLEIR

SEQ ID NO: 2 - protein, 10 aa, Homo Sapiens
SASSSVSYMY

SEQ ID NO: 3 - protein, 7 aa, Homo Sapiens
LTTNLAS

SEQ ID NO: 4 - protein, 9 aa, Homo Sapiens
QQWSSNPFT

SEQ ID NO: 5 - protein, 118 aa, Homo Sapiens
QVQLQQPGAELVKPGASVKLSCKASGYTFTSYWMHWVKQRPGQGLEWIGMIHPNSGSTNYNEKFKSKATLTVDKSSSTAYMRLSSLTSEDSAVYYCARGGNMVGGGYWGQGTTLTVSS

SEQ ID NO: 6 - protein, 10 aa, Homo Sapiens
GYTFTSYWMH

SEQ ID NO: 7 - protein, 17 aa, Homo Sapiens
MIHPNSGSTNYNEKFKS

SEQ ID NO: 8 - protein, 10 aa, Homo Sapiens
RGGNMVGGGY

SEQ ID NO: 9 - DNA, 318 bp, Homo Sapiens
caaattgttctcacccagtctccagcactcatgtctgcatctccaggggagaaggtcaccatgacctgcagtgccagctcaagtgtaagttacatgtactggtaccagcagaagccaagatcctcccccaaaccctggatttatctcacaaccaacctggcttctggagtccctgctcgcttcagtggcagtgggtctgggacctcttactctctcacaatcagcagcatggaggctgaagatgctgccacttattactgccagcagtggagtagtaacccattcacgttcggctcggggacaaagttggaaataaga

SEQ ID NO: 10 - DNA, 30 bp, Homo Sapiens
agtgccagctcaagtgtaagttacatgtac

SEQ ID NO: 11 - DNA, 21 bp, Homo Sapiens
ctcacaaccaacctggcttct

SEQ ID NO: 12 - DNA, 27 bp, Homo Sapiens
cagcagtggagtagtaacccattcacg

SEQ ID NO: 13 - DNA, 354 bp, Homo Sapiens
caggtccaactgcagcagcctggggctgagctggtaaagcctggggcttcagtgaagttgtcctgcaaggcttctggctacactttcaccagctactggatgcactgggtgaaacaaaggcctggacaaggccttgagtggattgggatgattcatcctaatagtggtagtactaactacaatgagaagttcaagagcaaggccacactgactgtagacaaatcctccagcacagcctacatgcgactcagcagcctgacatctgaggactctgcggtctattactgtgcaagagggggtaacatggtaggggggggctactggggccaaggcaccactctcacagtctcctca

SEQ ID NO: 14 - DNA, 30 bp, Homo Sapiens
ggctacactttcaccagctactggatgcac

SEQ ID NO: 15 - DNA, 51 bp, Homo Sapiens
atgattcatcctaatagtggtagtactaactacaatgagaagttcaagagc

SEQ ID NO: 16 - DNA, 30 bp, Homo Sapiens
agagggggtaacatggtaggggggggctac

SEQ ID NO: 17 - protein, 107 aa, Homo Sapiens
DIVLTQSPATLSVTPGDSVNLSCRASQSISNNLHWYQQKSHESPRLLIKYVFQSISGIPSRFSGSGSGTDFTLSINSVETEDFGMYFCQQSNSWPLTFGAGTKLELK

SEQ ID NO: 18 - protein, 11 aa, Homo Sapiens
RASQSISNNLH

SEQ ID NO: 19 - protein, 7 aa, Homo Sapiens
YVFQSIS

SEQ ID NO: 20 - protein, 9 aa, Homo Sapiens
QQSNSWPLT

SEQ ID NO: 21 - protein, 120 aa, Homo Sapiens
EVKLVESGGGLVQPGGSLSLSCAASGFTFTDYSMNWVRQPPGKALEWLGFIRNKANDYTTEYSASVKGRFTISRDNSQSILYLQMNALRAEDSATYYCVRYPRYHAMDSWGQGTSVTVSS

SEQ ID NO: 22 - protein, 10 aa, Homo Sapiens
GFTFTDYSMN

SEQ ID NO: 23 - protein, 19 aa, Homo Sapiens
FIRNKANDYTTEYSASVKG

SEQ ID NO: 24 - protein, 9 aa, Homo Sapiens
YPRYHAMDS

SEQ ID NO: 25 - DNA, 321 bp, Homo Sapiens
gatattgtgctaactcagtctccagccaccctgtctgtgactccaggagatagcgtcaatctttcctgcagggccagccaaagtattagcaacaacctacactggtatcaacaaaaatcacatgagtctccaaggcttctcatcaagtatgttttccagtccatctctgggatcccctccaggttcagtggcagtggatcagggacagatttcactctcagtatcaacagtgtggagactgaagattttggaatgtatttctgtcaacagagtaacagctggccgctcacgttcggtgctgggaccaagctggagctgaaa

SEQ ID NO: 26 - DNA, 33 bp, Homo Sapiens
agggccagccaaagtattagcaacaacctacac

SEQ ID NO: 27 - DNA, 21 bp, Homo Sapiens
tatgttttccagtccatctct

SEQ ID NO: 28 - DNA, 27 bp, Homo Sapiens
caacagagtaacagctggccgctcacg

SEQ ID NO: 29 - DNA, 360 bp, Homo Sapiens
gaggtgaagctggtggagtctggaggaggcttggtacagcctgggggttctctgagtctctcctgtgcagcttctggattcaccttcactgattactccatgaactgggtccgccagcctccagggaaggcacttgagtggttgggttttattagaaacaaagctaatgattacacaacagagtacagtgcatctgtgaagggtcggttcaccatctccagagataattcccaaagcatcctctatcttcaaatgaatgccctgagagctgaggacagtgccacttattactgtgtaagataccctaggtatcatgctatggactcctggggtcaaggaacctcagtcaccgtctcctca

SEQ ID NO: 30 - DNA, 30 bp, Homo Sapiens
ggattcaccttcactgattactccatgaac

SEQ ID NO: 31 - DNA, 57 bp, Homo Sapiens
tttattagaaacaaagctaatgattacacaacagagtacagtgcatctgtgaagggt

SEQ ID NO: 32 - DNA, 27 bp, Homo Sapiens
taccctaggtatcatgctatggactcc

SEQ ID NO: 33 - protein, 107 aa, Homo Sapiens
DIKMTQSPSSMYASLGERVTITCKASQDINNYLSWFQQKPGKSPKTLIYRANRLVDGVPSRFSGSGSGQDYSLTISSLEYEDMGIYYCLKYDEFPYTFGGGTKLEIK

SEQ ID NO: 34 - protein, 11 aa, Homo Sapiens
KASQDINNYLS

SEQ ID NO: 35 - protein, 7 aa, Homo Sapiens
RANRLVD

SEQ ID NO: 36 - protein, 9 aa, Homo Sapiens
LKYDEFPYT

SEQ ID NO: 37 - protein, 115 aa, Homo Sapiens
DVKLVESGGGLVKPGGSLKLSCAASGFTFSSYTMSWVRQTPEKRLEWVATISSGGTYTYYPDSVKGRFTISRDNAKNTLYLQMSSLKSEDTAMYYCTREAIFTYWGQGTLVTVSA

SEQ ID NO: 38 - protein, 10 aa, Homo Sapiens
GFTFSSYTMS

SEQ ID NO: 39 - protein, 17 aa, Homo Sapiens
TISSGGTYTYYPDSVKG

SEQ ID NO: 40 - protein, 6 aa, Homo Sapiens
EAIFTY

SEQ ID NO: 41 - DNA, 321 bp, Homo sapiens
gacatcaagatgacccagtctccatcttccatgtatgcatctctaggagagagagtcactatcacttgcaaggcgagtcaggacattaataactatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctgaaatatgatgagtttccgtacacgttcggaggggggaccaagctggaaataaaa

SEQ ID NO: 42 - DNA, 33 bp, Homo sapiens
aaggcgagtcaggacattaataactatttaagc

SEQ ID NO: 43 - DNA, 21 bp, Homo sapiens
cgtgcaaacagattggtagat

SEQ ID NO: 44 - DNA, 27 bp, Homo sapiens
ctgaaatatgatgagtttccgtacacg

SEQ ID NO: 45 - DNA, 345 bp, Homo sapiens
gacgtgaagctggtggagtctgggggaggcttagtgaagcctggagggtccctgaaactctcctgtgcagcctctggattcactttcagtagctataccatgtcttgggttcgccagactccggagaagaggctggagtgggtcgcaaccattagtagtggtggtacttacacctactatccagacagtgtgaagggccgattcaccatctccagagacaatgccaagaacaccctgtacctgcaaatgagcagtctgaagtctgaggacacagccatgtattactgtacaagagaagctatctttacttactggggccaagggactctggtcactgtctctgca

SEQ ID NO: 46 - DNA, 30 bp, Homo sapiens
ggattcactttcagtagctataccatgtct

SEQ ID NO: 47 - DNA, 51 bp, Homo sapiens
accattagtagtggtggtacttacacctactatccagacagtgtgaagggc

SEQ ID NO: 48 - DNA, 18 bp, Homo sapiens
gaagctatctttacttac

SEQ ID NO: 49 - protein, 112 aa, Homo sapiens
DVVMTQTPLTLSVTIGQPASISCKSSQSLLYSNGKTYLNWLLQRPGQSPKRLIYLVSKLDSGVPDRFTGSGSGTDFTLKISRVEAEDLGVYYCVQGSHFPWTFGGGTKLEIK

SEQ ID NO: 50 - protein, 16 aa, Homo sapiens
  VL CDR1 of EA5.12
KSSQSLLYSNGKTYLN

SEQ ID NO: 51 - protein, 7 aa, Homo sapiens
LVSKLDS

SEQ ID NO: 52 - protein, 9 aa, Homo sapiens
VQGSHFPWT

SEQ ID NO: 53 - protein, 115 aa, Homo sapiens
EVQLQQSGPELVKTGASVKISCKASGYSFTGYYMHWVKQSHGKSLEWIGYISCYNGVTSYNQKFKGKATFTVDTSSSTAYMQFNSLTSEDSAVYYCARSHAMDYWGQGTSVTVSS

SEQ ID NO: 54 - protein, 5 aa, Homo sapiens
GYYMH

SEQ ID NO: 55 - protein, 17 aa, Homo sapiens
YISCYNGVTSYNQKFKG

SEQ ID NO: 56 - protein, 6 aa, Homo sapiens
SHAMDY

SEQ ID NO: 57 - DNA, 336 bp, Homo sapiens
gatgtkgtkatgacbcagactccactcactttgtcggttaccattggacaaccagcctctatctcttgcaagtcaagtcagagcctcttatatagtaatggaaaaacctatttgaattggttgttacagaggccaggccagtctccaaagcgcctaatctatctggtgtctaaactggactctggagtccctgacaggttcactggcagtggatcaggaacagattttacactgaaaatcagcagagtggaggctgaggatttgggagtttattactgcgtgcaaggttcacattttccgtggacgttcggtggaggcaccaagctggaaatcaaa

SEQ ID NO: 58 - DNA, 48 bp, Homo sapiens
aagtcaagtcagagcctcttatatagtaatggaaaaacctatttgaat

SEQ ID NO: 59 - DNA, 21 bp, Homo sapiens
ctggtgtctaaactggactct

SEQ ID NO: 60 - DNA, 27 bp, Homo sapiens
gtgcaaggttcacattttccgtggacg

SEQ ID NO: 61 - DNA, 345 bp, Homo sapiens
gaggtccagctgcagcagtctggacctgagctagtgaagactggggcttcagtgaagatatcctgcaaggcttctggttactcattcactggttactacatgcactgggtcaagcagagccatggaaagagccttgagtggattggatatattagttgttacaatggtgttactagctacaaccagaagttcaagggcaaggccacatttactgtagacacatcctccagcacagcctacatgcagttcaacagcctgacatctgaagactctgcggtctattactgtgcaagatctcatgctatggactactggggtcaaggaacctcagtcaccgtctcctca

SEQ ID NO: 62 - DNA, 15 bp, Homo sapiens
ggttactacatgcac

SEQ ID NO: 63 - DNA, 51 bp, Homo sapiens
tatattagttgttacaatggtgttactagctacaaccagaagttcaagggc

SEQ ID NO: 64 - DNA, 18 bp, Homo sapiens
tctcatgctatggactac

SEQ ID NO: 65 - protein, 107 aa, Homo Sapiens
  VL sequence of antibody EA44
EIVLTQSPATLSVSPGERATLSCRASQSVSSNLAWYQQKPGQAPRLLIYGASTRATGIPDRFSASGSGTDFTLTISRVEPEDFAVYYCQQYGSSWTFGQGTKVEIKR

SEQ ID NO: 66 - protein, 11 aa, Homo sapiens
  CDR1 of VL region of EA44
RASQSVSSNLA

SEQ ID NO: 67 - protein, 7 aa, Homo sapiens
  CDR2 of VL region of EA44
GASTRAT

SEQ ID NO: 68 - protein, 8 aa, Homo sapiens
  CDR3 of VL region of EA44
QQYGSSWT

SEQ ID NO: 69 - protein, 123 aa, Homo Sapiens
  VH sequence of antibody EA44
MAQVQLLQSGAEVKKPGASVKVPCKASGYTFTSYAMSWVRQAPGQGLEWMGWINTNTGNPTYAQGFTGRFVFSLDTSVSTAYLQISSLKAEDTAVYYCARVRTTVYGDGMDVWGQGTLVTVSS

SEQ ID NO: 70 - protein, 5 aa, Homo sapiens
  CDR1 of VH region of EA44
SYAMS

SEQ ID NO: 71 - protein, 17 aa, Homo sapiens
  CDR2 of VH region of EA44
WINTNTGNPTYAQGFTG

SEQ ID NO: 72 - protein, 12 aa, Homo sapiens
  CDR3 of VH region of EA44
VRTTVYGDGMDV

SEQ ID NO: 73 - DNA, 321 bp, Homo Sapiens
  VL sequence of antibody EA44
gaaattgtgctgactcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagacaggttcagcgccagtgggtctgggacggatttcactctcaccatcagcagagtggaacctgaagattttgcagtttattactgtcagcaatatggtagttcatggacattcggccaagggaccaaggtggaaatcaaacgt

SEQ ID NO: 74 - DNA, 33 bp, Homo sapiens
  CDR1 of VL region of EA44
agggccagtcagagtgttagcagcaacttagcc

SEQ ID NO: 75 - DNA, 21 bp, Homo sapiens
  CDR2 of VL region of EA44
ggtgcatccaccagggccact

SEQ ID NO: 76 - DNA, 24 bp, Homo sapiens
  CDR3 of VL region of EA44
cagcaatatggtagttcatggaca

SEQ ID NO: 77 - DNA, 369 bp, Homo Sapiens
  VH sequence of antibody EA44
atggcacaggtgcagctgttgcagtctggagctgaggtgaagaagcctggggcctcagtgaaggttccctgcaaggcttctggatacaccttcactagctatgctatgagttgggtgcgacaggcccctggacaagggcttgagtggatgggatggatcaacaccaacactgggaacccaacgtatgcccagggcttcacaggacggtttgtcttctccttggacacctctgtcagcacggcatatctgcagatcagcagcctaaaggctgaggacactgccgtgtattactgtgcgagagtccggactacggtgtatggggacggtatggacgtctggggccaaggcaccctggtcaccgtctcctca

SEQ ID NO: 78 - DNA, 15 bp, Homo sapiens
  CDR1 of VH region of EA44
agctatgctatgagt

SEQ ID NO: 79 - DNA, 51 bp, Homo sapiens
  CDR2 of VH region of EA44
tggatcaacaccaacactgggaacccaacgtatgcccagggcttcacagga

SEQ ID NO: 80 - DNA, 36 bp, Homo sapiens
  CDR3 of VH region of EA44
gtccggactacggtgtatggggacggtatggacgtc

SEQ ID NO: 81 - protein, 15 aa, Homo sapiens
GGGGSGGGGSGGGGS

SEQ ID NO: 82 - protein, 15 aa, Homo sapiens
ESGRSGGGGSGGGGS

SEQ ID NO: 83 - protein, 14 aa, Homo sapiens
EGKSSGSGSESKST

SEQ ID NO: 84 - protein, 15 aa, Homo sapiens
EGKSSGSGSESKSTQ

SEQ ID NO: 85 - protein, 14 aa, Homo sapiens
EGKSSGSGSESKVD

SEQ ID NO: 86 - protein, 14 aa, Homo sapiens
GSTSGSGKSSEGKG

SEQ ID NO: 87 - protein, 18 aa, Homo sapiens
KESGSVSSEQLAQFRSLD

SEQ ID NO: 88 - protein, 16 aa, Homo sapiens
ESGSVSSEELAFRSLD

SEQ ID NO: 89 - protein, 4 aa, Homo sapiens
KDEL

SEQ ID NO: 90 - protein, 4 aa, Homo sapiens
DDEL

SEQ ID NO: 91 - protein, 4 aa, Homo sapiens
DEEL

SEQ ID NO: 92 - protein, 4 aa, Homo sapiens
QEDL

SEQ ID NO: 93 - protein, 4 aa, Homo sapiens
RDEL

SEQ ID NO: 94 - protein, 7 aa, Homo sapiens
PKKKRKV

SEQ ID NO: 95 - protein, 7 aa, Homo sapiens
PQKKIKS

SEQ ID NO: 96 - protein, 5 aa, Homo sapiens
QPKKP

SEQ ID NO: 97 - protein, 4 aa, Homo sapiens
RKKR

SEQ ID NO: 98 - protein, 5 aa, Homo sapiens
KKKRK

SEQ ID NO: 99 - protein, 12 aa, Homo sapiens
RKKRRQRRRAHQ

SEQ ID NO: 100 - protein, 16 aa, Homo sapiens
RQARRNRRRRWRERQR

SEQ ID NO: 101 - protein, 19 aa, Homo sapiens
MPLTRRRPAASQALAPPTP

SEQ ID NO: 102 - protein, 15 aa, Homo sapiens
MDDQRDLISNNEQLP

SEQ ID NO: 103 - protein, 32 aa, Homo sapiens
  misc_feature (7)..(8): Xaa can be any naturally occurring amino acid
MLFNLRXXLNNAAFRHGHNFMVRNFRCGQPLX

SEQ ID NO: 104 - protein, 3 aa, Homo sapiens
AKL

SEQ ID NO: 105 - protein, 6 aa, Homo sapiens
SDYQRL

SEQ ID NO: 106 - protein, 8 aa, Homo sapiens
GCVCSSNP

SEQ ID NO: 107 - protein, 8 aa, Homo sapiens
GQTVTTPL

SEQ ID NO: 108 - protein, 8 aa, Homo sapiens
GQELSQHE

SEQ ID NO: 109 - protein, 8 aa, Homo sapiens
GNSPSYNP

SEQ ID NO: 110 - protein, 8 aa, Homo sapiens
GVSGSKGQ

SEQ ID NO: 111 - protein, 8 aa, Homo sapiens
GQTITTPL

SEQ ID NO: 112 - protein, 8 aa, Homo sapiens
GQTLTTPL

SEQ ID NO: 113 - protein, 8 aa, Homo sapiens
GQIFSRSA

SEQ ID NO: 114 - protein, 8 aa, Homo sapiens
GQIHGLSP

SEQ ID NO: 115 - protein, 8 aa, Homo sapiens
GARASVLS

SEQ ID NO: 116 - protein, 8 aa, Homo sapiens
GCTLSAEE

SEQ ID NO: 117 - protein, 16 aa, Homo sapiens
AAVALLPAVLLALLAP

SEQ ID NO: 118 - protein, 12 aa, Homo sapiens
AAVLLPVLLAAP

SEQ ID NO: 119 - protein, 15 aa, Homo sapiens
VTVLALGALAGVGVG

SEQ ID NO: 120 - DNA, 31 bp, Homo sapiens
ccagcagtaccgcttccttgccctgcggccg

SEQ ID NO: 121 - DNA, 30 bp, Homo sapiens
ccagcagtaccacttccttgccctgcgccg

SEQ ID NO: 122 - DNA, 30 bp, Homo sapiens
gccgcgtcccgttccttcaccatgacgacc

SEQ ID NO: 123 - DNA, 31 bp, Homo sapiens; identical to SEQ ID NO: 120

SEQ ID NO: 124 - DNA, 30 bp, Homo sapiens; identical to SEQ ID NO: 122

SEQ ID NO: 125 - DNA, 35 bp, Homo sapiens
aatttaaagcttccatggcggaacaggctaccaag

SEQ ID NO: 126 - DNA, 34 bp, Homo sapiens
cgttcttggagaaggcccactgagaattcttcgt

SEQ ID NO: 127 - DNA, 28 bp, Homo sapiens
gcgcgcggatcctcagtgggccttctcc